SEQUENCE LISTING (7 sequences)

SEQ ID NO: 1 - DNA, 34 bp, synthetic construct
  misc_feature 1..34: note = Linker sequence
  source 1..34: mol_type = other DNA
ataacttcgtatagcatacattatacgaagttat

SEQ ID NO: 2 - DNA, 31 bp, synthetic construct
  misc_feature 1..31: note = US10-39SpeI Primer
  source 1..31: mol_type = other DNA
gctcactagtggcctagcctggctcatggcc

SEQ ID NO: 3 - DNA, 33 bp, synthetic construct
  misc_feature 1..33: note = US10-59PacI Primer
  source 1..33: mol_type = other DNA
gtccttaattaagacgtggttgtggtcaccgaa

SEQ ID NO: 4 - DNA, 33 bp, synthetic construct
  misc_feature 1..33: note = US9-39PmeI Primer
  source 1..33: mol_type = other DNA
ctcggtttaaacgacgtgaggcgctccgtcacc

SEQ ID NO: 5 - DNA, 31 bp, synthetic construct
  misc_feature 1..31: note = US-59ClaI Primer
  source 1..31: mol_type = other DNA
ttgcatcgatacggtgtgagataccacgatg

SEQ ID NO: 6 - DNA, 20 bp, synthetic construct
  misc_feature 1..20: note = US9[198789] Primer
  source 1..20: mol_type = other DNA
tgacgcgagtattacgtgtc

SEQ ID NO: 7 - DNA, 19 bp, synthetic construct
  misc_feature 1..19: note = US10[199100] Primer
  source 1..19: mol_type = other DNA
ctcctcctgatatgcggtt

(19) United States
(12) Patent Application Publication
(10) Pub. No.: US 2025/0205242 A1
(43) Pub. Date: Jun. 26, 2025

(54) CDK7 INHIBITORS FOR ANTIVIRAL TREATMENT

(71) Applicants: QURIENT CO., LTD., GYEONGGI-DO (KR); LEAD DISCOVERY CENTER GMBH, DORTMUND (DE)

(72) Inventors: Kiyean NAM, GYEONGGI-DO (KR); Jaeseung KIM, SEOUL (KR); Yeejin JEON, GYEONGGI-DO (KR); Donghoon YU, GYEONGGI-DO (KR); Seung-Joo LEE, SEONGNAM-SI (KR); Jan EICKHOFF, HERDECKE (DE); Gunther ZISCHINSKY, DORTMUND (DE); Uwe KOCH, DORTMUND (DE); Bert KLEBL, DORTMUND (DE)

(21) Appl. No.: 18/848,272

(22) PCT Filed: Mar. 17, 2023
(86) PCT No.: PCT/EP2023/056950

§ 371 Date: Sep. 18, 2024

Related U.S. Application Data
(60) Provisional application No. 63/321,163, filed on Mar. 18, 2022.

(51) Int. Cl. A61K 31/53; A61K 31/55; A61P 31/20

(52) U.S. Cl. CPC A61K 31/53; A61K 31/55; A61P 31/20

(57) ABSTRACT

The present invention relates to inhibitors of cyclin-dependent kinase 7 (CDK7) and their uses in the treatment of viral infections, in particular infections by DNA-viruses, such as Herpesviridae or Papillomaviridae. The present invention also relates to methods of treatment of viral infections using such inhibitors of cyclin-dependent kinase 7.

The present invention relates to inhibitors of cyclin-dependent kinase 7 (CDK7) and their uses in the treatment of viral infections, in particular infections by DNA-viruses, such as Herpesviridae or Papillomaviridae. The present invention also relates to methods of treatment of viral infections using such inhibitors of cyclin-dependent kinase 7.

### BACKGROUND OF THE INVENTION

Antiviral therapy is commonly used in intensive immunosuppressive settings, for example, for the management of rejections in recipients of solid organ transplants (SOT), or in the management of graft-versus-host disease (GVHD) in recipients of hematopoietic stem cell transplants (HSCT). Unfortunately, in many such scenarios, drug resistant viruses are increasingly encountered. Prolonged antiviral drug exposure and sustained viral replication due to immunosuppression are key factors in the development of antiviral drug resistance which may manifest itself as persistent or increasing viremia or disease in spite of therapy. Most, if not all of the currently licensed drugs for systemic therapy of, for example, Herpes virus infections share the same target, namely viral DNA polymerase. Because they act on viral components, in this case, viral DNA polymerase, these drugs are also named “direct-acting antiviral agents (DAAs)”. However, because such DAAs act directly on viral proteins, in many instances, they have a low genetic barrier to drug resistance, and the resulting selective pressure facilitates viral mutations during virus replication which, in turn, makes the virus refractory to treatment by DAAs. Moreover, since viral proteins generally do not share a structural similarity among different species, any particular antiviral agent targeting a specific viral protein is not necessarily able to confer the same inhibitory effects on other viruses. Therefore, there is still a great unmet need for the treatment of viral infections and new antiviral agents having broad-spectrum antiviral activities.

Because, in many instances, antivirals are administered systemically, the likelihood of unwanted side effects is relatively high. Therefore, there is furthermore a need for antiviral agents that can be used at low concentrations so as to minimize unwanted side effects, whilst, at the same time achieving a high antiviral activity.

Herpesviridae are a large family of DNA-containing viruses which are responsible for various diseases in mammals, including humans. Infections by these viruses manifest themselves inter alia in cutaneous lesions, blisters and/or skin flares. At least five Herpes virus types are known to infect human beings, and include Herpes simplex viruses 1 and 2 (HSV-1 and HSV-2) causing orolabial Herpes and genital Herpes, Varicella zoster virus causing chickenpox and shingles, Epstein-Barr virus (EBV) causing mononucleosis, and human cytomegalovirus (HCMV) causing complications in particular in immune suppressed patients. Infections by some of these viruses are treated using nucleoside analogues or nucleobase analogues, but only to a limited effect. In addition, in the last decade the emergence of Ganciclovir (GCV)-resistant HCMV strains during treatment has become a substantial problem in patients with AIDS, in transplant recipients and in other immunosuppressed patients. Mutations in the UL97-encoded phosphotransferase (pUL97) have been shown by marker transfer to play a key role in altering HCMV drug susceptibility via inhibition of intracellular GCV monophosphorylation. In the intervening decades, it has been established that the most common UL97 mutations conferring clinical ganciclovir resistance are clustered at codons 460, 520 and 590-607, with a relatively small number accounting for >80% of genotypically diagnosed cases of ganciclovir resistance. These canonical pUL97 resistance mutations were also able to disrupt susceptibility of Maribavir (MBV) which is an inhibitor of pUL97 kinase activity through specific mutations at pUL97 codons upstream of 460.

Papillomaviridae, including Human papillomaviruses (HPVs) are a large and diverse group of epitheliotropic double-stranded DNA viruses that predominantly infect epithelial tissues of external skin and mucosal surfaces. Up to 225 different types of HPV have been listed so far. Based on epidemiological data, about 15 alphapapillomaviruses (alpha HPVs) has been referred to as high-risk (HR) HPV types, including HPV-16, -18, or -31, which can cause, or are associated with, invasive cancers of the cervix and other mucosal anongenital tract sites and head and neck cancers. If not cleared, HR-HPV infections can persist for years or even decades, and these persistent HR-HPV infections are a major risk factor for subsequent cancer development.

Although the mechanism of cancer progression by HR-HPV infection is still unclear, HPV type and viral load may be involved, and HPV infection may cause abnormal growth and transformation of infected cells potentially leading to cancerous tumors. Infection with other genotypes, called low-risk (LR) including HPV-6 or -11, can cause benign or low-grade cervical tissue changes and genital warts, condyloma acuminata, which grow on the cervix, vagina, vulva and anus in women and the penis, scrotum or anus in men. Although not life-threatening, these LR-HPVs can be passed from mother to child during birth and cause a persistent tracheal infection, where condyloma acuminata growth can block the airway. Because HPV infections are not systemic and often localized to easily accessible regions of the skin and mucosa, various cytopathic options are available. However, if the cells within the infection are not extensively removed, recurrence rates can be substantial, often requiring repeated and costly treatments.

There is therefore also a need to provide for alternative and, possibly, more efficient treatment modalities and compounds that are capable of treating infections by the aforementioned viruses in a more efficient manner.

In a first aspect, the present invention relates to a compound having the general formula I

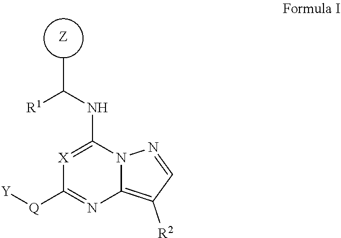

or an enantiomer, stereoisomeric form, mixture of enantiomers, diastereomer, mixture of diastereomers, racemate or a pharmaceutically acceptable salt thereof, for use in a method of treating a DNA-virus infection in a subject, said DNA-virus preferably being selected from Herpesviridae and Papillomaviridae;
wherein in said compound
X is, independently at each occurrence, selected from CH and N;
Q is either absent or independently, at each occurrence, selected from the group consisting of —NH—, —NH(CH₂)—, —NH(CH₂)₂—, —NH(C═O)—, —NHSO₂—, —O—, —O(CH₂)—, —(C═O)—, —(C═O)NH— and —(C═O)(CH₂)—;
Y is, independently at each occurrence, selected from the group consisting of halogen, C1-C3 haloalkyl, C3-C8 cycloalkyl, aryl, heteroaryl, heterocyclyl, —S(═O)₂R³, C1-C6 alkyl and C1-C6 alkyl substituted with one or two of —OR⁵, —N(R⁵)R⁵, aryl, heteroaryl and heterocyclyl;

Wherein C3-C8 cycloalkyl is optionally substituted with one or two of R³, R⁴ and —(C═O)R⁵, wherein heterocyclyl is optionally substituted with one or two of R³, R⁴ and —(C═O)R⁵, and wherein aryl or heteroaryl is optionally substituted with one or two of R³, C1-C6 alkyl, —OR⁵, —N(R⁵)R⁵, —(C═O)R⁵, halogen, heteroaryl and heterocyclyl;
R¹ is, at each occurrence, independently selected from the group consisting of hydrogen and methyl;
R² is, at each occurrence, independently selected from the group consisting of halogen, C1-C6 alkyl, C3-C10 cycloalkyl, —CN, —(C═O)CH₃ and C1-C3 haloalkyl, any of which is optionally substituted;
R³ is either absent or independently, at each occurrence, selected from the group consisting of hydrogen, —OR⁵, halogen, C1-C3 haloalkyl, —CN, —N(R⁵)R⁵, (═O), —NH(C═O)R⁵, —(C═O)NH₂, —S(═O)₂N(R⁵)R⁵, aryl, heteroaryl, heterocyclyl, C1-C6 alkyl and C1-C6 alkyl substituted with —OR₅, —NH₂ or —S(═O)₂N(R⁵)R⁵;
R⁴ is, independently, at each occurrence, selected from the group consisting of hydrogen, halogen, C1-C3 haloalkyl, —CN, —OR⁵, —N(R⁵)R⁵, (═O), S(═O)₂N(R⁵)R⁵, aryl, heteroaryl, heterocyclyl, C1-C6 alkyl and C1-C6 alkyl substituted with —OH, —NH₂ or —S(═O)₂N(R⁵)R⁵;
wherein both R³ and R⁴ are (═O) if attached to a single sulfur atom that forms part of Y being a heterocycle;
or wherein R³ and R⁴, together with the structure to which they are attached, form an aromatic ring, a heteroaromatic ring, a saturated or unsaturated heterocyclic ring, or a fused or bridged ring structure of any of an aromatic ring, a heteroaromatic ring, and a saturated or unsaturated heterocyclic ring;
R⁵ is, at each occurrence, independently selected from the group consisting of hydrogen, C1-C6 alkyl, C3-C6 cycloalkyl, C1-C3 haloalkyl, heteroaryl, heterocyclyl, heteroaryl substituted with one or two of halogen, —OR¹¹, —N(R¹¹)R¹¹, C1-C6 alkyl and C1-C6 alkyl substituted with —OH, —NH₂; heterocyclyl substituted with one or two of halogen, —OR¹¹, —N(R¹¹)R¹¹, C1-C6 alkyl and C1-C6 alkyl substituted with —OH or —NH₂;
Z is any structure of the following group A;

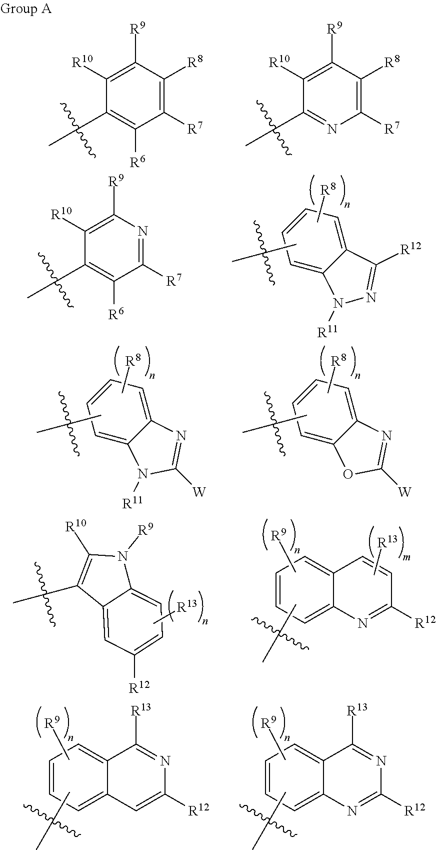

Wherein n=1, 2, or 3; m=1, or 2;
R⁶ and R⁷ are, at each occurrence, independently selected from the group consisting of hydrogen, —NH(C═O)R¹⁴, —NHR¹⁴, —OR¹⁴ and any structure of the following group B, with the
proviso that, when Z is

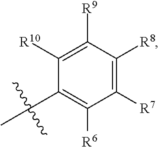

one of R⁶ and R⁷ is not H;

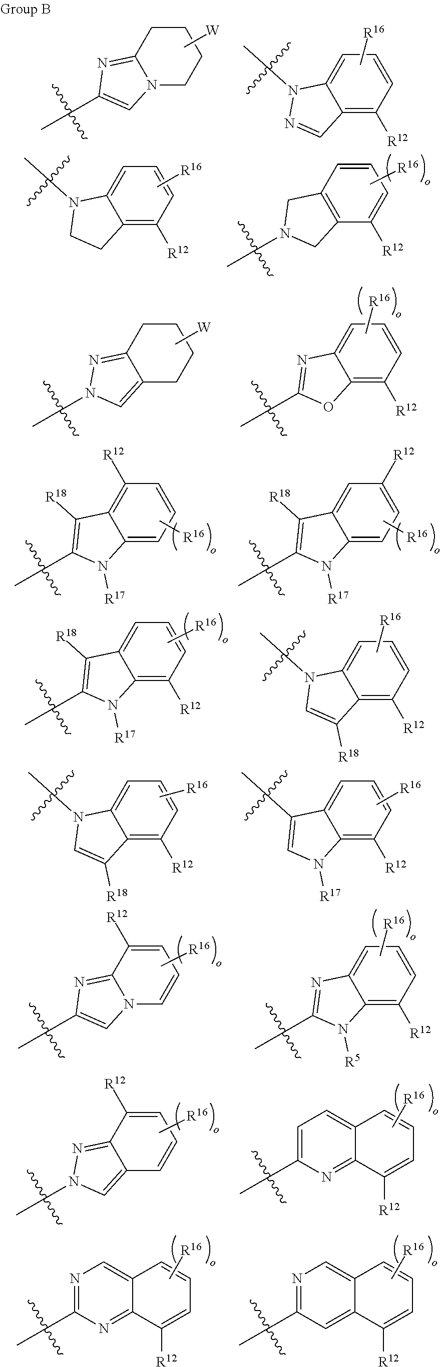

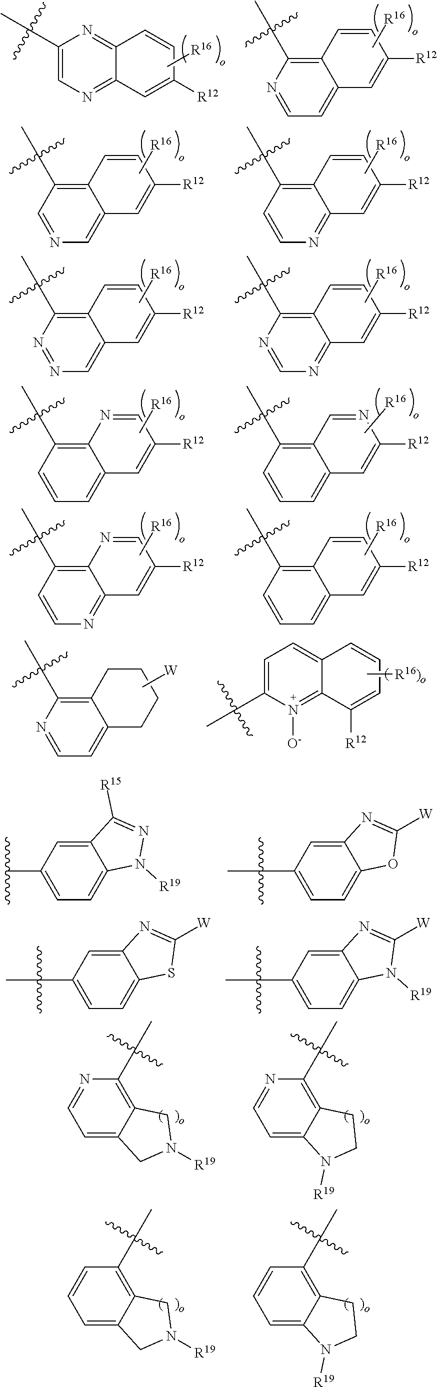

wherein o is, independently at each occurrence, selected from 1, 2 and 3;
W is any structure of the following group C;

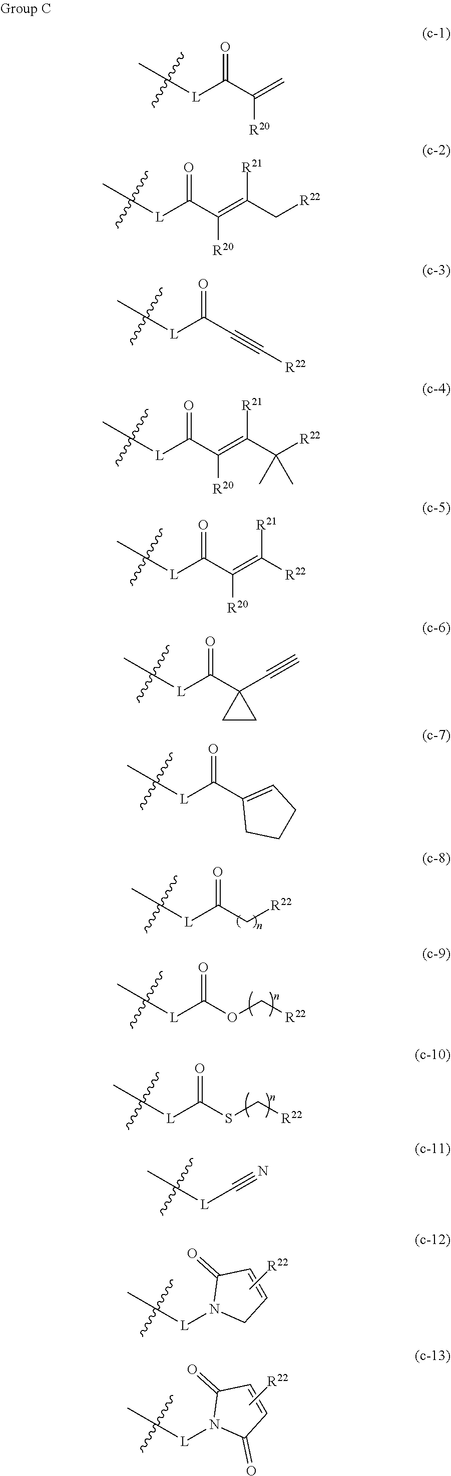

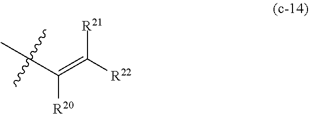

L is absent or, at each occurrence, independently selected from the group consisting of —O— and —NH—;
wherein n is, independently at each occurrence, selected from 1, 2 and 3;
R⁸, R⁹ and R¹⁰ are, at each occurrence, independently selected from the group consisting of hydrogen, halogen, C1-C6 alkyl, C1-C3 haloalkyl, —OR⁵, —CN and C1-C6 alkyl substituted with —OH, —OR⁵ or —NHR⁵;
R¹¹ is, at each occurrence, independently selected from the group consisting of hydrogen, C1-C6 alkyl, C3-C10 cycloalkyl and W, as defined above;
R¹² is, at each occurrence, independently selected from hydrogen and W, as defined above;
Wherein if R¹¹ is W, R¹² is hydrogen;
R¹³ is, at each occurrence, independently selected from the group consisting of hydrogen, halogen, C1-C6 alkyl, C1-C3 haloalkyl, —NH₂, —OR⁵, —CN and W, as defined above;
Wherein if R¹³ is W, R¹² is hydrogen;
R¹⁴ is any structure of group D;

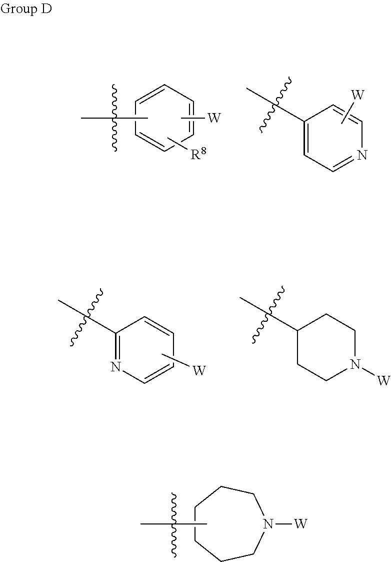

R¹⁵ is, at each occurrence, independently selected from hydrogen and W, as defined above;
R¹⁶ is, at each occurrence, independently selected from the group consisting of hydrogen, halogen, C1-C6 alkyl, C1-C3 haloalkyl, —NH₂, —OR⁵, —CN and W, as defined above;
Wherein if R¹⁶ is W, R¹² is hydrogen;
R¹⁷ is, at each occurrence, independently selected from the group consisting of hydrogen, C1-C6 alkyl and C1-C3 haloalkyl;
R¹⁸ is, at each occurrence, independently selected from the group consisting of hydrogen, halogen, C1-C6 alkyl, C1-C3 haloalkyl, —NH₂, —OR⁵ and —CN;
R¹⁹ is, at each occurrence, independently selected from the group consisting of hydrogen, C1-C6 alkyl, C3-C10 cycloalkyl and W, as defined above;
Wherein if R¹⁹ is W, R¹⁵ is hydrogen;
R²⁰ and R²¹ are, at each occurrence, independently selected from the group consisting of hydrogen, halogen, C1-C6 alkyl, C1-C3 haloalkyl, —OR⁵, heterocyclyl and —CN;
R²² is, at each occurrence, independently selected from the group consisting of hydrogen, halogen, C1-C6 alkyl, C3-C10 cycloalkyl, —N(R⁵)₂, —NR¹⁹R²⁰, —NR¹⁹CH₂(CO)NH₂, heterocyclyl, —OR⁵ and —CN.

In one embodiment, said DNA-virus infection is a Herpesviridae infection, and said Herpesviridae infection is an infection by a member from a Herpesviridae subfamily, such subfamily being selected from Alphaherpesvirinae, Betaherpesvirinae, and Gammaherpesvirinae, wherein preferably said member is selected from human Herpes-simplex-virus-1 (HSV-1), human Herpes-simplex-virus-2 (HSV-2), Varicella zoster virus, human cytomegalovirus (HCMV), and Epstein-Barr-Virus (EBV).

In one embodiment, said DNA-virus infection is a Herpesviridae infection is an infection, and said Herpesviridae infection by human cytomegalovirus (HCMV).

In one embodiment, said DNA-virus infection is a Herpesviridae infection is an infection, and said Herpesviridae infection by human Herpes-simplex-virus-1 (HSV-1).

In one embodiment, said DNA-virus infection is a Herpesviridae infection, and said Herpesviridae infection is an infection by Epstein-Barr-Virus (EBV).

In one embodiment, said DNA-virus infection is a Herpesvirirdae infection by a virus that is resistant against nucleobase analogues or nuceloside analogues or inhibitors of viral DNA synthesis.

In one embodiment, said DNA-virus infection is a human cytomegalovirus (HCMV) infection by an HCM virus (HCMV), or is a human Herpes-simplex-virus-1 (HSV-1) infection or a human Herpes-simplex-virus-2 (HSV-2) infection, or is a Epstein-Barr-Virus (EBV), wherein said HCMV, said HSV-1, said HSV-2 and said EBV is resistant against a nucleobase analogue, in particular a guanine analogue, preferably against ganciclovir, aciclovir and/or penciclovir or against an inhibitor of viral DNA synthesis, in particular an inhibitor of pUL97 kinase activity, more particularly maribavir; or wherein said HCMV, said HSV-1 and said HSV-2 is resistant against a nucleoside analogue, in particular selected from analogues of deoxyadenosine, adenosine, deoxycytidine, guanosine, deoxyguanosine, thymdine, deoxythymidine, and/or deoxyuridine, wherein, preferably, said nucleoside analogue is selected from didanosine, vidarabine, galidesivir, remdesivir, cytarabine, gemcitabine, emtricitabine, lamivudine, zalcitabine, abacavir, entecavir, stavudine, telbivudine, zidovudine, idoxuridine, and trifluridine.

In one embodiment, said DNA-virus infection is a Papillomaviridae infection, and said Papillomaviridae infection is an infection by a human papillomavirus (HPV), selected from alphapapillomavirus, betapapillomavirus andgammapapillomavirus, wherein, preferably, said human papillomavirus is selected from alphapapillomavirus, in particular types HPV-6, HPV-16, HPV-2, HPV-7, HPV-10, HPV-18, HPV-26, HPV-32, HPV-34, HPV-53, HPV-61, HPV-71, HPV-cand90, and the respective subtypes of the foregoing; wherein, more preferably, said human papillomavirus is selected from types HPV-6 and HPV-16, and corresponding subtypes of HPV-6, namely subtypes HPV-6, HPV-11, HPV-13, HPV-44 and HPV-74; and corresponding subtypes of HPV-16, namely HPV-16, HPV-31, HPV-33, HPV-35, HPV-52, HPV58, and HPV-67.

In one embodiment, said method is for the treatment and/or prevention of a cancer caused by or associated with HPV, said cancer being selected from cervical cancer, oropharyngeal cancer, anal cancer, penile cancer, vaginal cancer and vulvar cancer.

In one embodiment, said method is performed on a subject who is a non-responder, or fails to respond adequately, to HPV-vaccination, or said method is performed on a subject who cannot be vaccinated against HPV In one embodiment, said method comprises administering a compound having the general formula I

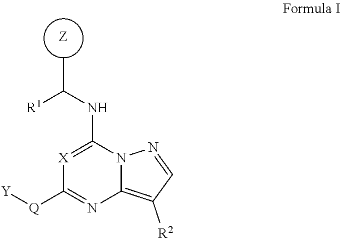

As defined herein, to a subject having, or suspected of having, a Herpesviridae infection or a Papillomaviridae infection.

In one embodiment, said compound is administered at an early stage of infection in said subject and/or prior to onset of any symptoms in said subject.

In one embodiment, said subject is a non-responder to a previous course of treatment with a nucleobase analogue or a nucleoside analogue or an inhibitor of viral DNA synthesis, in particular an inhibitor of pUL97 kinase activity.

In one embodiment, said compound is administered systemically or topically.

In one embodiment, said compound is a compound
having the general formula Ia

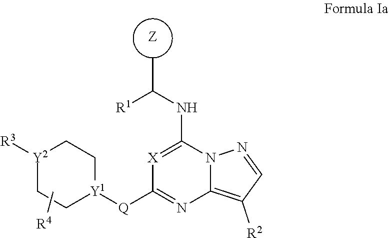

wherein
X is, independently at each occurrence, selected from CH and N;
Y¹ is, independently at each occurrence, selected from CH, C(OH) and N;
Y² is, independently at each occurrence, selected from CH, C(OH) and N;
Q is absent or, at each occurrence, independently selected from the group consisting of —NH—, —NH(CH₂)—, —NH(C═O)—, —NHSO₂—, —O—, —O(CH₂)—, —(C═O)— and —(C═O)(CH₂)—;
R¹ is, at each occurrence, independently selected from the group consisting of hydrogen and methyl;
R² is, at each occurrence, independently selected from the group consisting of halogen, C1-C6 alkyl, C3-C10 cycloalkyl, —CN, —(C═O)CH₃ and C1-C3 haloalkyl, any of which is optionally substituted;
R³ is either absent or independently, at each occurrence, selected from the group consisting of hydrogen, —OR⁵, halogen, C1-C3 haloalkyl, —CN, —N(R⁵)R⁵, (═O), —NH(C═O)R⁵, —(C═O)NH₂, —S(═O)₂N(R⁵)R⁵, aryl, heteroaryl, heterocyclyl, C1-C6 alkyl and C1-C6 alkyl substituted with —OR₅, —NH₂ or —S(═O)₂N(R⁵)R⁵;
R⁴ is, independently, at each occurrence, selected from the group consisting of hydrogen, halogen, C1-C3 haloalkyl, —CN, —OR⁵, —N(R⁵)R⁵, (═O), S(═O)₂N(R⁵)R⁵, aryl, heteroaryl, heterocyclyl, C1-C6 alkyl and C1-C6 alkyl substituted with —OH, —NH₂ or —S(═O)₂N(R⁵)R⁵;
wherein both R³ and R⁴ are (═O) if attached to a single sulfur atom that forms part of Y being a heterocycle;
or wherein R³ and R⁴, together with the structure to which they are attached, form an aromatic ring, a heteroaromatic ring, a saturated or unsaturated heterocyclic ring, or a fused or bridged ring structure of any of an aromatic ring, a heteroaromatic ring, and a saturated or unsaturated heterocyclic ring;
R⁵ is, at each occurrence, independently selected from the group consisting of hydrogen, C1-C6 alkyl, C3-C6 cycloalkyl, C1-C3 haloalkyl, heteroaryl, heterocyclyl, heteroaryl substituted with one or two of halogen, —OR¹¹, —N(R¹¹)R¹¹, C1-C6 alkyl and C1-C6 alkyl substituted with —OH, —NH₂; heterocyclyl substituted with one or two of halogen, —OR¹¹, —N(R¹¹)R¹¹, C1-C6 alkyl and C1-C6 alkyl substituted with —OH or —NH₂;
Z is any structure of the following group A;

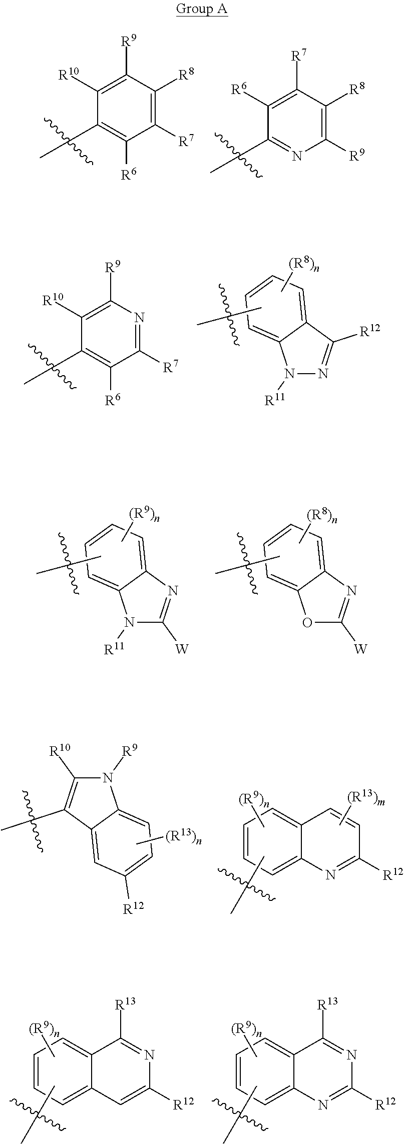

wherein n=1, 2, or 3; m=1, or 2;
R⁶ and R⁷ are, at each occurrence, independently selected from the group consisting of hydrogen, —NH(C═O)R¹⁴, —NHR¹⁴, —OR¹⁴ and any structure of the following group B, with the
proviso that, when Z is

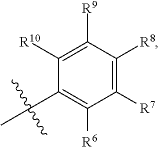

one of R6 and R7 is not H;

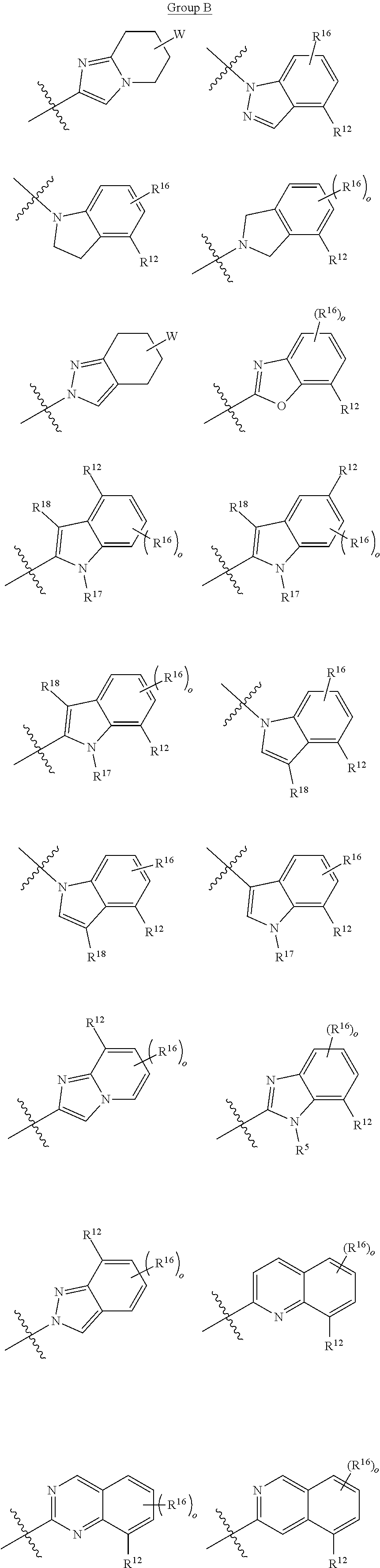

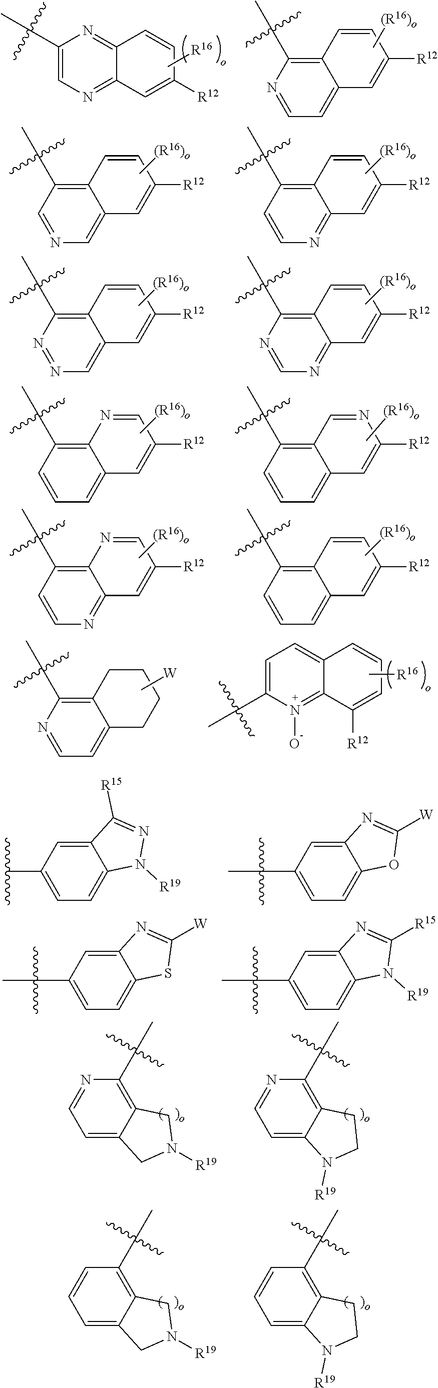

wherein o=1, 2 or 3;
W is any structure of the following group C;

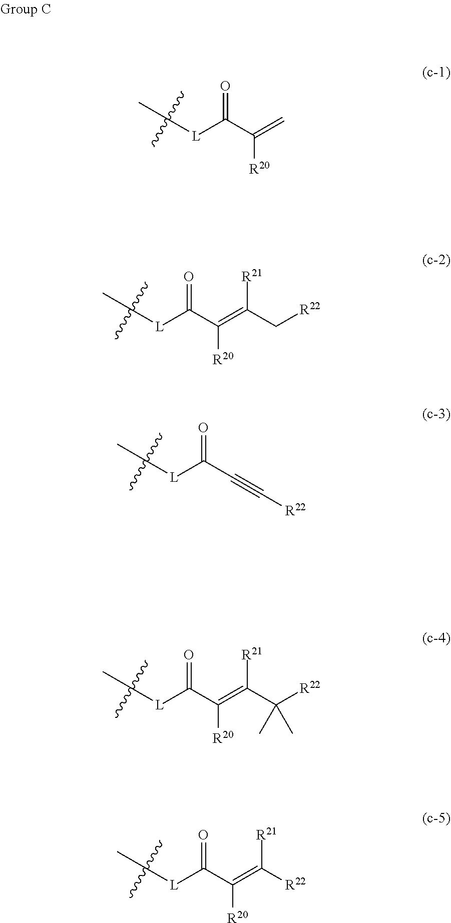

L is absent or, at each occurrence, independently selected from the group consisting of —O— and —NH—;
R⁸, R⁹ and R¹⁰ are, at each occurrence, independently selected from the group consisting of hydrogen, halogen, C1-C6 alkyl, C1-C3 haloalkyl, —OR⁵, —CN and C1-C6 alkyl substituted with —OH, —OR⁵ or —NHR⁵;
R¹¹ is, at each occurrence, independently selected from the group consisting of hydrogen, C1-C6 alkyl, C3-C10 cycloalkyl and W, as defined above;
R¹² is, at each occurrence, independently selected from hydrogen and W, as defined above;
wherein if R¹¹ is W, R¹² is hydrogen;
R¹³ is, at each occurrence, independently selected from the group consisting of hydrogen, halogen, C1-C6 alkyl, C1-C3 haloalkyl, —NH₂, —OR⁵, —CN and W, as defined above;
wherein if R¹³ is W, R¹² is hydrogen;
R¹⁴ is any structure of group D;

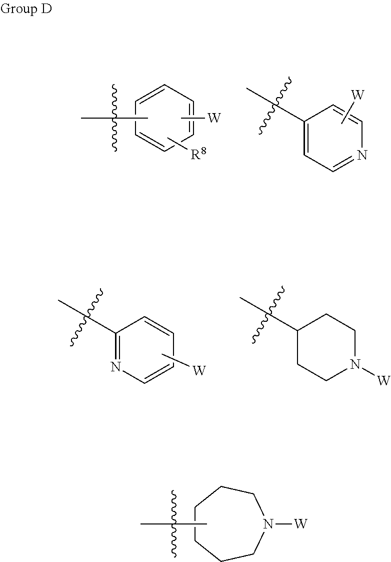

R¹⁵ is, at each occurrence, independently selected from hydrogen and W, as defined above;
R¹⁶ is, at each occurrence, independently selected from the group consisting of hydrogen, halogen, C1-C6 alkyl, C1-C3 haloalkyl, —NH₂, —OR⁵, —CN and W, as defined above;
wherein if R¹⁶ is W, R¹² is hydrogen;
R¹⁷ is, at each occurrence, independently selected from the group consisting of hydrogen, C1-C6 alkyl and C1-C3 haloalkyl;
R¹⁸ is, at each occurrence, independently selected from the group consisting of hydrogen, halogen, C1-C6 alkyl, C1-C3 haloalkyl, —NH₂, —OR⁵ and —CN;
R¹⁹ is, at each occurrence, independently selected from the group consisting of hydrogen, C1-C6 alkyl, C3-C10 cycloalkyl and W, as defined above;
wherein if R¹⁹ is W, R¹⁵ is hydrogen;
R²⁰ and R²¹ are, at each occurrence, independently selected from the group consisting of hydrogen, halogen, C1-C6 alkyl, C1-C3 haloalkyl, —OR⁵, heterocyclyl and —CN;
R²² is, at each occurrence, independently selected from the group consisting of hydrogen, halogen, C1-C6 alkyl, C3-C10 cycloalkyl, —N(R⁵)₂, —NR¹⁹R²⁰, heterocyclyl, —OR⁵ and —CN;

In one embodiment, at least one of Z, R⁶, R⁷, R¹¹, R¹², R¹³, R¹⁵, R¹⁶ and R¹⁹ is W, as defined herein, or is a structure containing W, as defined herein.

In one embodiment, exactly one of Z, R⁶, R⁷, R¹¹, R¹², R¹³, R¹⁵, R¹⁶ and R¹⁹ is W, as defined herein, or is a structure containing W, as defined herein.

In one embodiment, R¹ is hydrogen and
the compound has the general formula II

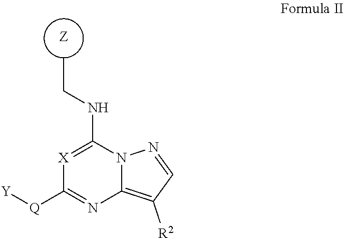

wherein X, Y, Z, R² and Q are as defined herein.

In one embodiment, said compound has the general formula III

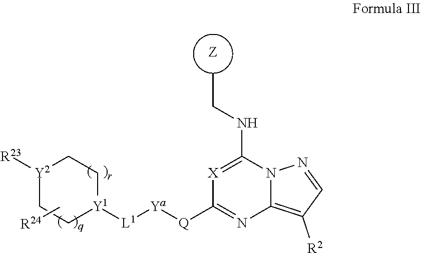

wherein X, Z, R² and Q are as defined herein, and
Y^{a} is either absent or independently, at each occurrence, selected from the group consisting of aryl, heteroaryl, heterocyclyl, aryl substituted with one or two of C1-C6 alkyl, —OR⁵, —N(R⁵)R⁵, and halogen, heteroaryl substituted with one or two of C1-C6 alkyl, —OR⁵, —N(R⁵)R⁵ and halogen, heterocyclyl substituted with one or two of R²³ and R²⁴;
R²³ is either absent or independently, at each occurrence, selected from the group consisting of hydrogen, —OR⁵, halogen, —N(R⁵)R⁵, —NH(C═O)R⁵, —(C═O)NH₂, aryl, heteroaryl, heterocyclyl, C1-C6 alkyl and C1-C6 alkyl substituted with —OH or —NH₂;
R²⁴ is, independently, at each occurrence, selected from the group consisting of hydrogen, halogen, —OR⁵, —N(R⁵)R⁵, (═O), aryl, heteroaryl, heterocyclyl, C1-C6 alkyl and C1-C6 alkyl substituted with —OH or —NH₂;
wherein R⁵ is as defined herein;
L¹ is either absent or independently, at each occurrence, selected from the group consisting of —NH—, —NH(CH₂)—, —NH(C═O)—, —NHSO₂—, —O—, —O(CH₂)—, —(C═O)—, —(C═O)NH— and —(C═O)(CH₂)—;
Y¹ is, independently at each occurrence, selected from CH, C(OH) and N;
Y² is, independently at each occurrence, selected from CH, C(OH), 0 and N;
q is, independently at each occurrence, selected from 0, 1 and 2;
r is, independently at each occurrence, selected from 0, 1, 2 and 3.

In one embodiment, Z is Z¹, and Z¹ is any structure of the following group E;

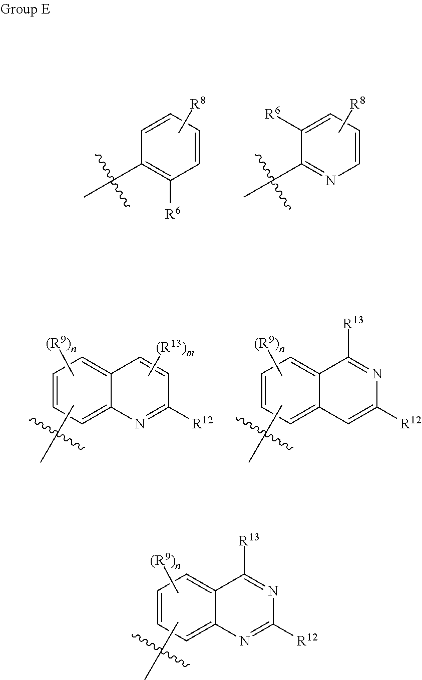

wherein m is, independently at each occurrence, selected from 1 and 2; and
n is as defined herein;
R⁸, R⁹, R¹² and R¹³ are as defined herein;
R⁶ is any structure of group B as defined herein.

In one embodiment, Z is

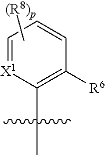

p is, independently at each occurrence, selected from 0, 1, 2 and 3;
X¹ is, independently at each occurrence, selected from CR⁸ and N;
R⁶ is any structure of group B, as defined herein; and
R⁸ is as defined herein.

In one embodiment, Z is

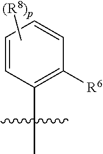

### or Z is

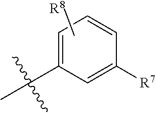

wherein R⁶-R⁸ are as defined herein.

In one embodiment, Z is

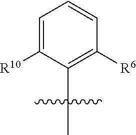

wherein R⁶ and R¹⁰ are as defined herein.

In one embodiment, in particular the embodiment of any of claims 1, 15, 18 or 19,
R¹ is hydrogen, Z is

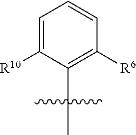

and R⁶ and R¹⁰ are as defined herein.

In one embodiment, said compound has the general formula IV

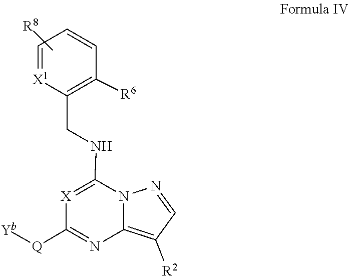

wherein X, X¹, R⁶, R⁸ and Q are as defined herein, and
X¹ is as defined herein, in particular in claim 21;
wherein Y^{b} is any structure of the following group F;

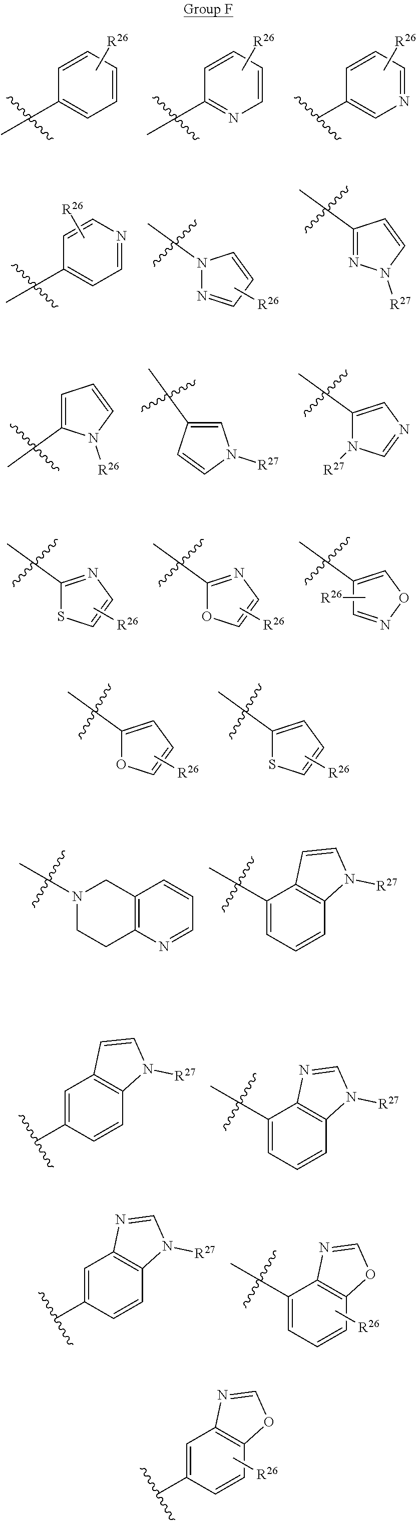

R²⁶ and R²⁷ is either absent or independently, at each occurrence, selected from the group consisting of hydrogen, —OR⁵, halogen, —N(R⁵)R⁵, —NH(C═O)R⁵, —(C═O)NH₂, aryl, heteroaryl, heterocyclyl, C1-C6 alkyl and C1-C6 alkyl substituted with —OH or —NH₂;
wherein R⁵ is as defined herein.

In one embodiment, R² is C1-C6 alkyl or C1-C3 haloalkyl.

In one embodiment, R⁶ is

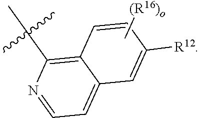

In one embodiment, R¹⁶ is hydrogen; o is 1; R¹² is W; W is (c−1) or (c−2) or (c−3), preferably (c−1); L is —NH—; R²⁰ and R²¹ are, independently, at each occurrence, hydrogen, halogen, or C1-C6 alkyl, wherein, preferably, R²⁰ is halogen; wherein R²² is hydrogen, halogen, C1-C6 alkyl, —N(R⁵)₂, —NR¹⁹R²⁰, wherein, preferably, R²² is —N(R⁵)₂ or —NR¹⁹R²⁰.

In one embodiment, said compound is a compound having a structure selected from structures 1 216, as defined in the following table:

| #cpds | Structure |
|---|---|
| 1 | 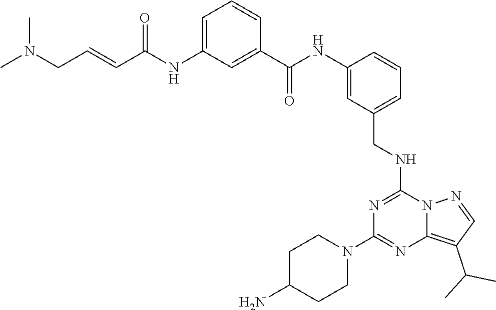 [SMILES: CC(C)c1cnn2c(NCc3cccc(NC(=O)c4cccc(NC(=O)/C=C/CN(C)C)c4)c3)nc(N3CCC(N)CC3)nc12] |
| 2 | 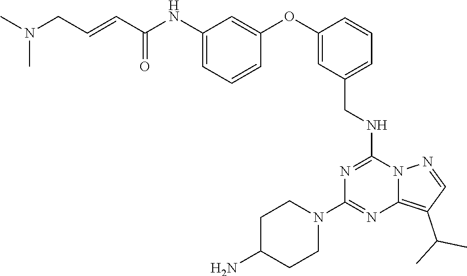 [SMILES: CC(C)c1cnn2c(NCc3cccc(Oc4cccc(NC(=O)/C=C/CN(C)C)c4)c3)nc(N3CCC(N)CC3)nc12] |
| 3 | 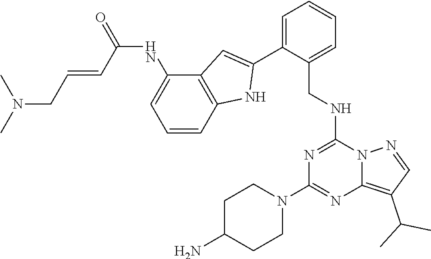 [SMILES: CC(C)c1cnn2c(NCc3ccccc3-c3cc4c(NC(=O)/C=C/CN(C)C)cccc4[nH]3)nc(N3CCC(N)CC3)nc12] |
| 4 | 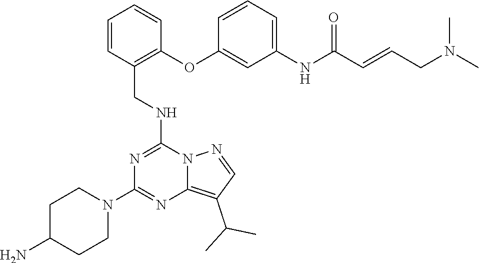 [SMILES: CC(C)c1cnn2c(NCc3ccccc3Oc3cccc(NC(=O)/C=C/CN(C)C)c3)nc(N3CCC(N)CC3)nc12] |
| 5 | 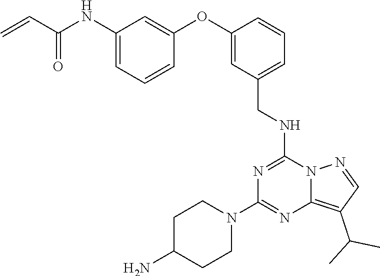 [SMILES: C=CC(=O)Nc1cccc(Oc2cccc(CNc3nc(N4CCC(N)CC4)nc4c(C(C)C)cnn34)c2)c1] |
| 6 | 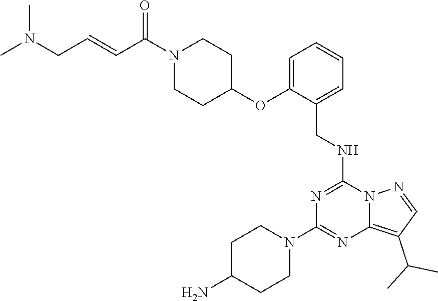 [SMILES: CC(C)c1cnn2c(NCc3ccccc3OC3CCN(C(=O)/C=C/CN(C)C)CC3)nc(N3CCC(N)CC3)nc12] |
| 7 | 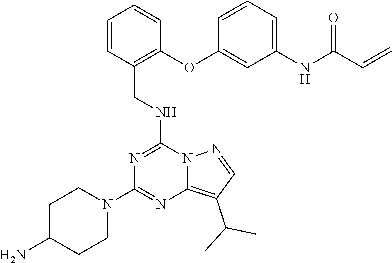 [SMILES: C=CC(=O)Nc1cccc(Oc2ccccc2CNc2nc(N3CCC(N)CC3)nc3c(C(C)C)cnn23)c1] |
| 8 | 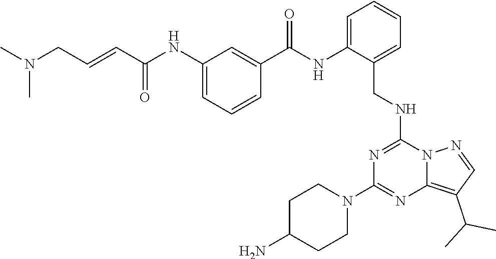 [SMILES: CC(C)c1cnn2c(NCc3ccccc3NC(=O)c3cccc(NC(=O)/C=C/CN(C)C)c3)nc(N3CCC(N)CC3)nc12] |
| 9 | 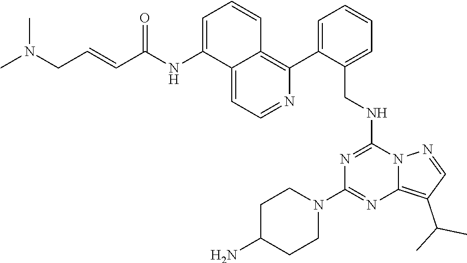 [SMILES: CC(C)c1cnn2c(NCc3ccccc3-c3nccc4c(NC(=O)/C=C/CN(C)C)cccc34)nc(N3CCC(N)CC3)nc12] |
| 10 | 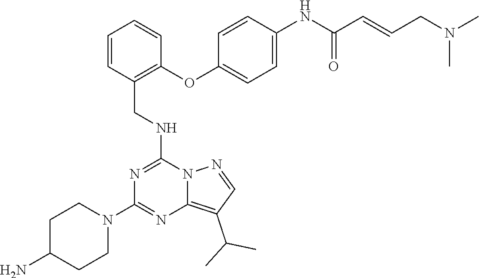 [SMILES: CC(C)c1cnn2c(NCc3ccccc3Oc3ccc(NC(=O)/C=C/CN(C)C)cc3)nc(N3CCC(N)CC3)nc12] |
| 11 | 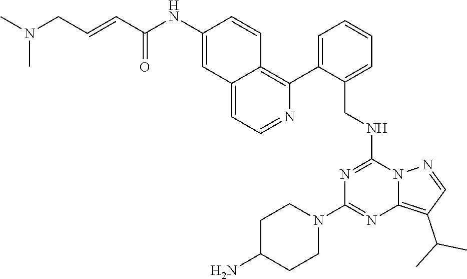 [SMILES: CC(C)c1cnn2c(NCc3ccccc3-c3nccc4cc(NC(=O)/C=C/CN(C)C)ccc34)nc(N3CCC(N)CC3)nc12] |
| 12 | 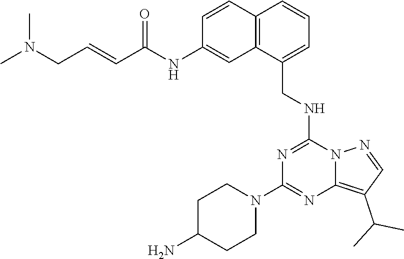 [SMILES: CC(C)c1cnn2c(NCc3cccc4ccc(NC(=O)/C=C/CN(C)C)cc34)nc(N3CCC(N)CC3)nc12] |
| 13 | 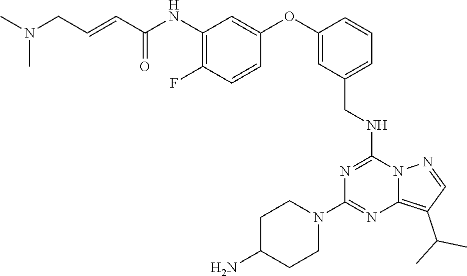 [SMILES: CC(C)c1cnn2c(NCc3cccc(Oc4ccc(F)c(NC(=O)/C=C/CN(C)C)c4)c3)nc(N3CCC(N)CC3)nc12] |
| 14 | 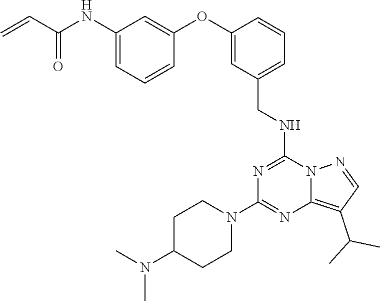 [SMILES: C=CC(=O)Nc1cccc(Oc2cccc(CNc3nc(N4CCC(N(C)C)CC4)nc4c(C(C)C)cnn34)c2)c1] |
| 15 | 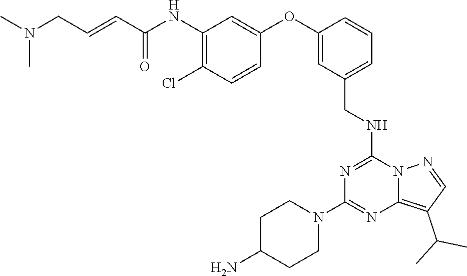 [SMILES: CC(C)c1cnn2c(NCc3cccc(Oc4ccc(Cl)c(NC(=O)/C=C/CN(C)C)c4)c3)nc(N3CCC(N)CC3)nc12] |
| 16 | 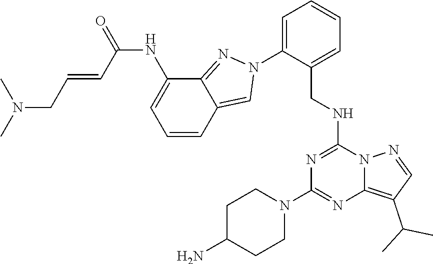 [SMILES: CC(C)c1cnn2c(NCc3ccccc3-n3cc4cccc(NC(=O)/C=C/CN(C)C)c4n3)nc(N3CCC(N)CC3)nc12] |
| 17 | 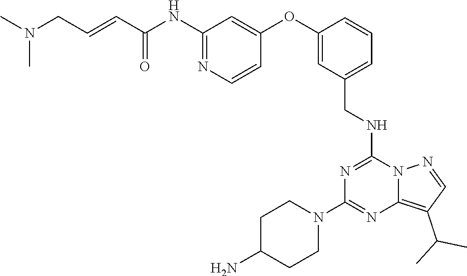 [SMILES: CC(C)c1cnn2c(NCc3cccc(Oc4ccnc(NC(=O)/C=C/CN(C)C)c4)c3)nc(N3CCC(N)CC3)nc12] |
| 18 | 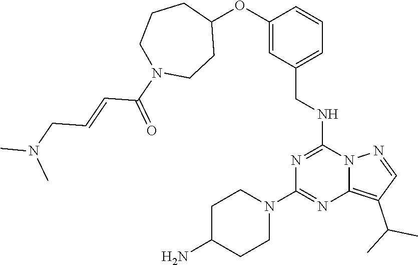 [SMILES: CC(C)c1cnn2c(NCc3cccc(OC4CCCN(C(=O)/C=C/CN(C)C)CC4)c3)nc(N3CCC(N)CC3)nc12] |
| 19 | 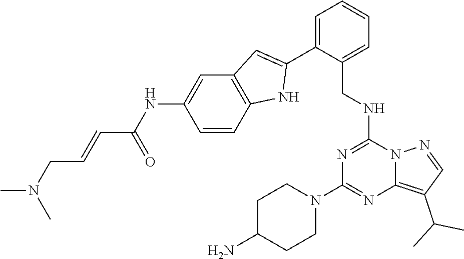 [SMILES: CC(C)c1cnn2c(NCc3ccccc3-c3cc4cc(NC(=O)/C=C/CN(C)C)ccc4[nH]3)nc(N3CCC(N)CC3)nc12] |
| 20 | 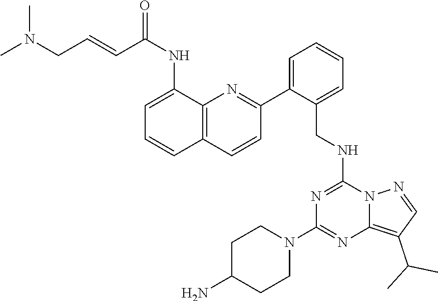 [SMILES: CC(C)c1cnn2c(NCc3ccccc3-c3ccc4cccc(NC(=O)/C=C/CN(C)C)c4n3)nc(N3CCC(N)CC3)nc12] |
| 21 | 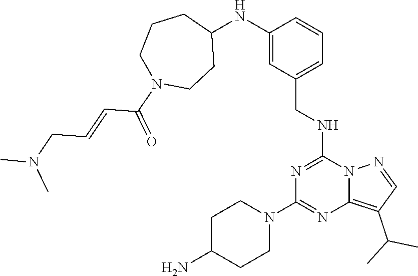 [SMILES: CC(C)c1cnn2c(NCc3cccc(NC4CCCN(C(=O)/C=C/CN(C)C)CC4)c3)nc(N3CCC(N)CC3)nc12] |
| 22 | 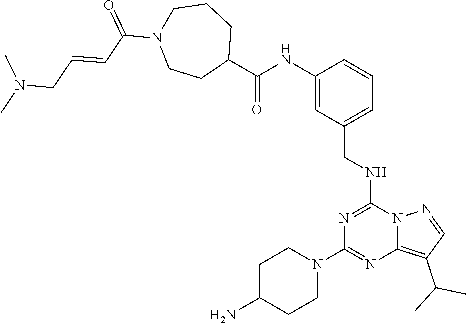 [SMILES: CC(C)c1cnn2c(NCc3cccc(NC(=O)C4CCCN(C(=O)/C=C/CN(C)C)CC4)c3)nc(N3CCC(N)CC3)nc12] |
| 23 | 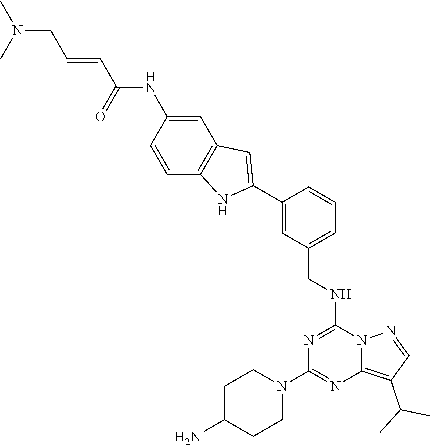 [SMILES: CC(C)c1cnn2c(NCc3cccc(-c4cc5cc(NC(=O)/C=C/CN(C)C)ccc5[nH]4)c3)nc(N3CCC(N)CC3)nc12] |
| 24 | 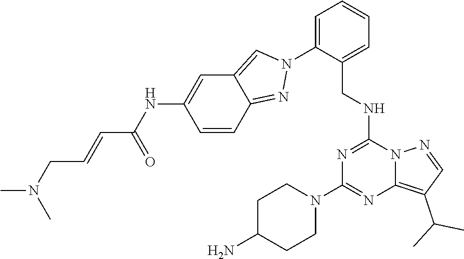 [SMILES: CC(C)c1cnn2c(NCc3ccccc3-n3cc4cc(NC(=O)/C=C/CN(C)C)ccc4n3)nc(N3CCC(N)CC3)nc12] |
| 25 | 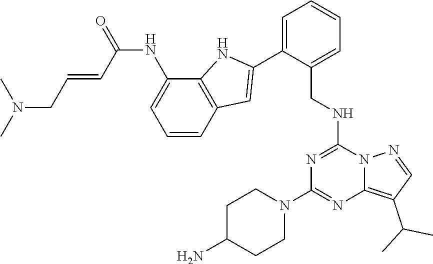 [SMILES: CC(C)c1cnn2c(NCc3ccccc3-c3cc4cccc(NC(=O)/C=C/CN(C)C)c4[nH]3)nc(N3CCC(N)CC3)nc12] |
| 26 | 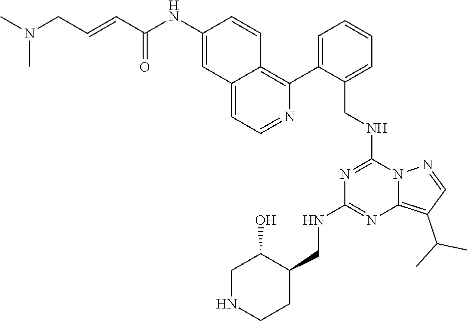 [SMILES: CC(C)c1cnn2c(NCc3ccccc3-c3nccc4cc(NC(=O)/C=C/CN(C)C)ccc34)nc(NC[C@H]3CCNC[C@@H]3O)nc12] |
| 27 | 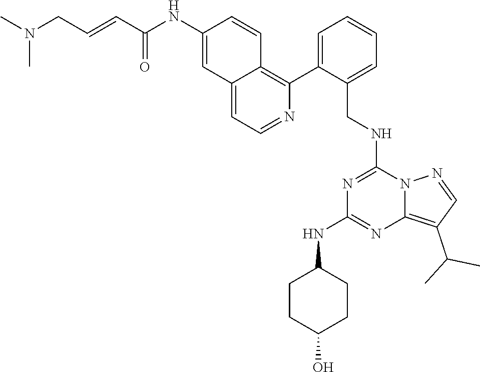 |
| 28 | 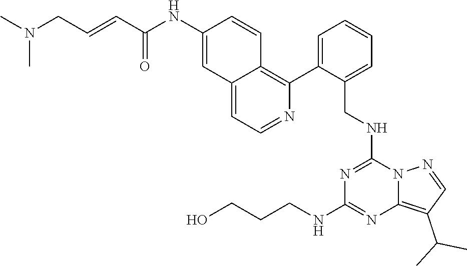 [SMILES: CC(C)c1cnn2c(NCc3ccccc3-c3nccc4cc(NC(=O)/C=C/CN(C)C)ccc34)nc(NCCCO)nc12] |
| 29 | 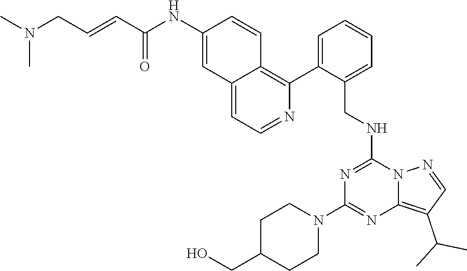 [SMILES: CC(C)c1cnn2c(NCc3ccccc3-c3nccc4cc(NC(=O)/C=C/CN(C)C)ccc34)nc(N3CCC(CO)CC3)nc12] |
| 30 | 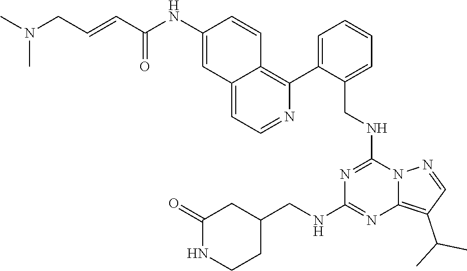 [SMILES: CC(C)c1cnn2c(NCc3ccccc3-c3nccc4cc(NC(=O)/C=C/CN(C)C)ccc34)nc(NCC3CCNC(=O)C3)nc12] |
| 31 | 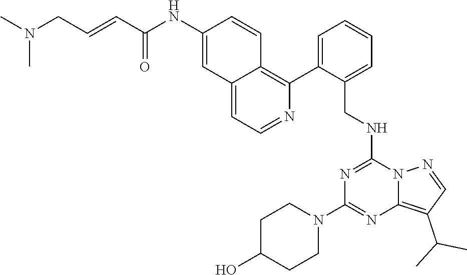 [SMILES: CC(C)c1cnn2c(NCc3ccccc3-c3nccc4cc(NC(=O)/C=C/CN(C)C)ccc34)nc(N3CCC(O)CC3)nc12] |
| 32 | 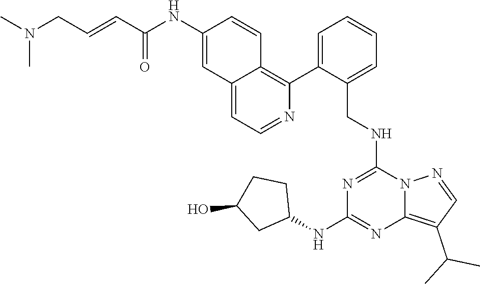 |
| 33 | 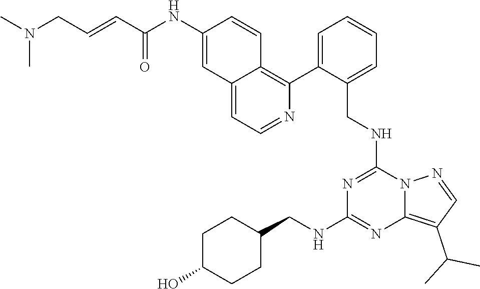 |
| 34 | 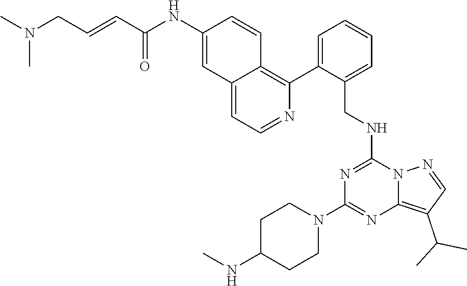 [SMILES: CNC1CCN(c2nc(NCc3ccccc3-c3nccc4cc(NC(=O)/C=C/CN(C)C)ccc34)n3ncc(C(C)C)c3n2)CC1] |
| 35 | 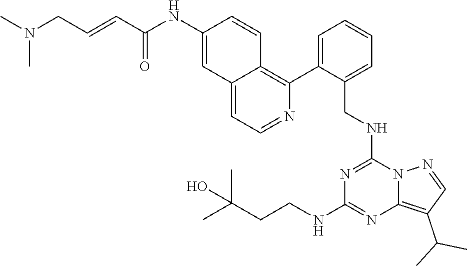 [SMILES: CC(C)c1cnn2c(NCc3ccccc3-c3nccc4cc(NC(=O)/C=C/CN(C)C)ccc34)nc(NCCC(C)(C)O)nc12] |
| 36 | 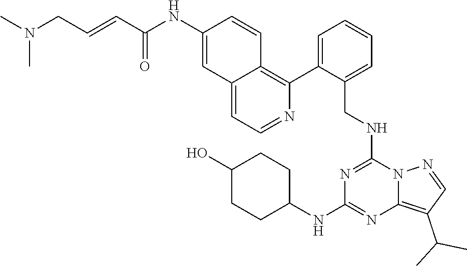 [SMILES: CC(C)c1cnn2c(NCc3ccccc3-c3nccc4cc(NC(=O)/C=C/CN(C)C)ccc34)nc(NC3CCC(O)CC3)nc12] |
| 37 | 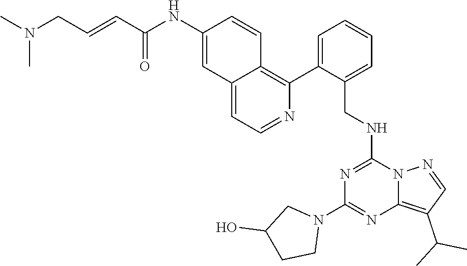 [SMILES: CC(C)c1cnn2c(NCc3ccccc3-c3nccc4cc(NC(=O)/C=C/CN(C)C)ccc34)nc(N3CCC(O)C3)nc12] |
| 38 | 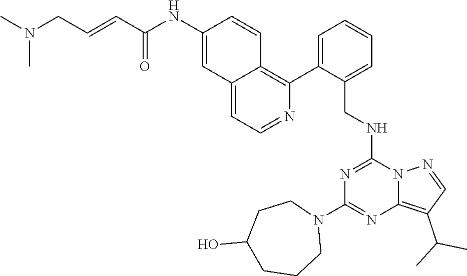 [SMILES: CC(C)c1cnn2c(NCc3ccccc3-c3nccc4cc(NC(=O)/C=C/CN(C)C)ccc34)nc(N3CCCC(O)CC3)nc12] |
| 39 | 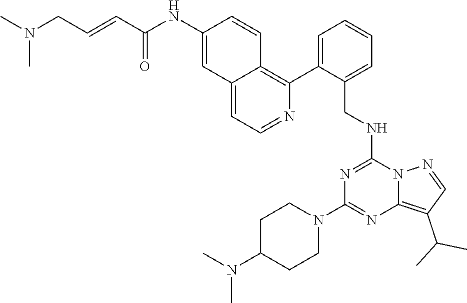 [SMILES: CC(C)c1cnn2c(NCc3ccccc3-c3nccc4cc(NC(=O)/C=C/CN(C)C)ccc34)nc(N3CCC(N(C)C)CC3)nc12] |
| 40 | 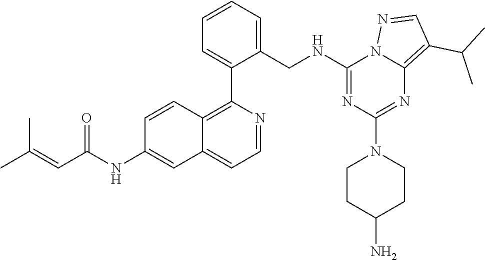 [SMILES: CC(C)=CC(=O)Nc1ccc2c(-c3ccccc3CNc3nc(N4CCC(N)CC4)nc4c(C(C)C)cnn34)nccc2c1] |
| 41 | 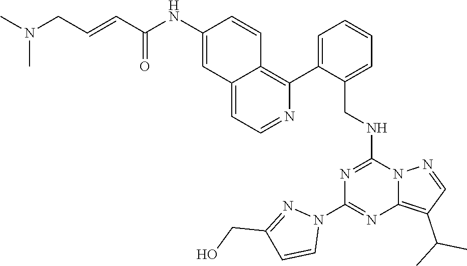 [SMILES: CC(C)c1cnn2c(NCc3ccccc3-c3nccc4cc(NC(=O)/C=C/CN(C)C)ccc34)nc(-n3ccc(CO)n3)nc12] |
| 42 | 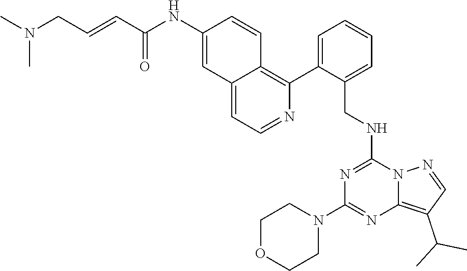 [SMILES: CC(C)c1cnn2c(NCc3ccccc3-c3nccc4cc(NC(=O)/C=C/CN(C)C)ccc34)nc(N3CCOCC3)nc12] |
| 43 | 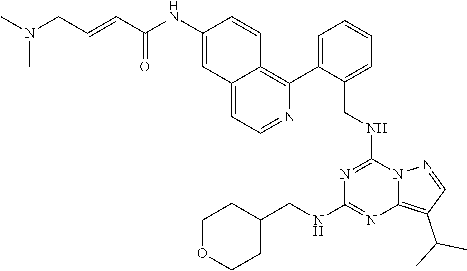 [SMILES: CC(C)c1cnn2c(NCc3ccccc3-c3nccc4cc(NC(=O)/C=C/CN(C)C)ccc34)nc(NCC3CCOCC3)nc12] |
| 44 | 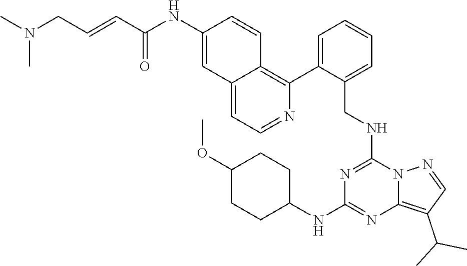 [SMILES: COC1CCC(Nc2nc(NCc3ccccc3-c3nccc4cc(NC(=O)/C=C/CN(C)C)ccc34)n3ncc(C(C)C)c3n2)CC1] |
| 45 | 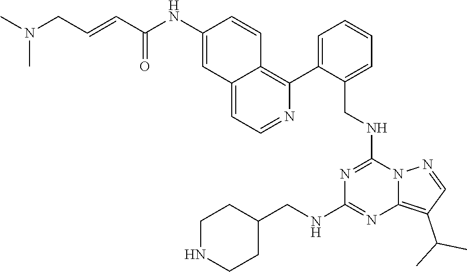 [SMILES: CC(C)c1cnn2c(NCc3ccccc3-c3nccc4cc(NC(=O)/C=C/CN(C)C)ccc34)nc(NCC3CCNCC3)nc12] |
| 46 | 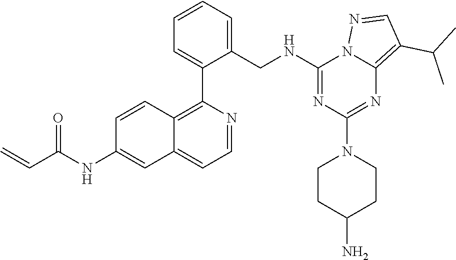 [SMILES: C=CC(=O)Nc1ccc2c(-c3ccccc3CNc3nc(N4CCC(N)CC4)nc4c(C(C)C)cnn34)nccc2c1] |
| 47 | 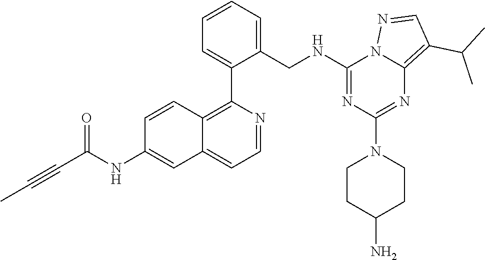 [SMILES: CC#CC(=O)Nc1ccc2c(-c3ccccc3CNc3nc(N4CCC(N)CC4)nc4c(C(C)C)cnn34)nccc2c1] |
| 48 | 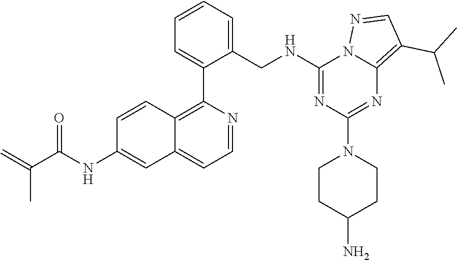 [SMILES: C=C(C)C(=O)Nc1ccc2c(-c3ccccc3CNc3nc(N4CCC(N)CC4)nc4c(C(C)C)cnn34)nccc2c1] |
| 49 | 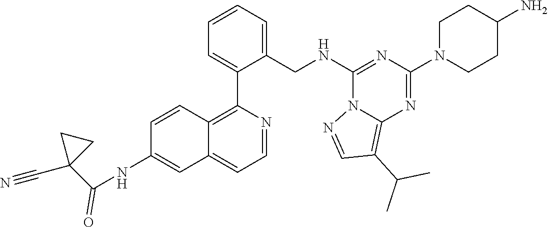 [SMILES: CC(C)c1cnn2c(NCc3ccccc3-c3nccc4cc(NC(=O)C5(C#N)CC5)ccc34)nc(N3CCC(N)CC3)nc12] |
| 50 | 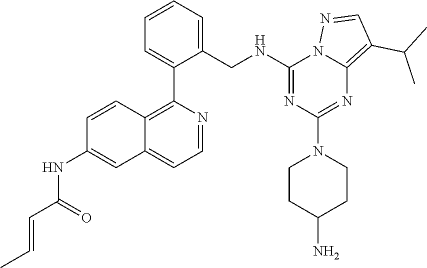 [SMILES: C/C=C/C(=O)Nc1ccc2c(-c3ccccc3CNc3nc(N4CCC(N)CC4)nc4c(C(C)C)cnn34)nccc2c1] |
| 51 | 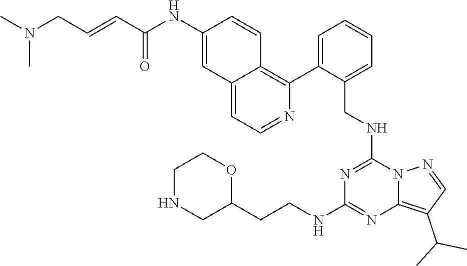 [SMILES: CC(C)c1cnn2c(NCc3ccccc3-c3nccc4cc(NC(=O)/C=C/CN(C)C)ccc34)nc(NCCC3CNCCO3)nc12] |
| 52 | 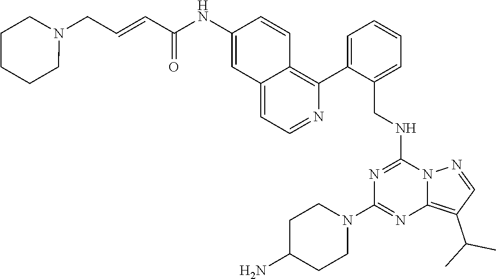 [SMILES: CC(C)c1cnn2c(NCc3ccccc3-c3nccc4cc(NC(=O)/C=C/CN5CCCCC5)ccc34)nc(N3CCC(N)CC3)nc12] |
| 53 | 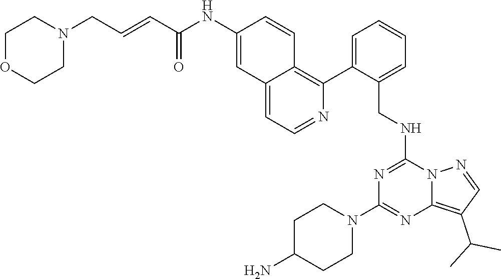 [SMILES: CC(C)c1cnn2c(NCc3ccccc3-c3nccc4cc(NC(=O)/C=C/CN5CCOCC5)ccc34)nc(N3CCC(N)CC3)nc12] |
| 54 | 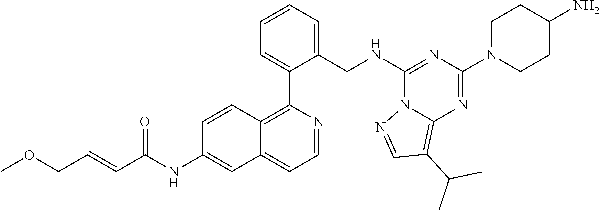 [SMILES: COC/C=C/C(=O)Nc1ccc2c(-c3ccccc3CNc3nc(N4CCC(N)CC4)nc4c(C(C)C)cnn34)nccc2c1] |
| 55 | 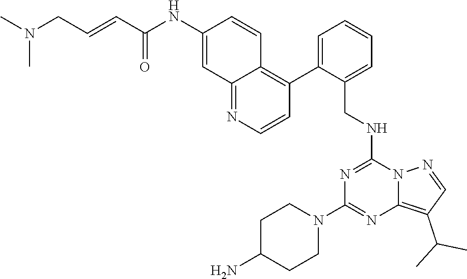 [SMILES: CC(C)c1cnn2c(NCc3ccccc3-c3ccnc4cc(NC(=O)/C=C/CN(C)C)ccc34)nc(N3CCC(N)CC3)nc12] |
| 56 | 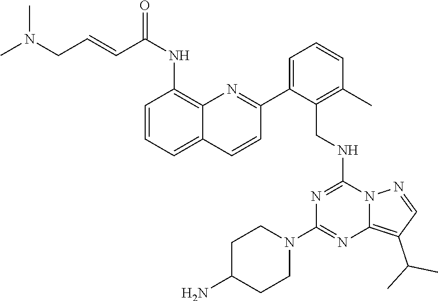 [SMILES: Cc1cccc(-c2ccc3cccc(NC(=O)/C=C/CN(C)C)c3n2)c1CNc1nc(N2CCC(N)CC2)nc2c(C(C)C)cnn12] |
| 57 | 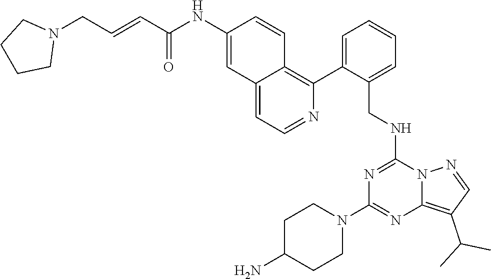 [SMILES: CC(C)c1cnn2c(NCc3ccccc3-c3nccc4cc(NC(=O)/C=C/CN5CCCC5)ccc34)nc(N3CCC(N)CC3)nc12] |
| 58 | 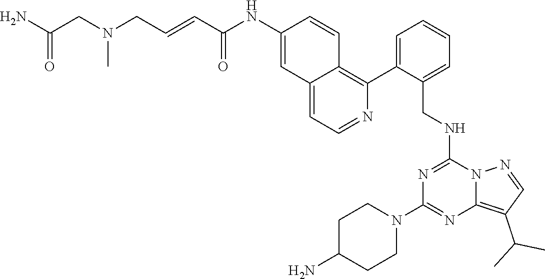 [SMILES: CC(C)c1cnn2c(NCc3ccccc3-c3nccc4cc(NC(=O)/C=C/CN(C)CC(N)=O)ccc34)nc(N3CCC(N)CC3)nc12] |
| 59 | 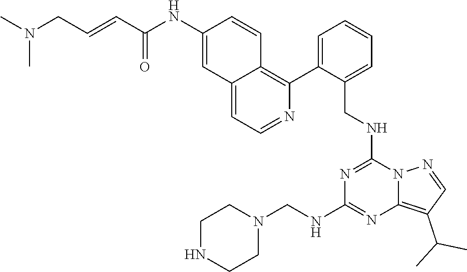 [SMILES: CC(C)c1cnn2c(NCc3ccccc3-c3nccc4cc(NC(=O)/C=C/CN(C)C)ccc34)nc(NCN3CCNCC3)nc12] |
| 60 | 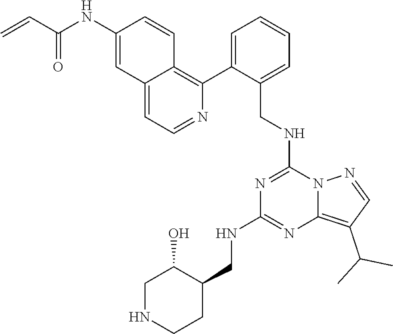 [SMILES: C=CC(=O)Nc1ccc2c(-c3ccccc3CNc3nc(NC[C@H]4CCNC[C@@H]4O)nc4c(C(C)C)cnn34)nccc2c1] |
| 61 | 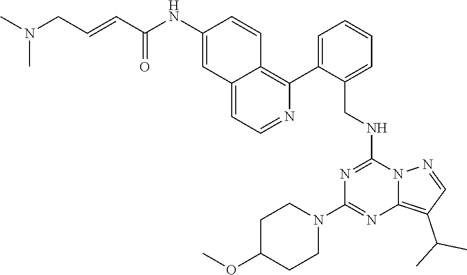 [SMILES: COC1CCN(c2nc(NCc3ccccc3-c3nccc4cc(NC(=O)/C=C/CN(C)C)ccc34)n3ncc(C(C)C)c3n2)CC1] |
| 62 | 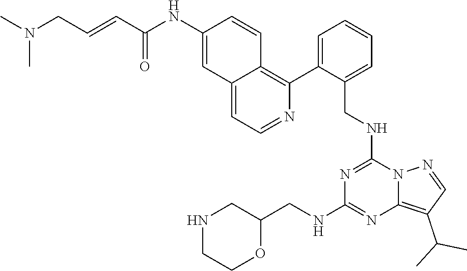 [SMILES: CC(C)c1cnn2c(NCc3ccccc3-c3nccc4cc(NC(=O)/C=C/CN(C)C)ccc34)nc(NCC3CNCCO3)nc12] |
| 63 | 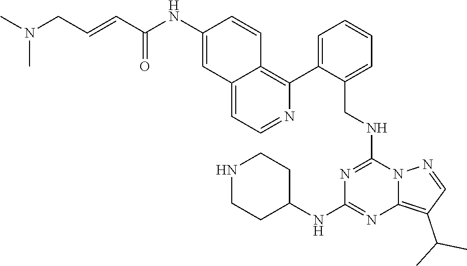 [SMILES: CC(C)c1cnn2c(NCc3ccccc3-c3nccc4cc(NC(=O)/C=C/CN(C)C)ccc34)nc(NC3CCNCC3)nc12] |
| 64 | 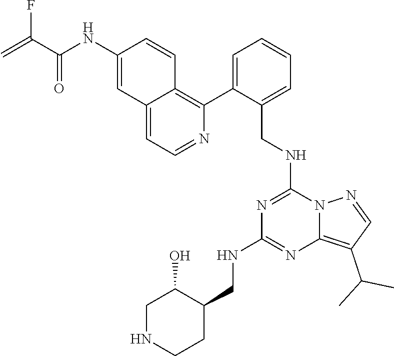 [SMILES: C=C(F)C(=O)Nc1ccc2c(-c3ccccc3CNc3nc(NC[C@H]4CCNC[C@@H]4O)nc4c(C(C)C)cnn34)nccc2c1] |
| 65 | 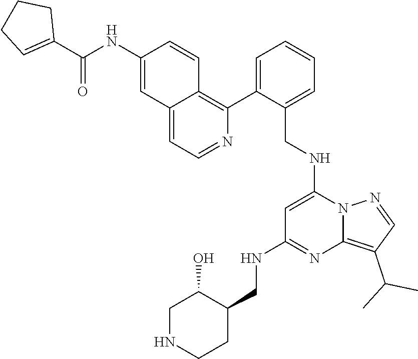 [SMILES: CC(C)c1cnn2c(NCc3ccccc3-c3nccc4cc(NC(=O)C5=CCCC5)ccc34)cc(NC[C@H]3CCNC[C@@H]3O)nc12] |
| 66 | 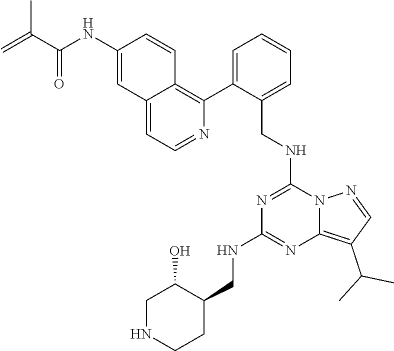 [SMILES: C=C(C)C(=O)Nc1ccc2c(-c3ccccc3CNc3nc(NC[C@H]4CCNC[C@@H]4O)nc4c(C(C)C)cnn34)nccc2c1] |
| 67 | 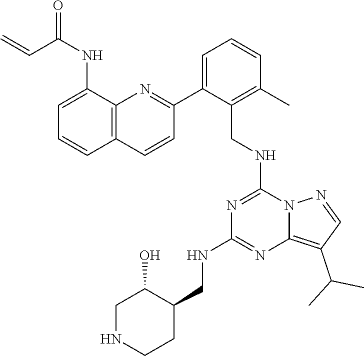 [SMILES: C=CC(=O)Nc1cccc2ccc(-c3cccc(C)c3CNc3nc(NC[C@H]4CCNC[C@@H]4O)nc4c(C(C)C)cnn34)nc12] |
| 68 | 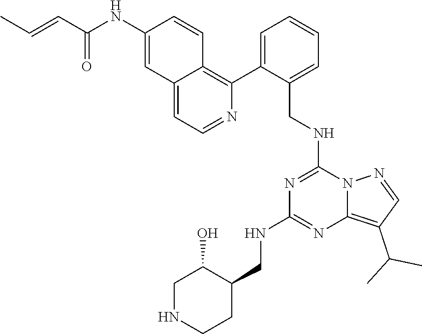 [SMILES: C/C=C/C(=O)Nc1ccc2c(-c3ccccc3CNc3nc(NC[C@H]4CCNC[C@@H]4O)nc4c(C(C)C)cnn34)nccc2c1] |
| 69 | 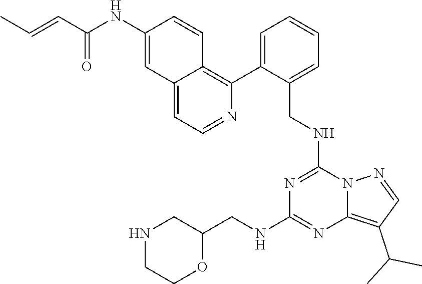 [SMILES: C/C=C/C(=O)Nc1ccc2c(-c3ccccc3CNc3nc(NCC4CNCCO4)nc4c(C(C)C)cnn34)nccc2c1] |
| 70 | 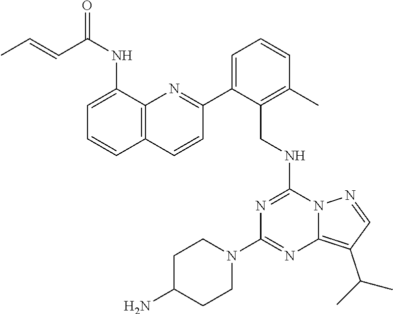 [SMILES: C/C=C/C(=O)Nc1cccc2ccc(-c3cccc(C)c3CNc3nc(N4CCC(N)CC4)nc4c(C(C)C)cnn34)nc12] |
| 71 | 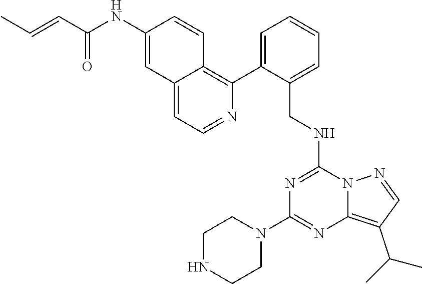 [SMILES: C/C=C/C(=O)Nc1ccc2c(-c3ccccc3CNc3nc(N4CCNCC4)nc4c(C(C)C)cnn34)nccc2c1] |
| 72 | 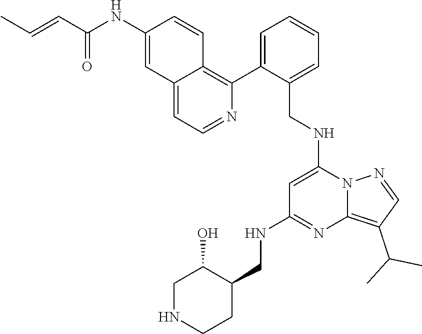 [SMILES: C/C=C/C(=O)Nc1ccc2c(-c3ccccc3CNc3cc(NC[C@H]4CCNC[C@@H]4O)nc4c(C(C)C)cnn34)nccc2c1] |
| 73 | 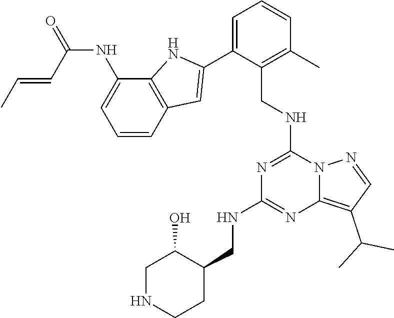 [SMILES: C/C=C/C(=O)Nc1cccc2cc(-c3cccc(C)c3CNc3nc(NC[C@H]4CCNC[C@@H]4O)nc4c(C(C)C)cnn34)[nH]c12] |
| 74 | 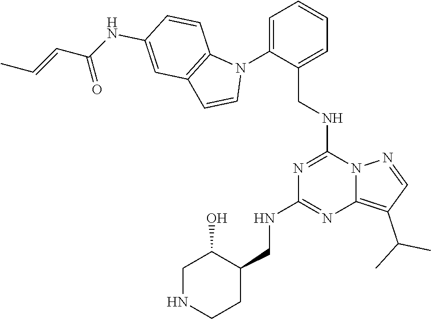 [SMILES: C/C=C/C(=O)Nc1ccc2c(ccn2-c2ccccc2CNc2nc(NC[C@H]3CCNC[C@@H]3O)nc3c(C(C)C)cnn23)c1] |
| 75 | 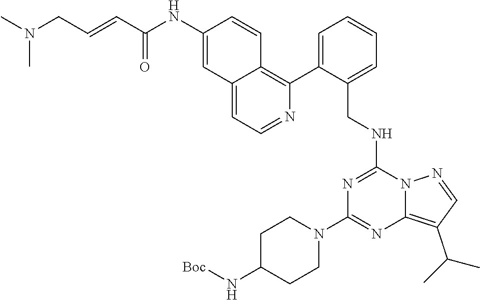 [SMILES: CC(C)c1cnn2c(NCc3ccccc3-c3nccc4cc(NC(=O)/C=C/CN(C)C)ccc34)nc(N3CCC(NC(=O)OC(C)(C)C)CC3)nc12] |
| 76 | 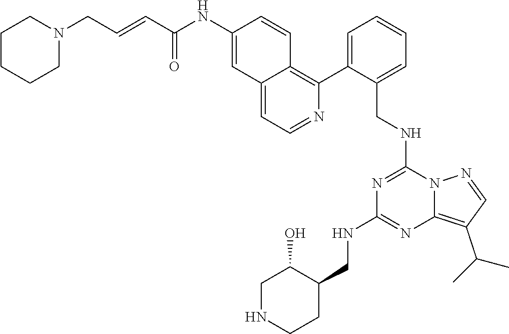 [SMILES: CC(C)c1cnn2c(NCc3ccccc3-c3nccc4cc(NC(=O)/C=C/CN5CCCCC5)ccc34)nc(NC[C@H]3CCNC[C@@H]3O)nc12] |
| 77 | 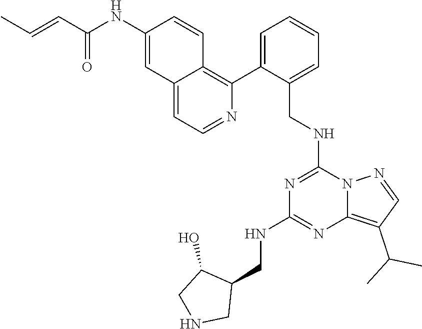 [SMILES: C/C=C/C(=O)Nc1ccc2c(-c3ccccc3CNc3nc(NC[C@H]4CNC[C@@H]4O)nc4c(C(C)C)cnn34)nccc2c1] |
| 78 | 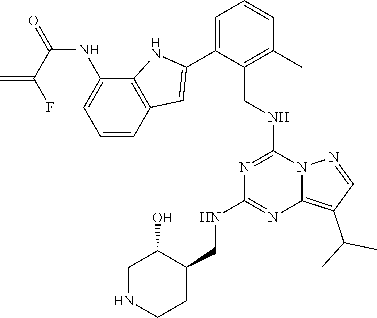 [SMILES: C=C(F)C(=O)Nc1cccc2cc(-c3cccc(C)c3CNc3nc(NC[C@H]4CCNC[C@@H]4O)nc4c(C(C)C)cnn34)[nH]c12] |
| 79 | 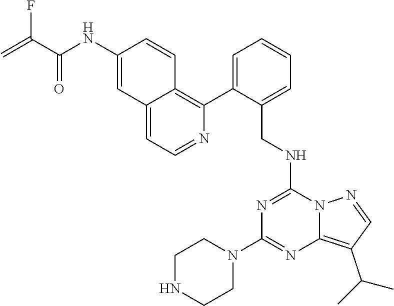 [SMILES: C=C(F)C(=O)Nc1ccc2c(-c3ccccc3CNc3nc(N4CCNCC4)nc4c(C(C)C)cnn34)nccc2c1] |
| 80 | 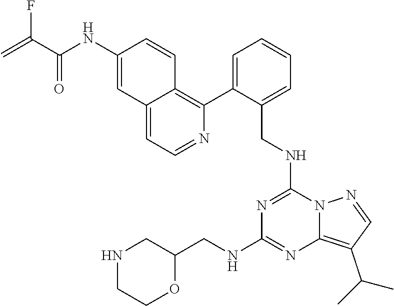 [SMILES: C=C(F)C(=O)Nc1ccc2c(-c3ccccc3CNc3nc(NCC4CNCCO4)nc4c(C(C)C)cnn34)nccc2c1] |
| 81 | 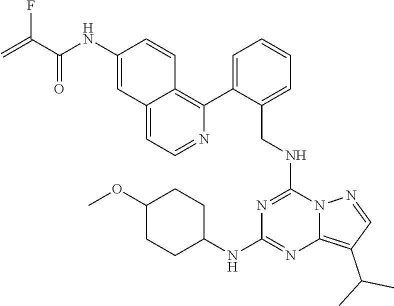 [SMILES: C=C(F)C(=O)Nc1ccc2c(-c3ccccc3CNc3nc(NC4CCC(OC)CC4)nc4c(C(C)C)cnn34)nccc2c1] |
| 82 | 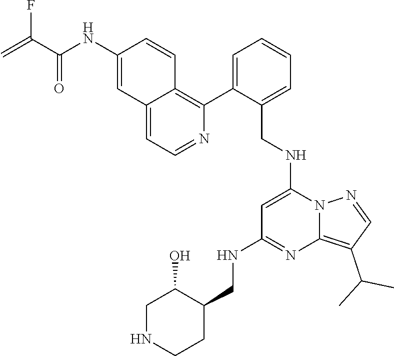 [SMILES: C=C(F)C(=O)Nc1ccc2c(-c3ccccc3CNc3cc(NC[C@H]4CCNC[C@@H]4O)nc4c(C(C)C)cnn34)nccc2c1] |
| 83 | 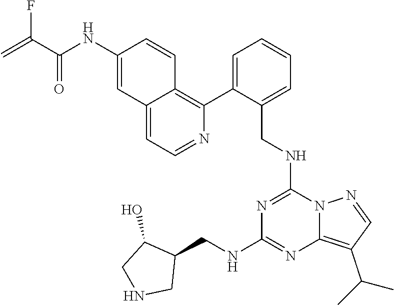 [SMILES: C=C(F)C(=O)Nc1ccc2c(-c3ccccc3CNc3nc(NC[C@H]4CNC[C@@H]4O)nc4c(C(C)C)cnn34)nccc2c1] |
| 84 | 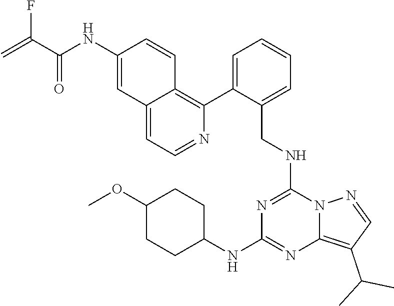 [SMILES: C=C(F)C(=O)Nc1ccc2c(-c3ccccc3CNc3nc(NC4CCC(OC)CC4)nc4c(C(C)C)cnn34)nccc2c1] |
| 85 | 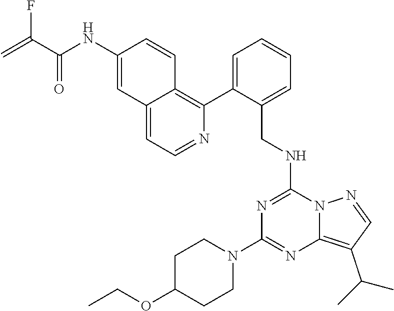 [SMILES: C=C(F)C(=O)Nc1ccc2c(-c3ccccc3CNc3nc(N4CCC(OCC)CC4)nc4c(C(C)C)cnn34)nccc2c1] |
| 86 | 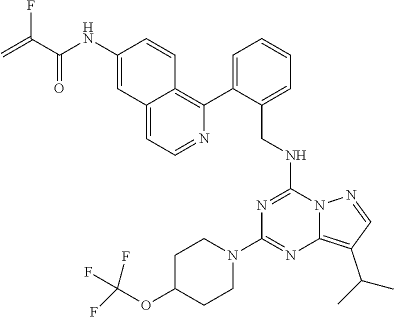 [SMILES: C=C(F)C(=O)Nc1ccc2c(-c3ccccc3CNc3nc(N4CCC(OC(F)(F)F)CC4)nc4c(C(C)C)cnn34)nccc2c1] |
| 87 | 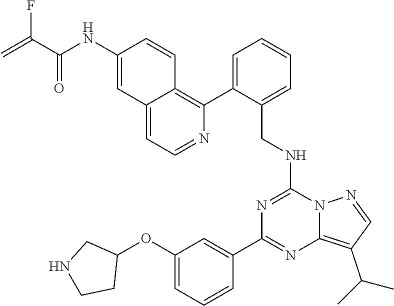 [SMILES: C=C(F)C(=O)Nc1ccc2c(-c3ccccc3CNc3nc(-c4cccc(OC5CCNC5)c4)nc4c(C(C)C)cnn34)nccc2c1] |
| 88 | 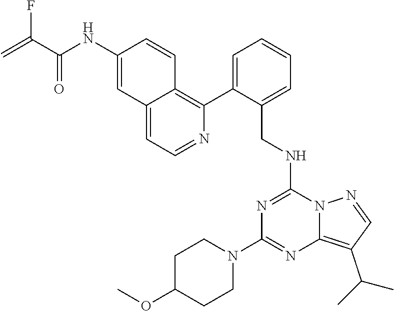 [SMILES: C=C(F)C(=O)Nc1ccc2c(-c3ccccc3CNc3nc(N4CCC(OC)CC4)nc4c(C(C)C)cnn34)nccc2c1] |
| 89 | 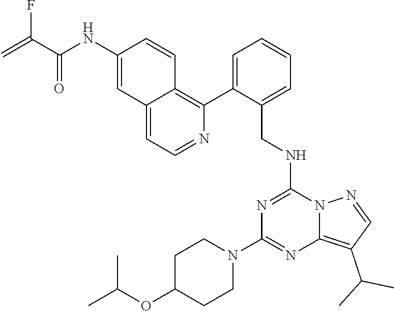 [SMILES: C=C(F)C(=O)Nc1ccc2c(-c3ccccc3CNc3nc(N4CCC(OC(C)C)CC4)nc4c(C(C)C)cnn34)nccc2c1] |
| 90 | 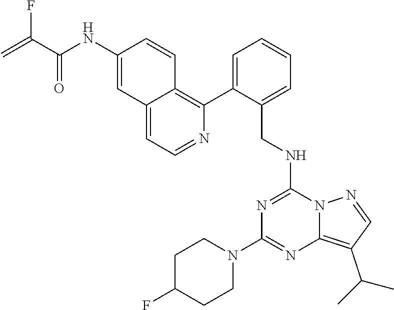 [SMILES: C=C(F)C(=O)Nc1ccc2c(-c3ccccc3CNc3nc(N4CCC(F)CC4)nc4c(C(C)C)cnn34)nccc2c1] |
| 91 | 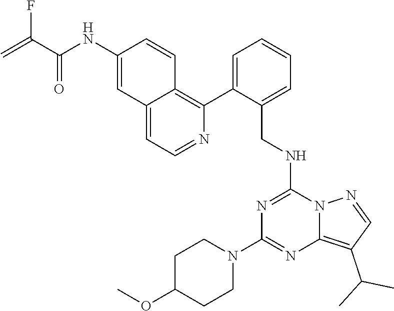 [SMILES: C=C(F)C(=O)Nc1ccc2c(-c3ccccc3CNc3nc(N4CCC(OC)CC4)nc4c(C(C)C)cnn34)nccc2c1] |
| 92 | 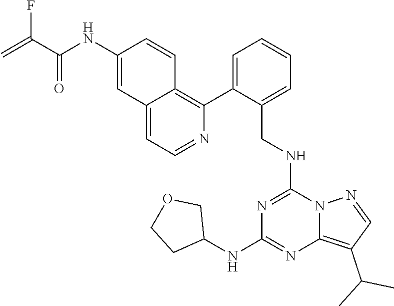 [SMILES: C=C(F)C(=O)Nc1ccc2c(-c3ccccc3CNc3nc(NC4CCOC4)nc4c(C(C)C)cnn34)nccc2c1] |
| 93 | 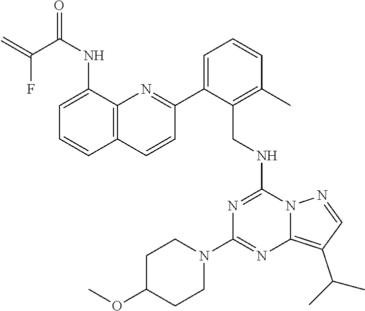 [SMILES: C=C(F)C(=O)Nc1cccc2ccc(-c3cccc(C)c3CNc3nc(N4CCC(OC)CC4)nc4c(C(C)C)cnn34)nc12] |
| 94 | 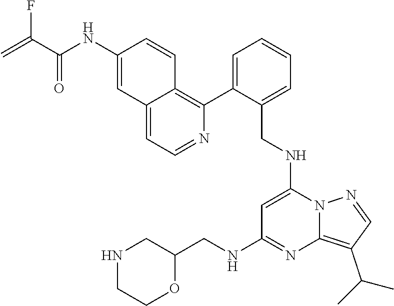 [SMILES: C=C(F)C(=O)Nc1ccc2c(-c3ccccc3CNc3cc(NCC4CNCCO4)nc4c(C(C)C)cnn34)nccc2c1] |
| 95 | 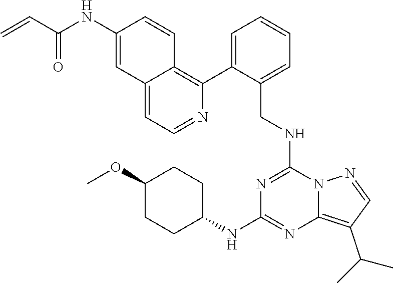 [SMILES: C=CC(=O)Nc1ccc2c(-c3ccccc3CNc3nc(N[C@H]4CC[C@H](OC)CC4)nc4c(C(C)C)cnn34)nccc2c1] |
| 96 | 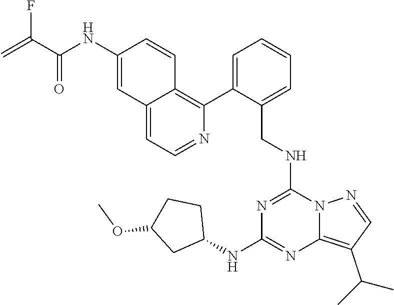 |
| 97 | 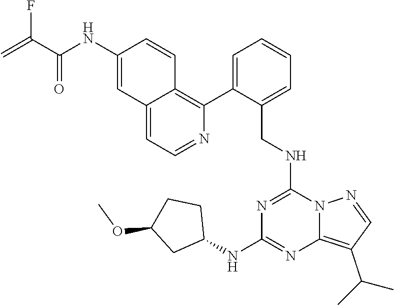 [SMILES: C=C(F)C(=O)Nc1ccc2c(-c3ccccc3CNc3nc(N[C@H]4CC[C@H](OC)C4)nc4c(C(C)C)cnn34)nccc2c1] |
| 98 | 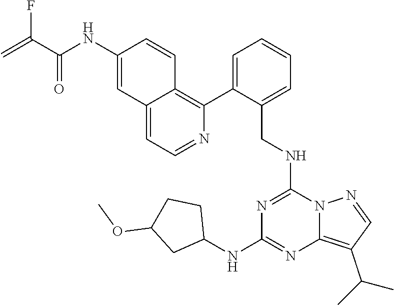 [SMILES: C=C(F)C(=O)Nc1ccc2c(-c3ccccc3CNc3nc(NC4CCC(OC)C4)nc4c(C(C)C)cnn34)nccc2c1] |
| 99 | 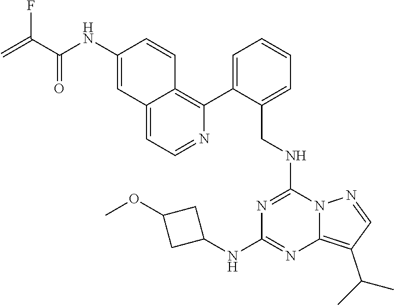 [SMILES: C=C(F)C(=O)Nc1ccc2c(-c3ccccc3CNc3nc(NC4CC(OC)C4)nc4c(C(C)C)cnn34)nccc2c1] |
| 100 | 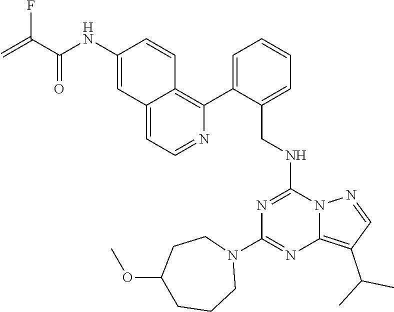 [SMILES: C=C(F)C(=O)Nc1ccc2c(-c3ccccc3CNc3nc(N4CCCC(OC)CC4)nc4c(C(C)C)cnn34)nccc2c1] |
| 101 | 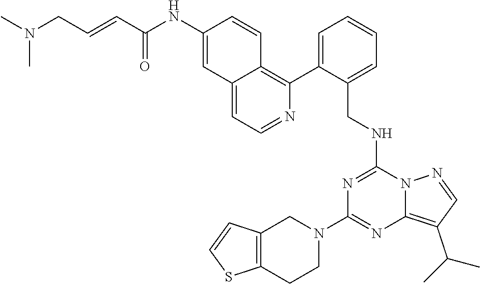 [SMILES: CC(C)c1cnn2c(NCc3ccccc3-c3nccc4cc(NC(=O)/C=C/CN(C)C)ccc34)nc(N3CCc4sccc4C3)nc12] |
| 102 | 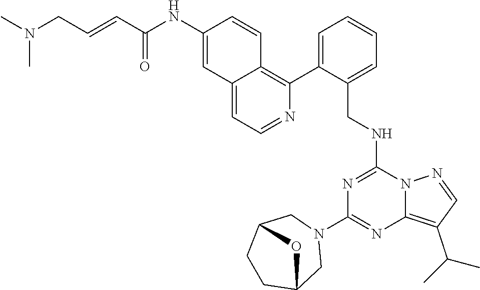 [SMILES: CC(C)c1cnn2c(NCc3ccccc3-c3nccc4cc(NC(=O)/C=C/CN(C)C)ccc34)nc(N3C[C@H]4CC[C@@H](C3)O4)nc12] |
| 103 | 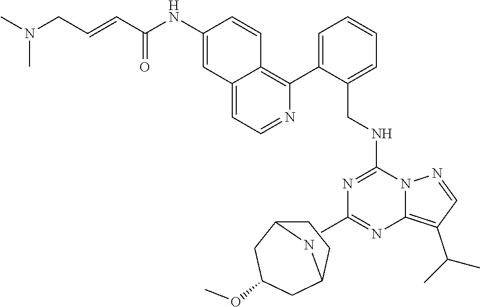 |
| 104 | 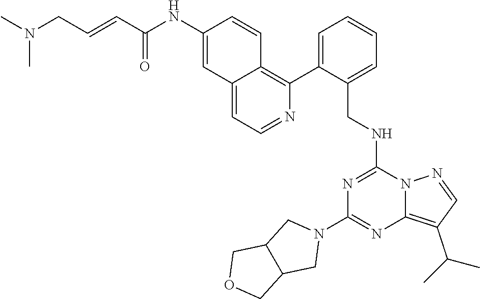 [SMILES: CC(C)c1cnn2c(NCc3ccccc3-c3nccc4cc(NC(=O)/C=C/CN(C)C)ccc34)nc(N3CC4COCC4C3)nc12] |
| 105 | 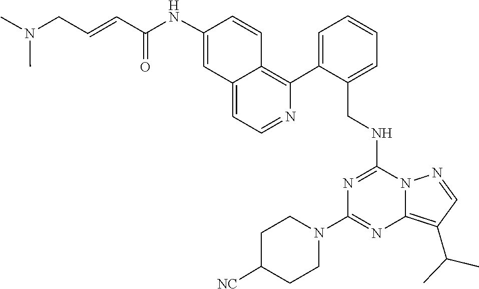 [SMILES: CC(C)c1cnn2c(NCc3ccccc3-c3nccc4cc(NC(=O)/C=C/CN(C)C)ccc34)nc(N3CCC(C#N)CC3)nc12] |
| 106 | 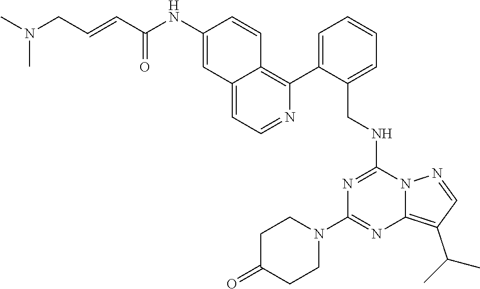 [SMILES: CC(C)c1cnn2c(NCc3ccccc3-c3nccc4cc(NC(=O)/C=C/CN(C)C)ccc34)nc(N3CCC(=O)CC3)nc12] |
| 107 | 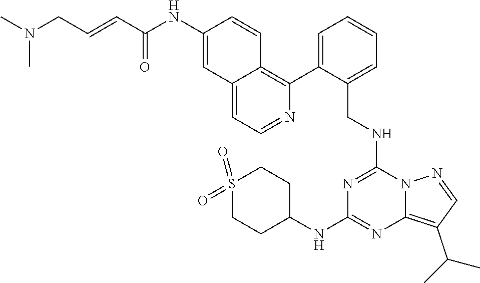 [SMILES: CC(C)c1cnn2c(NCc3ccccc3-c3nccc4cc(NC(=O)/C=C/CN(C)C)ccc34)nc(NC3CCS(=O)(=O)CC3)nc12] |
| 108 | 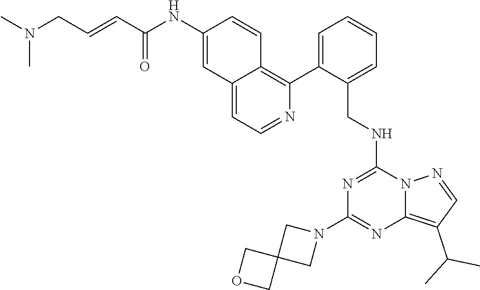 [SMILES: CC(C)c1cnn2c(NCc3ccccc3-c3nccc4cc(NC(=O)/C=C/CN(C)C)ccc34)nc(N3CC4(COC4)C3)nc12] |
| 109 | 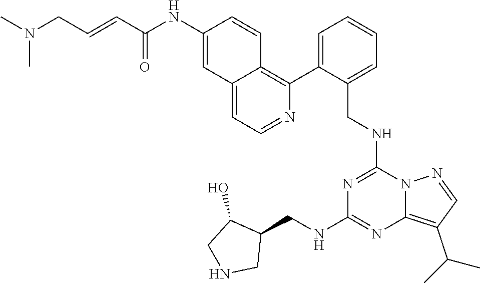 [SMILES: CC(C)c1cnn2c(NCc3ccccc3-c3nccc4cc(NC(=O)/C=C/CN(C)C)ccc34)nc(NC[C@H]3CNC[C@@H]3O)nc12] |
| 110 | 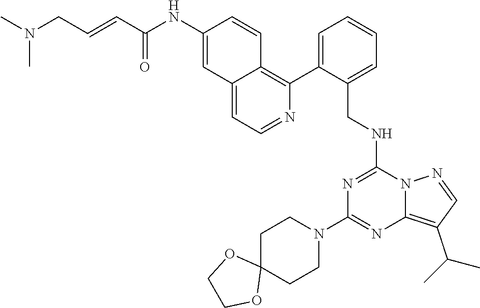 [SMILES: CC(C)c1cnn2c(NCc3ccccc3-c3nccc4cc(NC(=O)/C=C/CN(C)C)ccc34)nc(N3CCC4(CC3)OCCO4)nc12] |
| 111 | 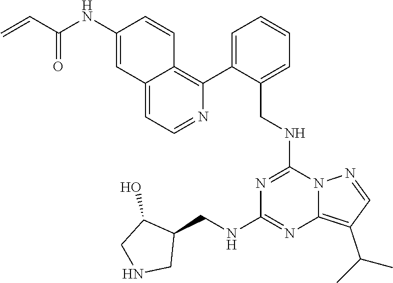 [SMILES: C=CC(=O)Nc1ccc2c(-c3ccccc3CNc3nc(NC[C@H]4CNC[C@@H]4O)nc4c(C(C)C)cnn34)nccc2c1] |
| 112 | 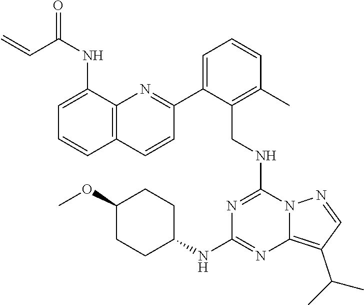 [SMILES: C=CC(=O)Nc1cccc2ccc(-c3cccc(C)c3CNc3nc(N[C@H]4CC[C@H](OC)CC4)nc4c(C(C)C)cnn34)nc12] |
| 113 | 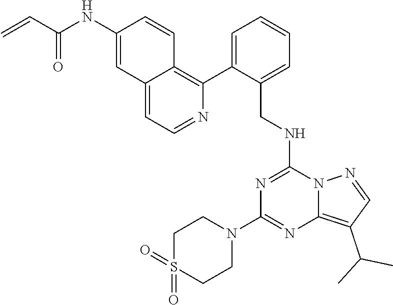 [SMILES: C=CC(=O)Nc1ccc2c(-c3ccccc3CNc3nc(N4CCS(=O)(=O)CC4)nc4c(C(C)C)cnn34)nccc2c1] |
| 114 | 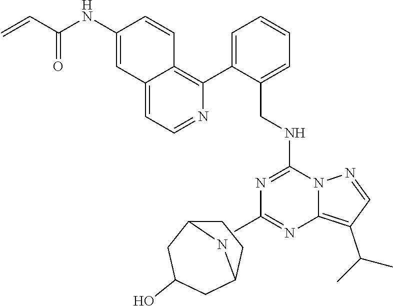 |
| 115 | 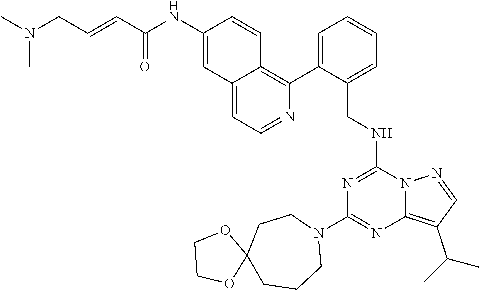 [SMILES: CC(C)c1cnn2c(NCc3ccccc3-c3nccc4cc(NC(=O)/C=C/CN(C)C)ccc34)nc(N3CCCC4(CC3)OCCO4)nc12] |
| 116 | 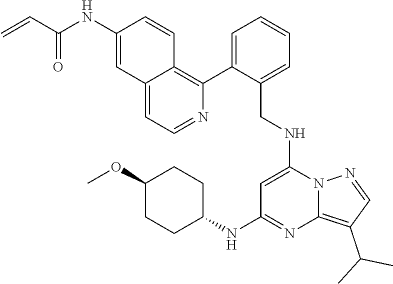 [SMILES: C=CC(=O)Nc1ccc2c(-c3ccccc3CNc3cc(N[C@H]4CC[C@H](OC)CC4)nc4c(C(C)C)cnn34)nccc2c1] |
| 117 | 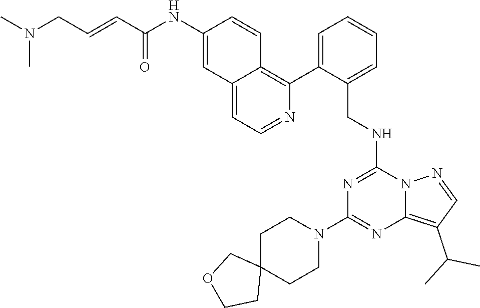 [SMILES: CC(C)c1cnn2c(NCc3ccccc3-c3nccc4cc(NC(=O)/C=C/CN(C)C)ccc34)nc(N3CCC4(CCOC4)CC3)nc12] |
| 118 | 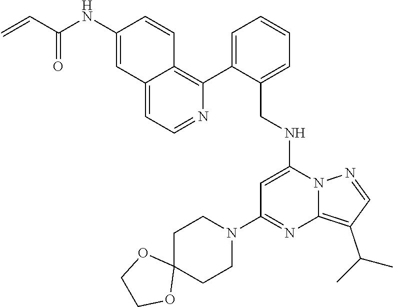 [SMILES: C=CC(=O)Nc1ccc2c(-c3ccccc3CNc3cc(N4CCC5(CC4)OCCO5)nc4c(C(C)C)cnn34)nccc2c1] |
| 119 | 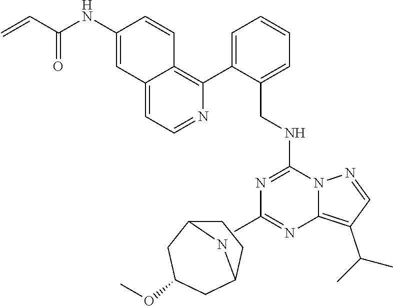 |
| 120 | 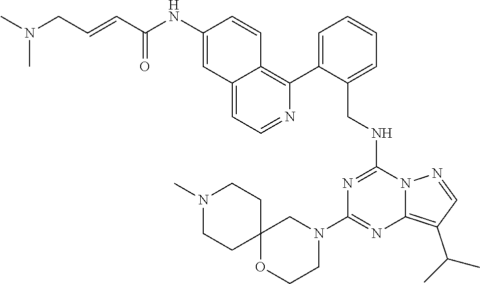 [SMILES: CC(C)c1cnn2c(NCc3ccccc3-c3nccc4cc(NC(=O)/C=C/CN(C)C)ccc34)nc(N3CCOC4(CCN(C)CC4)C3)nc12] |
| 121 | 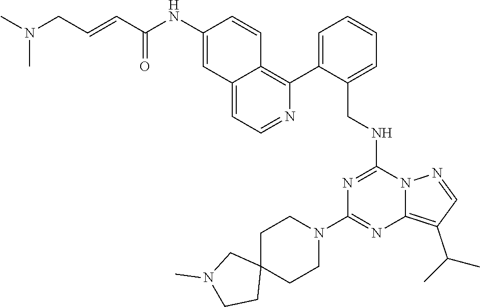 [SMILES: CC(C)c1cnn2c(NCc3ccccc3-c3nccc4cc(NC(=O)/C=C/CN(C)C)ccc34)nc(N3CCC4(CCN(C)C4)CC3)nc12] |
| 122 | 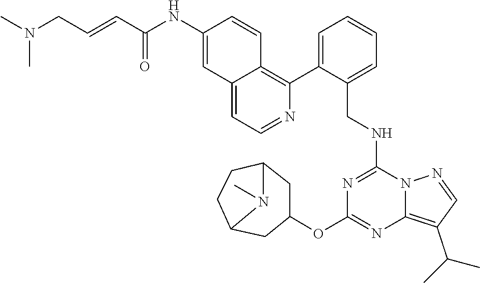 [SMILES: CC(C)c1cnn2c(NCc3ccccc3-c3nccc4cc(NC(=O)/C=C/CN(C)C)ccc34)nc(OC3CC4CCC(C3)N4C)nc12] |
| 123 | 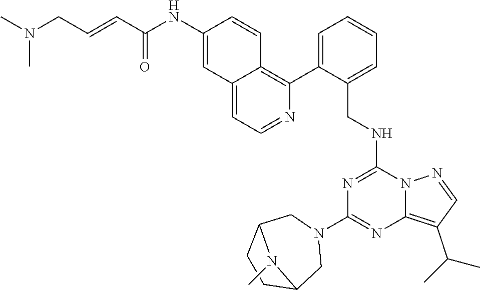 [SMILES: CC(C)c1cnn2c(NCc3ccccc3-c3nccc4cc(NC(=O)/C=C/CN(C)C)ccc34)nc(N3CC4CCC(C3)N4C)nc12] |
| 124 | 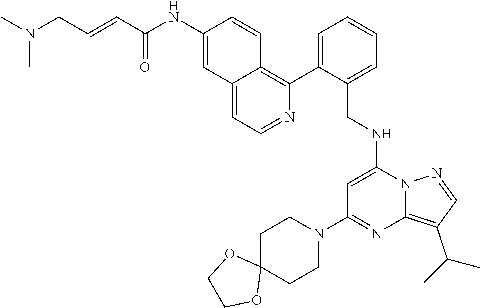 [SMILES: CC(C)c1cnn2c(NCc3ccccc3-c3nccc4cc(NC(=O)/C=C/CN(C)C)ccc34)cc(N3CCC4(CC3)OCCO4)nc12] |
| 125 | 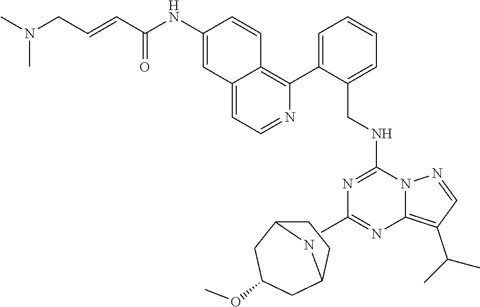 |
| 126 | 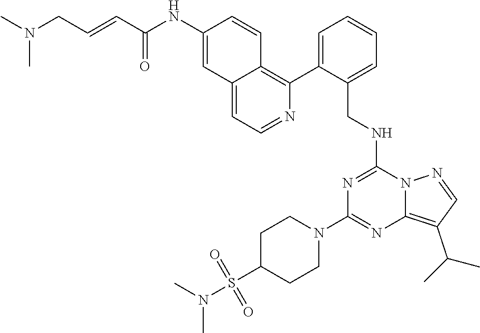 [SMILES: CC(C)c1cnn2c(NCc3ccccc3-c3nccc4cc(NC(=O)/C=C/CN(C)C)ccc34)nc(N3CCC(S(=O)(=O)N(C)C)CC3)nc12] |
| 127 | 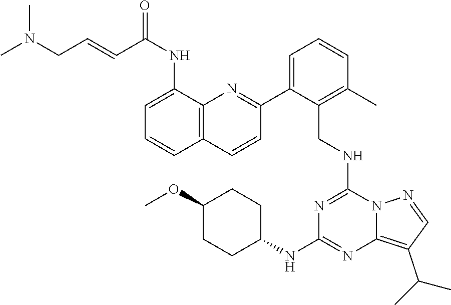 [SMILES: CO[C@H]1CC[C@H](Nc2nc(NCc3c(C)cccc3-c3ccc4cccc(NC(=O)/C=C/CN(C)C)c4n3)n3ncc(C(C)C)c3n2)CC1] |
| 128 | 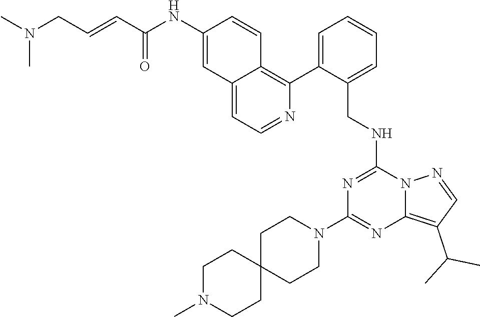 [SMILES: CC(C)c1cnn2c(NCc3ccccc3-c3nccc4cc(NC(=O)/C=C/CN(C)C)ccc34)nc(N3CCC4(CCN(C)CC4)CC3)nc12] |
| 129 | 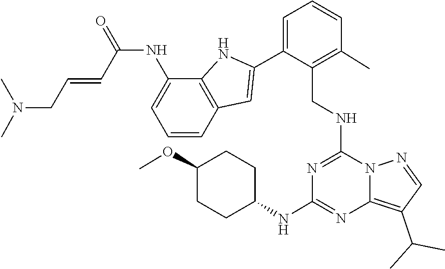 [SMILES: CO[C@H]1CC[C@H](Nc2nc(NCc3c(C)cccc3-c3cc4cccc(NC(=O)/C=C/CN(C)C)c4[nH]3)n3ncc(C(C)C)c3n2)CC1] |
| 130 | 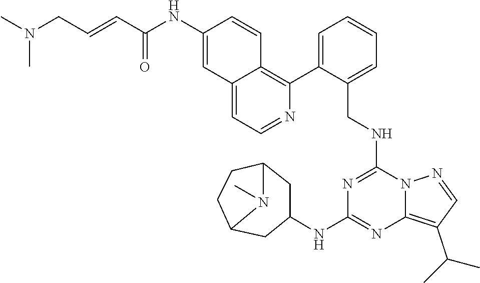 [SMILES: CC(C)c1cnn2c(NCc3ccccc3-c3nccc4cc(NC(=O)/C=C/CN(C)C)ccc34)nc(NC3CC4CCC(C3)N4C)nc12] |
| 131 | 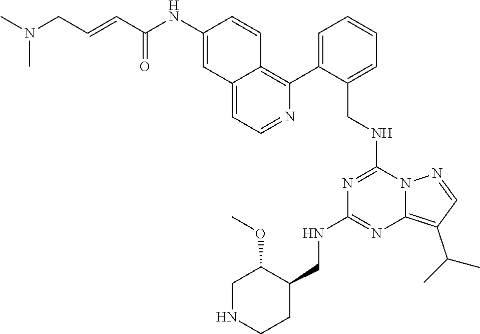 [SMILES: CO[C@H]1CNCC[C@@H]1CNc1nc(NCc2ccccc2-c2nccc3cc(NC(=O)/C=C/CN(C)C)ccc23)n2ncc(C(C)C)c2n1] |
| 132 | 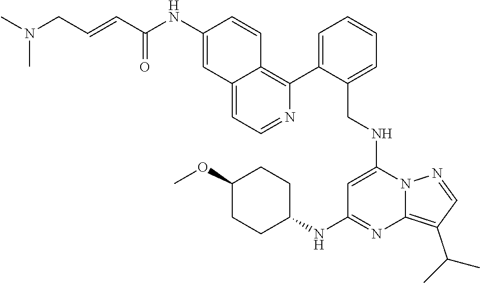 [SMILES: CO[C@H]1CC[C@H](Nc2cc(NCc3ccccc3-c3nccc4cc(NC(=O)/C=C/CN(C)C)ccc34)n3ncc(C(C)C)c3n2)CC1] |
| 133 | 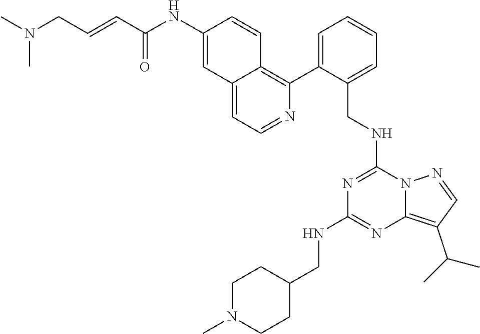 [SMILES: CC(C)c1cnn2c(NCc3ccccc3-c3nccc4cc(NC(=O)/C=C/CN(C)C)ccc34)nc(NCC3CCN(C)CC3)nc12] |
| 134 | 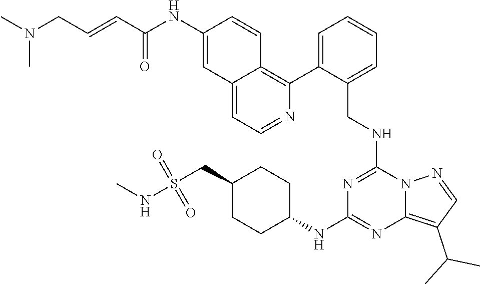 [SMILES: CNS(=O)(=O)C[C@H]1CC[C@H](Nc2nc(NCc3ccccc3-c3nccc4cc(NC(=O)/C=C/CN(C)C)ccc34)n3ncc(C(C)C)c3n2)CC1] |
| 135 | 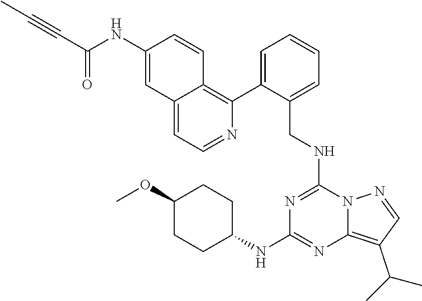 [SMILES: CC#CC(=O)Nc1ccc2c(-c3ccccc3CNc3nc(N[C@H]4CC[C@H](OC)CC4)nc4c(C(C)C)cnn34)nccc2c1] |
| 136 | 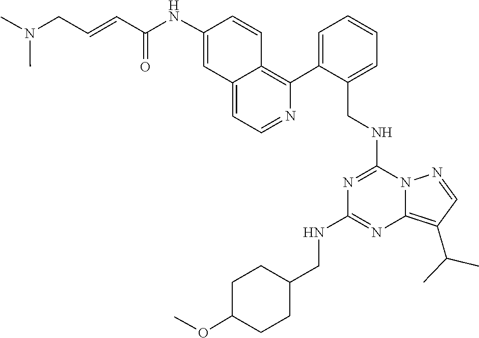 [SMILES: COC1CCC(CNc2nc(NCc3ccccc3-c3nccc4cc(NC(=O)/C=C/CN(C)C)ccc34)n3ncc(C(C)C)c3n2)CC1] |
| 137 | 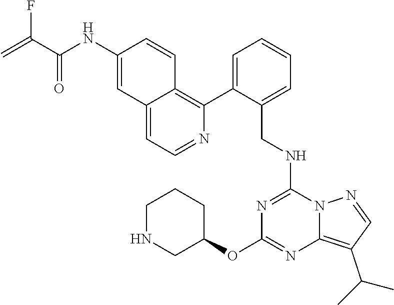 [SMILES: C=C(F)C(=O)Nc1ccc2c(-c3ccccc3CNc3nc(O[C@@H]4CCCNC4)nc4c(C(C)C)cnn34)nccc2c1] |
| 138 | 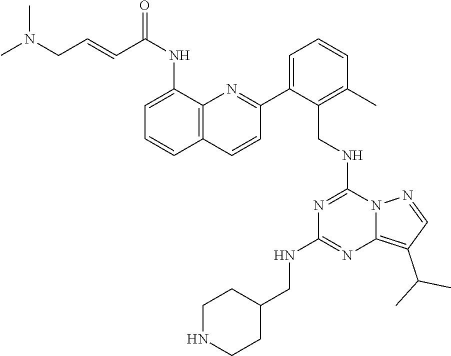 [SMILES: Cc1cccc(-c2ccc3cccc(NC(=O)/C=C/CN(C)C)c3n2)c1CNc1nc(NCC2CCNCC2)nc2c(C(C)C)cnn12] |
| 139 | 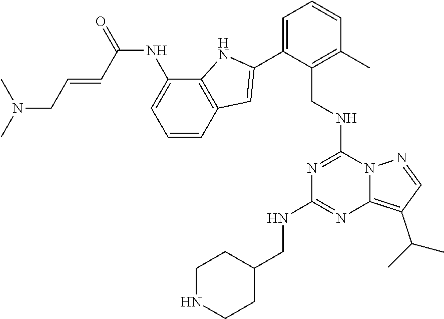 [SMILES: Cc1cccc(-c2cc3cccc(NC(=O)/C=C/CN(C)C)c3[nH]2)c1CNc1nc(NCC2CCNCC2)nc2c(C(C)C)cnn12] |
| 140 | 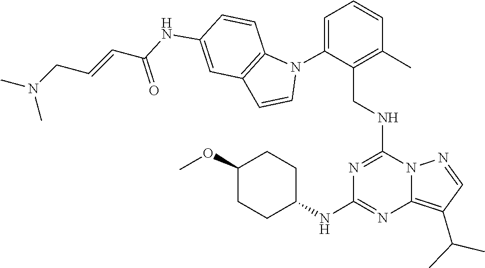 [SMILES: CO[C@H]1CC[C@H](Nc2nc(NCc3c(C)cccc3-n3ccc4cc(NC(=O)/C=C/CN(C)C)ccc43)n3ncc(C(C)C)c3n2)CC1] |
| 141 | 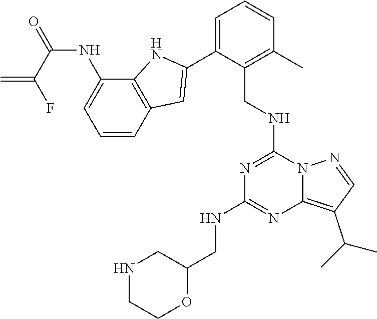 [SMILES: C=C(F)C(=O)Nc1cccc2cc(-c3cccc(C)c3CNc3nc(NCC4CNCCO4)nc4c(C(C)C)cnn34)[nH]c12] |
| 142 | 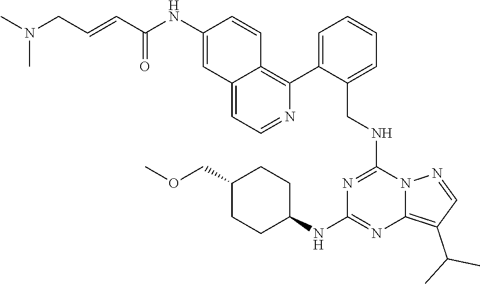 [SMILES: COC[C@H]1CC[C@H](Nc2nc(NCc3ccccc3-c3nccc4cc(NC(=O)/C=C/CN(C)C)ccc34)n3ncc(C(C)C)c3n2)CC1] |
| 143 | 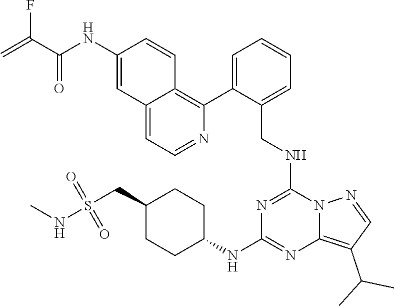 [SMILES: C=C(F)C(=O)Nc1ccc2c(-c3ccccc3CNc3nc(N[C@H]4CC[C@H](CS(=O)(=O)NC)CC4)nc4c(C(C)C)cnn34)nccc2c1] |
| 144 | 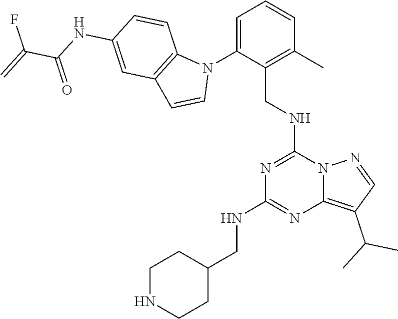 [SMILES: C=C(F)C(=O)Nc1ccc2c(ccn2-c2cccc(C)c2CNc2nc(NCC3CCNCC3)nc3c(C(C)C)cnn23)c1] |
| 145 | 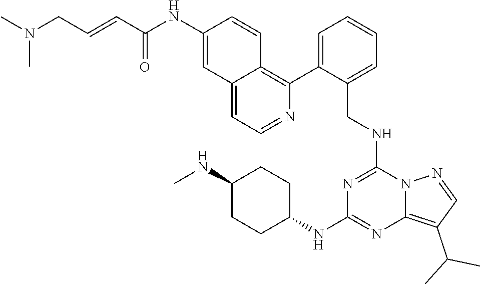 [SMILES: CN[C@H]1CC[C@H](Nc2nc(NCc3ccccc3-c3nccc4cc(NC(=O)/C=C/CN(C)C)ccc34)n3ncc(C(C)C)c3n2)CC1] |
| 146 | 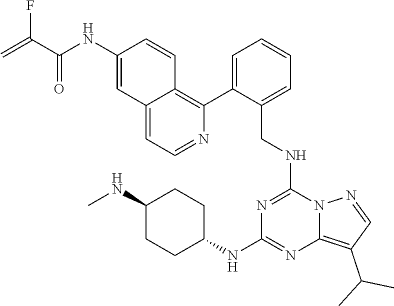 [SMILES: C=C(F)C(=O)Nc1ccc2c(-c3ccccc3CNc3nc(N[C@H]4CC[C@H](NC)CC4)nc4c(C(C)C)cnn34)nccc2c1] |
| 147 | 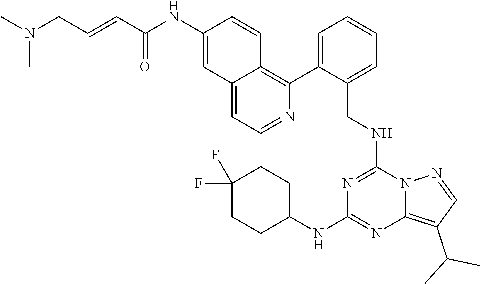 [SMILES: CC(C)c1cnn2c(NCc3ccccc3-c3nccc4cc(NC(=O)/C=C/CN(C)C)ccc34)nc(NC3CCC(F)(F)CC3)nc12] |
| 148 | 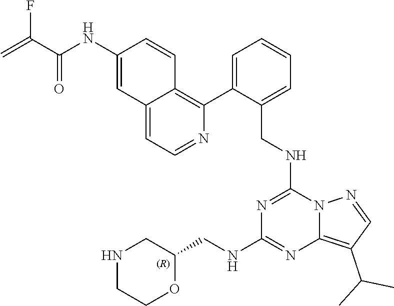 |
| 149 | 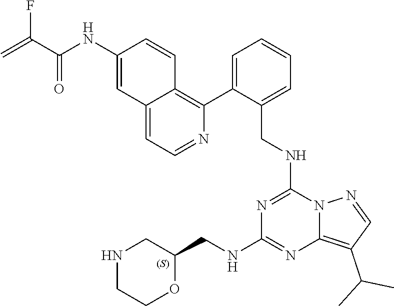 |
| 150 | 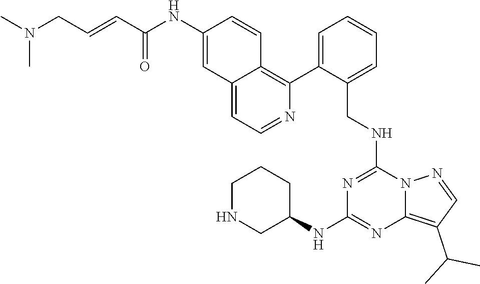 [SMILES: CC(C)c1cnn2c(NCc3ccccc3-c3nccc4cc(NC(=O)/C=C/CN(C)C)ccc34)nc(N[C@@H]3CCCNC3)nc12] |
| 151 | 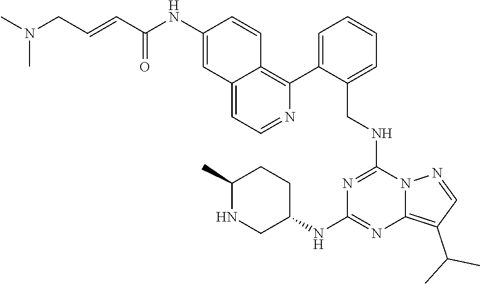 [SMILES: CC(C)c1cnn2c(NCc3ccccc3-c3nccc4cc(NC(=O)/C=C/CN(C)C)ccc34)nc(N[C@H]3CC[C@H](C)NC3)nc12] |
| 152 | 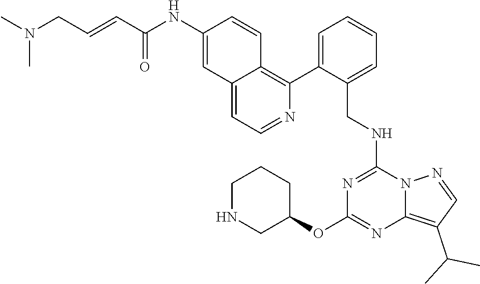 [SMILES: CC(C)c1cnn2c(NCc3ccccc3-c3nccc4cc(NC(=O)/C=C/CN(C)C)ccc34)nc(O[C@@H]3CCCNC3)nc12] |
| 153 | 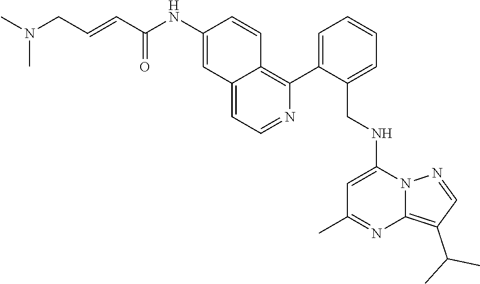 [SMILES: Cc1cc(NCc2ccccc2-c2nccc3cc(NC(=O)/C=C/CN(C)C)ccc23)n2ncc(C(C)C)c2n1] |
| 154 | 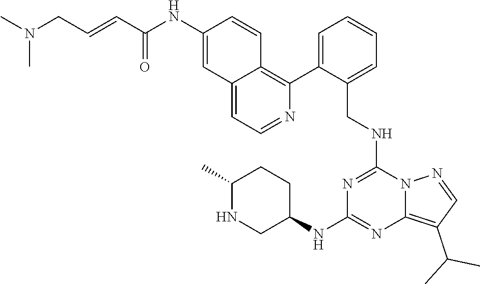 [SMILES: CC(C)c1cnn2c(NCc3ccccc3-c3nccc4cc(NC(=O)/C=C/CN(C)C)ccc34)nc(N[C@@H]3CC[C@@H](C)NC3)nc12] |
| 155 | 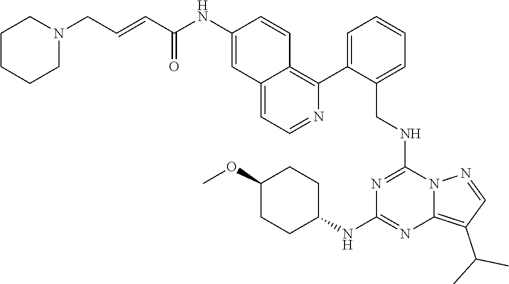 [SMILES: CO[C@H]1CC[C@H](Nc2nc(NCc3ccccc3-c3nccc4cc(NC(=O)/C=C/CN5CCCCC5)ccc34)n3ncc(C(C)C)c3n2)CC1] |
| 156 | 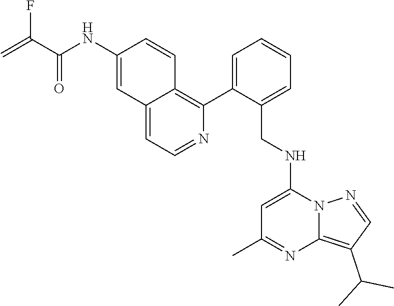 [SMILES: C=C(F)C(=O)Nc1ccc2c(-c3ccccc3CNc3cc(C)nc4c(C(C)C)cnn34)nccc2c1] |
| 157 | 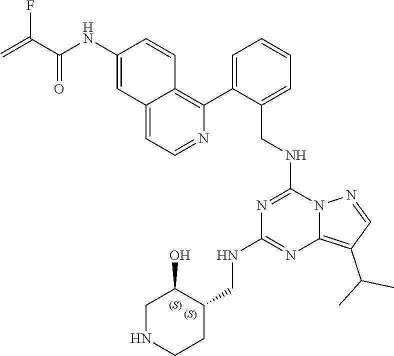 |
| 158 | 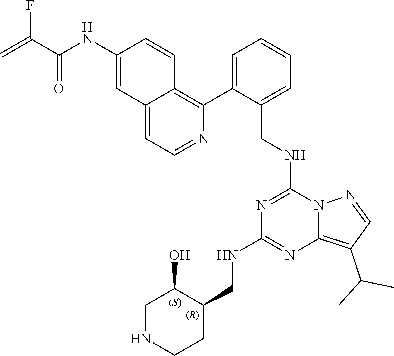 |
| 159 | 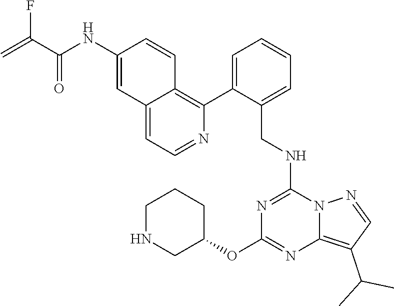 [SMILES: C=C(F)C(=O)Nc1ccc2c(-c3ccccc3CNc3nc(O[C@H]4CCCNC4)nc4c(C(C)C)cnn34)nccc2c1] |
| 160 | 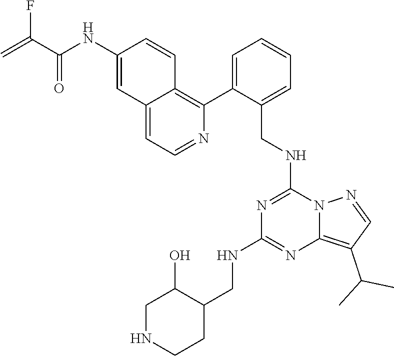 [SMILES: C=C(F)C(=O)Nc1ccc2c(-c3ccccc3CNc3nc(NCC4CCNCC4O)nc4c(C(C)C)cnn34)nccc2c1] |
| 161 | 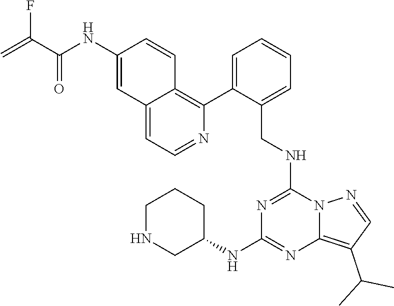 [SMILES: C=C(F)C(=O)Nc1ccc2c(-c3ccccc3CNc3nc(N[C@H]4CCCNC4)nc4c(C(C)C)cnn34)nccc2c1] |
| 162 | 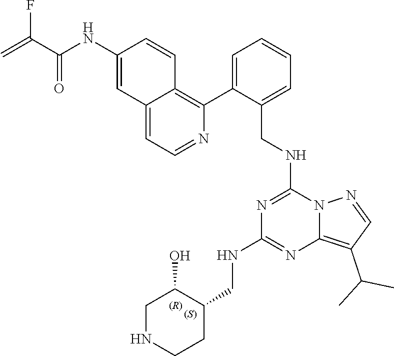 |
| 163 | 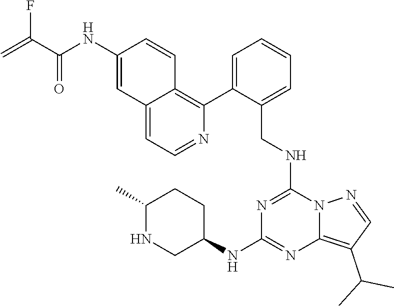 [SMILES: C=C(F)C(=O)Nc1ccc2c(-c3ccccc3CNc3nc(N[C@@H]4CC[C@@H](C)NC4)nc4c(C(C)C)cnn34)nccc2c1] |
| 164 | 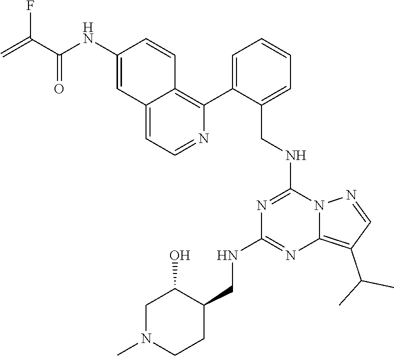 [SMILES: C=C(F)C(=O)Nc1ccc2c(-c3ccccc3CNc3nc(NC[C@H]4CCN(C)C[C@@H]4O)nc4c(C(C)C)cnn34)nccc2c1] |
| 165 | 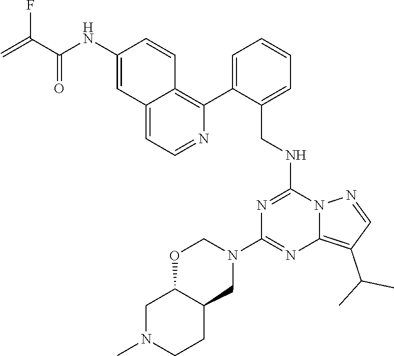 [SMILES: C=C(F)C(=O)Nc1ccc2c(-c3ccccc3CNc3nc(N4CO[C@H]5CN(C)CC[C@@H]5C4)nc4c(C(C)C)cnn34)nccc2c1] |
| 166 | 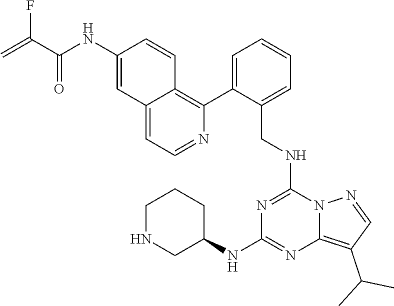 [SMILES: C=C(F)C(=O)Nc1ccc2c(-c3ccccc3CNc3nc(N[C@@H]4CCCNC4)nc4c(C(C)C)cnn34)nccc2c1] |
| 167 | 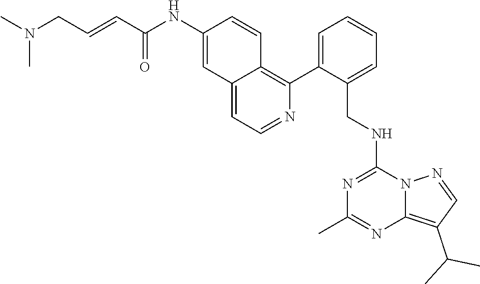 [SMILES: Cc1nc(NCc2ccccc2-c2nccc3cc(NC(=O)/C=C/CN(C)C)ccc23)n2ncc(C(C)C)c2n1] |
| 168 | 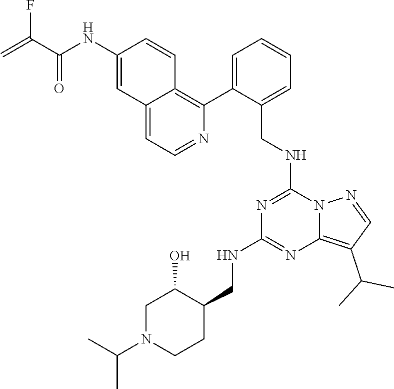 [SMILES: C=C(F)C(=O)Nc1ccc2c(-c3ccccc3CNc3nc(NC[C@H]4CCN(C(C)C)C[C@@H]4O)nc4c(C(C)C)cnn34)nccc2c1] |
| 169 | 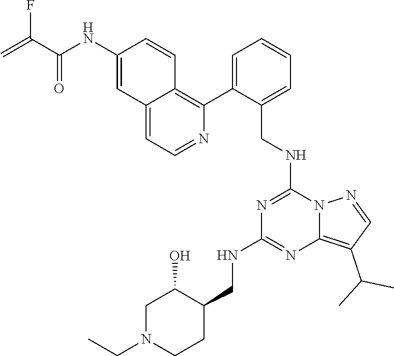 [SMILES: C=C(F)C(=O)Nc1ccc2c(-c3ccccc3CNc3nc(NC[C@H]4CCN(CC)C[C@@H]4O)nc4c(C(C)C)cnn34)nccc2c1] |
| 170 | 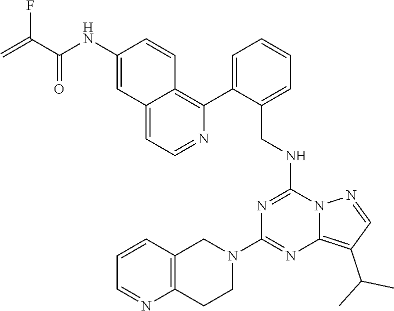 [SMILES: C=C(F)C(=O)Nc1ccc2c(-c3ccccc3CNc3nc(N4CCc5ncccc5C4)nc4c(C(C)C)cnn34)nccc2c1] |
| 171 | 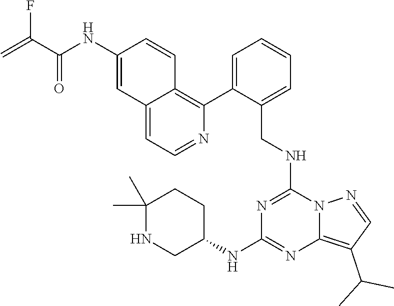 [SMILES: C=C(F)C(=O)Nc1ccc2c(-c3ccccc3CNc3nc(N[C@H]4CCC(C)(C)NC4)nc4c(C(C)C)cnn34)nccc2c1] |
| 172 | 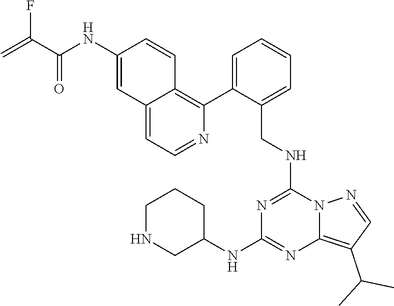 [SMILES: C=C(F)C(=O)Nc1ccc2c(-c3ccccc3CNc3nc(NC4CCCNC4)nc4c(C(C)C)cnn34)nccc2c1] |
| 173 | 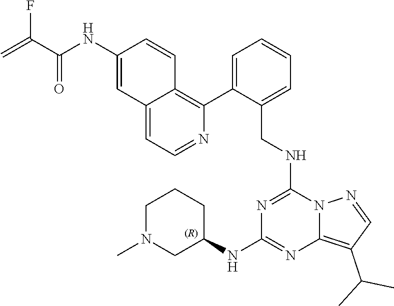 |
| 174 | 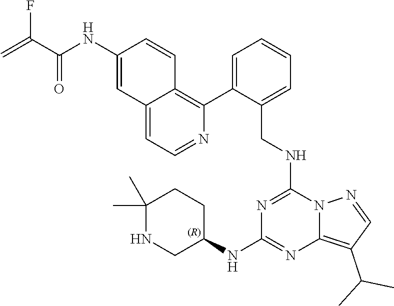 |
| 175 | 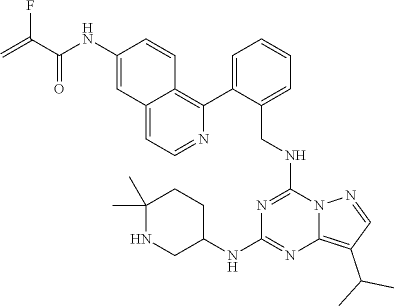 [SMILES: C=C(F)C(=O)Nc1ccc2c(-c3ccccc3CNc3nc(NC4CCC(C)(C)NC4)nc4c(C(C)C)cnn34)nccc2c1] |
| 176 | 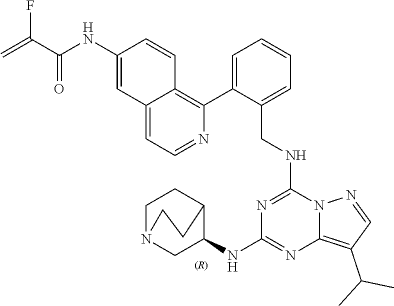 |
| 177 | 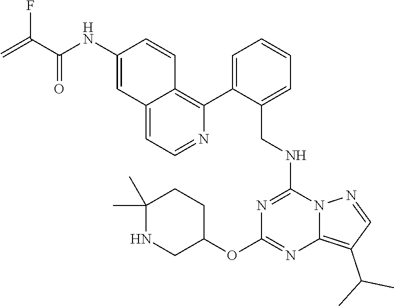 [SMILES: C=C(F)C(=O)Nc1ccc2c(-c3ccccc3CNc3nc(OC4CCC(C)(C)NC4)nc4c(C(C)C)cnn34)nccc2c1] |
| 178 | 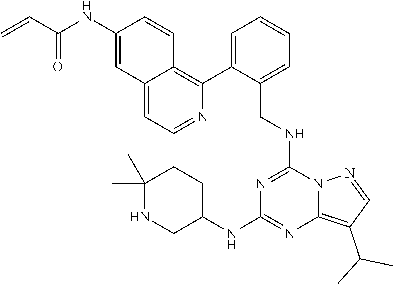 [SMILES: C=CC(=O)Nc1ccc2c(-c3ccccc3CNc3nc(NC4CCC(C)(C)NC4)nc4c(C(C)C)cnn34)nccc2c1] |
| 179 | 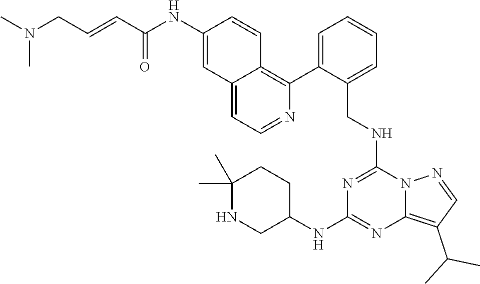 [SMILES: CC(C)c1cnn2c(NCc3ccccc3-c3nccc4cc(NC(=O)/C=C/CN(C)C)ccc34)nc(NC3CCC(C)(C)NC3)nc12] |
| 180 | 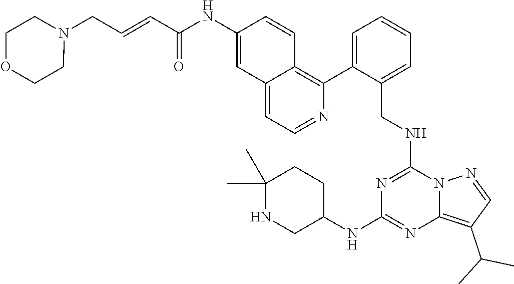 [SMILES: CC(C)c1cnn2c(NCc3ccccc3-c3nccc4cc(NC(=O)/C=C/CN5CCOCC5)ccc34)nc(NC3CCC(C)(C)NC3)nc12] |
| 181 | 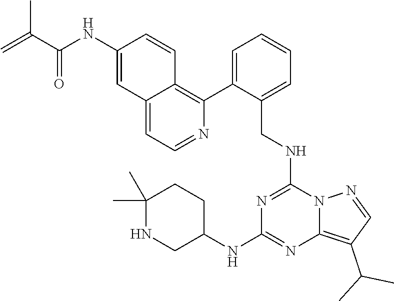 [SMILES: C=C(C)C(=O)Nc1ccc2c(-c3ccccc3CNc3nc(NC4CCC(C)(C)NC4)nc4c(C(C)C)cnn34)nccc2c1] |
| 182 | 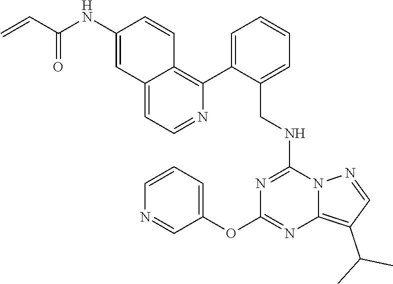 [SMILES: C=CC(=O)Nc1ccc2c(-c3ccccc3CNc3nc(Oc4cccnc4)nc4c(C(C)C)cnn34)nccc2c1] |
| 183 | 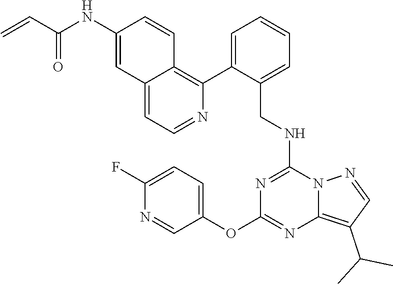 [SMILES: C=CC(=O)Nc1ccc2c(-c3ccccc3CNc3nc(Oc4ccc(F)nc4)nc4c(C(C)C)cnn34)nccc2c1] |
| 184 | 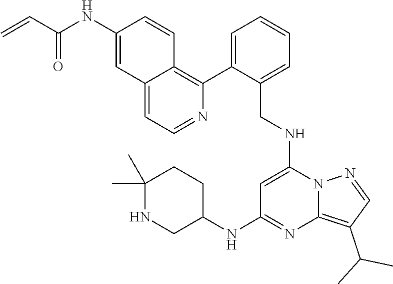 [SMILES: C=CC(=O)Nc1ccc2c(-c3ccccc3CNc3cc(NC4CCC(C)(C)NC4)nc4c(C(C)C)cnn34)nccc2c1] |
| 185 | 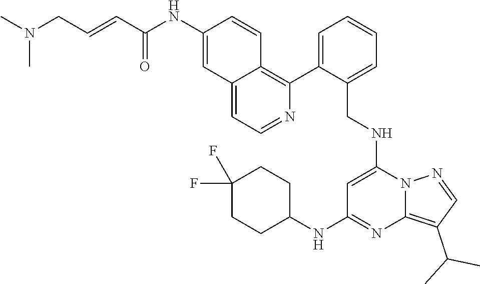 [SMILES: CC(C)c1cnn2c(NCc3ccccc3-c3nccc4cc(NC(=O)/C=C/CN(C)C)ccc34)cc(NC3CCC(F)(F)CC3)nc12] |
| 186 | 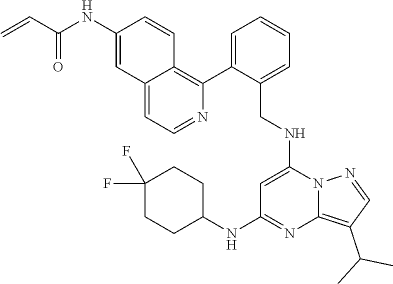 [SMILES: C=CC(=O)Nc1ccc2c(-c3ccccc3CNc3cc(NC4CCC(F)(F)CC4)nc4c(C(C)C)cnn34)nccc2c1] |
| 187 | 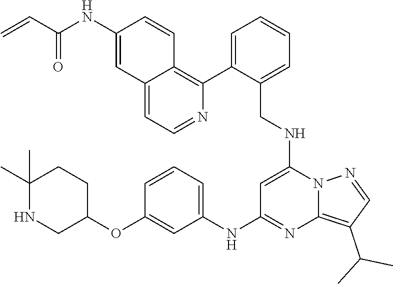 [SMILES: C=CC(=O)Nc1ccc2c(-c3ccccc3CNc3cc(Nc4cccc(OC5CCC(C)(C)NC5)c4)nc4c(C(C)C)cnn34)nccc2c1] |
| 188 | 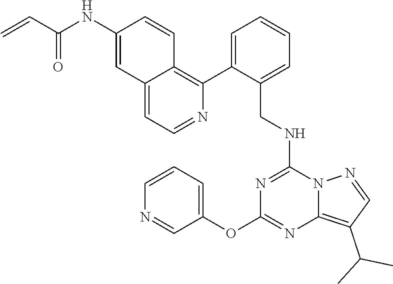 [SMILES: C=CC(=O)Nc1ccc2c(-c3ccccc3CNc3nc(Oc4cccnc4)nc4c(C(C)C)cnn34)nccc2c1] |
| 189 | 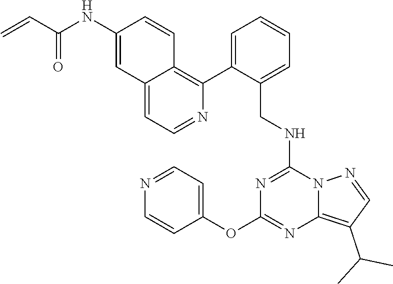 [SMILES: C=CC(=O)Nc1ccc2c(-c3ccccc3CNc3nc(Oc4ccncc4)nc4c(C(C)C)cnn34)nccc2c1] |
| 190 | 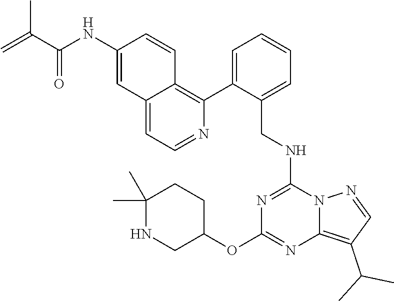 [SMILES: C=C(C)C(=O)Nc1ccc2c(-c3ccccc3CNc3nc(OC4CCC(C)(C)NC4)nc4c(C(C)C)cnn34)nccc2c1] |
| 191 | 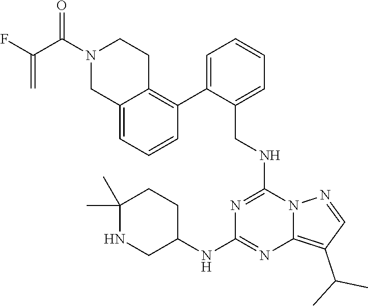 [SMILES: C=C(F)C(=O)N1CCc2c(cccc2-c2ccccc2CNc2nc(NC3CCC(C)(C)NC3)nc3c(C(C)C)cnn23)C1] |
| 192 | 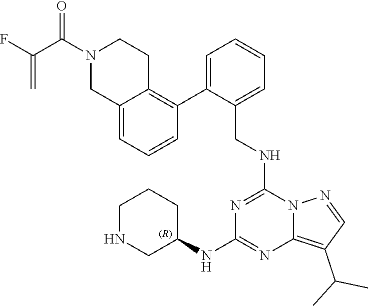 |
| 193 | 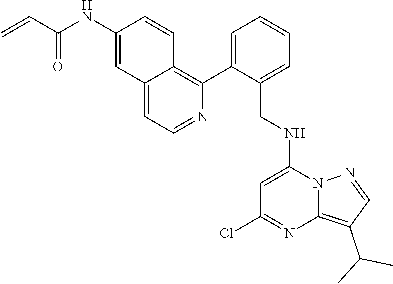 [SMILES: C=CC(=O)Nc1ccc2c(-c3ccccc3CNc3cc(Cl)nc4c(C(C)C)cnn34)nccc2c1] |
| 194 | 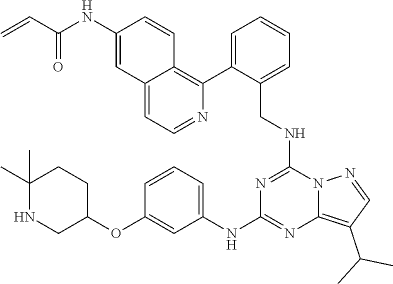 [SMILES: C=CC(=O)Nc1ccc2c(-c3ccccc3CNc3nc(Nc4cccc(OC5CCC(C)(C)NC5)c4)nc4c(C(C)C)cnn34)nccc2c1] |
| 195 | 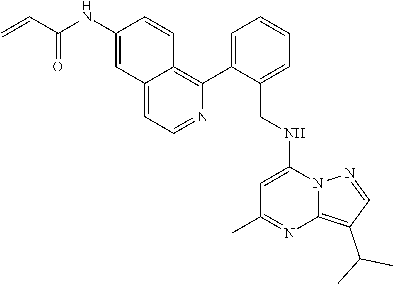 [SMILES: C=CC(=O)Nc1ccc2c(-c3ccccc3CNc3cc(C)nc4c(C(C)C)cnn34)nccc2c1] |
| 196 | 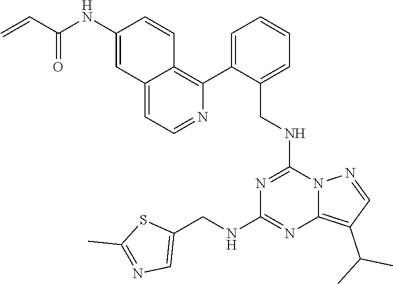 [SMILES: C=CC(=O)Nc1ccc2c(-c3ccccc3CNc3nc(NCc4cnc(C)s4)nc4c(C(C)C)cnn34)nccc2c1] |
| 197 | 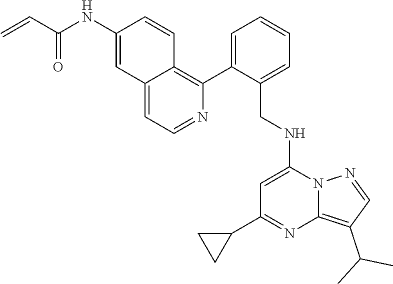 [SMILES: C=CC(=O)Nc1ccc2c(-c3ccccc3CNc3cc(C4CC4)nc4c(C(C)C)cnn34)nccc2c1] |
| 198 | 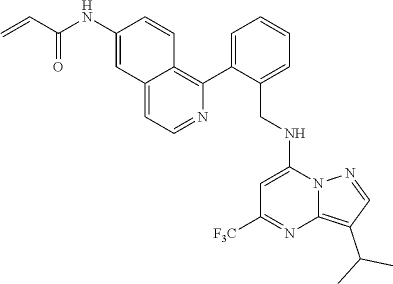 [SMILES: C=CC(=O)Nc1ccc2c(-c3ccccc3CNc3cc(C(F)(F)F)nc4c(C(C)C)cnn34)nccc2c1] |
| 199 | 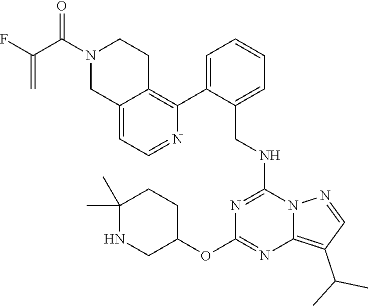 [SMILES: C=C(F)C(=O)N1CCc2c(ccnc2-c2ccccc2CNc2nc(OC3CCC(C)(C)NC3)nc3c(C(C)C)cnn23)C1] |
| 200 | 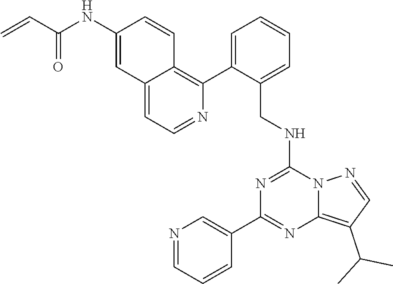 [SMILES: C=CC(=O)Nc1ccc2c(-c3ccccc3CNc3nc(-c4cccnc4)nc4c(C(C)C)cnn34)nccc2c1] |
| 201 | 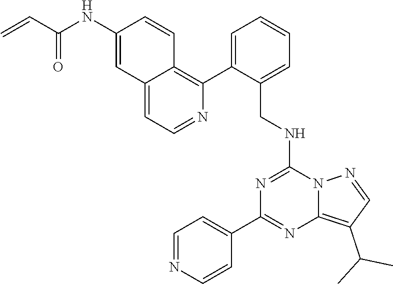 [SMILES: C=CC(=O)Nc1ccc2c(-c3ccccc3CNc3nc(-c4ccncc4)nc4c(C(C)C)cnn34)nccc2c1] |
| 202 | 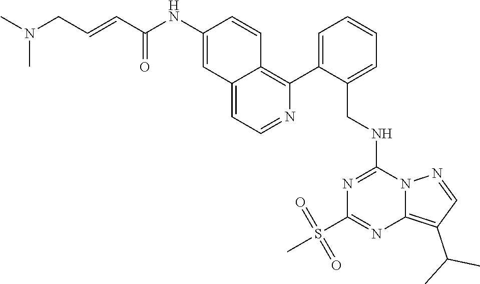 [SMILES: CC(C)c1cnn2c(NCc3ccccc3-c3nccc4cc(NC(=O)/C=C/CN(C)C)ccc34)nc(S(C)(=O)=O)nc12] |
| 203 | 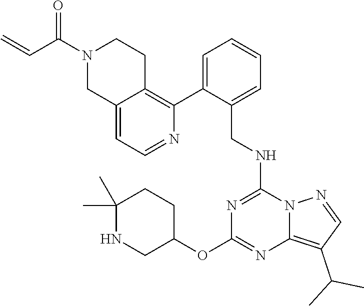 [SMILES: C=CC(=O)N1CCc2c(ccnc2-c2ccccc2CNc2nc(OC3CCC(C)(C)NC3)nc3c(C(C)C)cnn23)C1] |
| 204 | 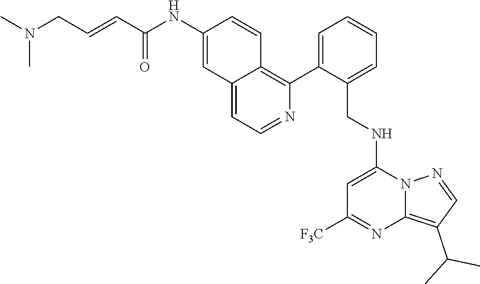 [SMILES: CC(C)c1cnn2c(NCc3ccccc3-c3nccc4cc(NC(=O)/C=C/CN(C)C)ccc34)cc(C(F)(F)F)nc12] |
| 205 | 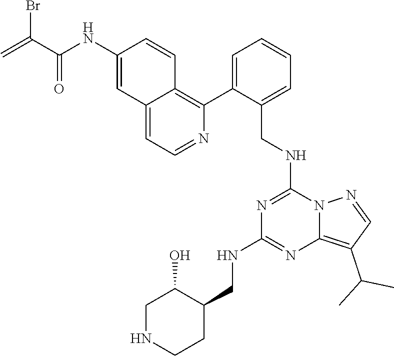 [SMILES: C=C(Br)C(=O)Nc1ccc2c(-c3ccccc3CNc3nc(NC[C@H]4CCNC[C@@H]4O)nc4c(C(C)C)cnn34)nccc2c1] |
| 206 | 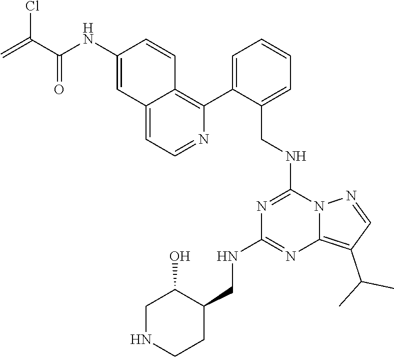 [SMILES: C=C(Cl)C(=O)Nc1ccc2c(-c3ccccc3CNc3nc(NC[C@H]4CCNC[C@@H]4O)nc4c(C(C)C)cnn34)nccc2c1] |
| 207 | 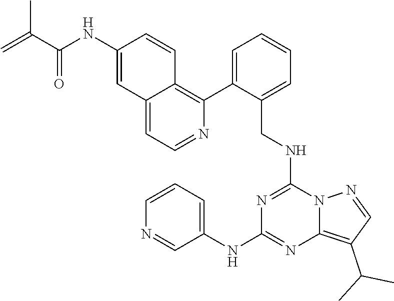 [SMILES: C=C(C)C(=O)Nc1ccc2c(-c3ccccc3CNc3nc(Nc4cccnc4)nc4c(C(C)C)cnn34)nccc2c1] |
| 208 | 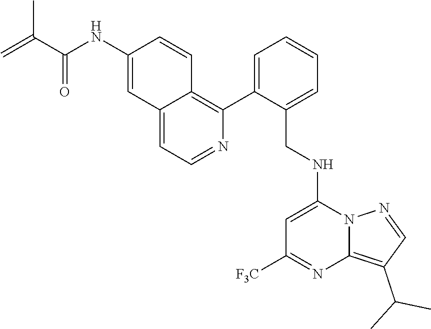 [SMILES: C=C(C)C(=O)Nc1ccc2c(-c3ccccc3CNc3cc(C(F)(F)F)nc4c(C(C)C)cnn34)nccc2c1] |
| 209 | 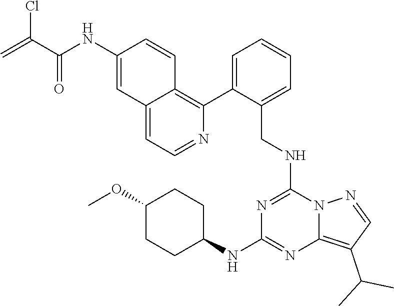 [SMILES: C=C(Cl)C(=O)Nc1ccc2c(-c3ccccc3CNc3nc(N[C@H]4CC[C@H](OC)CC4)nc4c(C(C)C)cnn34)nccc2c1] |
| 210 | 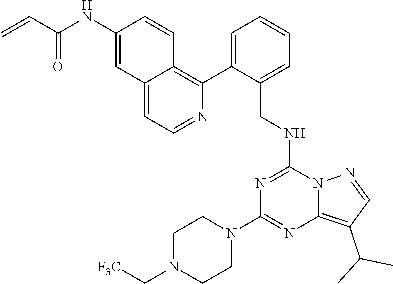 [SMILES: C=CC(=O)Nc1ccc2c(-c3ccccc3CNc3nc(N4CCN(CC(F)(F)F)CC4)nc4c(C(C)C)cnn34)nccc2c1] |
| 211 | 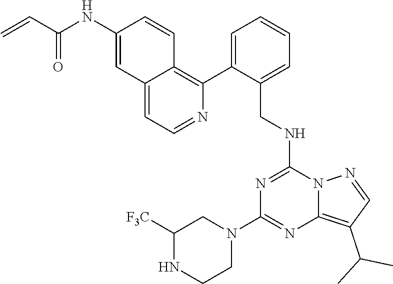 [SMILES: C=CC(=O)Nc1ccc2c(-c3ccccc3CNc3nc(N4CCNC(C(F)(F)F)C4)nc4c(C(C)C)cnn34)nccc2c1] |
| 212 | 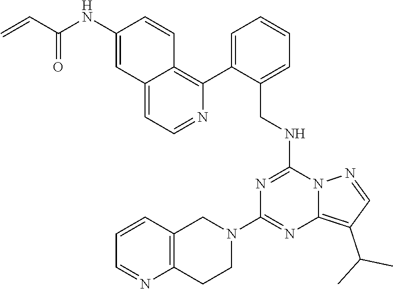 [SMILES: C=CC(=O)Nc1ccc2c(-c3ccccc3CNc3nc(N4CCc5ncccc5C4)nc4c(C(C)C)cnn34)nccc2c1] |
| 213 | 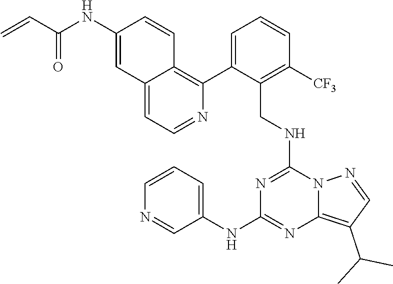 [SMILES: C=CC(=O)Nc1ccc2c(-c3cccc(C(F)(F)F)c3CNc3nc(Nc4cccnc4)nc4c(C(C)C)cnn34)nccc2c1] |
| 214 | 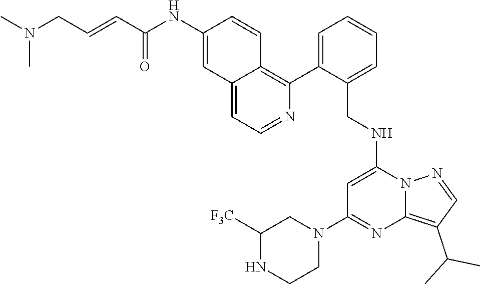 [SMILES: CC(C)c1cnn2c(NCc3ccccc3-c3nccc4cc(NC(=O)/C=C/CN(C)C)ccc34)cc(N3CCNC(C(F)(F)F)C3)nc12] |
| 215 | 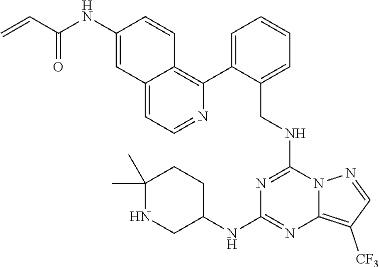 [SMILES: C=CC(=O)Nc1ccc2c(-c3ccccc3CNc3nc(NC4CCC(C)(C)NC4)nc4c(C(F)(F)F)cnn34)nccc2c1] |
| 216 | 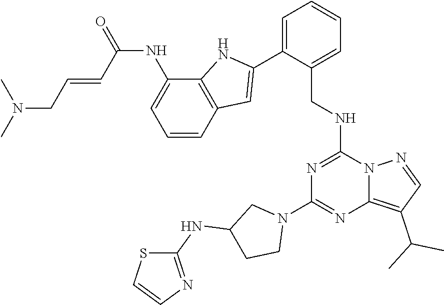 [SMILES: CC(C)c1cnn2c(NCc3ccccc3-c3cc4cccc(NC(=O)/C=C/CN(C)C)c4[nH]3)nc(N3CCC(Nc4nccs4)C3)nc12] |

In one embodiment, said compound is a compound having a structure selected from structures 44, 64, 95, 134, 147, 164, 174, 175, 177, and 178, as defined herein, wherein, preferably, said compound is a compound having a structure selected from structures 64, 134, 164, 174, 175, 177 and 178, as defined herein, wherein, more preferably, said compound is a compound having a structure selected from 174, 175, and 177, as defined herein.

In a further aspect, the present invention also relates to a method of treatment of aDNA-virus infection in a subject, said DNA-virus preferably being selected from Herpesviridae and Papillomaviridae, said method comprising administering a compound having the general formula I

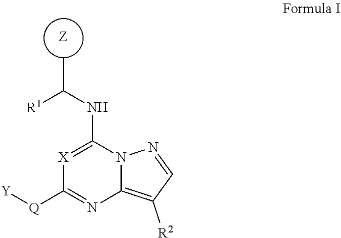

or an enantiomer, stereoisomeric form, mixture of enantiomers, diastereomer, mixture of diastereomers, racemate, or a pharmaceutically acceptable salt thereof, to a subject in need thereof;
wherein, in said compound,
X is, independently at each occurrence, selected from CH and N;
Q is either absent or independently, at each occurrence, selected from the group consisting of —NH—, —NH(CH₂)—, —NH(CH₂)₂—, —NH(C═O)—, —NHSO₂—, —O—, —O(CH₂)—, —(C═O)—, —(C═O)NH—and —(C═O)(CH₂)—;
Y is, independently at each occurrence, selected from the group consisting of halogen, C1-C3 haloalkyl, C3-C8 cycloalkyl, aryl, heteroaryl, heterocyclyl, —S(═O)₂R³, C1-C6 alkyl and C1-C6 alkyl substituted with one or two of —OR⁵, —N(R⁵)R⁵, aryl, heteroaryl and heterocyclyl;
Wherein C3-C8 cycloalkyl is optionally substituted with one or two of R³, R⁴ and —(C═O)R⁵, wherein heterocyclyl is optionally substituted with one or two of R³, R⁴ and —(C═O)R⁵, and wherein aryl or heteroaryl is optionally substituted with one or two of R³, C1-C6 alkyl, —OR⁵, —N(R⁵)R⁵, —(C═O)R⁵, halogen, heteroaryl and heterocyclyl;
R¹ is, at each occurrence, independently selected from the group consisting of hydrogen and methyl; R² is, at each occurrence, independently selected from the group consisting of halogen, C1-C6 alkyl, C3-C10 cycloalkyl, —CN, —(C═O)CH₃ and C1-C3 haloalkyl, any of which is optionally substituted;
R³ is either absent or independently, at each occurrence, selected from the group consisting of hydrogen, —OR⁵, halogen, C1-C3 haloalkyl, —CN, —N(R⁵)R⁵, (═O), —NH(C═O)R⁵, —(C═O)NH₂, —S(═O)₂N(R⁵)R⁵, aryl, heteroaryl, heterocyclyl, C1-C6 alkyl and C1-C6 alkyl substituted with —OR₅, —NH₂ or —S(═O)₂N(R⁵)R⁵;
R⁴ is, independently, at each occurrence, selected from the group consisting of hydrogen, halogen, C1-C3 haloalkyl, —CN, —OR⁵, —N(R⁵)R⁵, (═O), S(═O)₂N(R⁵)R⁵, aryl, heteroaryl, heterocyclyl, C1-C6 alkyl and C1-C6 alkyl substituted with —OH, —NH₂ or —S(═O)₂N(R⁵)R⁵;
wherein both R³ and R⁴ are (═O) if attached to a single sulfur atom that forms part of Y being a heterocycle;
or wherein R³ and R⁴, together with the structure to which they are attached, form an aromatic ring, a heteroaromatic ring, a saturated or unsaturated heterocyclic ring, or a fused or bridged ring structure of any of an aromatic ring, a heteroaromatic ring, and a saturated or unsaturated heterocyclic ring;
R⁵ is, at each occurrence, independently selected from the group consisting of hydrogen, C1-C6 alkyl, C3-C6 cycloalkyl, C1-C3 haloalkyl, heteroaryl, heterocyclyl, heteroaryl substituted with one or two of halogen, —OR¹¹, —N(R¹¹)R¹¹, C1-C6 alkyl and C1-C6 alkyl substituted with —OH, —NH₂; heterocyclyl substituted with one or two of halogen, —OR¹¹, —N(R¹¹)R¹¹, C1-C6 alkyl and C1-C6 alkyl substituted with —OH or —NH₂;
Z is any structure of the following group A;

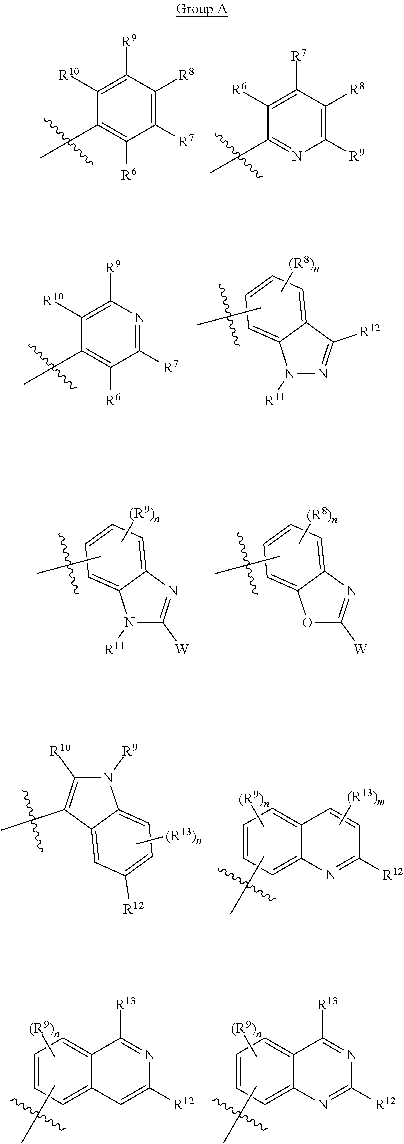

Wherein n=1, 2, or 3; m=1, or 2;
R⁶ and R⁷ are, at each occurrence, independently selected from the group consisting of hydrogen, —NH(C═O)R¹⁴, —NHR¹⁴, —OR¹⁴ and any structure of the following group B, with the
proviso that, when Z is

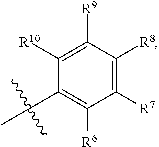

one of R⁶ and R⁷ is not H;

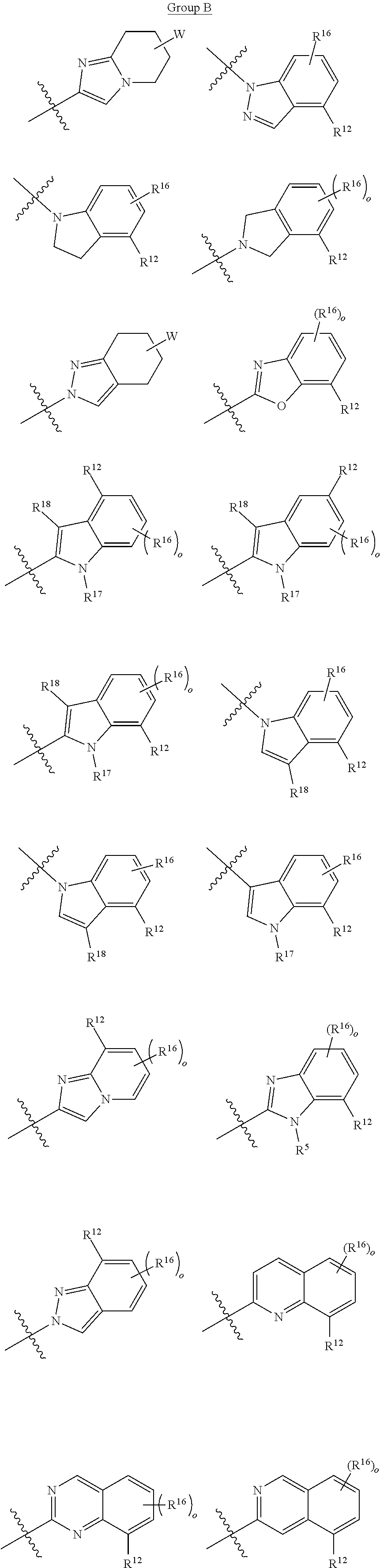

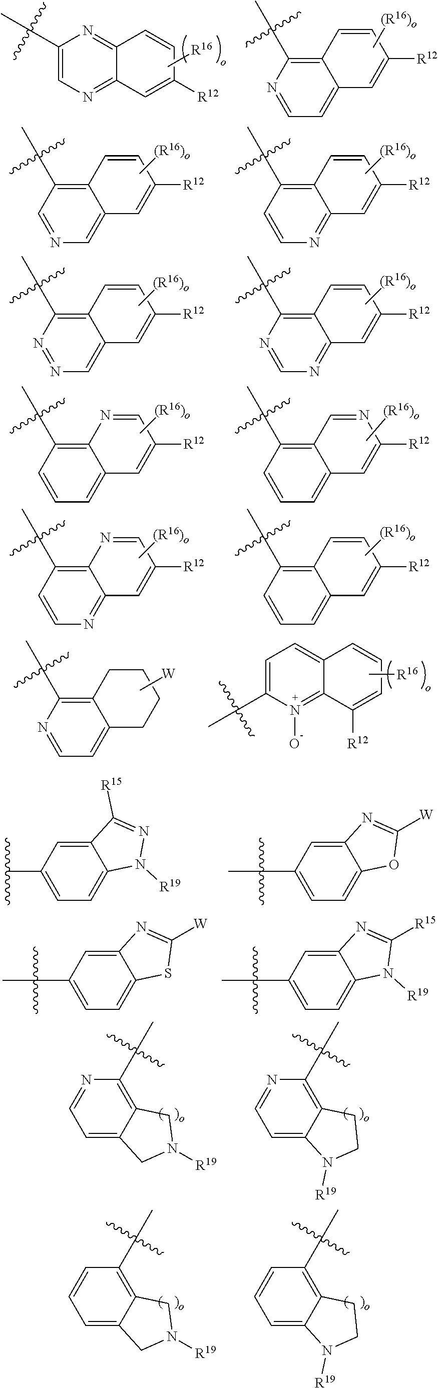

wherein o is, independently at each occurrence, selected from 1, 2 and 3;
W is any structure of the following group C;

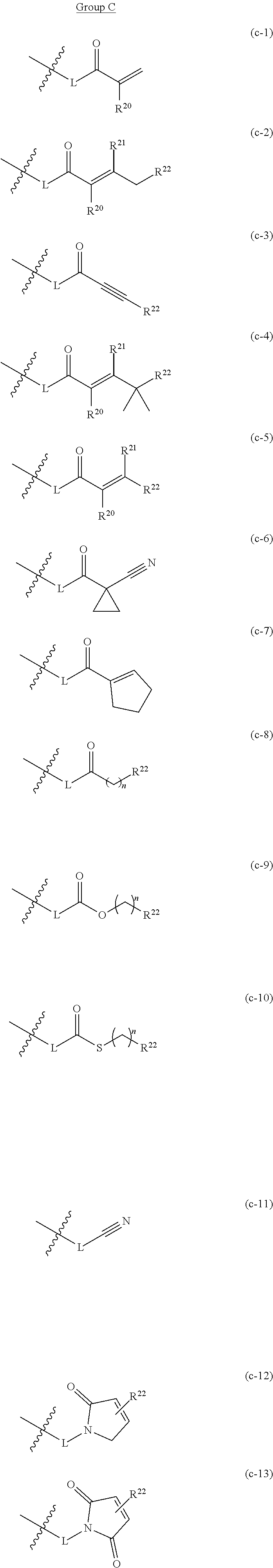

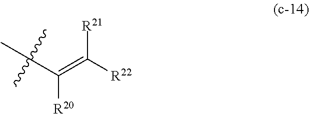

L is absent or, at each occurrence, independently selected from the group consisting of —O— and —NH—;
wherein n is, independently at each occurrence, selected from 1, 2 and 3;
R⁸, R⁹ and R¹⁰ are, at each occurrence, independently selected from the group consisting of hydrogen, halogen, C1-C6 alkyl, C1-C3 haloalkyl, —OR⁵, —CN and C1-C6 alkyl substituted with —OH, —OR⁵ or —NHR⁵;
R¹¹ is, at each occurrence, independently selected from the group consisting of hydrogen, C1-C6 alkyl, C3-C10 cycloalkyl and W, as defined above;
R¹² is, at each occurrence, independently selected from hydrogen and W, as defined above; Wherein if R¹¹ is W, R¹² is hydrogen;
R¹³ is, at each occurrence, independently selected from the group consisting of hydrogen, halogen, C1-C6 alkyl, C1-C3 haloalkyl, —NH₂, —OR⁵, —CN and W, as defined above;
Wherein if R¹³ is W, R¹² is hydrogen;
R¹⁴ is any structure of group D;

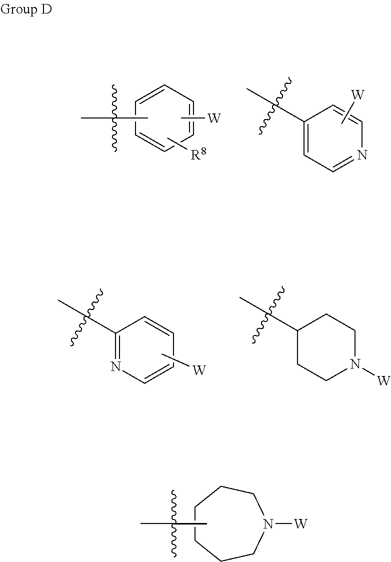

R¹⁵ is, at each occurrence, independently selected from hydrogen and W, as defined above;
R¹⁶ is, at each occurrence, independently selected from the group consisting of hydrogen, halogen, C1-C6 alkyl, C1-C3 haloalkyl, —NH₂, —OR⁵, —CN and W, as defined above;
Wherein if R¹⁶ is W, R¹² is hydrogen;
R¹⁷ is, at each occurrence, independently selected from the group consisting of hydrogen, C1-C6 alkyl and C1-C3 haloalkyl;
R¹⁸ is, at each occurrence, independently selected from the group consisting of hydrogen, halogen, C1-C6 alkyl, C1-C3 haloalkyl, —NH₂, —OR⁵ and —CN;
R¹⁹ is, at each occurrence, independently selected from the group consisting of hydrogen, C1-C6 alkyl, C3-C10 cycloalkyl and W, as defined above;
Wherein if R¹⁹ is W, R¹⁵ is hydrogen;
R²⁰ and R²¹ are, at each occurrence, independently selected from the group consisting of hydrogen, halogen, C1-C6 alkyl, C1-C3 haloalkyl, —OR⁵, heterocyclyl and —CN;
R²² is, at each occurrence, independently selected from the group consisting of hydrogen, halogen, C1-C6 alkyl, C3-C10 cycloalkyl, —N(R⁵)₂, —NR¹⁹R²⁰, —NR¹⁹CH₂(CO)NH₂, heterocyclyl, —OR⁵ and —CN.

In such method of treatment according to the invention, the compounds, DNA-viruses and the patient/subject to which such compound(s) is(are) administered, are as defined herein.

In one embodiment of the method of treatment, said method is for the treatment and/or prevention of a cancer caused by or associated with HPV, said cancer being selected from cervical cancer, oropharyngeal cancer, anal cancer, penile cancer, vaginal cancer and vulvar cancer.

In yet another aspect, the present invention relates to the use of a compound having the general formula I

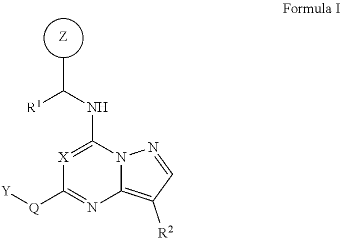

or an enantiomer, stereoisomeric form, mixture of enantiomers, diastereomer, mixture of diastereomers, racemate, or a pharmaceutically acceptable salt thereof, for the manufacture of a medicament for the treatment of a DNA-virus infection in a subject, said DNA-virus preferably being selected from Herpesviridae and Papillomaviridae; wherein in said compound
wherein
X is, independently at each occurrence, selected from CH and N;
Q is either absent or independently, at each occurrence, selected from the group consisting of —NH—, —NH(CH₂)—, —NH(CH₂)₂—, —NH(C═O)—, —NHSO₂—, —O—, —O(CH₂)—, —(C═O)—, —(C═O)NH—and —(C═O)(CH₂)—;
Y is, independently at each occurrence, selected from the group consisting of halogen, C1-C3 haloalkyl, C3-C8 cycloalkyl, aryl, heteroaryl, heterocyclyl, —S(═O)₂R³, C1-C6 alkyl and C1-C6 alkyl substituted with one or two of —OR⁵, —N(R⁵)R⁵, aryl, heteroaryl and heterocyclyl;
Wherein C3-C8 cycloalkyl is optionally substituted with one or two of R³, R⁴ and —(C═O)R⁵, wherein heterocyclyl is optionally substituted with one or two of R³, R⁴ and —(C═O)R⁵, and wherein aryl or heteroaryl is optionally substituted with one or two of R³, C1-C6 alkyl, —OR⁵, —N(R⁵)R⁵, —(C═O)R⁵, halogen, heteroaryl and heterocyclyl;
R¹ is, at each occurrence, independently selected from the group consisting of hydrogen and methyl;
R² is, at each occurrence, independently selected from the group consisting of halogen, C1-C6 alkyl, C3-C10 cycloalkyl, —CN, —(C═O)CH₃ and C1-C3 haloalkyl, any of which is optionally substituted;
R³ is either absent or independently, at each occurrence, selected from the group consisting of hydrogen, —OR⁵, halogen, C1-C3 haloalkyl, —CN, —N(R⁵)R⁵, (═O), —NH(C═O)R⁵, —(C═O)NH₂, —S(═O)₂N(R⁵)R⁵, aryl, heteroaryl, heterocyclyl, C1-C6 alkyl and C1-C6 alkyl substituted with —OR₅, —NH₂ or —S(═O)₂N(R⁵)R⁵;
R⁴ is, independently, at each occurrence, selected from the group consisting of hydrogen, halogen, C1-C3 haloalkyl, —CN, —OR⁵, —N(R⁵)R⁵, (═O), S(═O)₂N(R⁵)R⁵, aryl, heteroaryl, heterocyclyl, C1-C6 alkyl and C1-C6 alkyl substituted with —OH, —NH₂ or —S(═O)₂N(R⁵)R⁵;
wherein both R³ and R⁴ are (═O) if attached to a single sulfur atom that forms part of Y being a heterocycle;
or wherein R³ and R⁴, together with the structure to which they are attached, form an aromatic ring, a heteroaromatic ring, a saturated or unsaturated heterocyclic ring, or a fused or bridged ring structure of any of an aromatic ring, a heteroaromatic ring, and a saturated or unsaturated heterocyclic ring;
R⁵ is, at each occurrence, independently selected from the group consisting of hydrogen, C1-C6 alkyl, C3-C6 cycloalkyl, C1-C3 haloalkyl, heteroaryl, heterocyclyl, heteroaryl substituted with one or two of halogen, —OR¹¹, —N(R¹¹)R¹¹, C1-C6 alkyl and C1-C6 alkyl substituted with —OH, —NH₂; heterocyclyl substituted with one or two of halogen, —OR¹¹, —N(R¹¹)R¹¹, C1-C6 alkyl and C1-C6 alkyl substituted with —OH or —NH₂;
Z is any structure of the following group A;

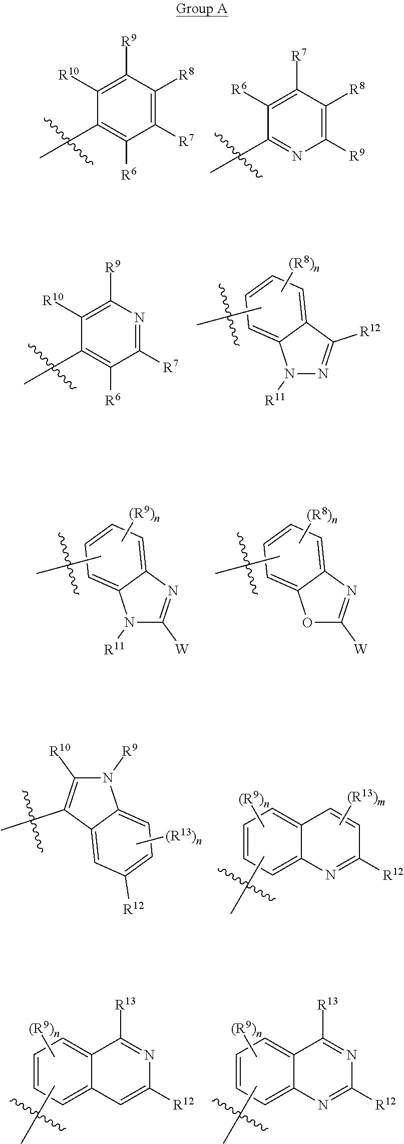

Wherein n=1, 2, or 3; M=1, or 2;
R⁶ and R⁷ are, at each occurrence, independently selected from the group consisting of hydrogen, —NH(C═O)R¹⁴, —NHR¹⁴, —OR¹⁴ and any structure of the following group B, with the
proviso that, when Z is

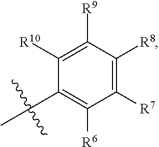

one of R⁶ and R⁷ is not H;

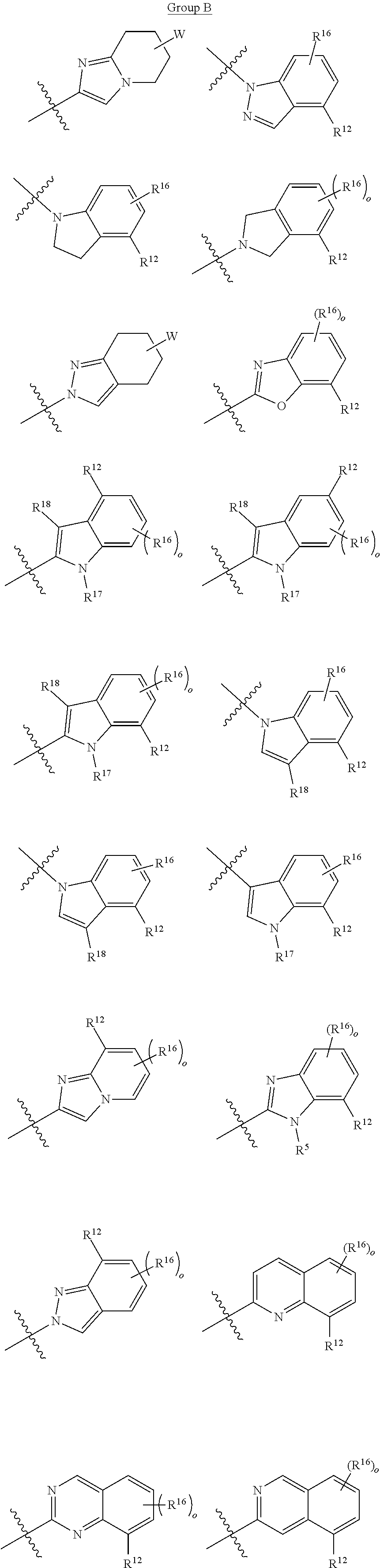

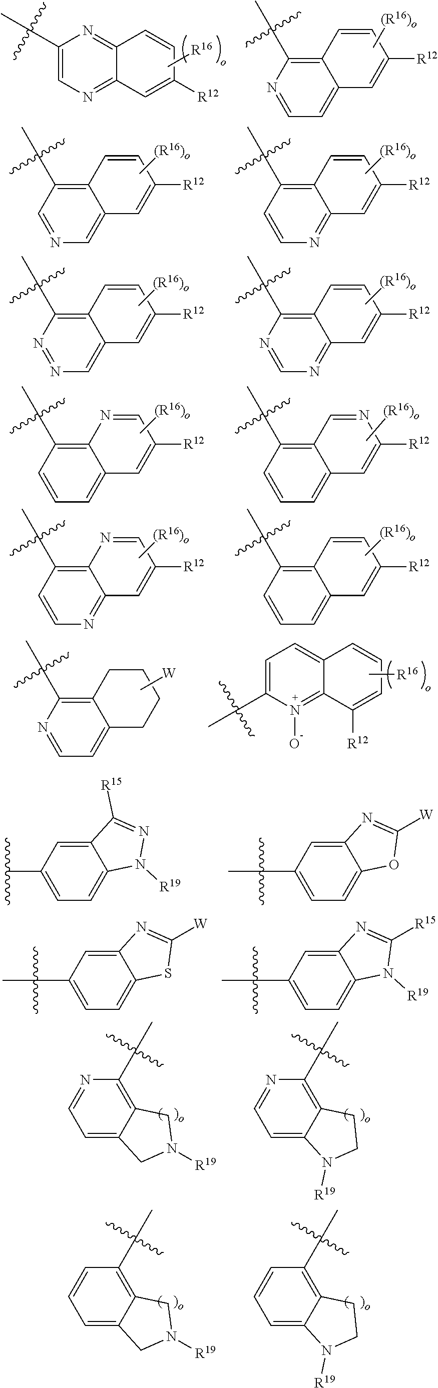

wherein o is, independently at each occurrence, selected from 1, 2 and 3;
W is any structure of the following group C;

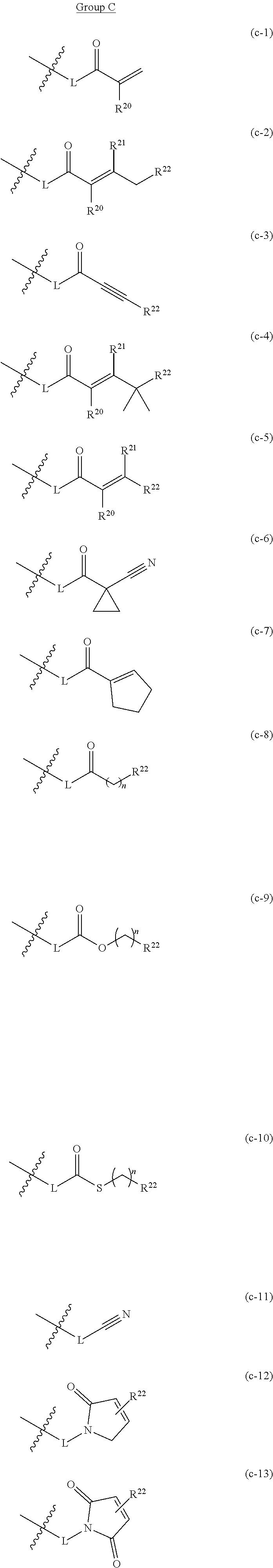

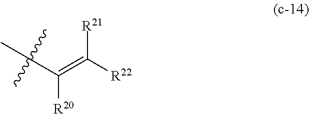

L is absent or, at each occurrence, independently selected from the group consisting of —O— and —NH—;
wherein n is, independently at each occurrence, selected from 1, 2 and 3;
R⁸, R⁹ and R¹⁰ are, at each occurrence, independently selected from the group consisting of hydrogen, halogen, C1-C6 alkyl, C1-C3 haloalkyl, —OR⁵, —CN and C1-C6 alkyl substituted with —OH, —OR⁵ or —NHR⁵;
R¹¹ is, at each occurrence, independently selected from the group consisting of hydrogen, C1-C6 alkyl, C3-C10 cycloalkyl and W, as defined above;
R¹² is, at each occurrence, independently selected from hydrogen and W, as defined above;
Wherein if R¹¹ is W, R¹² is hydrogen;
R¹³ is, at each occurrence, independently selected from the group consisting of hydrogen, halogen, C1-C6 alkyl, C1-C3 haloalkyl, —NH₂, —OR⁵, —CN and W, as defined above;
Wherein if R¹³ is W, R¹² is hydrogen;
R¹⁴ is any structure of group D;

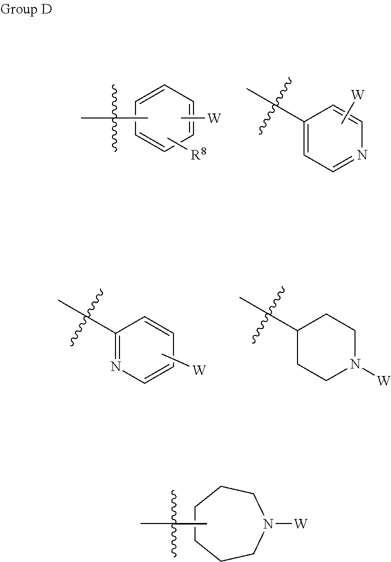

R¹⁵ is, at each occurrence, independently selected from hydrogen and W, as defined above;
R¹⁶ is, at each occurrence, independently selected from the group consisting of hydrogen, halogen, C1-C6 alkyl, C1-C3 haloalkyl, —NH₂, —OR⁵, —CN and W, as defined above;
Wherein if R¹⁶ is W, R¹² is hydrogen;
R¹⁷ is, at each occurrence, independently selected from the group consisting of hydrogen, C1-C6 alkyl and C1-C3 haloalkyl;
R¹⁸ is, at each occurrence, independently selected from the group consisting of hydrogen, halogen, C1-C6 alkyl, C1-C3 haloalkyl, —NH₂, —OR⁵ and —CN;
R¹⁹ is, at each occurrence, independently selected from the group consisting of hydrogen, C1-C6 alkyl, C3-C10 cycloalkyl and W, as defined above;
Wherein if R¹⁹ is W, R¹⁵ is hydrogen;
R²⁰ and R²¹ are, at each occurrence, independently selected from the group consisting of hydrogen, halogen, C1-C6 alkyl, C1-C3 haloalkyl, —OR⁵, heterocyclyl and —CN;
R²² is, at each occurrence, independently selected from the group consisting of hydrogen, halogen, C1-C6 alkyl, C3-C10 cycloalkyl, —N(R⁵)₂, —NR¹⁹R²⁰, —NR¹⁹CH₂(CO)NH₂, heterocyclyl, —OR⁵ and —CN.

In such use according to the invention, the compounds, DNA-viruses and the patient/subject to which such compound(s) is(are) administered, are as defined herein.

In one embodiment of such use, said method is for the treatment and/or prevention of a cancer caused by or associated with HPV, said cancer being selected from cervical cancer, oropharyngeal cancer, anal cancer, penile cancer, vaginal cancer and vulvar cancer.

The compounds of the present invention are highly efficient inhibitors of CDK7 which is a threonine/serine kinase that forms a trimeric complex with cyclin H (CycH) and MAT1, i.e. CDK7/MAT1/CycH. The inventive compounds are suitable for the use as a pharmaceutically active agent in the treatment and management of infections by DNA-viruses, such as Herpesviridae viruses and Papillomaviridae, and in methods of treatment of such infections wherein the respective compound is administered to a subject in need thereof. Moreover, they are also useful in the treatment or prevention of cancers caused by or associated with infections by DNA-viruses, such as HPV.

Based on their findings, the present inventors conclude that the selective CDK7 inhibitors according to the present invention exert excellent therapeutic effects on infections by DNA-viruses, such as Herpesviridae viruses and Papillomaviridae, and moreover also a therapeutic and/or prophylactic effect in various cancer types caused by or associated with HPV infections, in particular high-risk HPV-infections (HR-HPV). The CDK7-specific inhibitors in accordance with the present invention therefore also represent novel alternative treatment options for patients with DNA-virus infections and/or HPV-induced/associated cancers who cannot be vaccinated or do not respond well to HPV vaccines such as Cervarix or Gardasil.

The inventive compounds are also useful in the manufacture of a medicament or of a pharmaceutical composition for the treatment of disorders associated with, accompanied by, caused by and/or induced by CDK7-complex, in particular a hyperfunction or dysfunction thereof. The inventive compounds are further used in the manufacture of a medicament or of a pharmaceutical composition for the treatment and/or prevention of infections by Herpesviridae viruses.

The present inventors have found that in particular in those embodiments of the present invention wherein the compounds according to the present invention contain a W-group, as defined above, they are able to bind covalently to —SH-groups of cysteine residues within cyclin-dependent kinase(s), especially CDK7, thus forming a covalent bond and an adduct between the compound and the kinase and thus inhibiting the kinase(s). This concerns in particular those embodiments wherein at least one of Z, R⁶, R⁷, R¹¹, R¹², R¹³, R¹⁵, R¹⁶ and R¹⁹ is W, as defined above or herein, or is a structure containing W, as defined above or herein.

Furthermore it concerns those embodiments wherein exactly one of Z, R⁶, R⁷, R¹¹, R¹², R¹³, R¹⁵, R¹⁶ and R¹⁹ is W, as defined above or herein, or is a structure containing W, as defined above or herein. This is because all W-structures as defined above or herein contain a double or triple bond allowing a reaction with a sulfhydryl group within the kinase and allowing the formation of an adduct between the compound and the kinase. Through the covalent binding of a compound in accordance with the present invention, the kinase is inhibited. The term “exactly one”, as used in this context, means that it is only one (and no more) of the recited groups/residues which is W or a structure containing W, as defined above or herein.

The term “optionally substituted” as used herein is meant to indicate that a hydrogen atom where present and attached to a member atom within a group, or several such hydrogen atoms, may be replaced by a suitable group, such as halogen including fluorine, C₁-C₃ alkyl, C₁-C₃ haloalkyl, methylhydroxyl, COOMe, C(O)H, COOH, OMe, or OCF₃.

The term “alkyl” refers to a monovalent straight, branched or cyclic chain, saturated aliphatic hydrocarbon radical having a number of carbon atoms in the specified range. Thus, for example, “C₁-C₆ alkyl” refers to any of the hexyl alkyl and pentyl alkyl isomers as well as n-, iso-, sec-, and t-butyl, n- and isopropyl, cyclic propyl, ethyl and methyl.

The term “alkenyl” refers to a monovalent straight or branched chain aliphatic hydrocarbon radical containing one carbon-carbon double bond and having a number of carbon atoms in the specified range. Thus, for example, “C₂-C₆ alkenyl” refers to all of the hexenyl and pentenyl isomers as well as 1-butenyl, 2-butenyl, 3-butenyl, isobutenyl, 1-propenyl, 2-propenyl, and ethenyl (or vinyl).

The term “cycloalkyl”, alone or in combination with any other term, refers to a group, such as optionally substituted or non-substituted cyclic hydrocarbon, having from three to eight carbon atoms, unless otherwise defined. Thus, for example, “C₃-C₈ cycloalkyl” refers to cyclopropyl, cyclobutyl, cyclopentyl, cyclohexyl, cycloheptyl, and cyclooctyl.

The term “haloalkyl” refers to an alkyl group, as defined herein that is substituted with at least one halogen. Examples of straight or branched chained “haloalkyl” groups useful in the present invention include, but are not limited to, methyl, ethyl, propyl, isopropyl, n-butyl, and t-butyl substituted independently with one or more halogens. The term “haloalkyl” should be interpreted to include such substituents such as —CHF₂, —CF₃, —CH₂—CH₂—F, —CH₂—CF₃, and the like.

The term “heteroalkyl” refers to an alkyl group where one or more carbon atoms have been replaced with a heteroatom, such as, O, N, or S. For example, if the carbon atom of alkyl group which is attached to the parent molecule is replaced with a heteroatom (e.g., O, N, or S) the resulting heteroalkyl groups are, respectively, an alkoxy group (e.g., —OCH₃, etc.), an amine (e.g., —NHCH₃, —N(CH₃)₂, etc.), or thioalkyl group (e.g., —SCH₃, etc.). If a non-terminal carbon atom of the alkyl group which is not attached to the parent molecule is replaced with a heteroatom (e.g., O, N, or S) and the resulting heteroalkyl groups are, respectively, an alkyl ether (e.g., —CH₂CH₂—O—CH₃, etc.), alkyl amine (e.g., —CH₂NHCH₃, —CH₂N(CH₃)₂, etc.), or thioalkyl ether (e.g., —CH₂—S—CH₃).

The term “halogen” refers to fluorine, chlorine, bromine, or iodine.

The term “phenyl” as used herein is meant to indicate that optionally substituted or non-substituted phenyl group.

The term “benzyl” as used herein is meant to indicate that optionally substituted or non-substituted benzyl group.

The term “heteroaryl” refers to (i) optionally substituted 5- and 6-membered heteroaromatic rings and (ii) optionally substituted 9- and 10-membered bicyclic, fused ring systems in which at least one ring is aromatic, wherein the heteroaromatic ring or the bicyclic, fused ring system contains from 1 to 4 heteroatoms independently selected from N, O, and S, where each N is optionally in the form of an oxide and each S in a ring which is not aromatic is optionally S(O) or S(O)₂. Suitable 5- and 6-membered heteroaromatic rings include, for example, pyridyl, pyrrolyl, pyrazinyl, pyrimidinyl, pyridazinyl, triazinyl, thienyl, furanyl, imidazolyl, pyrazolyl, triazolyl, tetrazolyl, oxazolyl, isooxazolyl, oxadiazolyl, thiazolyl, isothiazolyl, and thiadiazolyl. Suitable 9- and 10-membered heterobicyclic, fused ring systems include, for example, benzofuranyl, indolyl, indazolyl, naphthyridinyl, isobenzofuranyl, benzopiperidinyl, benzisoxazolyl, benzoxazolyl, chromenyl, quinolinyl, isoquinolinyl, cinnolinyl, quinazolinyl, tetrahydroquinolinyl, tetrahydroisoquinolinyl, isoindolyl, benzodioxolyl, benzofuranyl, imidazo[1,2-a]pyridinyl, benzotriazolyl, dihydroindolyl, dihydroisoindolyl, indazolyl, indolinyl, isoindolinyl, quinoxalinyl, quinazolinyl, 2,3-dihydrobenzofuranyl, and 2,3-dihydrobenzo-1,4-dioxinyl.

The term “heterocyclyl” refers to (i) optionally substituted 4- to 8-membered, saturated and unsaturated but non-aromatic monocyclic rings containing at least one carbon atom and from 1 to 4 heteroatoms, (ii) optionally substituted bicyclic ring systems containing from 1 to 6 heteroatoms, and (iii) optionally substituted tricyclic ring systems, wherein each ring in (ii) or (iii) is independent of fused to, or bridged with the other ring or rings and each ring is saturated or unsaturated but nonaromatic, and wherein each heteroatom in (i), (ii), and (iii) is independently selected from N, O, and S, wherein each N is optionally in the form of an oxide and each S is optionally oxidized to S(O) or S(O)₂. Suitable 4- to 8-membered saturated heterocyclyls include, for example, azetidinyl, piperidinyl, morpholinyl, thiomorpholinyl, thiazolidinyl, isothiazolidinyl, oxazolidinyl, isoxazolidinyl, pyrrolidinyl, imidazolidinyl, piperazinyl, tetrahydrofuranyl, tetrahydrothienyl, pyrazolidinyl, hexahydropyrimidinyl, thiazinanyl, thiazepanyl, azepanyl, diazepanyl, tetrahydropyranyl, tetrahydrothiopyranyl, dioxanyl, and azacyclooctyl. Suitable unsaturated heterocyclic rings include those corresponding to the saturated heterocyclic rings listed in the above sentence in which a single bond is replaced with a double bond. It is understood that the specific rings and ring systems suitable for use in the present invention are not limited to those listed in this and the preceding paragraphs. These rings and ring systems are merely representative.

The term “non-responder” to a vaccination is meant to refer to a patient or subject who, in spite of having undergone a vaccination, does not develop or show an immune response against a subsequent infection by the respective pathogen or against exposure to the respective antigen of the respective pathogen. In a patient who “fails to respond adequately to a vaccination”, the respective immune response mounted by such vaccinated patient against a subsequent infection with the respective pathogen or against exposure to the respective antigen, is not sufficient to offer immunity and protection against such infection or exposure.

A patient who “cannot be vaccinated against a viral infection” is a patient for whom the potential benefits of a vaccination are outweighed by the expected side effects or drawbacks of such vaccination. This may be due to, for example, age, health conditions, or other factors preventing a patient from being vaccinated, e.g. pregnancy, serious infection or illness, or sensitivity to one or several components within the vaccine. For example, such patient may be an immune-compromised patient for whom it may be detrimental to undergo vaccination, because the expected immune response mounted by the patient's immune system may be too weak to offer sufficient protection against infection, whilst at the same time the vaccination itself may cause other serious side-effects in the patient that outweigh any positive effects of the vaccination.

### Pharmaceutically Acceptable Salts

Examples of pharmaceutically acceptable addition salts include, without limitation, the non-toxic inorganic and organic acid addition salts such as the acetate derived from acetic acid, the aconate derived from aconitic acid, the ascorbate derived from ascorbic acid, the benzenesulfonate derived from benzensulfonic acid, the benzoate derived from benzoic acid, the cinnamate derived from cinnamic acid, the citrate derived from citric acid, the embonate derived from embonic acid, the enantate derived from enanthic acid, the formate derived from formic acid, the fumarate derived from fumaric acid, the glutamate derived from glutamic acid, the glycolate derived from glycolic acid, the hydrochloride derived from hydrochloric acid, the hydrobromide derived from hydrobromic acid, the lactate derived from lactic acid, the maleate derived from maleic acid, the malonate derived from malonic acid, the mandelate derived from mandelic acid, the methanesulfonate derived from methane sulphonic acid, the naphthalene-2-sulphonate derived from naphtalene-2-sulphonic acid, the nitrate derived from nitric acid, the perchlorate derived from perchloric acid, the phosphate derived from phosphoric acid, the phthalate derived from phthalic acid, the salicylate derived from salicylic acid, the sorbate derived from sorbic acid, the stearate derived from stearic acid, the succinate derived from succinic acid, the sulphate derived from sulphuric acid, the tartrate derived from tartaric acid, the toluene-p-sulphonate derived from p-toluene sulphonic acid, and the like. Such salts may be formed by procedures well known and described in the art.

Other acids such as oxalic acid, which may not be considered pharmaceutically acceptable, may be useful in the preparation of salts useful as intermediates in obtaining a chemical compound of the invention and its pharmaceutically acceptable acid addition salt.

In another embodiment, the compounds of the invention are used in their respective free base form according to the present invention.

Metal salts of a chemical compound of the invention include alkali metal salts, such as the sodium salt of a chemical compound of the invention containing a carboxy group.

The chemical compounds of the invention may be provided in unsolvated or solvated forms together with a pharmaceutically acceptable solvent(s) such as water, ethanol, and the like.

Solvated forms may also include hydrated forms such as the monohydrate, the dihydrate, the hemihydrate, the trihydrate, the tetrahydrate, and the like. In general, solvated forms are considered equivalent to unsolvated forms for the purposes of this invention.

Further aspects of the present invention are illustrated and exemplified by the following schemes, examples, tables and procedural descriptions which are given merely to illustrate, not to limit the present invention. The scope of protection for the present invention is merely limited by the appended claims.

Furthermore, reference is made to the following figures, wherein

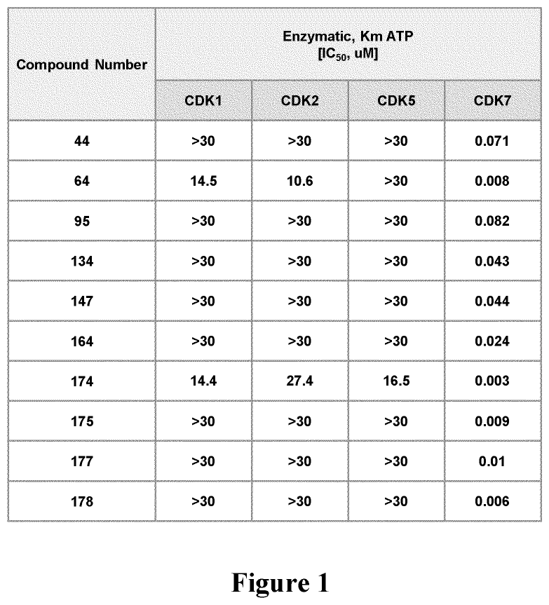

FIG. 1 shows activity data of selected exemplary compounds according to the present invention in terms of their inhibition of different cyclin-dependent kinases.

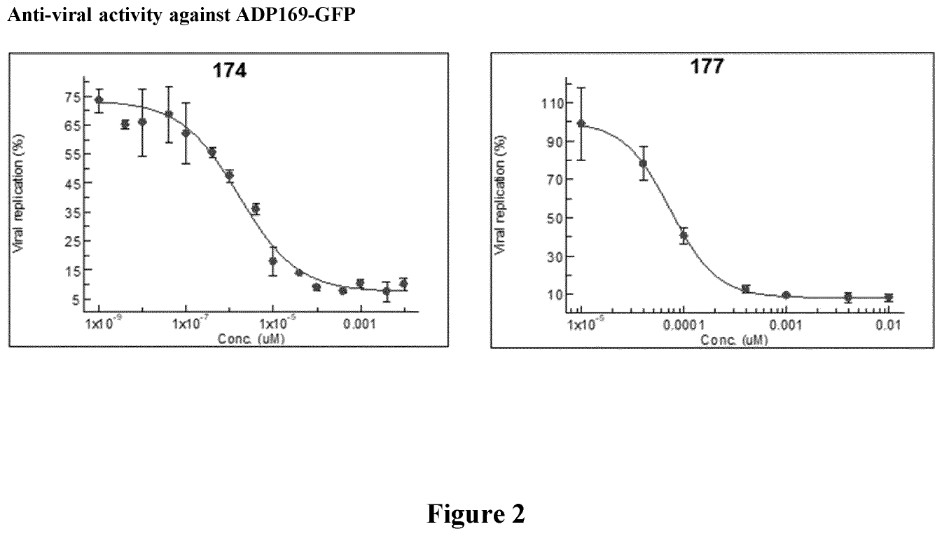

FIG. 2 shows selected data for two compounds (174 and 177) in terms of their antiviral activity against replication of HCMV (HCMV strain ADP169-GFP).

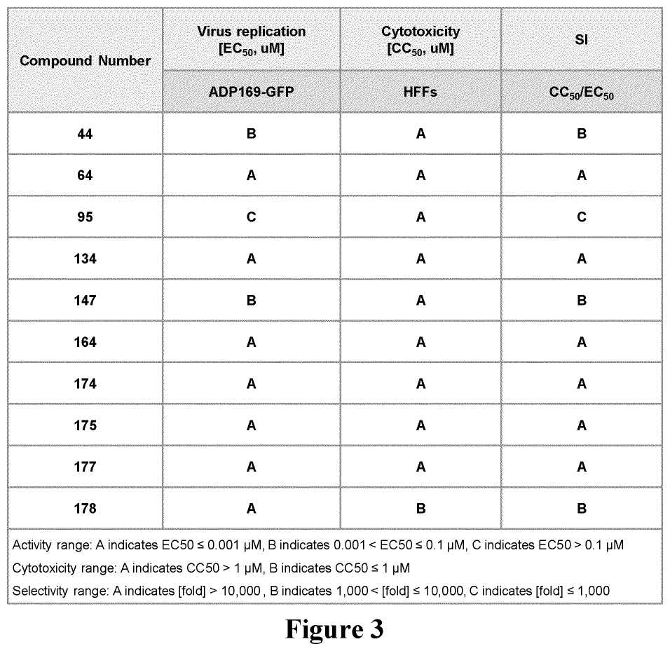

FIG. 3 shows antiviral activities of selected exemplary compounds according to the present invention in terms of their inhibition capabilities of human cytomegalovirus (HCMV) (HCMV strain ADP169-GFP).

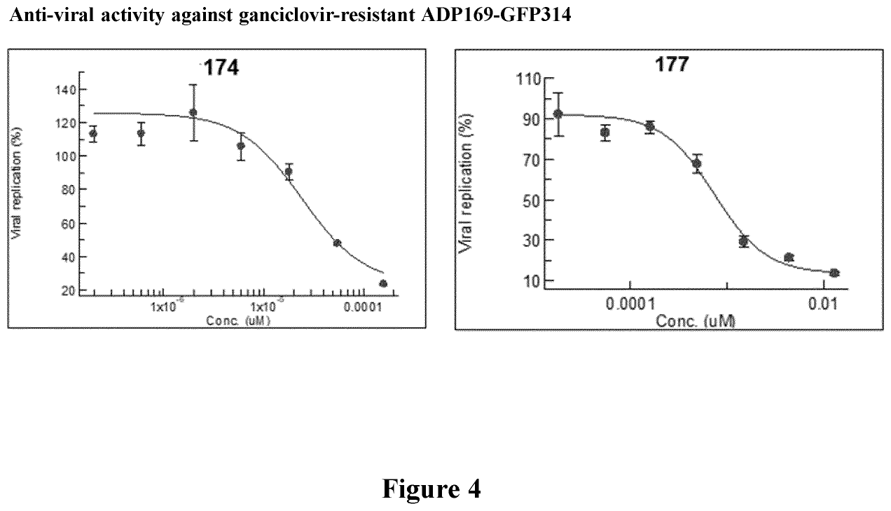

FIG. 4 shows selected data for exemplary compounds according to the present invention (compounds 174 and 177) in terms of their antiviral activity against replication of a nucleobase resistant HCMV.

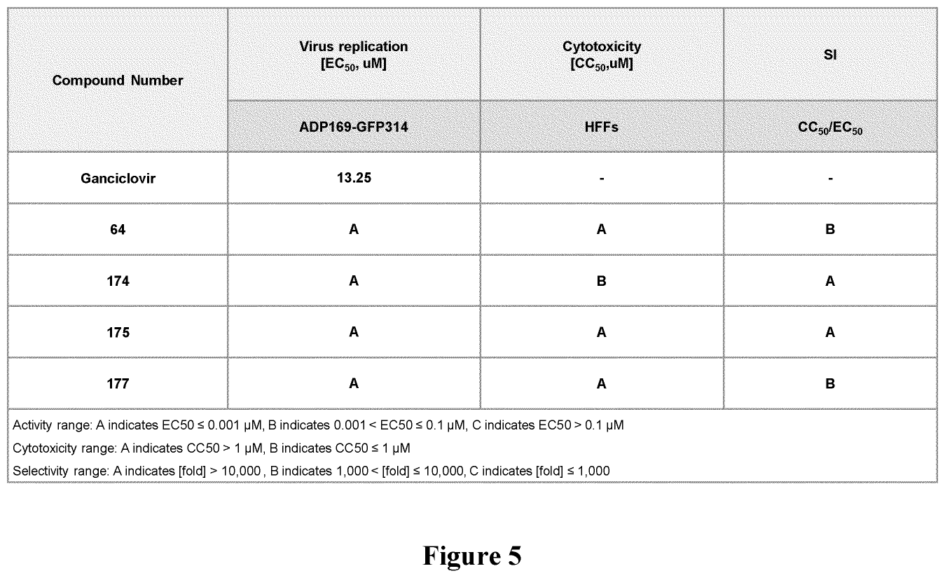

FIG. 5 shows antiviral activities of selected exemplary compounds according to the present invention in terms of their antiviral activities against human cytomegalovirus that is resistant against a nucleobase analogue, ganciclovir (ganciclovir-resistant HCMV strain ADP169-GFP314).

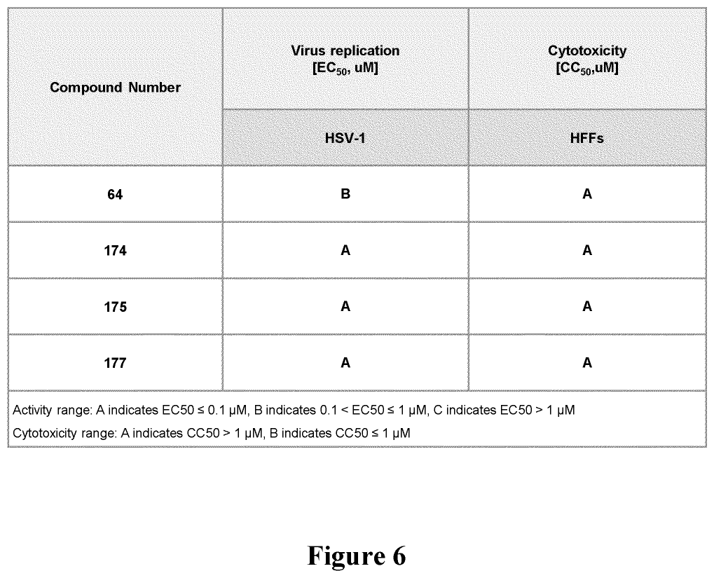

FIG. 6 shows antiviral activities of selected exemplary compounds according to the present invention in terms of their inhibitory activity against replication of human Herpes simplex virus 1 (HSV-1).

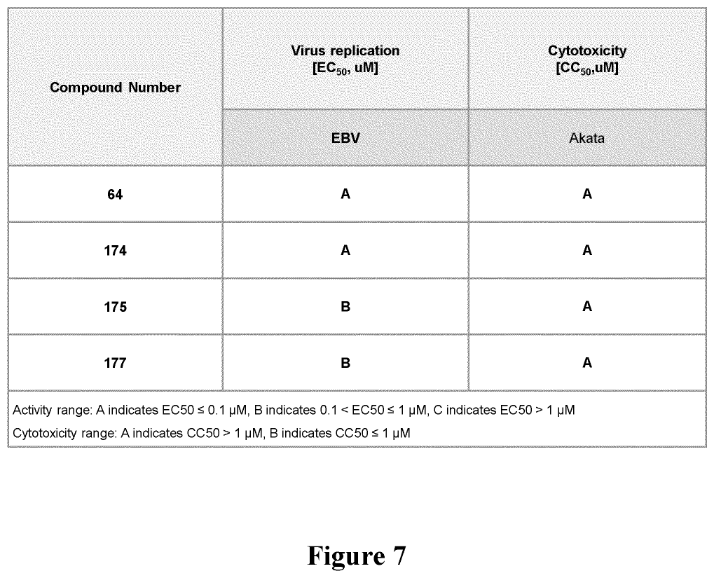

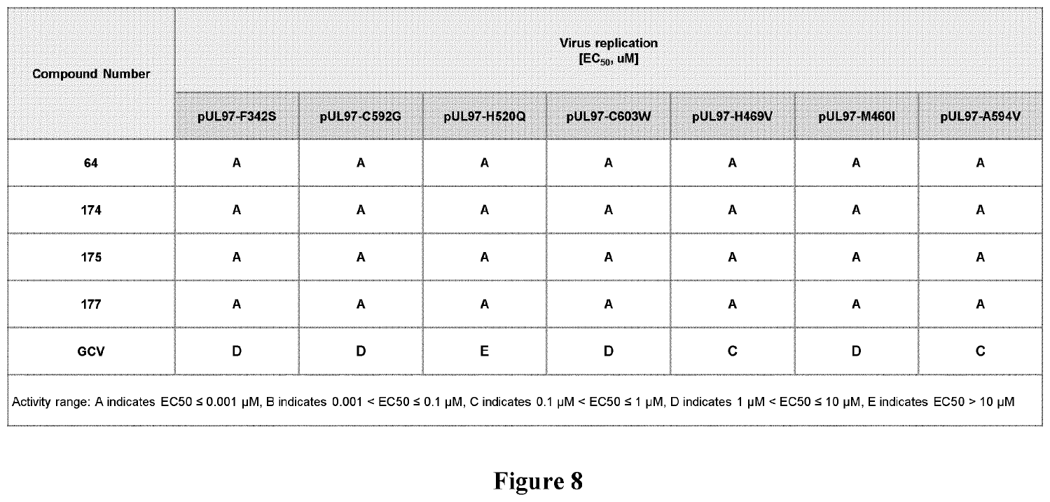

FIG. 7 shows antiviral activities of selected exemplary compounds according to the present invention in terms of their inhibitory activity against replication of Epstein-Barr virus (EBV) FIG. 8 shows the highly inhibitory effects of selected compounds according to the present invention on GCV-resistant pUL97-mutated HCMVs.

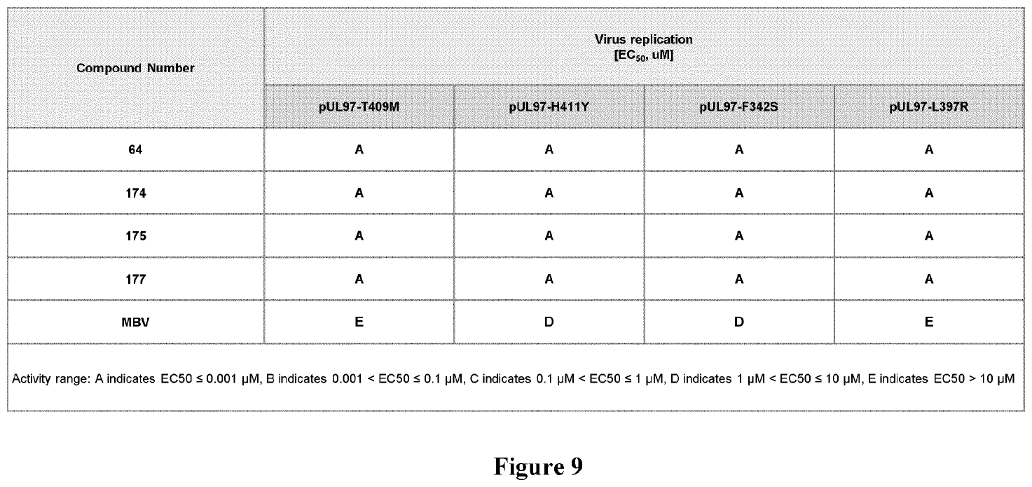

FIG. 9 shows the highly inhibitory effects of selected compounds according to the present invention on MBV-resistant pUL97-mutated HCMVs.

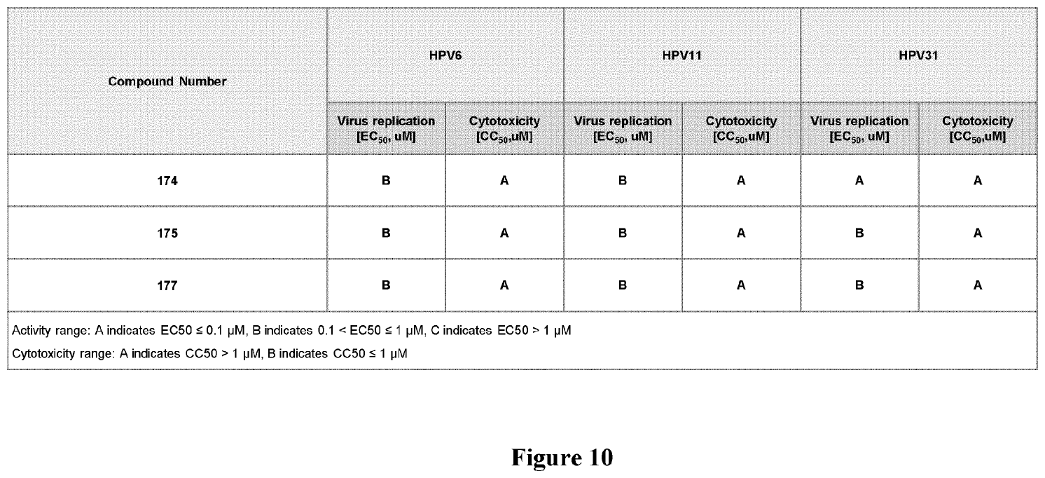

FIG. 10 shows the highly inhibitory effects of selected compounds according to the present invention on replication of different HPVs.

Moreover, table 1 summarizes exemplary compounds according to the present invention that may be used for the treatment of Herpesviridae infections, namely compounds 1-216 in terms of their structures and corresponding characteristics.

TABLE 1
Summarized compounds 1-216 in terms of their structures and corresponding
characteristics

| #cpds | Structure | Characterization Data |
|---|---|---|
| 1 | 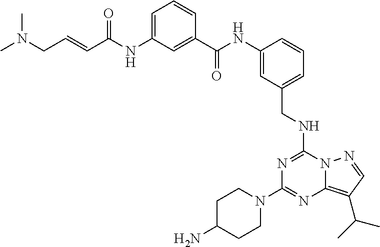 [SMILES: CC(C)c1cnn2c(NCc3cccc(NC(=O)c4cccc(NC(=O)/C=C/CN(C)C)c4)c3)nc(N3CCC(N)CC3)nc12] | white powder; ¹H-NMR (DMSO-d6, 400 MHz): δ 10.28 (1H, brs), 10.25 (1H, brs), 8.86 (1H, brs), 8.13 (1H, s), 7.88 (1H, d, J = 8.0 Hz), 7.80 (1H, s), 7.72 (1H, s), 7.65 (1H, d, J = 7.6 Hz), 7.60 (1H, d, J = 7.6 Hz), 7.44 (1H, t, J = 8.0 Hz), 7.28 (1H, t, J = 7.6 Hz), 7.12 (1H, d, J = 7.2 Hz), 6.75 (1H, td, J = 15.6, 6.0 Hz,), 6.28 (1H, d, J = 15.6 Hz), 4.59 (2H, s), 4.42-4.54 (2H, m), 3.06 (2H, d, J = 5.2 Hz), 2.85-2.98 (3H, m), 2.65-2.75 (1H, m), 2.18 (6H, s), 1.64-1.76 (2H, m), 1.23 (6H, d, J = 7.2 Hz), 1.03-1.15 (2H, m); LCMS: 95.8%, MS (ESI): m/z 611.3[M + H]+. |
| 2 | 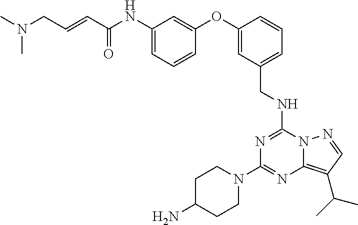 [SMILES: CC(C)c1cnn2c(NCc3cccc(Oc4cccc(NC(=O)/C=C/CN(C)C)c4)c3)nc(N3CCC(N)CC3)nc12] | white powder; ¹H-NMR (DMSO-d6, 400 MHz): δ 10.12 (1H, brs), 8.85 (1H, brs), 7.70 (1H, s), 7.36-7.42 (2H, m), 7.24-7.36 (2H, m), 7.17 (1H, d, J = 7.6 Hz), 7.07 (1H, s), 6.90 (1H, dd, J = 8.0, 1.6 Hz), 6.63-6.74 (2H, m), 6.21 (1H, d, J = 15.2 Hz), 4.55 (2H, s), 4.39- 4.50 (2H, m), 2.99-3.08 (2H, m), 2.81-2.97 (3H, m), 2.66-2.77 (1H, m), 2.16 (6H, s), 1.61-1.76 (2H, m), 1.22 (6H, d, J = 7.2 Hz), 0.99-1.13 (2H, m); LCMS: 100%, MS (ESI): m/z 584.2[M + H]+. |
| 3 | 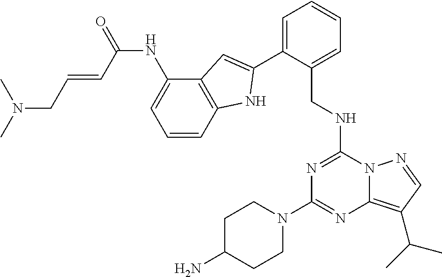 [SMILES: CC(C)c1cnn2c(NCc3ccccc3-c3cc4c(NC(=O)/C=C/CN(C)C)cccc4[nH]3)nc(N3CCC(N)CC3)nc12] | White powder; ¹H-NMR (DMSO-d₆, 400 MHz): δ 8.65 (1H, brs), 7.89 (1H, brs), 7.62 (1H, s), 7.48-7.59 (3H, m), 7.31-7.43 (2H, m), 7.16-7.22 (1H, m), 7.09-7.15 (1H, m), 6.95-7.06 (1H, m), 6.61 (1H, t, J = 5.6 Hz), 6.56 (1H, s), 6.21 (1H, d, J = 15.2 Hz), 4.83 (2H, d, J = 5.2 Hz), 4.55-4.65 (2H, m), 3.10-3.20 (2H, m), 2.98- 3.08 (1H, m), 2.80-2.89 (3H, m), 2.30 (6H, s), 1.77- 1.79 (2H, m), 1.28-1.32 (8H, m); LCMS: 100%, MS (ESI): m/z 629.4[M + Na]⁺ |
| 4 | 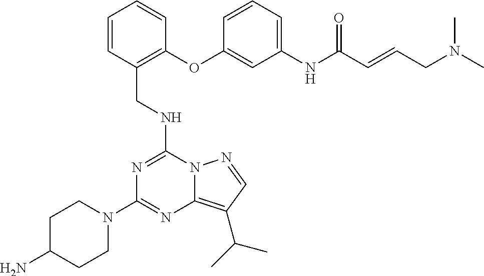 [SMILES: CC(C)c1cnn2c(NCc3ccccc3Oc3cccc(NC(=O)/C=C/CN(C)C)c3)nc(N3CCC(N)CC3)nc12] | white powder; ¹H-NMR (CDCl₃, 400 MHz): δ 7.56 (1H, s), 7.42-7.48 (2H, m), 7.26-7.32 (1H, m), 7.17-7.24 (1H, m), 7.14 (1H, brs), 7.07-7.12 (1H, m), 6.88-6.98 (2H, m), 6.60-6.70 (2H, m), 6.07 (1H, d, J = 15.2 Hz), 4.77 (2H, d, J = 6.0 Hz), 4.68-4.76 (2H, m), 3.09 (2H, d, J = 5.6 Hz), 2.96-3.04 (1H, m), 2.86-2.96 (3H, m), 2.26 (6H, s), 1.76-1.90 (2H, m), 1.23-1.34 (8H, m); LCMS: 100%, MS (ESI): m/z 584.4[M + H]+. |
| 5 | 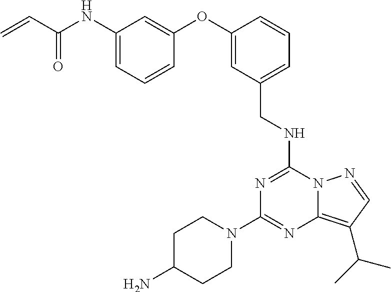 [SMILES: C=CC(=O)Nc1cccc(Oc2cccc(CNc3nc(N4CCC(N)CC4)nc4c(C(C)C)cnn34)c2)c1] | white powder; ¹H-NMR (CDCl₃, 400 MHz): δ 7.79 (1H, brs), 7.60 (1H, s), 7.40-7.50 (1H, m), 7.26-7.33 (2H, m), 7.19 (1H, s), 7.05-7.12 (2H, m), 6.93 (1H, d, J = 7.6 Hz), 6.75 (1H, d, J = 7.6 Hz), 6.71 (1H, brs), 6.41 (1H, d, J = 16.4 Hz), 6.24-6.34 (1H, m), 5.74 (1H, d, J = 10.0 Hz), 4.62-4.75 (4H, m), 2.96-3.06 (1H, m), 2.83- 2.95 (3H, m), 1.80-1.84 (2H, m), 1.21-1.33 (8H, m); LCMS: 100%, MS (ESI): m/z 527.3[M + H]+. |
| 6 | 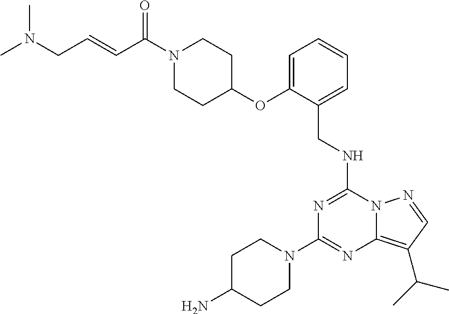 [SMILES: CC(C)c1cnn2c(NCc3ccccc3OC3CCN(C(=O)/C=C/CN(C)C)CC3)nc(N3CCC(N)CC3)nc12] | white powder; ¹H-NMR (DMSO-d6, 400 MHz): δ 7.56 (1H, s), 7.33 (1H, d, J = 8.0 Hz), 7.24-7.28 (1H, m), 6.92 (1H, d, J = 6.8 Hz), 6.80-6.88 (2H, m), 6.63 (1H, t, J = 4.8 Hz), 6.53 (1H, d, J = 16.0 Hz), 4.76-4.83 (2H, m), 4.63-4.70 (2H, m), 3.65-3.80 (3H, m), 3.53-3.56 (1H, m), 3.21-3.25 (2H, m), 3.09-3.14 (1H, m), 2.91- 3.03 (3H, m), 2.35 (6H, s), 1.94-1.97 (2H, m), 1.86- 1.90 (4H, m), 1.45-1.48 (2H, m), 1.27 (6H, d, J = 7.2 Hz); LCMS: 100%, MS (ESI): m/z 576.3[M + H]+. |
| 7 | 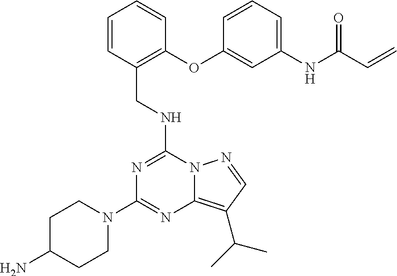 [SMILES: C=CC(=O)Nc1cccc(Oc2ccccc2CNc2nc(N3CCC(N)CC3)nc3c(C(C)C)cnn23)c1] | white powder; ¹H-NMR (CDCl₃, 400 MHz): δ 7.49 (1H, s), 7.38-7.40 (2H, m), 7.14-7.22 (3H, m), 7.04- 7.06 (2H, m), 6.87 (1H, d, J = 8.0 Hz), 6.58-6.59 (1H, m), 6.53 (1H, t, J = 4.8 Hz), 6.33-6.37 (1H, d, J = 16.8 Hz), 6.12-6.14 (1H, m), 5.69 (1H, d, J = 10.8 Hz), 4.64- 4.71 (4H, m), 2.82-2.95 (4H, m), 1.79-1.82 (2H, m), 1.19-1.27 (8H, m); LCMS: 91.6%, MS (ESI): m/z 527.3[M + H]+. |
| 8 | 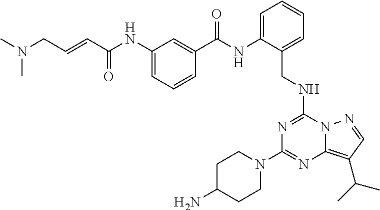 [SMILES: CC(C)c1cnn2c(NCc3ccccc3NC(=O)c3cccc(NC(=O)/C=C/CN(C)C)c3)nc(N3CCC(N)CC3)nc12] | white powder; ¹H-NMR (DMSO-d6, 400 MHz): δ 10.26-10.27 (1H, s), 10.12-10.14 (1H, s), 8.85-8.69 (1H, brs), 8.21 (1H, s), 7.92 (1H, d, J = 8.0 Hz), 7.72 (1H, s), 7.65-7.68 (1H, m), 7.37-7.49 (3H, m), 7.27-7.31 (1H, m), 7.19-7.23 (1H, m), 6.73-6.76 (1H, m), 6.28 (1H, d, J = 15.6 Hz), 4.63 (2H, s), 4.42-4.47 (2H, m), 3.06 (2H, d, J = 5.2 Hz), 2.82-2.94 (3H, m), 2.70-2.77 (1H, m), 2.17 (6H, s), 1.64-1.69 (2H, m), 1.22 (6H, d, J = 7.2 Hz), 1.02-1.11 (2H, m); LCMS: 100.0%, MS (ESI): m/z 611.3[M + H]+. |
| 9 | 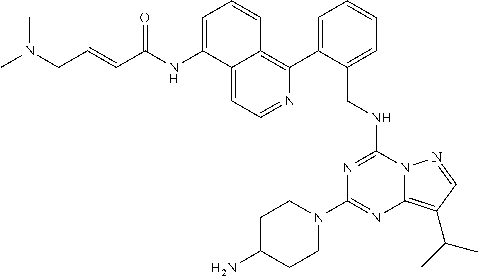 [SMILES: CC(C)c1cnn2c(NCc3ccccc3-c3nccc4c(NC(=O)/C=C/CN(C)C)cccc34)nc(N3CCC(N)CC3)nc12] | white powder; ¹H-NMR (CDCl₃, 400 MHz): δ 8.47- 8.49 (2H, s), 7.97-7.99 (1H, m), 7.61-7.63 (1H, m), 7.55-7.58 (1H, m), 7.52 (1H, s), 7.38-7.49 (4H, m), 7.22-7.25 (1H, m), 7.04-7.11 (1H, m), 6.63-6.65 (1H, m), 6.35-6.40 (1H, m), 4.72-4.77 (1H, m), 4.78-4.55 (2H, m), 4.26-4.30 (1H, m), 3.19-3.21 (2H, m), 2.92- 3.02 (2H, m), 2.76-2.83 (2H, m), 2.35 (6H, s), 1.79- 7.82 (2H, m), 1.14-1.29 (8H, m); LCMS: 95.3%, MS (ESI): m/z 619.4[M + H]+. |
| 10 | 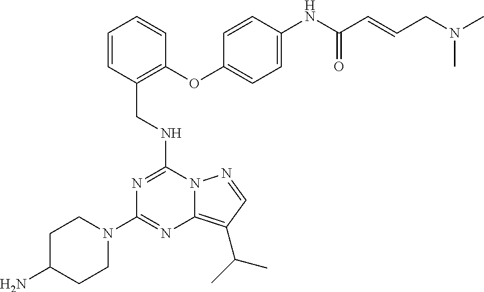 [SMILES: CC(C)c1cnn2c(NCc3ccccc3Oc3ccc(NC(=O)/C=C/CN(C)C)cc3)nc(N3CCC(N)CC3)nc12] | white powder; ¹H-NMR (CDCl₃, 400 MHz): δ 7.60 (1H, s), 7.44-7.53 (4H, m), 7.22-7.26 (1H, m), 7.08 (1H, t, J = 7.6 Hz), 6.93-7.00 (3H, m), 6.86 (1H, d, J = 7.6 Hz), 6.71 (1H, t, J = 6.2 Hz), 6.13 (1H, d, J = 15.6 Hz), 4.79 (2H, d, J = 6.4 Hz), 4.71-4.75 (2H, m), 3.12- 3.15 (2H, m), 2.99-3.06 (1H, m), 2.89-2.96 (3H, m), 2.30 (6H, s), 1.86-1.89 (2H, m), 1.24-1.33 (8H, m); LCMS: 100.0%, MS (ESI): m/z 584.4[M + H]+. |
| 11 | 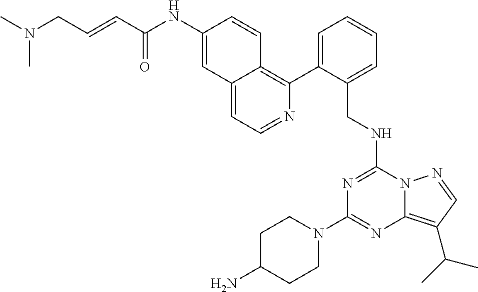 [SMILES: CC(C)c1cnn2c(NCc3ccccc3-c3nccc4cc(NC(=O)/C=C/CN(C)C)ccc34)nc(N3CCC(N)CC3)nc12] | white powder; ¹H-NMR (DMSO-d6, 400 MHz): δ 10.51 (1H, brs), 8.44-8.50 (3H, m), 7.75 (1H, d, J = 5.6 Hz), 7.64 (1H, s), 7.55-7.60 (1H, m), 7.46-7.53 (3H, m), 7.42 (1H, t, J = 8.0 Hz), 7.33 (1H, d, J = 7.2 Hz), 6.78- 6.85 (1H, m), 6.36 (1H, d, J = 15.2 Hz), 4.25-4.43 (4H, m), 3.08 (2H, d, J = 5.2 Hz), 2.72-2.86 (4H, m), 2.19 (6H, s), 1.60-1.70 (2H, m), 1.19 (6H, d, J = 7.2 Hz), 1.05-1.10 (2H, m); LCMS: 98.9%, MS (ESI): m/z 619.4[M + H]+. |
| 12 | 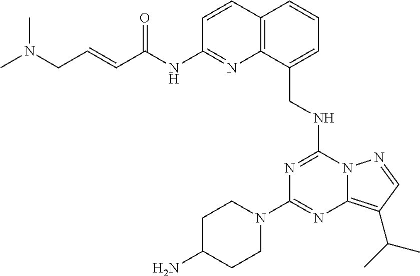 [SMILES: CC(C)c1cnn2c(NCc3cccc4ccc(NC(=O)/C=C/CN(C)C)nc34)nc(N3CCC(N)CC3)nc12] | white powder; ¹H-NMR (CDCl₃, 400 MHz): δ 9.36 (1H, brs), 8.51 (1H, d, J = 9.2 Hz), 8.14-8.17 (2H, m), 7.70 (1H, d, J = 6.8 Hz), 7.66 (1H, d, J = 6.8 Hz), 7.60 (1H, s), 7.34-7.38 (1H, m), 7.02-7.11 (1H, m), 6.32 (1H, d, J = 16.0 Hz), 5.16 (2H, d, J = 6.0 Hz), 4.78-4.84 (2H, m), 3.18 (2H, d, J = 6.4 Hz), 2.94-3.03 (4H, m), 2.33 (6H, s), 1.92-1.97 (2H, m), 1.30-1.45 (2H, m), 1.25 (6H, d, J = 6.8 Hz); LCMS: 95.9%, MS (ESI): m/z 565.3[M + Na]+. |
| 13 | 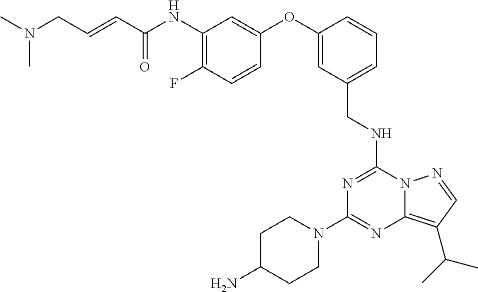 [SMILES: CC(C)c1cnn2c(NCc3cccc(Oc4ccc(F)c(NC(=O)/C=C/CN(C)C)c4)c3)nc(N3CCC(N)CC3)nc12] | white powder; ¹HNMR(DMSO-d₆, 400 MHz): δ 9.93 (1H, brs), 8.89 (1H, brs), 7.81-7.83 (1H, m), 7.72 (1H, s), 7.23-7.31 (2H, m), 7.15 (1H, d, J = 8.0 Hz), 7.06 (1H, s), 6.86 (1H, dd, J = 7.6, 2.0 Hz), 6.69-6.75 (2H, m), 6.46 (1H, d, J = 15.6), 4.50-4.55 (4H, m), 3.03 (2H, d, J = 4.8 Hz) 2.84-2.93 (4H, m), 2.16 (6H, s), 1.75-1.80 (2H, m), 1.18-1.23 (8H, m); LCMS: 100%, MS (ESI): m/z 624.3[M + Na]⁺. |
| 14 | 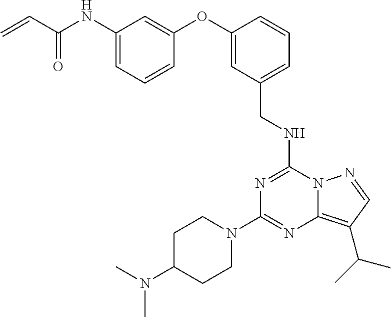 [SMILES: C=CC(=O)Nc1cccc(Oc2cccc(CNc3nc(N4CCC(N(C)C)CC4)nc4c(C(C)C)cnn34)c2)c1] | yellow powder; ¹H-NMR (CDCl₃, 400 MHz): δ 7.74 (1H, brs), 7.60 (1H, s), 7.41 (1H, d, J = 8.0 Hz), 7.23- 7.31 (3H, m), 7.05-7.13 (2H, m), 6.93 (1H, dd, J = 8.4, 1.6 Hz), 6.75 (1H, dd, J = 8.4, 1.6 Hz), 6.69 (1H, t, J = 5.6 Hz), 6.42 (1H, dd, J = 16.8, 1.6 Hz), 6.21-6.31 (1H, m), 5.75 (1H, d, J = 10.4), 4.78-4.88 (2H, m), 4.68 (2H, d, J = 6.0 Hz), 2.98-3.09 (1H, m), 2.72-2.85 (2H, m), 2.39-2.49 (1H, m), 2.31 (6H, s), 1.82-1.88 (2H, m), 1.34-1.47 (2H, m), 1.28 (6H, d, J = 6.8 Hz); LCMS: 94.2%, MS (ESI): m/z 55.3[M + H]⁺. |
| 15 | 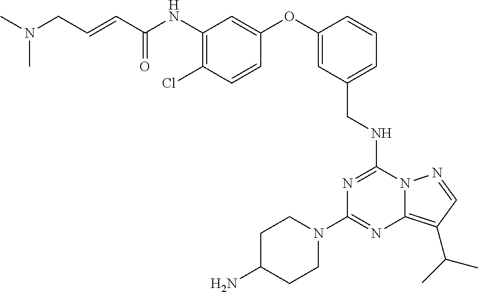 [SMILES: CC(C)c1cnn2c(NCc3cccc(Oc4ccc(Cl)c(NC(=O)/C=C/CN(C)C)c4)c3)nc(N3CCC(N)CC3)nc12] | yellow powder; ¹H-NMR (CDCl₃, 400 MHz): δ 8.18 (1H, s), 7.80 (1H, brs), 7.62 (1H, s), 7.27-7.32 (2H, m), 7.07-7.11 (2H, m), 6.92-7.05 (2H, m), 6.69-6.74 (2H, m), 6.24 (1H, d, J = 15.2 Hz), 4.67-4.76 (4H, m), 3.20- 3.21 (2H, m), 3.01-3.04 (2H, m), 2.88-2.94 (2H, m), 2.33 (6H, s), 1.98-2.01 (2H, m), 1.36-1.45 (2H, m), 1.28 (6H, d, J = 6.8 Hz); LCMS: 95.4%, MS (ESI): m/z 618.3[M + H]⁺. |
| 16 | 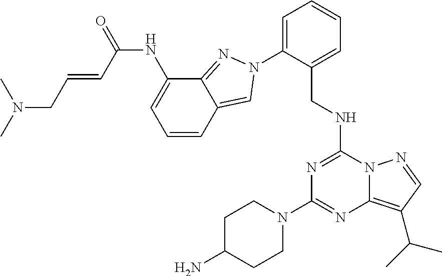 [SMILES: CC(C)c1cnn2c(NCc3ccccc3-n3cc4cccc(NC(=O)/C=C/CN(C)C)c4n3)nc(N3CCC(N)CC3)nc12] | yellow powder; ¹H-NMR (CDCl₃, 400 MHz): δ 9.22 (1H, brs), 8.59 (1H, t, J = 5.8 Hz), 8.42 (1H, d, J = 7.6 Hz), 8.19 (1H, s), 7.70 (1H, s), 7.66 (1H, d, J = 6.8 Hz), 7.40-7.47 (4H, m), 7.16-7.20 (1H, m), 7.04-7.08 (1H, m), 6.61 (1H, d, J = 15.2 Hz), 4.83-4.88 (2H, m), 4.58 (2H, d, J = 6.0 Hz), 3.38-3.40 (1H, m), 3.27 (2H, d, J = 6.4 Hz), 2.98-3.05 (3H, m), 2.56 (6H, s), 2.21-2.38 (2H, m), 1.65-1.68 (2H, m), 1.25-1.29 (8H, m); LCMS: 95.6%, MS (ESI): m/z 608.3[M + H]⁺. |
| 17 | 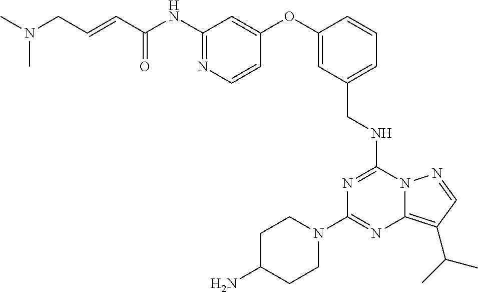 [SMILES: CC(C)c1cnn2c(NCc3cccc(Oc4ccnc(NC(=O)/C=C/CN(C)C)c4)c3)nc(N3CCC(N)CC3)nc12] | white powder; ¹H-NMR (DMSO-d₆, 400 MHz): δ 11.13 (1H, brs), 10.76 (1H, brs), 9.08 (1H, brs), 8.24 (1H, d, J = 6.0 Hz), 8.00-8.22 (3H, m), 7.79 (1H, s), 7.73 (1H, s), 7.43 (1H, d, J = 8.0 Hz), 7.33 (1H, d, J = 7.2 Hz), 7.23 (1H, s), 7.05-7.15 (1H, m), 6.80-6.90 (1H, m), 6.73 (1H, d, J = 3.6 Hz), 6.56 (1H, d, J = 16.0 Hz), 4.55-4.70 (4H, m), 3.85-3.95 (2H, m), 2.85-3.00 (4H, m), 2.74 (6H, d, J = 3.6 Hz), 1.85-2.00 (2H, m), 1.30-1.50 (2H, m), 1.22 (6H, d, J = 6.4 Hz); LCMS: 100%, MS (ESI): m/z 607.3 [M + Na]⁺ |
| 18 | 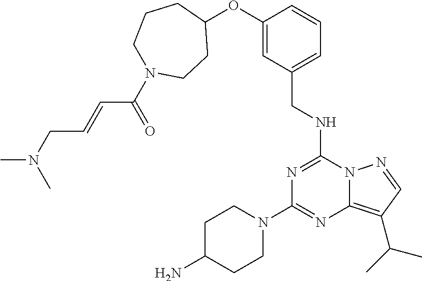 [SMILES: CC(C)c1cnn2c(NCc3cccc(OC4CCCN(C(=O)/C=C/CN(C)C)CC4)c3)nc(N3CCC(N)CC3)nc12] | Racemic mixture; white powder; ¹H-NMR (CDCl₃, 400 MHz): δ 7.61 (1H, s), 7.24 (1H, t, J = 7.8 Hz), 6.88-6.96 (3H, m), 6.78-6.81 (1H, m), 6.60-6.65 (1H, m), 6.40-6.45 (1H, m), 4.69-4.76 (4H, m), 4.48-4.51 (1H, m), 3.83-3.90 (1H, m), 3.43-3.72 (3H, m), 2.90- 3.11 (6H, m), 2.27 (6H, d, J = 5.6 Hz), 1.99-2.08 (4H, m), 1.73-1.91 (4H, m), 1.25-1.37 (8H, m); LCMS: 94.9%, MS (ESI): m/z 612.5[M + Na]⁺. |
| 19 | 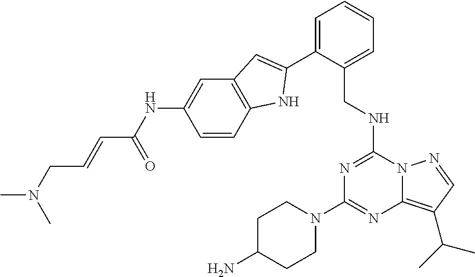 [SMILES: CC(C)c1cnn2c(NCc3ccccc3-c3cc4cc(NC(=O)/C=C/CN(C)C)ccc4[nH]3)nc(N3CCC(N)CC3)nc12] | white powder; ¹H-NMR (DMSO-d₆, 400 MHz): δ 8.72 (1H, s), 7.87 (1H, s), 7.58 (2H, s), 7.46-7.54 (2H, m), 7.33-7.40 (2H, m), 7.18-7.26 (2H, m), 6.90-7.01 (1H, m), 6.64 (1H, brs), 6.54 (1H, s), 6.13 (1H, d, J = 15.6 Hz), 4.82 (2H, s), 4.55-4.65 (2H, m), 3.11 (2H, d, J = 5.2 Hz), 2.98-3.05 (1H, m), 2.79-2.88 (3H, m), 2.28 (6H, s), 1.70-1.80 (2H, m), 1.29 (8H, d, J = 6.8 Hz); LCMS: 100%, MS (ESI): m/z 629.2[M + Na]⁺ |
| 20 | 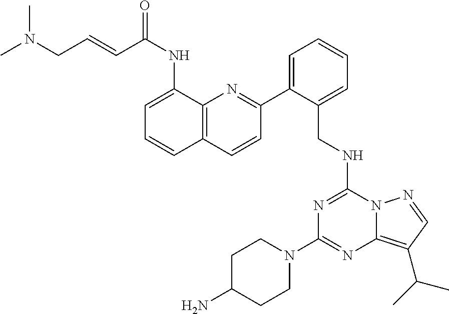 [SMILES: CC(C)c1cnn2c(NCc3ccccc3-c3ccc4cccc(NC(=O)/C=C/CN(C)C)c4n3)nc(N3CCC(N)CC3)nc12] | yellow powder; ¹H NMR (DMSO-d₆, 400 MHz): δ 10.08 (1H, brs), 8.89 (1H, t, J = 5.6 Hz), 8.69 (1H, d, J = 7.6 Hz), , 8.50 (1H, d, J = 8.4 Hz), 7.88 (1H, d, J = 8.4 Hz), 7.60-7.85 (7H, m), 7.43-7.49 (2H, m), 6.73- 6.80 (1H, m), 6.50 (1H, d, J = 16.0 Hz), 5.04 (2H, d, J = 6.4 Hz), 4.30-4.33 (2H, m), 3.11-3.17 (1H, m), 3.00 (2H, d, J = 6.4 Hz), 2.83-2.92 (1H, m), 2.62-2.67 (2H, m), 2.11 (6H, s), 1.71-1.75 (2H, m), 1.07-1.27 (8H, m); LCMS: 100%, MS (ESI): m/z 619.4[M + H]⁺. |
| 21 | 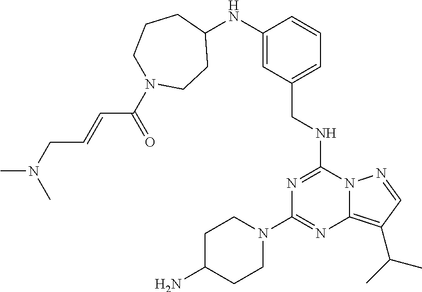 [SMILES: CC(C)c1cnn2c(NCc3cccc(NC4CCCN(C(=O)/C=C/CN(C)C)CC4)c3)nc(N3CCC(N)CC3)nc12] | Racemic mixture; white powder; ¹H-NMR (CDCl₃, 400 MHz): δ 7.60 (1H, s), 7.12 (1H, t, J = 7.2 Hz), 6.82-6.93 (1H, m), 6.64-6.69 (1H, m), 6.60-6.63 (1H, m), 6.40-6.52 (3H, m), 4.70-4.80 (2H, m), 4.56-4.70 (2H, m), 3.37-3.83 (6H, m), 2.89-3.16 (6H, m), 2.25- 2.31 (6H, m), 1.93-2.01 (3H, m), 1.59-1.76 (2H, m), 1.39-1.48 (2H, m), 1.23-1.33 (8H, m); LCMS: 100%, MS (ESI): m/z 611.5[M + Na]⁺. |
| 22 | 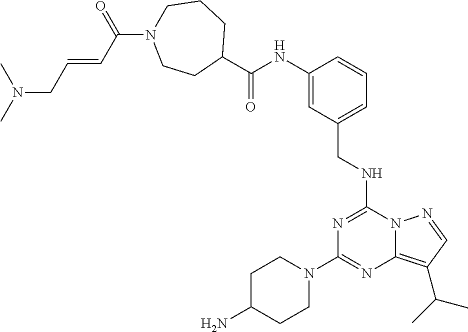 [SMILES: CC(C)c1cnn2c(NCc3cccc(NC(=O)C4CCCN(C(=O)/C=C/CN(C)C)CC4)c3)nc(N3CCC(N)CC3)nc12] | Racemic mixture; white powder; ¹H-NMR (DMSO-d₆, 400 MHz): δ 7.89 (1H, brs), 7.59 (1H, s), 7.42-7.56 (2H, m), 7.21-7.26 (1H, m), 7.06 (1H, t, J = 6.0 Hz), 6.83-6.92 (1H, m), 6.65-6.75 (1H, m), 6.40 (1H, d, J = 15.6 Hz), 4.65-4.77 (4H, m), 3.86-4.03 (1H, m), 3.75- 3.85 (1H, m), 3.63 (1H, t, J = 5.6 Hz), 3.42-3.53 (1H, m), 3.18-3.36 (1H, m), 2.88-3.12 (6H, m), 2.32-2.46 (1H, m), 2.20-2.30 (7H, m), 1.83-1.92 (2H, m), 1.54- 1.82 (2H, m), 1.19-1.40 (10H, m); LCMS: 100%, MS (ESI): m/z 617.5[M + H]⁺. |
| 23 | 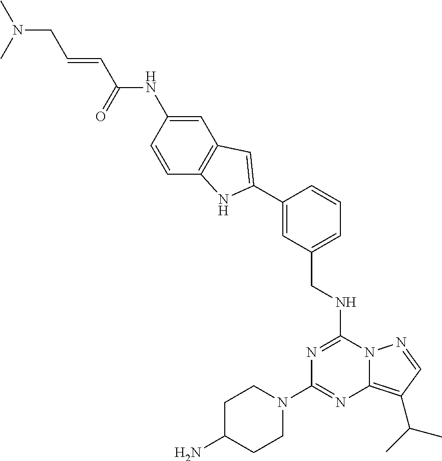 [SMILES: CC(C)c1cnn2c(NCc3cccc(-c4cc5cc(NC(=O)/C=C/CN(C)C)ccc5[nH]4)c3)nc(N3CCC(N)CC3)nc12] | white powder; ¹H NMR (DMSO-d₆, 400 MHz): δ 11.52 (1H, brs), 9.98 (1H, brs), 9.01 (1H, t, J = 5.4 Hz), 8.00 (1H, s), 7.91 (1H, s), 7.77 (1H, s), 7.73 (1H, d, J = 7.2 Hz), 7.39 (1H, t, J = 8.0 Hz), 7.31-7.34 (2H, m), 7.26 (1H, d, J = 8.0 Hz), 6.83 (1H, s), 6.67-6.73 (1H, m), 6.33 (1H, d, J = 15.2 Hz), 4.63-4.67 (4H, m), 3.13-3.19 (3H, m), 2.86-2.94 (3H, m), 2.26 (6H, s), 1.85-1.91 (2H, m), 1.29-1.36 (2H, m), 1.23 (6H, d, J = 6.8 Hz); LCMS: 100%, MS (ESI): m/z 607.3[M + H]⁺. |
| 24 | 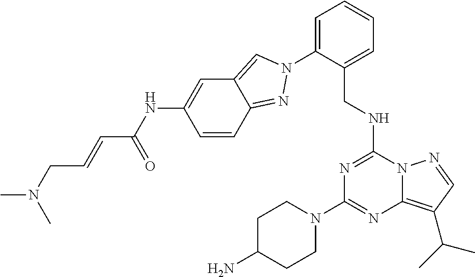 [SMILES: CC(C)c1cnn2c(NCc3ccccc3-n3cc4cc(NC(=O)/C=C/CN(C)C)ccc4n3)nc(N3CCC(N)CC3)nc12] | brown powder; ¹H-NMR (DMSO-d₆, 400 MHz): δ 10.2 (1H, brs), 8.93-8.97 (1H, m), 8.71 (1H, s), 8.38 (1H, d, J = 2.8 Hz), 7.77 (1H, s), 7.67 (1H, d, J = 9.6 Hz), 7.56- 7.72 (2H, m), 7.45-7.52 (2H, m), 7.39-7.44 (1H, m), 6.71-6.80 (1H, m), 6.38 (1H, d, J = 15.2 Hz), 4.61 (2H, d, J = 5.6 Hz), 4.39-4.50 (2H, m), 3.15-3.26 (2H, m), 2.85-2.93 (2H, m), 2.72-2.83(2H, m), 2.17-2.35 (6H, m), 1.81-1.90 (2H, m), 1.14-1.34 (8H, m); LCMS: 96.5%, MS (ESI): m/z = 608.3 [M + H]⁺. |
| 25 | 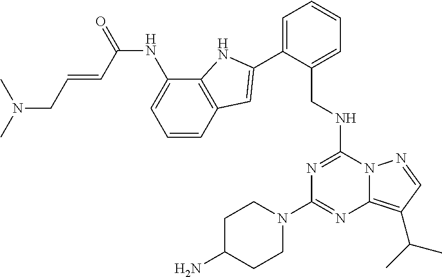 [SMILES: CC(C)c1cnn2c(NCc3ccccc3-c3cc4cccc(NC(=O)/C=C/CN(C)C)c4[nH]3)nc(N3CCC(N)CC3)nc12] | white powder; ¹H-NMR (CDCl₃, 400 MHz): δ 10.44 (1H, brs), 7.61 (1H, brs), 7.54-7.63 (3H, m), 7.48 (1H, d, J = 7.6 Hz), 7.32-7.41 (2H, m), 6.93-7.07 (2H, m), 6.79 (1H, d, J = 7.2 Hz), 6.57-6.68 (2H, m), 6.21 (1H, d, J = 14.8 Hz), 4.90 (2H, d, J = 5.6 Hz), 4.58-4.68 (2H, m), 3.12 (2H, d, J = 5.6 Hz), 3.98-3.06 (1H, m), 2.80-2.91 (3H, m), 2.29 (6H, s), 1.74-1.84 (2H, m), 1.29 (6H, d, J = 6.8 Hz), 1.20-1.26 (2H, m); LCMS: 100%, MS (ESI): m/z 629.3[M + Na]⁺. |
| 26 | 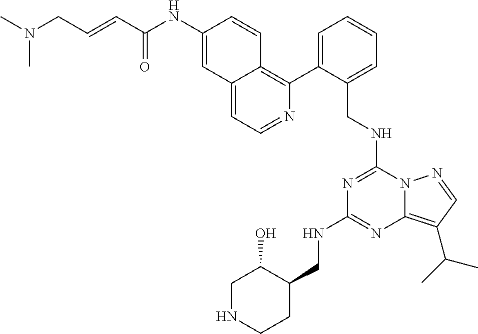 [SMILES: CC(C)c1cnn2c(NCc3ccccc3-c3nccc4cc(NC(=O)/C=C/CN(C)C)ccc34)nc(NC[C@H]3CCNC[C@@H]3O)nc12] | (3R, 4R); pale-yellow powder; ¹H NMR (400 MHz, DMSO-d₆): δ 10.91-11.17 (2H, m), 9.03 (1H, br s), 8.82 (1H, br s), 8.38-8.58 (3H, m), 7.79-7.89 (1H, m), 7.70- 7.77 (1H, m), 7.62-7.67 (1H, m), 7.50-7.57 (2H, m), 7.42-7.49 (1H, m), 7.37 (1H, br d, J = 7.2 Hz), 6.85- 6.98 (1H, m), 6.63 (1H, d, J = 15.6 Hz), 5.67-5.96 (1H, m), 4.33-4.50 (2H, m), 3.96 (2H, d, J = 6.8 Hz), 3.42- 3.57 (2H, m), 2.97-3.25 (3H, m), 2.81-2.90 (1H, m), 2.77 (6H, s), 2.58-2.69 (1H, m), 1.39-1.90 (3H, m), 1.19 (6H, br d, J = 6.4 Hz); LCMS: 100%, MS (ESI): m/z 649.3[M + H]⁺ |
| 27 | 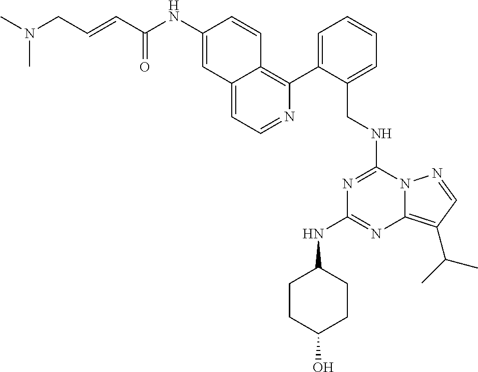 | (1R, 4R); yellow powder; ¹H-NMR (400 MHz, CD₃OD): δ 8.67 (1H, s), 8.42 (1H, br d, J = 6.4 Hz), 8.24 (1H, br d, J = 6.4 Hz), 7.84-7.93 (2H, m), 7.78 (1H, d, J = 9.2 Hz), 7.68 (1H, br s), 7.49-7.62 (3H, m), 6.95-7.10 (1H, m), 6.72 (1H, br d, J = 15.2 Hz), 4.70- 5.10 (2H, m), 4.07 (2H, br d, J = 6.8 Hz) 3.47-3.65 (2H, m), 2.91-3.01 (6H, m), 2.77-2.87 (1H, m), 1.87- 2.10 (3H, m), 1.65-1.72 (1H, m), 1.30-1.51 (4H, m), 1.12-1.23 (6H, m); LCMS: 100%, MS (ESI): m/z 634.3[M + H]⁺. |
| 28 | 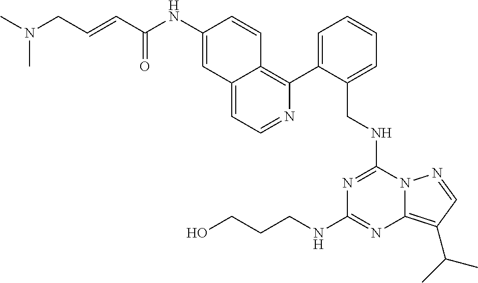 [SMILES: CC(C)c1cnn2c(NCc3ccccc3-c3nccc4cc(NC(=O)/C=C/CN(C)C)ccc34)nc(NCCCO)nc12] | yellow powder; ¹H NMR (400 MHz, CD₃OD): δ 8.42 (1H, s), 8.37 (1H, d, J = 6.0 Hz), 7.68-7.77 (2H, m), 7.61-7.67 (1H, m), 7.46-7.60 (4H, m), 7.40 (1H, d, J = 7.6 Hz), 6.92-7.02 (1H, m), 6.64 (1H, d, J = 15.2 Hz), 5.01-5.04 (1H, m), 4.39-4.43 (1H, m), 4.06 (2H, d, J = 6.4 Hz), 3.65 (2H, t, J = 6.0 Hz), 3.33-3.41 (2H, m), 2.97 (6H, s), 2.82-2.91 (1H, m), 1.74-1.80 (2H, m), 1.15-1.25 (6H, m); LCMS: 100%, MS (ESI): m/z 594.3[M + H]⁺. |
| 29 | 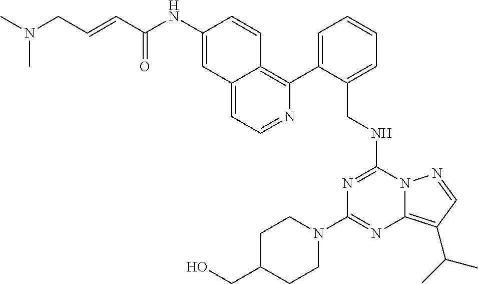 [SMILES: CC(C)c1cnn2c(NCc3ccccc3-c3nccc4cc(NC(=O)/C=C/CN(C)C)ccc34)nc(N3CCC(CO)CC3)nc12] | yellow solid; ¹H-NMR (400 MHz, CD₃OD): δ 8.66 (1H, s), 6.42-6.44 (1H, m), 8.21-8.23 (1H, m), 7.80-7.87 (3H, m), 7.59-7.71 (4H, m), 7.00-7.07 (1H, m), 6.74- 6.78 (1H, d, J = 15.6 Hz), 4.95-5.02 (1H, m), 4.71-4.81 (1H, m), 4.31-3.41 (2H, m), 4.08 (2H, d, J = 7.2 Hz), 3.46-3.47 (2H, m), 3.08-3.13 (2H, m), 2.96-2.97 (6H, m), 1.85-1.97 (2H, m), 1.29-1.38 (2H, m), 1.16-1.19 (6H, m); LCMS: 100.0%, MS (ESI): 634.3 m/z [(M + H)]⁺. |
| 30 | 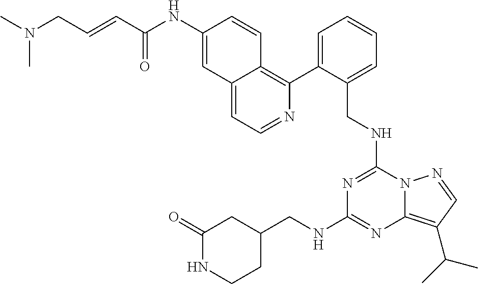 [SMILES: CC(C)c1cnn2c(NCc3ccccc3-c3nccc4cc(NC(=O)/C=C/CN(C)C)ccc34)nc(NCC3CCNC(=O)C3)nc12] | Racemic mixture; off-white powder; ¹H NMR (400 MHz, CD₃OD): δ 8.27-8.42 (2H, m) 7.25-7.75 (8H, m), 6.91-7.07 (1H, m), 6.33 (1H, d, J = 15.2 Hz), 4.80-4.87 (1H, m), 4.60-4.67 (2H, m), 4.28-4.37 (1H, m), 3.47- 3.56 (1H, m), 3.26 (2H, d, J = 6.4 Hz), 3.05-3.13 (1H, m), 2.85-2.92 (1H, m), 2.45-2.54 (2H, m), 2.36 (6H, s), 2.01-2.11 (1H, m), 1.65-1.75 (2H, m), 1.15-1.26 (6H, m); LCMS: 95.9%, MS (ESI): m/z 324.2 [M/2 + H]⁺. |
| 31 | 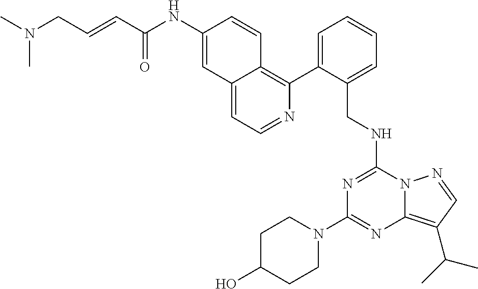 [SMILES: CC(C)c1cnn2c(NCc3ccccc3-c3nccc4cc(NC(=O)/C=C/CN(C)C)ccc34)nc(N3CCC(O)CC3)nc12] | light yellow powder; ¹H NMR (400 MHz, CD₃OD): δ 8.32 (1H, d, J = 6.0 Hz), 8.29 (1H, s), 7.63-7.68 (1H, m), 7.53-7.61 (2H, m), 7.49 (1H, t, J = 6.8 Hz), 7.36- 7.40 (2H, m), 7.31-7.35 (2H, m), 6.91-7.00 (1H, m), 6.38-6.42 (1H, m), 4.34-4.38 (1H, m), 4.13-4.25 (2H, m), 3.74-3.82 (1H, m), 3.49 (2H, d, J = 6.8 Hz), 2.98- 3.13 (2H, m), 2.85 (1H, m), 2.53 (6H, s), 1.81 (2H, m), 1.23-1.45 (3H, m), 1.19 (6H, d, J = 6.8 Hz); LCMS: 100%, MS (ESI): m/z 620.2 [M + H]⁺. |
| 32 | 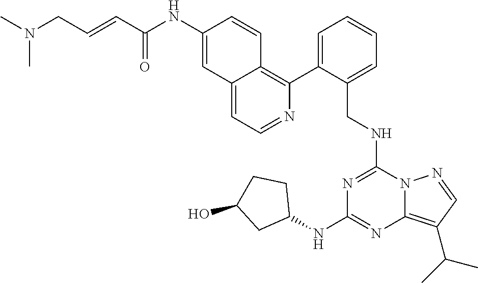 | (1S, 3S); light yellow solid; ¹H-NMR (MeOD, 400 MHz): δ 8.69-8.71 (1H, m), 8.42-8.45 (1H, m), 8.25- 8.27 (1H, m), 7.80-7.88 (3H, m), 7.58-7.72 (4H, m), 6.99-7.07 (1H, m), 6.73 (1H, d, J = 15.2 Hz), 5.01-5.06 (1H, m), 4.91-4.94 (1H, m), 4.71-4.78 (1H, m), 4.39- 4.44 (1H, m), 4.26-4.29 (1H, m), 4.07(2H, d, J = 7.2 Hz), 2.96 (6H, s), 2.84-2.88 (1H, m), 2.06-2.32 (2H, m), 1.95-1.99 (1H, m), 1.61-1.80 (3H, m), 1.18-1.21 (6H, m); LCMS: 99.7%, MS (ESI): 620.2 m/z [M + H]⁺. |
| 33 | 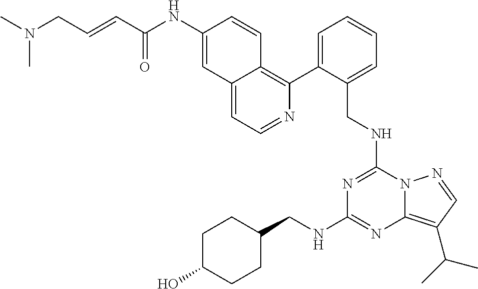 | (1R, 4R); white powder; ¹H NMR (CD₃OD, 400 MHz): δ 8.66 (1H, d, J = 2.0 Hz), 8.43 (1H, d, J = 6.8 Hz), 8.23 (1H, d, J = 6.8 Hz), 7.76-7.88 (3H, m), 7.66-7.74 (1H, m), 7.55-7.63 (3H, m), 6.97-7.08 (1H, m), 6.71 (1H, d, J = 15.6 Hz), 4.89-4.90 (2H, m), 4.07 (2H, d, J = 6.4 Hz), 3.45-3.54 (1H, m), 3.17-3.26 (1H, m), 3.04 (1H, m), 2.96 (6H, s), 2.84 (1H, m), 1.88-2.05 (2H, m), 1.67-1.83 (2H, m), 1.36-1.49 (1H, m), 1.28-1.35 (1H, m), 1.14-1.21 (7H, m), 0.84-1.11 (2H, m); LCMS: 99.7%, MS (ESI): m/z 648.2 [M + H]⁺; HPLC (254 nm): 100%. |
| 34 | 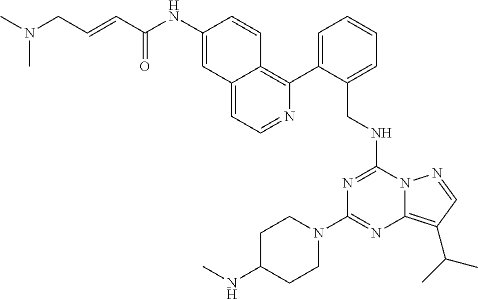 [SMILES: CNC1CCN(c2nc(NCc3ccccc3-c3nccc4cc(NC(=O)/C=C/CN(C)C)ccc34)n3ncc(C(C)C)c3n2)CC1] | yellow powder; ¹H NMR (400 MHz, CD₃OD): δ 8.67 (1H, s), 8.38-8.45 (1H, br s), 8.18-8.26 (1H, m), 7.78- 7.88 (3H, m), 7.55-7.73 (4H, m), 6.97-7.08 (1H, m), 6.74 (1H, d, J = 15.2 Hz), 4.95-5.03 (1H, m), 4.66-4.76 (1H, m), 4.48-4.59 (2H, m), 4.07 (2H, d, J = 6.8 Hz), 3.39-3.54 (1H, m), 3.03-3.24 (3H, m), 2.96 (6H, s), 2.76 (3H, s), 2.14-2.31 (2H, m), 1.46-1.80 (2H, m), 1.19 (6H, d, J = 6.8 Hz); LCMS: 99.7%, MS (ESI): m/z 633.3 [M + H]⁺ |
| 35 | 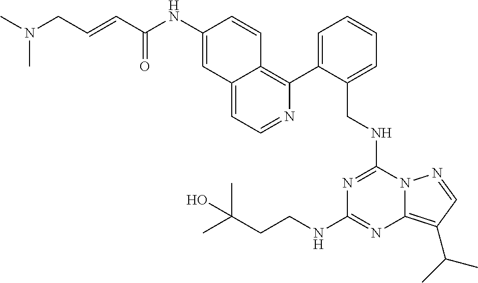 [SMILES: CC(C)c1cnn2c(NCc3ccccc3-c3nccc4cc(NC(=O)/C=C/CN(C)C)ccc34)nc(NCCC(C)(C)O)nc12] | light yellow solid; ¹H-NMR (CD₃OD, 400 MHz): δ 8.69 (1H, s), 8.44 (1H, d, J = 6.4 Hz), 8.23 (1H, d, J = 6.8 Hz), 7.81-7.87 (3H, m), 7.68-7.72 (2H, m), 7.59-7.60 (2H, m), 6.99-7.07 (1H, m), 6.73 (1H, d, J = 15.2 Hz), 5.04-5.08 (1H, m), 4.70-4.74 (1H, m), 4.07 (2H, d, J = 6.4 Hz), 3.41-3.44 (2H, m), 2.96 (6H, s), 2.82-2.86 (1H, m), 1.69-1.73 (2H, m), 1.28 (6H, s), 1.21 (6H, d, J = 6.8 Hz); LCMS: 98.2%, MS (ESI): 622.3 m/z [(M + H)]⁺. |
| 36 | 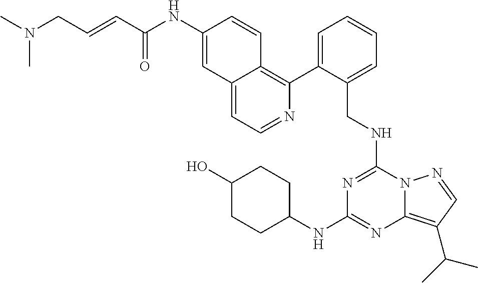 [SMILES: CC(C)c1cnn2c(NCc3ccccc3-c3nccc4cc(NC(=O)/C=C/CN(C)C)ccc34)nc(NC3CCC(O)CC3)nc12] | White solid; ¹H NMR (400 MHz, DMSO-d₆): δ 11.24- 11.45 (2H, m), 9.42-9.66 (1H, m), 8.61-8.66 (1H, m), 8.41-8.47 (1H, m), 8.18-8.23 (1H, m), 7.72-7.75 (2H, m), 7.67-7.69 (1H, m), 7.54-7.60 (1H, m), 7.41-7.52 (2H, m), 6.91-7.01 (1H, m), 6.65 (1H, m), 4.74-4.83 (1H, m), 4.50-4.61 (1H, m), 3.95-4.02 (2H, m), 2.72- 2.81 (6H, m), 1.76-1.91 (1H, m), 1.44-1.68 (5H, m), 1.21-1.38 (2H, m), 1.10-1.16 (6H, m); LCMS: 100%, MS (ESI): m/z 634.3 [M + H]⁺. |
| 37 | 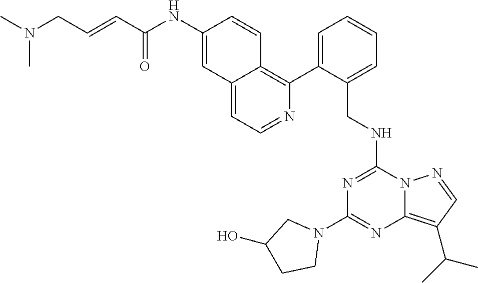 [SMILES: CC(C)c1cnn2c(NCc3ccccc3-c3nccc4cc(NC(=O)/C=C/CN(C)C)ccc34)nc(N3CCC(O)C3)nc12] | Racemic mixture; off-white powder; ¹H NMR (400 MHz, CD₃OD): δ 8.64-8.70 (1H, m), 8.40-8.51 (1H, m), 8.17-8.28 (1H, m), 7.81-7.92 (3H, m), 7.68-7.75 (2H, m), 7.58-7.66 (2H, m), 7.01-7.09 (1H, m), 6.77 (1H, d, J = 15.2 Hz), 5.11-5.20 (1H, m), 4.53-4.72 (2H, m), 4.09 (2H, d, J = 7.2 Hz), 3.38-3.86 (4H, m), 3.08- 3.21 (1H, m), 2.93 (6H, s), 2.03-2.32 (2H, m), 1.14-1.26 (6H, m); HPLC: 100%, MS (ESI): m/z 606.2 [M + H]⁺. |
| 38 | 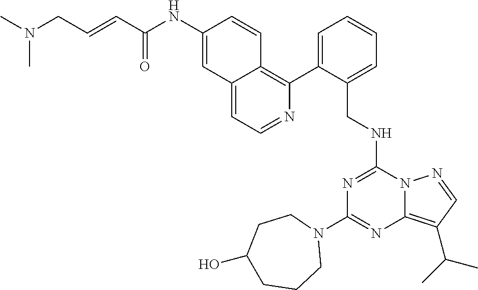 [SMILES: CC(C)c1cnn2c(NCc3ccccc3-c3nccc4cc(NC(=O)/C=C/CN(C)C)ccc34)nc(N3CCCC(O)CC3)nc12] | Racemic mixture; light yellow solid; ¹H-NMR (MeOD, 400 MHz): δ 8.67 (1H, s), 8.41-8.43 (1H, m), 8.18-8.23 (1H, m), 7.80-7.87 (3H, m), 7.59-7.68 (4H, m), 6.99- 7.07 (1H, m), 6.74 (1H, d, J = 15.2 Hz), 4.95-4.99 (1H, m), 4.72-4.81 (1H, m), 4.07 (2H, d, J = 7.2 Hz), 3.42- 3.93 (4H, m), 3.09-3.14 (1H, m), 2.96 (6H, s), 2.07- 2.22 (1H, m), 1.43-1.98 (5H, m), 1.16-1.19 (6H, m); LCMS: 99.8%, MS (ESI): 634.2 m/z [(M + H)]⁺ |
| 39 | 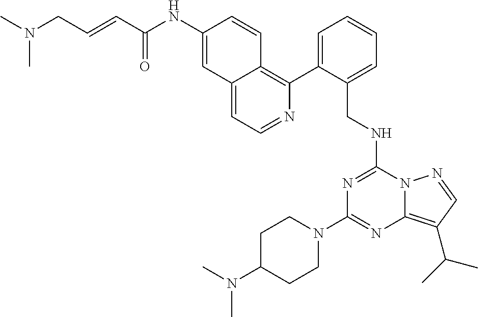 [SMILES: CC(C)c1cnn2c(NCc3ccccc3-c3nccc4cc(NC(=O)/C=C/CN(C)C)ccc34)nc(N3CCC(N(C)C)CC3)nc12] | Light yellow solid; ¹H NMR (400 MHz, CD₃OD): δ 8.68 (1H, s), 8.42 (1H, d, J = 6.0 Hz), 8.21 (1H, d, J = 6.4 Hz), 7.82-7.89 (3H, m), 7.65-7.75 (2H, m), 7.59- 7.64 (2H, m), 7.00-7.11 (1H, m), 6.74 (1H, d, J = 15.6 Hz), 4.98-5.07 (1H, m), 4.68-4.75 (1H, m), 4.54-4.62 (2H, m), 4.07 (2H, d, J = 6.8 Hz), 3.59-3.69 (1H, m), 3.08-3.28 (3H, m), 2.98 (6H, s), 2.94 (6H, s), 2.21- 2.29 (2H, m), 1.72-1.87 (2H, m), 1.29-1.33 (2H, m), 1.19-1.24 (6H, m); LCMS: 97.9%, MS (ESI): m/z 647.3 [M + H]⁺ |
| 40 | 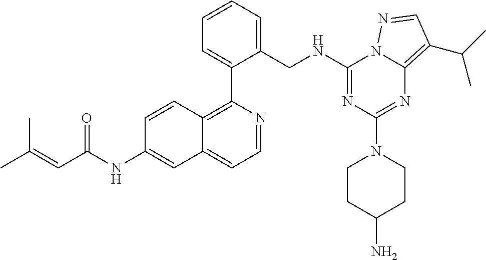 [SMILES: CC(C)=CC(=O)Nc1ccc2c(-c3ccccc3CNc3nc(N4CCC(N)CC4)nc4c(C(C)C)cnn34)nccc2c1] | yellow powder; ¹H NMR (400 MHz, CD₃OD): δ 8.58 (1H, s), 8.38 (1H, s), 8.10-8.19 (1H, m), 7.80-7.90 (2H, m), 7.66-7.74 (2H, m), 7.52-7.63 (3H, m), 6.02 (1H, s), 5.00-5.09 (1H, m), 4.65-4.72 (1H, m), 4.48-4.56 (2H, m), 3.46-3.59 (1H, m), 2.97-3.23 (3H, m), 2.33 (3H, s), 2.10-2.22 (2H, m), 2.01 (3H, s), 1.47-1.76 (2H, m), 1.21 (6H, d, J = 6.8 Hz); HPLC: 96.1%, MS (ESI): m/z 590.2[M + H]⁺ |
| 41 | 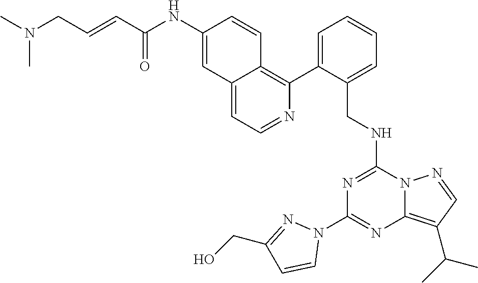 [SMILES: CC(C)c1cnn2c(NCc3ccccc3-c3nccc4cc(NC(=O)/C=C/CN(C)C)ccc34)nc(-n3ccc(CO)n3)nc12] | Yellow powder; ¹H NMR (CD₃OD, 400 MHz): δ 8.51 (1H, s), 8.37 (1H, d, J = 2.8 Hz), 8.11 (1H, d, J = 6.8 Hz), 7.77-7.89 (4H, m), 7.62-7.71 (3H, m), 7.57 (1H, d, J = 6.8 Hz), 6.97-7.05 (1H, m), 6.58-6.63 (2H, m), 5.03-5.10 (1H, m), 4.76-4.81 (1H, m), 4.74 (2H, s), 4.09 (2H, d, J = 7.2 Hz), 3.08-3.17 (1H, m), 2.99 (6H, s), 1.29-1.34 (6H, m); LCMS: 99.2%, MS (ESI): 617.2 m/z [M + H]⁺ |
| 42 | 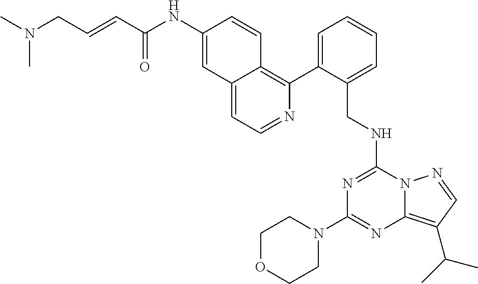 [SMILES: CC(C)c1cnn2c(NCc3ccccc3-c3nccc4cc(NC(=O)/C=C/CN(C)C)ccc34)nc(N3CCOCC3)nc12] | off-white powder; ¹H NMR (400 MHz, CD₃OD): δ 8.65 (1H, d, J = 1.6 Hz), 8.41 (1H, d, J = 6.8 Hz), 8.21 (1H, d, J = 6.4 Hz), 7.77-7.89 (3H, m), 7.55-7.73 (4H, m), 6.97-7.12 (1H, m), 6.75 (1H, d, J = 15.2 Hz), 4.97-5.03 (1H, m), 4.69-4.76 (1H, m), 4.07 (2H, d, J = 6.8 Hz), 3.66-3.85 (8H, m), 3.04-3.16 (1H, m), 2.96 (6H, s), 1.16-1.21 (6H, m); HPLC: 99.4%, MS (ESI): m/z 606.2 [M + H]⁺ |
| 43 | 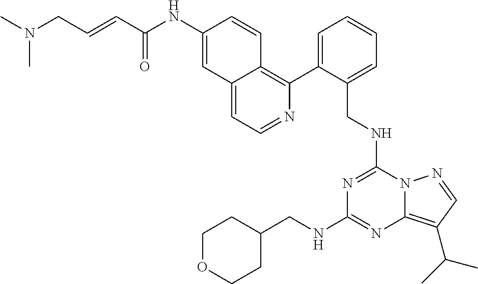 [SMILES: CC(C)c1cnn2c(NCc3ccccc3-c3nccc4cc(NC(=O)/C=C/CN(C)C)ccc34)nc(NCC3CCOCC3)nc12] | White powder; ¹H NMR (CD₃OD, 400 MHz): δ 8.70 (1H, s), 8.46 (1H, d, J = 7.2 Hz), 8.22-8.28 (1H, m), 7.82-7.89 (3H, m), 7.57-7.76 (4H, m), 7.00-7.09 (1H, m), 6.71 (1H, d, J = 15.2 Hz), 4.96-5.02 (1H, m), 4.75- 4.83 (1H, m), 4.08 (2H, d, J = 6.4 Hz), 3.84-4.01 (2H, m), 3.32-3.39 (2H, m), 3.23-3.28 (1H, m), 3.06-3.21 (1H, m), 2.98 (6H, s), 2.83-2.88 (1H, m), 1.73-1.80 (1H, m), 1.55-1.64 (2H, m), 1.25-1.32 (1H, m), 1.18- 1.24 (6H, m); LCMS: 100%, MS (ESI): m/z 634.2 [M + H]⁺ |
| 44 | 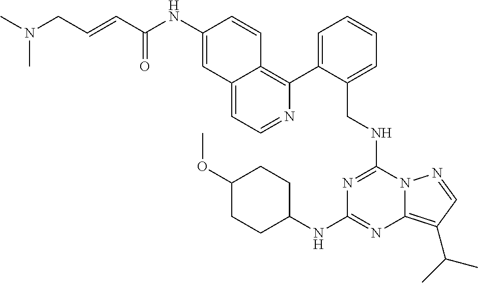 [SMILES: COC1CCC(Nc2nc(NCc3ccccc3-c3nccc4cc(NC(=O)/C=C/CN(C)C)ccc34)n3ncc(C(C)C)c3n2)CC1] | Light yellow solid; ¹H-NMR (CD₃OD, 400 MHz): δ 8.88-6.89 (1H, m), 8.41-8.43 (1H, m), 8.26-8.28 (1H, m), 7.78-7.88 (3H, m), 7.67-7.71 (1H, m), 7.55-7.60 (3H, m), 6.99-7.07 (1H, m), 6.71 (1H, d, J = 15.2 Hz), 4.95-4.99 (1H, m), 4.78-4.82 (1H, m), 4.07 (2H, d, J = 7.2 Hz), 3.52-3.57 (1H, m), 3.40 (3H, s), 3.22-3.28 (1H, m), 2.96 (6H, s), 2.80-2.87 (1H, m), 2.03-2.17 (3H, m), 1.71-1.73 (1H, m), 1.27-1.48 (4H, m), 1.16-1.19 (6H, m); LCMS: 100.0%, MS (ESI): 648.3 m/z [(M + H)]⁺ |
| 45 | 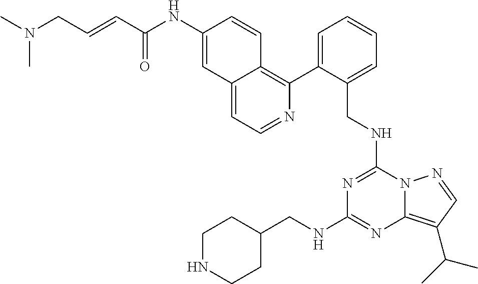 [SMILES: CC(C)c1cnn2c(NCc3ccccc3-c3nccc4cc(NC(=O)/C=C/CN(C)C)ccc34)nc(NCC3CCNCC3)nc12] | Light yellow solid; ¹H-NMR (CD₃OD, 400 MHz): δ 8.68 (1H, s), 8.45 (1H, d, J = 6.4 Hz), 8.23 (1H, d, J = 6.8 Hz), 7.82-7.83 (3H, m), 7.68-7.72 (1H, m), 7.59- 7.65 (3H, m), 6.99-7.05 (1H, m), 6.72 (1H, d, J = 15.2 Hz), 5.04-5.08 (1H, m), 4.77-4.81 (1H, m), 4.07 (2H, d, J = 6.8 Hz), 3.37-3.48 (3H, m), 3.18-3.27 (1H, m), 2.96-3.02 (8H, m), 2.83-2.88 (1H, m), 1.93-1.96 (m, 3H), 1.49-1.55 (2H, m), 1.18-1.21 (6H, m); LCMS: 100.0%, MS (ESI): 633.3 m/z [(M + H)]⁺ |
| 46 | 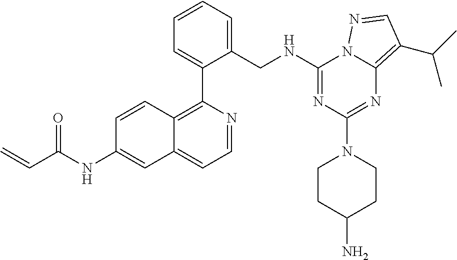 [SMILES: C=CC(=O)Nc1ccc2c(-c3ccccc3CNc3nc(N4CCC(N)CC4)nc4c(C(C)C)cnn34)nccc2c1] | off-white powder; ¹H NMR (400 MHz, CD₃OD): δ 8.59 (1H, d, J = 2.0 Hz), 8.38 (1H, d, J = 6.4 Hz), 8.16 (1H, d, J = 6.4 Hz), 7.79-7.88 (2H, m), 7.66-7.78 (2H, m), 7.57-7.62 (2H, m), 7.53 (1H, s), 6.51-6.58 (2H, m), 5.94 (1H, dd, J = 8.0, 3.6 Hz), 4.98-5.09 (1H, m), 4.65-4.74 (1H, m), 4.49-4.57 (2H, m), 3.43-3.54 (1H, m), 2.95- 3.21 (3H, m), 2.04-2.21 (2H, m), 1.44-1.76 (2H, m), 1.20 (6H, d, J = 6.8 Hz); HPLC: 100%, MS (ESI): m/z 562.2[M + H]⁺. |
| 47 | 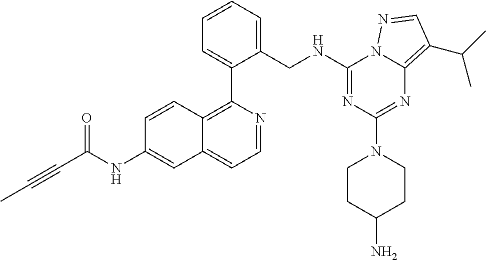 [SMILES: CC#CC(=O)Nc1ccc2c(-c3ccccc3CNc3nc(N4CCC(N)CC4)nc4c(C(C)C)cnn34)nccc2c1] | Light yellow powder; ¹H NMR (CD₃OD, 400 MHz): δ 8.43 (1H, s), 8.39 (1H, d, J = 6.4 Hz), 8.17 (1H, d, J = 6.4 Hz), 7.78-7.84 (2H, m), 7.62-7.71 (2H, m), 7.56-7.62 (2H, m), 7.55 (1H, s), 4.91-5.05 (1H, m), 4.63-4.71 (1H, m), 4.47-4.551 (2H, m), 3.43-3.56 (1H, m), 3.10-3.29 (2H, m), 3.00-3.08 (1H, m), 2.02-2.21 (5H, m), 1.63- 1.76 (1H, m), 1.51-1.59 (1H, m), 1.21-1.27 (6H, m); LCMS: 96.8%, MS (ESI): m/z 574.2 [M + H]⁺ |
| 48 | 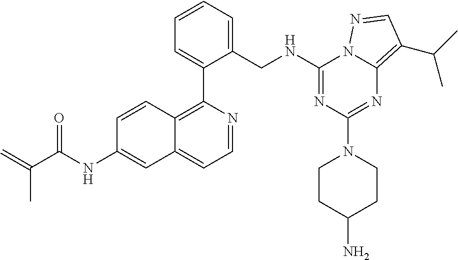 [SMILES: C=C(C)C(=O)Nc1ccc2c(-c3ccccc3CNc3nc(N4CCC(N)CC4)nc4c(C(C)C)cnn34)nccc2c1] | Pale yellow powder; ¹H NMR (CD₃OD, 400 MHz): δ 8.55 (1H, d, J = 2.0 Hz), 8.37-8.42 (1H, m), 8.20 (1H, d, J = 6.4 Hz), 7.77-7.92 (3H, m), 7.64-7.72 (1H, m), 7.54- 7.62 (3H, m), 5.98 (1H, s), 5.71 (1H, d, J = 1.2 Hz), 4.99-5.07 (1H, m), 4.67-4.73 (1H, m), 4.47-4.54 (1H, m), 3.44-3.56 (1H, m), 3.17-3.24 (1H, m), 3.03-3.09 (1H, m), 2.08-2.21 (2H, m), 2.06 (3H, s), 1.64-1.78 (1H, m), 1.55-1.60 (1H, m), 1.16-1.21 (6H, m); LCMS: 100%, MS (ESI): m/z 576.2 [M + H]⁺ |
| 49 | 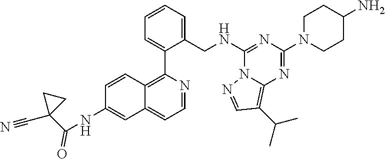 [SMILES: CC(C)c1cnn2c(NCc3ccccc3-c3nccc4cc(NC(=O)C5(C#N)CC5)ccc34)nc(N3CCC(N)CC3)nc12] | White powder; ¹H NMR (CD₃OD, 400 MHz): δ 8.48 (1H, d, J = 1.6 Hz), 8.40 (1H, d, J = 6.8 Hz), 8.19 (1H, d, J = 6.8 Hz), 7.80-7.89 (3H, m), 7.63-7.72 (2H, m), 7.55-7.62 (2H, m), 4.98-5.07 (1H, m), 4.68-4.75 (1H, m), 4.51-4.57 (2H, m), 3.46-3.52 (1H, m), 3.13-3.23 (2H, m), 3.04-3.09 (1H, m), 2.08-2.17 (2H, m), 1.74- 1.87 (4H, m), 1.55-1.72 (2H, m), 1.23-1.28 (6H, m); LCMS: 99.8%, MS (ESI): 601.2 m/z [M + H]⁺ |
| 50 | 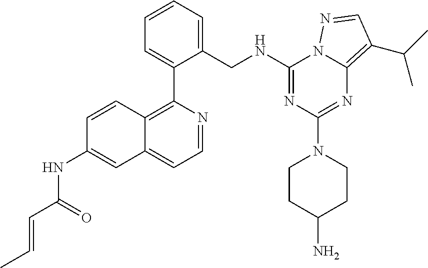 [SMILES: C/C=C/C(=O)Nc1ccc2c(-c3ccccc3CNc3nc(N4CCC(N)CC4)nc4c(C(C)C)cnn34)nccc2c1] | Yellow powder; ¹H NMR (CD₃OD, 400 MHz): δ 8.57 (1H, d, J = 2.0 Hz), 8.41 (1H, d, J = 6.4 Hz), 8.19 (1H, d, J = 6.8 Hz), 7.73-7.88 (3H, m), 7.66-7.71 (1H, m), 7.56-7.62 (3H, m), 7.06-7.17 (1H, m), 6.25 (1H, dd, J = 14.8, 2.0 Hz), 4.99-5.06 (1H, m), 4.66-4.73 (1H, m), 4.45-4.52 (2H, m), 3.47-3.58 (1H, m), 3.18-3.38 (2H, m, overlap with CD₃OD signal), 3.0-3.09 (1H, m), 2.10-2.19 (2H, m), 1.98 (3H, dd, J = 6.8, 1.6 Hz), 1.65-1.80 (1H, m), 1.49-1.64 (1H, m), 1.18 (6H, d, J = 6.8 Hz); LCMS: 100%, MS (ESI): m/z 576.3 [M + H]⁺ |
| 51 | 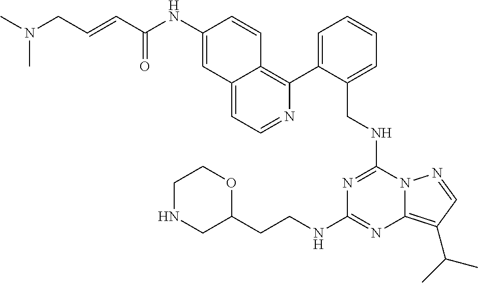 [SMILES: CC(C)c1cnn2c(NCc3ccccc3-c3nccc4cc(NC(=O)/C=C/CN(C)C)ccc34)nc(NCCC3CNCCO3)nc12] | Racemic mixture; off-white powder; ¹H NMR (400 MHz, CD₃OD): δ 8.71 (1H, s), 8.48 (1H, d, J = 6.4 Hz), 8.27 (1H, d, J = 6.0 Hz), 7.81-7.92 (3H, m), 7.56-7.75 (4H, m), 7.00-7.13 (1H, m), 6.77 (1H, d, J = 15.2 Hz), 5.06-5.14 (1H, m), 4.72-4.79 (1H, m), 4.06-4.21 (3H, m), 3.86-3.97 (2H, m), 3.43-3.59 (2H, m), 3.25-3.33 (3H, m), 3.14-3.26 (1H, m), 2.98 (6H, s), 2.83-2.94 (1H, m), 1.57-1.82 (2H, m), 1.21 (6H, d, J = 6.4 Hz); HPLC: 100%, MS (ESI): m/z 649.3[M + H]⁺ |
| 52 | 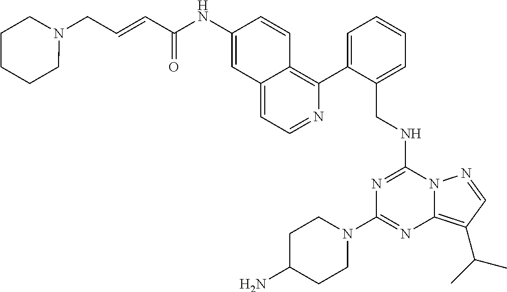 [SMILES: CC(C)c1cnn2c(NCc3ccccc3-c3nccc4cc(NC(=O)/C=C/CN5CCCCC5)ccc34)nc(N3CCC(N)CC3)nc12] | yellow powder; ¹H NMR (400 MHz, CD₃OD): δ 8.69 (1H, s), 8.47 (1H, s), 8.26 (1H, d, J = 5.2 Hz), 7.80-7.93 (3H, m), 7.56-7.76 (4H, m), 7.02-7.14 (1H, m), 6.76 (1H, d, J = 14.8 Hz), 5.08-5.18 (1H, m), 4.69-4.78 (1H, m), 4.47-4.56 (2H, m), 4.03-4.08 (2H, m), 3.48-3.68 (5H, m), 2.94-3.18 (3H, m), 2.11-2.23 (2H, m), 2.00- 2.07 (2H, m), 1.80-1.92 (4H, m), 1.51-1.67 (2H, m), 1.20 (6H, d, J = 6.0 Hz); HPLC: 97.1%, MS (ESI): m/z 659.3[M + H]⁺ |
| 53 | 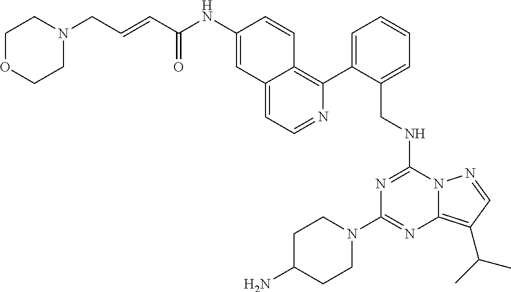 [SMILES: CC(C)c1cnn2c(NCc3ccccc3-c3nccc4cc(NC(=O)/C=C/CN5CCOCC5)ccc34)nc(N3CCC(N)CC3)nc12] | yellow powder; ¹H NMR (400 MHz, CD₃OD): δ 8.69 (1H, s), 8.45 (1H, d, J = 5.2 Hz), 8.25 (1H, d, J = 6.4 Hz), 7.81-7.96 (3H, m), 7.59-7.76 (4H, m), 7.02-7.17 (1H, m), 6.79 (1H, d, J = 15.2 Hz), 5.01-5.08 (1H, m), 4.73-4.78 (1H, m), 4.48-4.55 (2H, m), 4.05-4.21 (4H, m), 3.79-3.92 (2H, m), 3.48-3.63 (4H, m), 3.12-3.28 (4H, m), 2.11-2.27 (2H, m), 1.56-1.85 (2H, m), 1.20 (6H, d, J = 6.0 Hz); HPLC: 100%, MS (ESI): m/z 661.3[M + H]⁺ |
| 54 | 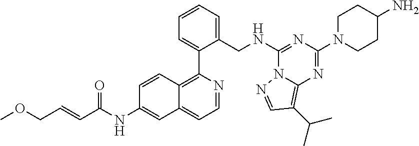 [SMILES: COC/C=C/C(=O)Nc1ccc2c(-c3ccccc3CNc3nc(N4CCC(N)CC4)nc4c(C(C)C)cnn34)nccc2c1] | off-white powder; ¹H NMR (CD₃OD, 400 MHz): δ 8.56 (1H, s), 8.31-8.36 (1H, m), 8.08-8.13 (1H, m), 7.77- 7.90 (2H, m), 7.42-7.75 (5H, m), 7.06-7.13 (1H, m), 6.46 (1H, d, J = 15.2 Hz), 4.64-4.71 (1H, m), 4.52-4.58 (2H, m), 4.14-4.23 (2H, m), 3.44-3.53 (4H, m), 2.87- 3.19 (3H, m), 2.04-2.13 (2H, m), 1.29-1.72 (3H, m), 1.20 (6H, d, J = 6.8 Hz); LCMS: 99.8%, MS (ESI): 606.2 m/z [M + H]+ |
| 55 | 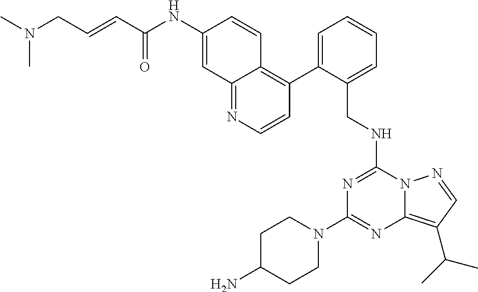 [SMILES: CC(C)c1cnn2c(NCc3ccccc3-c3ccnc4cc(NC(=O)/C=C/CN(C)C)ccc34)nc(N3CCC(N)CC3)nc12] | yellow powder; ¹H NMR (400 MHz, DMSO-d₆): δ ppm 10.95 (1H, brs) 8.87 (1H, d, J = 4.8 Hz), 8.65-8.72 (1H, m), 8.64 (1H, s), 8.13 (2H, brs), 7.65-7.75 (2H, m), 7.39-7.57 (3H, m), 7.24-7.38 (3H, m), 6.82-6.95 (1H, m), 6.62 (1H, d, J = 14.8 Hz), 4.18-4.48 (4H, m), 3.76- 3.88 (2H, m), 3.19-3.26 (1H, m), 2.83-2.94 (1H, m), 2.55-2.76 (9H, m), 1.86 (2H, m), 1.28-1.39 (2H, m), 1.20 (6H, d, J = 6.8 Hz); LCMS: 100%, MS (ESI): m/z 619.3 [M + H]⁺ |
| 56 | 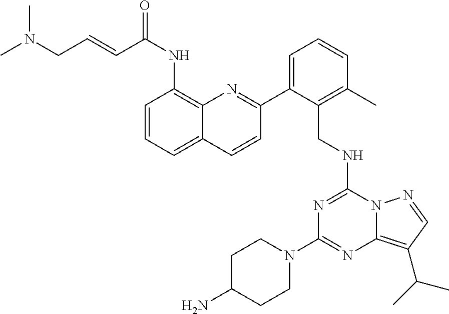 [SMILES: Cc1cccc(-c2ccc3cccc(NC(=O)/C=C/CN(C)C)c3n2)c1CNc1nc(N2CCC(N)CC2)nc2c(C(C)C)cnn12] | yellow solid; ¹H-NMR (CD₃OD, 400 MHz): δ 8.70-8.72 (1H, m), 8.29-8.33 (1H, m), 7.78 (1H, s), 7.61-7.72 (3H, m), 7.42-7.48 (2H, m), 7.32-7.34 (1H, m), 6.86-6.92 (1H, m), 6.72-6.75 (1H, m), 4.32-4.36 (2H, m), 4.03 (2H, d, J = 7.2 Hz), 3.45-3.49 (1H, m), 3.02-3.15 (3H, m), 2.94 (6H, s), 2.61 (3H, s), 2.09-2.12 (2H, m), 1.55- 1.61 (2H, m), 1.26 (6H, d, J = 6.8 Hz); LCMS: 100.0%, MS (ESI): 633.3 m/z [(M + H)]⁺ |
| 57 | 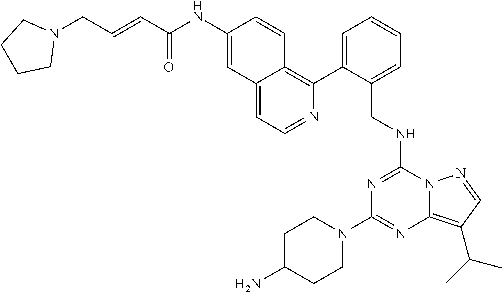 [SMILES: CC(C)c1cnn2c(NCc3ccccc3-c3nccc4cc(NC(=O)/C=C/CN5CCCC5)ccc34)nc(N3CCC(N)CC3)nc12] | off-white powder; ¹H NMR (400 MHz, CD₃OD): δ 8.63 (1H, s), 8.32-8.37 (1H, m), 8.14 (1H, d, J = 6.4 Hz), 7.80-7.87 (2H, m), 7.72-7.76 (1H, m), 7.68 (1H, t, J = 8.0 Hz), 7.54-7.63 (2H, m), 7.48 (1H, s), 6.96-7.15 (1H, m), 6.72 (1H, d, J = 15.20 Hz), 4.66-4.73 (2H, m), 4.53-4.59 (2H, m), 4.16 (2H, d, J = 6.8 Hz), 3.69-3.74 (2H, m), 3.42-3.53 (1H, m), 2.93-3.25 (5H, m), 2.03- 2.32 (6H, m), 1.43-1.69 (2H, m), 1.20 (6H, d, J = 7.2 Hz); HPLC: 100%, MS (ESI): m/z 645.3[M + H]⁺ |
| 58 | 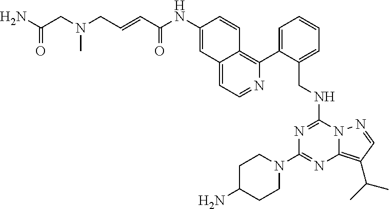 [SMILES: CC(C)c1cnn2c(NCc3ccccc3-c3nccc4cc(NC(=O)/C=C/CN(C)CC(N)=O)ccc34)nc(N3CCC(N)CC3)nc12] | yellow powder; ¹H NMR (400 MHz, CD₃OD): δ 8.63 (1H, s), 8.37 (1H, d, J = 6.4 Hz), 8.16 (1H, d, J = 6.2 Hz), 7.73-7.88 (3H, m), 7.50-7.71 (4H, m), 6.95-7.08 (1H, m), 6.72 (1H, d, J = 15.2 Hz), 4.95-5.03 (1H, m), 4.65-4.71 (1H, m), 4.46-4.54 (2H, m), 3.99-4.19 (4H, m), 3.42-3.50 (1H, m), 3.10-3.17 (2H, m), 2.91-3.07 (4H, m), 2.05-2.13 (2H, m), 1.43-1.74 (2H, m), 1.16 (6H, d, J = 6.8 Hz); HPLC: 100%, MS (ESI): m/z 662.3[M + H]⁺ |
| 59 | 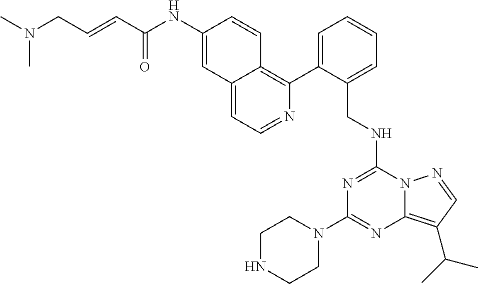 [SMILES: CC(C)c1cnn2c(NCc3ccccc3-c3nccc4cc(NC(=O)/C=C/CN(C)C)ccc34)nc(N3CCNCC3)nc12] | pale yellow powder; ¹H NMR (CD₃OD, 400 MHz): δ 8.61 (1H, d, J = 1.6 Hz), 8.33 (1H, d, J = 6.8 Hz), 8.11 (1H, d, J = 6.4 Hz), 7.77-7.85 (2H, m), 7.72-7.76 (1H, m), 7.64-7.70 (1H, m), 7.54-7.63 (2H, m), 7.48 (1H, s), 6.97-7.07 (1H, m), 6.72 (1H, d, J = 15.2 Hz), 4.96-5.03 (2H, m), 4.67-4.74 (1H, m), 4.05-4.09 (2H, m), 3.91- 4.00 (4H, m), 3.26-3.32 (4H, m), 2.94-2.99 (7H, m), 1.20 (6H, d, J = 7.2 Hz); LCMS: 100%, MS (ESI): m/z 605.6 [M + H]⁺ |
| 60 | 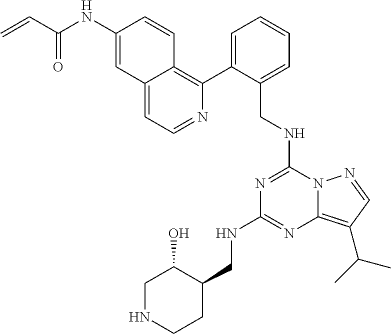 [SMILES: C=CC(=O)Nc1ccc2c(-c3ccccc3CNc3nc(NC[C@H]4CCNC[C@@H]4O)nc4c(C(C)C)cnn34)nccc2c1] | (3R, 4R); off-white powder; ¹H NMR (400 MHz, CD₃OD): δ 8.54 (1H, s), 8.33-8.37 (1H, m), 8.06-8.12 (1H, m), 7.67-7.78 (2H, m), 7.51-7.64 (3H, m), 7.44- 7.50 (2H, m), 6.38-6.44 (2H, m), 5.80-5.86 (1H, m), 4.94-5.05 (2H, m), 4.52-4.72 (2H, m), 3.49-3.75 (2H, m), 3.32-3.38 (1H, m), 2.64-2.95 (3H, m), 1.85-2.01 (1H, m), 1.71-1.79 (1H, m), 1.42-1.50 (1H, m), 1.09 (6H, d, J = 6.8 Hz); HPLC: 97.5%, MS (ESI): m/z 592.3[M + H]⁺ |
| 61 | 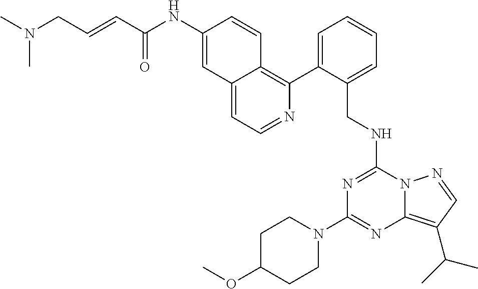 [SMILES: COC1CCN(c2nc(NCc3ccccc3-c3nccc4cc(NC(=O)/C=C/CN(C)C)ccc34)n3ncc(C(C)C)c3n2)CC1] | pale yellow powder; ¹H NMR (400 MHz, CD₃OD): δ 8.64 (1H, s), 8.37 (1H, d, J = 6.8 Hz), 8.16 (1H, d, J = 6.8 Hz), 7.76-7.86 (3H, m), 7.65-7.70 (1H, m), 7.54-7.64 (3H, m), 6.98-7.07 (1H, m), 6.72 (1H, d, J = 15.2 Hz), 4.95-5.01 (1H, m), 4.81-4.87 (1H, m), 4.68-4.74 (1H, m), 4.07 (2H, d, J = 7.2 Hz), 3.85-3.99 (2H, m), 3.52- 3.64 (3H, m), 3.40 (3H, s), 3.00-3.09 (1H, m), 2.96 (6H, s), 2.91 (1H, s), 1.83-2.05 (3H, m), 1.57-1.69 (2H, m), 1.15-1.20 (6H, m); LCMS: 99.4%, MS (ESI): m/z 634.3 [M + H]⁺ |
| 62 | 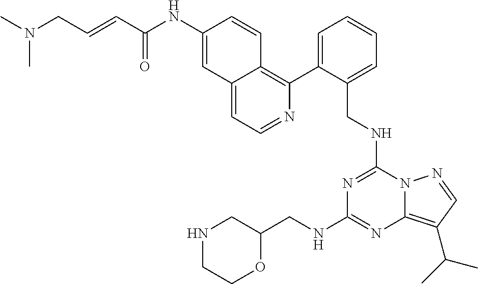 [SMILES: CC(C)c1cnn2c(NCc3ccccc3-c3nccc4cc(NC(=O)/C=C/CN(C)C)ccc34)nc(NCC3CNCCO3)nc12] | Racemic mixture; yellow solid; ¹H-NMR (CD₃OD, 400 MHz): δ 8.65-8.69 (1H, m), 8.43-8.47 (1H, m), 8.18- 8.24 (1H, m), 7.78-7.88 (3H, m), 7.65-7.72 (2H, m), 7.57-7.61 (2H, m), 6.98-7.06 (1H, m), 6.73 (1H, d, J = 15.2 Hz), 4.99-5.11 (2H, m), 4.70-4.81 (1H, m), 3.84- 4.12 (5H, m), 3.38-3.65 (3H, m), 2.81-3.24 (9H, m), 1.16-1.22 (6H, m); LCMS: 100%, MS (ESI): 657.2 m/z [M + Na]⁺ |
| 63 | 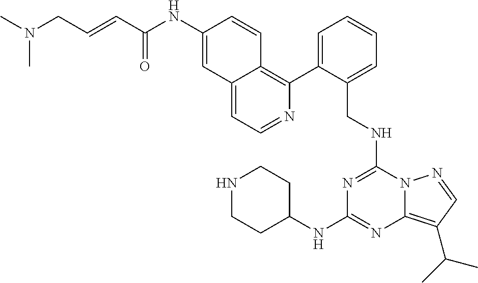 [SMILES: CC(C)c1cnn2c(NCc3ccccc3-c3nccc4cc(NC(=O)/C=C/CN(C)C)ccc34)nc(NC3CCNCC3)nc12] | yellow powder; ¹H NMR (CD₃OD, 400 MHz): δ 8.63 (1H, s), 8.39-8.45 (1H, m), 8.11-8.17 (1H, m), 7.50-7.91 (7H, m), 6.96-7.13 (1H, m), 6.72 (1H, d, J = 15.4 Hz), 5.09-5.16 (1H, m), 3.97-4.16 (3H, m), 3.37-3.62 (3H, m), 3.12-3.27 (1H, m), 2.77-3.05 (8H, m), 2.24-2.31 (1H, m), 1.68-2.05 (3H, m), 1.12-1.29 (6H, m); LCMS: 100%, MS (ESI): 619.3 m/z [M + H]⁺ |
| 64 | 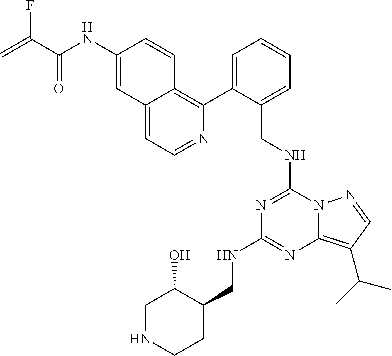 [SMILES: C=C(F)C(=O)Nc1ccc2c(-c3ccccc3CNc3nc(NC[C@H]4CCNC[C@@H]4O)nc4c(C(C)C)cnn34)nccc2c1] | (3R, 4R); yellow solid; ¹H-NMR (CD₃OD, 400 MHz): δ 8.64-8.68 (1H, m), 8.51 (1H, dd, J = 6.4, 3.2 Hz), 8.27- 8.31 (1H, m), 7.81-7.92 (3H, m), 7.65-7.72 (2H, m), 7.57-7.62 (2H, m), 5.87 (1H, dd, J = 46.0, 3.6 Hz), 5.46 (1H, dd, J = 14.8, 3.6 Hz), 5.06-5.13 (1H, m), 4.65-4.75 (1H, m), 3.69-3.76 (1H, m), 3.54-3.62 (1H, m), 3.34- 3.47 (3H, m), 2.78-3.01 (3H, m), 1.98-2.05 (1H, m), 1.81-1.92 (1H, m), 1.53-1.65 (1H, m), 1.20 (6H, d, J = 6.8 Hz); LCMS: 99.4%, MS (ESI): 610.2 m/z [M + H]⁺ |
| 65 | 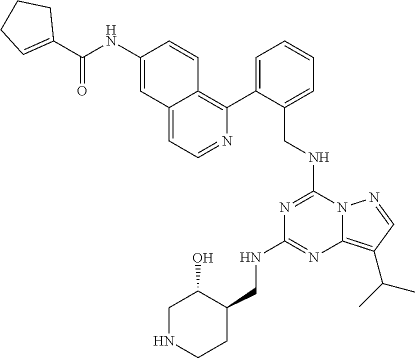 [SMILES: CC(C)c1cnn2c(NCc3ccccc3-c3nccc4cc(NC(=O)C5=CCCC5)ccc34)nc(NC[C@H]3CCNC[C@@H]3O)nc12] | (3R, 4R); yellow solid; ¹H-NMR (CD₃OD, 400 MHz): δ 8.58-8.61 (1H, m), 8.44 (1H, dd, J = 6.4, 2.4 Hz), 8.17- 8.22 (1H, m), 7.79-7.91 (3H, m), 7.56-7.72 (4H, m), 6.92 (1H, d, J = 2.0 Hz), 5.05-5.13 (1H, m), 4.68-4.75 (1H, m), 3.69-3.76 (1H, m), 3.37-3.59 (4H, m), 2.60- 3.01 (7H, m), 1.96-2.07 (3H, m), 1.79-1.88 (1H, m), 1.52-1.63 (1H, m), 1.18 (6H, d, J = 6.8 Hz); LCMS: 98.3%, MS (ESI): 632.2 m/z [M + H]⁺ |
| 66 | 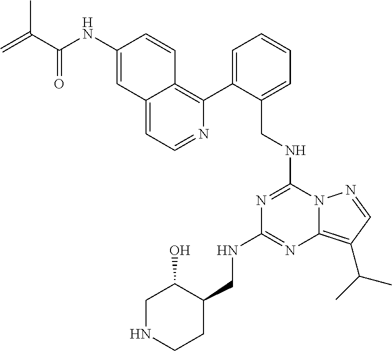 [SMILES: C=C(C)C(=O)Nc1ccc2c(-c3ccccc3CNc3nc(NC[C@H]4CCNC[C@@H]4O)nc4c(C(C)C)cnn34)nccc2c1] | (3R, 4R); yellow solid; ¹H-NMR (CD₃OD, 400 MHz): δ 8.52-8.59 (1H, m), 8.39-8.45 (1H, m), 8.04-8.13 (1H, m), 7.52-7.88 (7H, m), 5.94 (1H, s), 5.69 (1H, s), 5.08- 5.17 (1H, m), 4.62-4.73 (1H, m), 3.34-3.76 (5H, m), 2.76-3.05 (3H, m), 1.52-2.10 (6H, m), 1.20 (6H, d, J = 6.8 Hz); LCMS: 98.9%, MS (ESI): 606.2 m/z [M + H]⁺ |
| 67 | 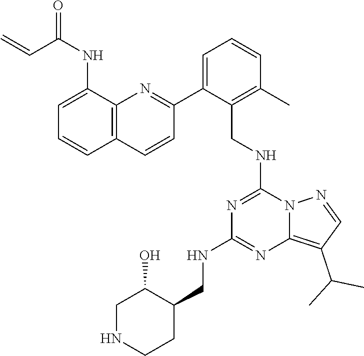 [SMILES: C=CC(=O)Nc1cccc2ccc(-c3cccc(C)c3CNc3nc(NC[C@H]4CCNC[C@@H]4O)nc4c(C(C)C)cnn34)nc12] | (3R, 4R); light yellow powder; ¹H-NMR (CD₃OD, 400 MHz): δ 8.73 (1H, d, J = 8.0 Hz), 8.32 (1H, d, J = 8.8 Hz), 7.74 (1H, s), 7.59-7.69 (3H, m), 7.46-7.48 (2H, m), 7.35-7.37 (1H, m), 6.49-6.56 (1H, m), 6.32-6.36 (1H, m), 5.69-5.76 (1H, m), 4.91-4.95 (3H, m), 4.84- 4.86 (1H, m), 3.60-3.66 (1H, m), 3.34-3.39 (1H, m), 3.25-3.28 (1H, m), 2.84-2.94 (2H, m), 2.69-2.75 (1H, m), 2.57 (3H, s), 1.88-1.94 (1H, m), 1.73-1.77 (1H, m), 1.43-1.47 (1H, m), 1.26 (6H, d, J = 6.8 Hz); LCMS: 100.0%, MS (ESI): 606.3 m/z [(M + H)]⁺ |
| 68 | 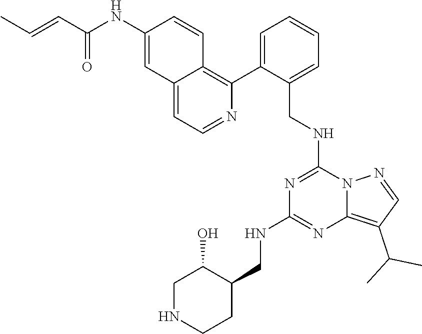 [SMILES: C/C=C/C(=O)Nc1ccc2c(-c3ccccc3CNc3nc(NC[C@H]4CCNC[C@@H]4O)nc4c(C(C)C)cnn34)nccc2c1] | (3R, 4R); yellow solid; ¹H-NMR (CD₃OD, 400 MHz): δ 8.59-8.63 (1H, m), 8.42-8.47 (1H, m), 8.17-8.22 (1H, m), 7.55-7.73 (7H, m), 7.06-7.12 (1H, m), 6.22 (1H, d, J = 15.2 Hz), 5.05-5.12 (1H, m), 4.64-4.75 (1H, m), 3.69-3.76 (1H, m), 3.30-3.58 (4H, m), 2.89-3.01 (1H, m), 2.75-2.84 (2H, m), 1.95-2.04 (4H, m), 1.80-1.91 (1H, m), 1.52-1.63 (1H, m), 1.18 (6H, d, J = 6.8 Hz); LCMS: 100%, MS (ESI): 606.3 m/z [M + H]⁺ |
| 69 | 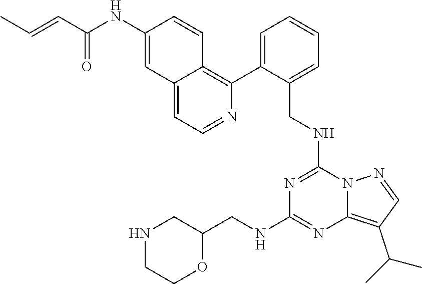 [SMILES: C/C=C/C(=O)Nc1ccc2c(-c3ccccc3CNc3nc(NCC4CNCCO4)nc4c(C(C)C)cnn34)nccc2c1] | Racemic mixture; yellow solid; ¹H-NMR (CD₃OD, 400 MHz): δ 8.54-8.61 (1H, m), 8.42 (1H, d, J = 6.8 Hz), 8.14-8.18 (1H, m), 7.55-7.87 (7H, m), 7.06-7.14 (1H, m), 6.22 (1H, d, J = 15.2 Hz), 5.03-5.13 (1H, m), 4.67- 4.72 (1H, m), 3.81-4.15 (3H, m), 3.34-3.61 (3H, m), 2.79-3.25 (3H, m), 1.97 (3H, d, J = 6.8 Hz), 1.16-1.21 (6H, m); LCMS: 100%, MS (ESI): 592.3 m/z [M + H]⁺ |
| 70 | 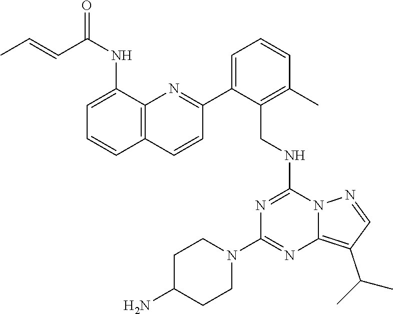 | Yellow powder; ¹H-NMR (CD₃OD, 400 MHz): δ 8.54 (1H, d, J = 7.6 Hz), 8.45 (1H, d, J = 8.8 Hz), 7.65-7.77 (4H, m), 7.45-7.52 (2H, m), 7.35-7.37 (1H, m), 6.91- 6.97 (1H, m), 6.22 (1H, d, J = 14.8 Hz), 4.93-4.94 (2H, m), 3.64-3.65 (1H, m), 3.32-3.36 (2H, m), 3.26-3.28 (2H, m), 2.85-2.92 (2H, m), 2.74-2.77 (1H, m), 2.62 (3H, s), 1.87-1.95 (3H, m), 1.70-1.77 (1H, m), 1.44-1.52 (1H, m), 1.25 (6H, d, J = 6.8 Hz), 0.85-0.91 (1H, m); LCMS: 98.5%, MS (ESI): 620.2 m/z [(M + H)]⁺ |
| 71 | 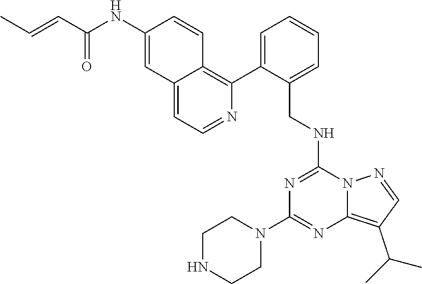 [SMILES: C/C=C/C(=O)Nc1ccc2c(-c3ccccc3CNc3nc(N4CCNCC4)nc4c(C(C)C)cnn34)nccc2c1] | yellow solid; ¹H-NMR (CD₃OD, 400 MHz): δ 8.57 (1H, d, J = 2.0 Hz), 8.38 (1H, d, J = 6.8 Hz), 8.16 (1H, d, J = 6.8 Hz), 7.79-7.86 (2H, m), 7.66-7.74 (2H, m), 7.57- 7.62 (3H, m), 7.07-7.14 (1H, m), 6.25 (1H, dd, J = 15.2, 1.6 Hz), 5.01-5.05 (1H, m), 4.68-4.72 (1H, m), 4.01- 4.05 (4H, m), 3.33-3.36 (4H, m), 3.00-3.07 (1H, m), 1.98 (3H, dd, J = 6.8, 1.6 Hz), 1.20 (6H, dd, J = 6.8, 1.6 Hz); LCMS: 100.0%, MS (ESI): 562.2 m/z [(M + H)]⁺ |
| 72 | 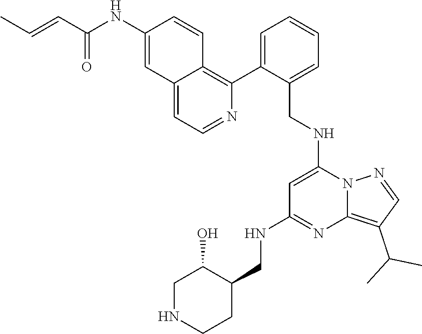 [SMILES: C/C=C/C(=O)Nc1ccc2c(-c3ccccc3CNc3cc(NC[C@H]4CCNC[C@@H]4O)nc4c(C(C)C)cnn34)nccc2c1] | (3R, 4R); yellow powder; ¹H-NMR (CD₃OD, 400 MHz): δ 8.46-8.52 (1H, m), 8.36-8.38 (1H, m), 8.05- 8.08 (1H, m), 7.78-7.87 (2H, m), 7.65-7.69 (2H, m), 7.49-7.55 (3H, m), 7.05-7.12 (1H, m), 6.24 (1H, dd, J = 15.2, 1.2 Hz), 4.99-5.03 (1H, m), 4.65-4.72 (1H, m), 3.67-3.73 (1H, m), 3.38-3.48 (4H, m), 2.98-3.05 (1H, m), 2.81-2.91 (2H, m), 2.4-2.09 (1H, m), 1.97 (3H, dd, J = 6.8, 1.2 Hz), 1.81-1.86 (1H, m), 1.62-1.68 (1H, m), 1.17-1.22 (6H, m); LCMS: 99.4%, MS (ESI): 605.3 m/z [(M + H)]⁺ |
| 73 | 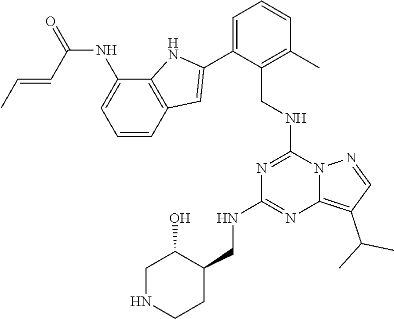 [SMILES: C/C=C/C(=O)Nc1cccc2cc(-c3cccc(C)c3CNc3nc(NC[C@H]4CCNC[C@@H]4O)nc4c(C(C)C)cnn34)[nH]c12] | (3R, 4R); off-white powder; ¹H NMR (CD₃OD, 400 MHz): δ 7.85 (1H, s), 7.36-7.45 (3H, m), 7.34 (1H, d, J = 8.0 Hz), 7.19 (1H, d, J = 7.6 Hz), 6.97-7.05 (1H, m), 6.60-6.74 (1H, m), 6.17 (1H, d, J = 15.2 Hz), 4.93-4.97 (2H, m), 3.55-3.61 (1H, m), 3.45-3.54 (1H, m), 3.35- 3.43 (1H, m), 3.23-3.27 (1H, m), 3.03-3.09 (1H, m), 2.94-3.01 (1H, m), 2.57-2.68 (2H, m), 2.54 (3H, s), 1.88 (3H, d, J = 6.4 Hz), 1.64-1.84 (2H, m), 1.34-1.45 (1H, m), 1.30 (6H, d, J = 6.8 Hz); LCMS: 100%, MS (ESI): m/z 608.3 [M + H]⁺ |
| 74 | 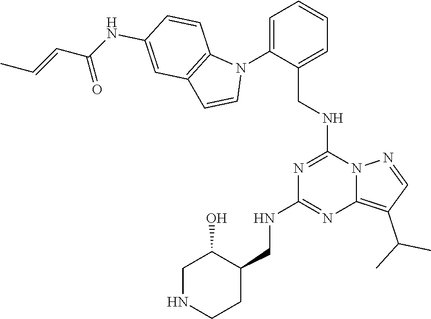 [SMILES: C/C=C/C(=O)Nc1ccc2c(ccn2-c2ccccc2CNc2nc(NC[C@H]3CCNC[C@@H]3O)nc3c(C(C)C)cnn23)c1] | (3R, 4R); white powder; ¹H NMR (CD₃OD, 400 MHz): δ 7.93 (1H, s), 7.68-7.84 (2H, m), 7.59 (2H, m), 7.36 (1H, d, J = 7.2 Hz), 7.23-7.30 (1H, m), 6.89-7.04 (2H, m), 6.77-6.83 (1H, m), 6.48-6.56 (1H, m), 6.18 (1H, d, J = 14.8 Hz), 4.51-4.69 (2H, m), 3.60-3.69 (2H, m), 3.41-3.52 (1H, m), 3.13-3.19 (1H, m), 2.70-3.02 (4H, m), 1.89-1.99 (4H, m), 1.74-1.82 (1H, m), 1.41-1.57 (1H, m), 1.20-1.34 (6H, m); LCMS: 99.9%, MS (ESI): 594.2 m/z [M + H]⁺ |
| 75 | 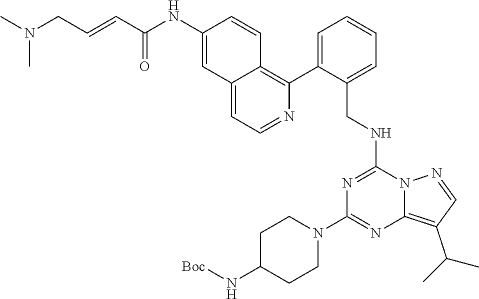 [SMILES: CC(C)c1cnn2c(NCc3ccccc3-c3nccc4cc(NC(=O)/C=C/CN(C)C)ccc34)nc(N3CCC(NC(=O)OC(C)(C)C)CC3)nc12] | white powder; ¹H NMR (400 MHz, CDCl₃): δ 8.33- 8.57 (3H, m), 7.53-7.65 (4H, m), 7.34-7.48 (4H, m), 6.95-7.12 (2H, m), 6.29-6.52 (1H, m), 4.69-4.88 (1H, m), 4.38-4.59 (3H, m), 4.17-4.36 (1H, m), 3.54-3.73 (1H, m), 3.25-3.40 (2H, m), 2.83-3.05 (3H, m), 2.48 (6H, s), 1.85-2.0 (2H, m), 1.46 (9H, s), 1.24-1.26 (8H, m); LCMS: 100%, MS (ESI): m/z 719.3 [M + H]⁺ |
| 76 | 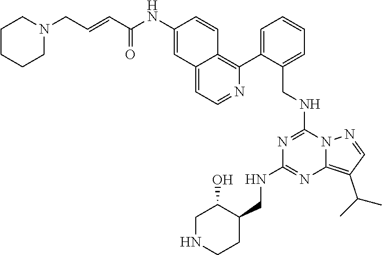 [SMILES: CC(C)c1cnn2c(NCc3ccccc3-c3nccc4cc(NC(=O)/C=C/CN5CCCCC5)ccc34)nc(NC[C@H]3CCNC[C@@H]3O)nc12] | (3R, 4R); Yellow solid; ¹H-NMR (CD₃OD, 400 MHz): δ 8.66-8.70 (1H, m), 8.45 (1H, dd, J = 6.8, 2.4 Hz), 8.19- 8.24 (1H, m), 7.80-7.88 (3H, m), 7.64-7.71 (2H, m), 7.56-7.61 (2H, m), 7.01-7.08 (1H, m), 6.70 (1H, d, J = 15.2 Hz), 5.02-5.07 (1H, m), 4.69-4.79 (1H, m), 4.02 (2H, d, J = 7.2 Hz), 3.70-3.76 (1H, m), 3.53-3.60 (3H, m), 3.31-3.46 (3H, m), 2.76-3.07 (5H, m), 1.96-2.03 (3H, m), 1.78-1.91 (4H, m), 1.51-1.62 (2H, m), 1.19 (6H, d, J = 7.2 Hz); LCMS: 100%, MS (ESI): 689.3 m/z [M + H]⁺. |
| 77 | 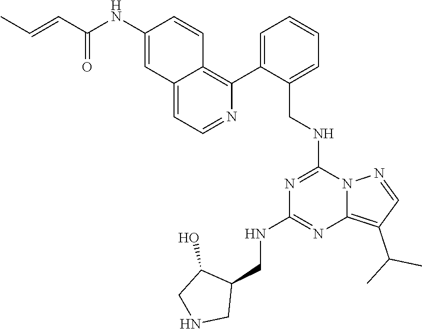 [SMILES: C/C=C/C(=O)Nc1ccc2c(-c3ccccc3CNc3nc(NC[C@H]4CNC[C@@H]4O)nc4c(C(C)C)cnn34)nccc2c1] | Mixture of 2 trans isomer; yellow solid; ¹H-NMR (CD₃OD, 400 MHz): δ 8.55-8.62 (1H, m), 8.41-8.46 (1H, m), 8.15-8.19 (1H, m), 7.56-7.87 (7H, m), 7.06- 7.18 (1H, m), 6.21 (1H, d, J = 16.0 Hz), 5.09-5.21 (1H, m), 4.67-4.82 (2H, m), 4.26-4.35 (1H, m), 3.41-3.62 (3H, m), 3.12-3.21 (2H, m), 2.80-2.86 (1H, m), 2.38- 2.59 (1H, m), 1.97 (3H, dd, J = 7.2, 1.6 Hz), 1.18 (6H, d, J = 6.8 Hz). LCMS: 100%, MS (ESI): 592.2 m/z [M + H]⁺. |
| 78 | 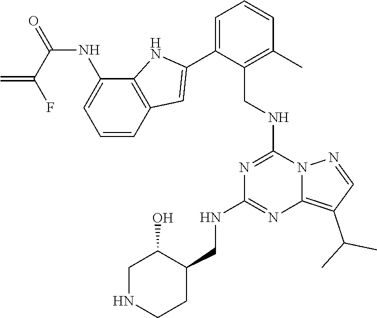 [SMILES: C=C(F)C(=O)Nc1cccc2cc(-c3cccc(C)c3CNc3nc(NC[C@H]4CCNC[C@@H]4O)nc4c(C(C)C)cnn34)[nH]c12] | (3R, 4R); gray powder; ¹H NMR (MeOD, 400 MHz): δ ppm 7.85 (1H, s), 7.34-7.48 (4H, m), 7.18 (1H, d, J = 7.6 Hz), 7.03-7.10 (1H, m), 6.49 (1H, s), 5.34-5.52 (1H, m), 5.21 (1H, dd, J = 15.2, 3.2 Hz), 4.92-4.95 (2H, m), 3.50- 3.62 (2H, m), 3.37-3.45 (1H, m), 3.20-3.27 (1H, m), 2.93-3.05 (2H, m), 2.50-2.61 (5H, m), 1.67-1.83 (2H, m), 1.33-1.42 (1H, m), 1.30 (6H, d, J = 6.8 Hz); LCMS: 100%, MS (ESI): m/z 612.3 [M + H]+ |
| 79 | 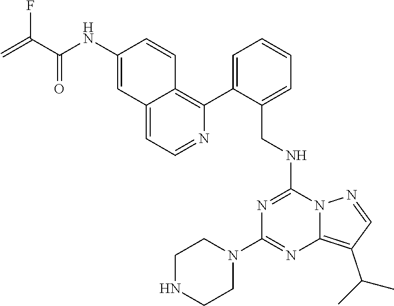 [SMILES: C=C(F)C(=O)Nc1ccc2c(-c3ccccc3CNc3nc(N4CCNCC4)nc4c(C(C)C)cnn34)nccc2c1] | Yellow solid; ¹H-NMR (CD₃OD, 400 MHz): δ 8.56 (1H, d, J = 2.0 Hz), 8.36 (1H, d, J = 6.4 Hz), 8.15 (1H, d, J = 6.8 Hz), 7.76-7.83 (3H, m), 7.61-7.67 (1H, m), 7.53- 7.60 (2H, m), 7.44 (1H, s), 5.85 (1H, dd, J = 46.0, 3.6 Hz), 5.44 (1H, dd, J = 15.2, 3.6 Hz), 5.00 (1H, d, J = 15.6 Hz), 4.63 (1H, d, J = 15.6 Hz), 3.89-4.00 (4H, m), 3.21-3.29 (4H, m, overlap with water signal), 2.87-2.95 (1H, m), 1.15-1.21 (6H, m); LCMS: 100%, MS (ESI): 566.2 m/z [M + H]⁺ |
| 80 | 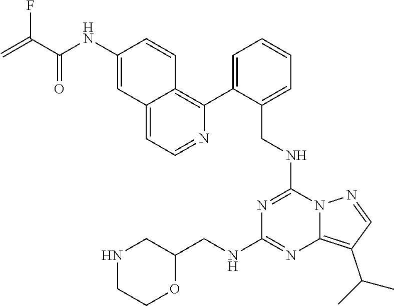 [SMILES: C=C(F)C(=O)Nc1ccc2c(-c3ccccc3CNc3nc(NCC4CNCCO4)nc4c(C(C)C)cnn34)nccc2c1] | Racemic mixture; Yellow solid; ¹H-NMR (CD₃OD, 400 MHz): δ 8.62-8.65 (1H, m), 8.44-8.49 (1H, m), 8.20- 8.24 (1H, m), 7.79-7.90 (3H, m), 7.65-7.71 (2H, m), 7.54-7.61 (2H, m), 5.85 (1H, dd, J = 46.4, 3.6 Hz), 5.44 (1H, dd, J = 14.8, 3.6 Hz), 5.03-5.13 (1H, m), 4.65-4.76 (1H, m), 3.81-4.12 (3H, m), 3.32-3.61 (3H, m), 2.78- 3.21 (4H, m), 1.16-1.21 (6H, m); LCMS: 99.3%, MS (ESI): 596.2 m/z [M + H]⁺ |
| 81 | 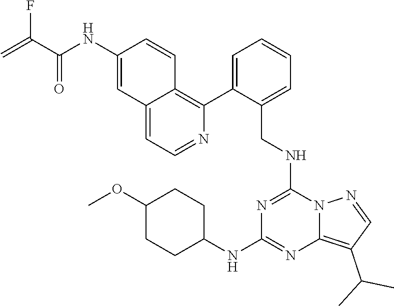 [SMILES: C=C(F)C(=O)Nc1ccc2c(-c3ccccc3CNc3nc(NC4CCC(OC)CC4)nc4c(C(C)C)cnn34)nccc2c1] | off-white powder; ¹H NMR (400 MHz, CD₃OD): δ 8.56 (1H, s), 8.44 (1H, d, J = 6.8 Hz), 8.24 (1H, d, J = 6.4 Hz), 7.92-8.00 (1H, m), 7.81-7.91 (2H, m), 7.73 (1H, t, J = 7.2 Hz), 7.62 (1H, d, J = 7.6 Hz), 7.52-7.56 (1H, m), 7.46 (1H, s), 5.85 (1H, dd, J = 46.4, 4.0 Hz), 5.48 (1H, dd, J = 15.2, 4.0 Hz), 4.90-5.02 (2H, m), 4.73-4.83 (1H, m), 3.41 (3H, s), 3.23-3.32 (2H, m), 2.85-2.94 (1H, m), 1.99-2.24 (3H, m), 1.65-1.76 (1H, m), 1.30- 1.55 (4H, m), 1.15-1.24 (6H, m); HPLC: 98.5% MS (ESI): m/z 608.2[M + H]⁺ |
| 82 | 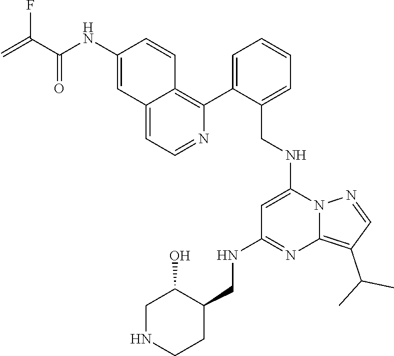 [SMILES: C=C(F)C(=O)Nc1ccc2c(-c3ccccc3CNc3cc(NC[C@H]4CCNC[C@@H]4O)nc4c(C(C)C)cnn34)nccc2c1] | (3R, 4R); light yellow solid; ¹H-NMR (CD₃OD, 400 MHz): δ 8.52-8.60 (1H, m), 8.44 (1H, J = 6.8 Hz), 8.22-8.26 (1H, m), 7.81-7.92 (3H, m), 7.67-7.72 (1H, m), 7.56-7.63 (2H, m), 7.48 (1H, s), 5.87 (1H, dd, J = 46.0, 2.8 Hz), 5.46 (1H, dd, J = 15.2, 4.0 Hz), 5.07-5.12 (1H, m), 4.73-4.79 (1H, m), 3.69-3.75 (1H, m), 3.37- 3.56 (4H, m), 2.99-3.12 (1H, m), 2.81-2.97 (1H, m), 2.06-2.12 (1H, m), 1.82-1.89 (1H, m), 1.64-1.73 (1H, m), 1.17-1.24 (6H, m); LCMS: 99.7%; MS (ESI): 609.2m/z [(M + H)]⁺ |
| 83 | 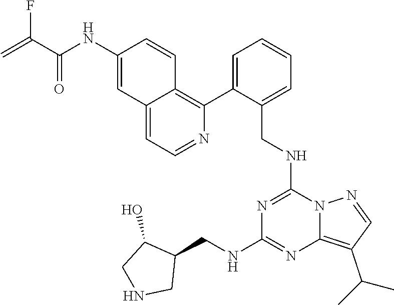 [SMILES: C=C(F)C(=O)Nc1ccc2c(-c3ccccc3CNc3nc(NC[C@H]4CNC[C@@H]4O)nc4c(C(C)C)cnn34)nccc2c1] | Mixture of 2 trans isomer; Yellow solid; ¹H-NMR (CD₃OD, 400 MHz): δ 8.60-8.67 (1H, m), 8.48-8.52 (1H, m), 8.24-8.28 (1H, m), 7.81-7.93 (3H, m), 7.56- 7.73 (4H, m), 5.85 (1H, dd, J = 46.0, 3.6 Hz), 5.44 (1H, dd, J = 14.8, 3.6 Hz), 5.09-5.22 (1H, m), 4.67-4.77 (1H, m), 4.36-4.35 (1H, m), 3.46-3.62 (3H, m), 3.10-3.21 (2H, m), 2.81-2.88 (1H, m), 2.41-2.62 (1H, m), 1.16- 1.22 (6H, m); LCMS: 100%, MS (ESI): 596.2 m/z [M + H]⁺ |
| 84 | 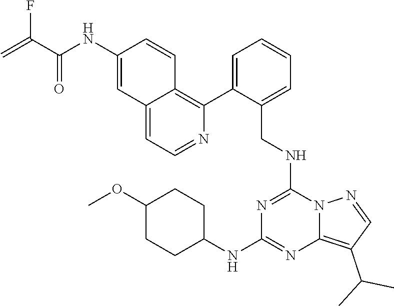 [SMILES: C=C(F)C(=O)Nc1ccc2c(-c3ccccc3CNc3nc(NC4CCC(OC)CC4)nc4c(C(C)C)cnn34)nccc2c1] | Yellow solid; ¹H-NMR (CD₃OD, 400 MHz): δ 8.63 (1H, s), 8.43 (1H, d, J = 6.8 Hz), 8.29 (1H, d, J = 6.8 Hz), 7.82-7.92 (3H, m), 7.65-7.71 (1H, m), 7.57-7.62 (2H, m), 7.54 (1H, s), 5.85 (1H, dd, J = 46.4, 3.6 Hz), 5.45 (1H, dd, J = 14.8, 3.6 Hz), 4.92-4.99 (1H, m), 4.72- 4.80 (1H, m), 3.51-3.58 (1H, m), 3.39 (3H, s), 3.22- 3.31 (1H, m, overlap with CD₃OD signal), 2.76-2.83 (1H, m), 2.01-2.18 (3H, m), 1.69-1.73 (1H, m), 1.27- 1.46 (4H, m), 1.15-1.21 (6H, m); LCMS: 100%, MS (ESI): 609.2 m/z [M + H]⁺ |
| 85 | 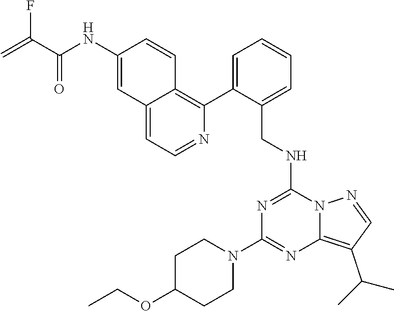 [SMILES: C=C(F)C(=O)Nc1ccc2c(-c3ccccc3CNc3nc(N4CCC(OCC)CC4)nc4c(C(C)C)cnn34)nccc2c1] | White powder; H NMR (CDCl₃, 400 MHz): δ 8.54 (1H, d, J = 5.6 Hz), 8.30 (1H, s), 8.04 (1H, brs), 7.59 (1H, d, J = 8.8 Hz), 7.55 (2H, d, J = 6.0 Hz), 7.44 (1H, s), 7.30-7.42 (3H, m), 7.25 (1H, d, J = 8.8 Hz), 6.92-6.96 (1H, m), 5.85 (1H, dd, J = 47.6, 3.6 Hz), 5.26 (1H, dd, J = 14.8, 3.6 Hz), 4.76-4.85 (1H, m), 4.12-4.31 (3H, m), 3.42-3.53 (3H, m), 3.09-3.18 (2H, m), 2.83-2.96 (1H, m), 1.80-1.87 (2H, m), 1.43-1.51 (2H, m, overlap with water signal), 1.15-1.21 (9H, m); LCMS: 98.8%, MS (ESI): m/z 609.3 [M + H]⁺ |
| 86 | 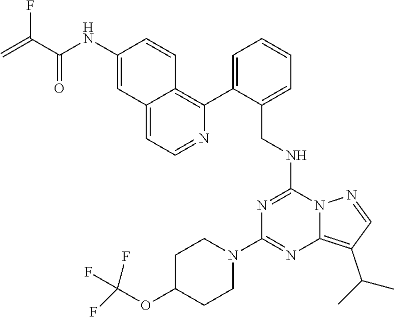 [SMILES: C=C(F)C(=O)Nc1ccc2c(-c3ccccc3CNc3nc(N4CCC(OC(F)(F)F)CC4)nc4c(C(C)C)cnn34)nccc2c1] | Off-white powder; ¹H NMR (CDCl₃, 400 MHz): δ 8.62 (1H, d, J = 6.0 Hz), 8.41 (1H, s), 8.06-8.15 (1H, m), 7.61-7.75 (3H, m), 7.31-7.57 (5H, m), 7.02-7.23 (1H, m), 5.95 (1H, dd, J = 47.6, 3.2 Hz), 5.37 (1H, dd, J = 14.8, 3.2 Hz), 4.85-4.96 (1H, m), 4.46-4.54 (1H, m), 4.07-4.36 (2H, m), 3.47-3.60 (2H, m), 29.5-3.04 (1H, m), 1.92-2.05 (2H, m), 1.68-1.80 (2H, m), 1.27 (6H, d, J = 6.8 Hz); LCMS: 97.3%, MS (ESI): 649.1 m/z [M + H]+ |
| 87 | 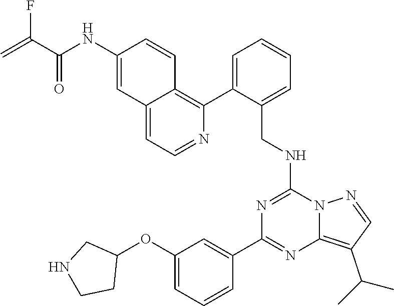 [SMILES: C=C(F)C(=O)Nc1ccc2c(-c3ccccc3CNc3nc(-c4cccc(OC5CCNC5)c4)nc4c(C(C)C)cnn34)nccc2c1] | Racemic mixture; off-white powder; ¹H NMR (400 MHz, CD₃OD) δ 8.39 (1H, s), 7.97 (1H, d, J = 6.4 Hz), 7.83-7.92 (2H, m), 7.73-7.80 (4H, m), 7.61-7.72 (3H, m), 7.56 (1H, d, J = 7.6 Hz), 7.44 (1H, t, J = 8.0 Hz), 7.12-7.16 (1H, m), 5.85 (1H, dd, J = 40.0, 4.0 Hz), 5.79 (1H, dd, J = 14.8, 3.6 Hz), 5.35-5.38 (1H, m), 5.03-5.07 (1H, m), 3.64-3.69 (1H, m), 3.18-3.27 (2H, m), 3.11- 3.17 (1H, m), 2.41-2.48 (2H, m), 2.02-2.08 (1H, m), 1.58-1.64 (1H, m), 1.28-1.34 (6H, m); HPLC: 96.9% MS (ESI): m/z 643.2[M + H]⁺ |
| 88 | 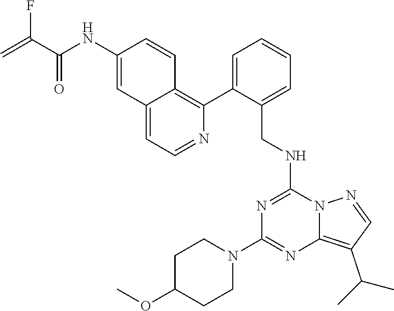 [SMILES: C=C(F)C(=O)Nc1ccc2c(-c3ccccc3CNc3nc(N4CCC(OC)CC4)nc4c(C(C)C)cnn34)nccc2c1] | Yellow powder; ¹H NMR (CDCl₃, 400 MHz): δ 8.47 (1H, d, J = 6.0 Hz), 8.28 (1H, d, J = 1.2 Hz), 8.04-8.08 (1H, m), 7.59 (1H, d, J = 9.2 Hz), 7.52- 7.57 (2H, m), 7.35-7.46 (3H, m), 7.26-7.34 (2H, m), 5.85 (1H, dd, J = 47.6, 3.6 Hz), 5.27 (1H, dd, J = 15.2, 3.6 Hz), 4.78-4.85 (1H, m), 4.08-4.24 (3H, m), 3.32-3.39 (4H, m), 3.19-3.30 (2H, m), 2.85-2.96 (1H, m), 1.79-1.84 (2H, m), 1.45-1.58 (2H, m, overlap with water signal), 1.17 (6H, d, J = 6.8 Hz); LCMS: 98%, MS (ESI): m/z 595.2 [M + H]⁺ |
| 89 | 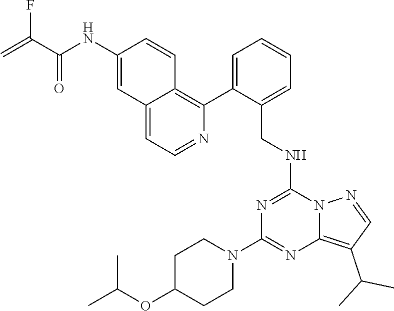 [SMILES: C=C(F)C(=O)Nc1ccc2c(-c3ccccc3CNc3nc(N4CCC(OC(C)C)CC4)nc4c(C(C)C)cnn34)nccc2c1] | Yellow powder; ¹H NMR (CD₃OD, 400 MHz): δ 8.62 (1H, d, J = 2.0 Hz), 8.42 (1H, d, J = 7.6 Hz), 8.21 (1H, d, J = 6.4 Hz), 7.79-7.91 (3H, m), 7.56-7.74 (4H, m), 5.94 (1H, dd, J = 46.4, 3.6 Hz), 5.48 (1H, dd, J = 14.8, 3.6 Hz), 4.94-5.02 (1H, m), 4.67-4.75 (1H, m), 3.91- 4.02 (2H, m), 3.74-3.88 (2H, m), 3.46-3.59 (2H, m), 2.98-3.07 (1H, m), 1.82-1.93 (2H, m), 1.47-1.65 (2H, m), 1.18-1.23 (12H, m); LCMS: 99.9%, MS (ESI): 623.2 m/z [M + H]+ |
| 90 | 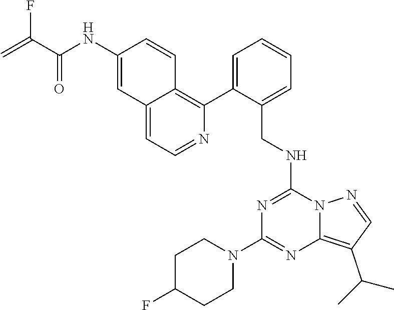 [SMILES: C=C(F)C(=O)Nc1ccc2c(-c3ccccc3CNc3nc(N4CCC(F)CC4)nc4c(C(C)C)cnn34)nccc2c1] | White solid; ¹H NMR (CDCl₃, 400 MHz,) δ 8.62 (1H, d, J = 5.6 Hz), 8.38 (1H, d, J = 2.0 Hz), 8.16-8.21 (1H, m), 7.69 (1H, d, J = 8.8 Hz), 7.60-7.65 (2H, m), 7.54 (1H, s), 7.43-7.49 (3H, m), 7.39-7.42 (1H, m), 7.09-7.16 (1H, m), 5.91 (1H, dd, J = 47.6, 3.6 Hz), 5.35 (1H, dd, J = 15.2, 3.6 Hz), 4.76-4.95 (2H, m), 4.18-4.29 (1H, m), 3.71-3.92 (4H, m), 2.93-3.05 (1H, m), 1.78-1.94 (4H, m), 1.26 (6H, d, J = 6.8 Hz); LCMS: 100.0%, MS (ESI): m/z 583.1 [M + H]+. |
| 91 | 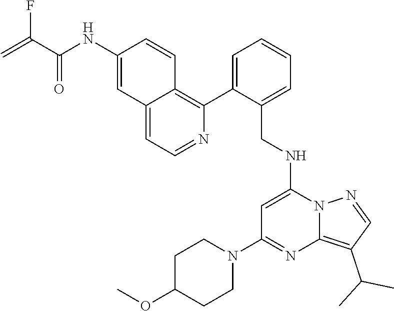 [SMILES: C=C(F)C(=O)Nc1ccc2c(-c3ccccc3CNc3cc(N4CCC(OC)CC4)nc4c(C(C)C)cnn34)nccc2c1] | Yellow powder; ¹H-NMR (CD₃OD, 400 MHz): δ 8.41 (1H, d, J = 5.6 Hz), 8.36 (1H, d, J = 1.6 Hz), 7.66-7.72 (2H, m), 7.54-7.59 (1H, m), 7.46-7.52 (2H, m), 7.35- 7.42 (3H, m), 5.78 (1H, dd, J = 46.4, 3.6 Hz), 5.36 (1H, dd, J = 14.8, 3.2 Hz), 5.02 (1H, s), 4.48-4.55 (1H, m), 4.35-4.41 (1H, m), 3.80-3.95 (2H, m), 3.42-3.48 (1H, m), 3.38 (3H, s), 3.04-3.15 (2H, m), 2.91-2.98 (1H, m), 1.86-1.92 (2H, m), 1.38-1.51 (2H, m), 1.22 (6H, d, J = 6.8 Hz); LCMS: 98.5%, MS (ESI): 594.2m/z [(M + H)]+. |
| 92 | 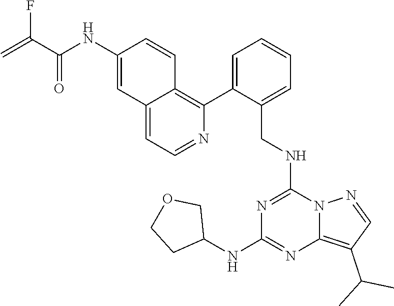 [SMILES: C=C(F)C(=O)Nc1ccc2c(-c3ccccc3CNc3nc(NC4CCOC4)nc4c(C(C)C)cnn34)nccc2c1] | White powder; ¹H NMR (CDCl₃, 400 MHz): δ 8.60 (1H, brs), 8.38-8.41 (1H, m), 8.23 (1H, brs), 7.72 (1H, d, J = 9.2 Hz), 7.58-7.63 (2H, m), 7.56 (1H, s), 7.38-7.51 (4H, m), 5.90 (1H, dd, J = 47.6, 3.6 Hz), 5.84 (1H, d, J = 15.2, 3.2 Hz), 4.73-4.89 (2H, m), 4.48-4.55 (1H, m), 4.19-4.28 (1H, m), 3.91-4.03 (2H, m), 3.83-3.89 (1H, m), 3.66-3.71 (1H, m), 2.92-3.03 (1H, m), 2.23-2.31 (1H, m), 1.79-1.87 (1H, m), 1.22-1.29 (7H, m); LCMS: 97.4%, MS (ESI): m/z 567.2 [M + H]+ |
| 93 | 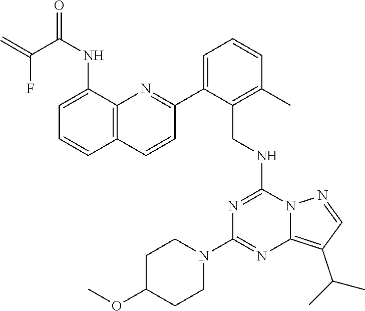 [SMILES: C=C(F)C(=O)Nc1cccc2ccc(-c3cccc(C)c3CNc3nc(N4CCC(OC)CC4)nc4c(C(C)C)cnn34)nc12] | White solid; ¹H-NMR (CDCl₃, 400 MHz): δ 10.59 (1H, brs), 8.82-8.84 (1H, m), 8.18 (1H, d, J = 8.8 Hz), 7.56- 7.61 (3H, m), 7.50 (1H, s), 7.35-7.40 (3H, m), 6.24 (1H, t, J = 4.8 Hz), 5.82 (1H, dd, J = 46.8, 3.2 Hz), 5.21 (1H, dd, J = 14.8, 3.2 Hz), 4.83-4.87 (2H, m), 4.14-4.19 (2H, m), 3.35-3.40 (4H, m), 3.14-3.21 (2H, m), 2.96-2.99 (1H, m), 2.55 (3H, s), 1.82-1.85 (2H, m), 1.41-1.49 (2H, m), 1.26 (6H, d, J = 6.8 Hz); LCMS: 100.0%, MS (ESI): 609.2m/z [(M + H)]+. |
| 94 | 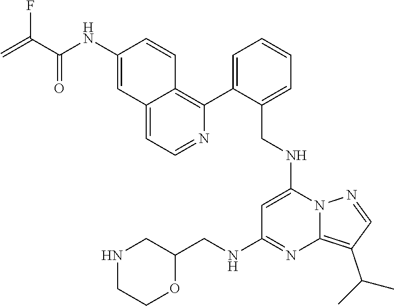 [SMILES: C=C(F)C(=O)Nc1ccc2c(-c3ccccc3CNc3cc(NCC4CNCCO4)nc4c(C(C)C)cnn34)nccc2c1] | off-white powder; ¹H NMR (400 MHz, CD₃OD) δ 8.53- 8.62 (1H, m), 8.41-8.45 (1H, m), 8.22 (1H, d, J = 6.4 Hz), 7.79-7.97 (3H, m), 7.68-7.74 (1H, m), 7.56-7.66 (2H, m), 7.50 (1H, d, J = 7.6 Hz), 5.79-5.97 (1H, m), 5.46 (1H, dd, J = 14.8, 3.6 Hz), 4.93-5.00 (2H, m, overlap with water signal), 3.96-4.17 (2H, m), 3.81- 3.88 (1H, m), 3.52-3.64 (1H, m), 3.41-3.51 (2H, m), 2.84-3.24 (4H, m), 1.10-1.29 (6H, m); HPLC: 96.4% MS (ESI): m/z 595.2[M + H]+. |
| 95 | 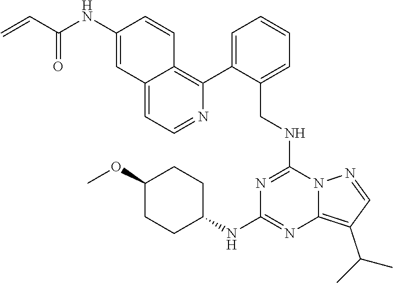 [SMILES: C=CC(=O)Nc1ccc2c(-c3ccccc3CNc3nc(N[C@H]4CC[C@H](OC)CC4)nc4c(C(C)C)cnn34)nccc2c1] | White solid; ¹H-NMR (CDCl₃, 400 MHz): δ 8.58 (1H, d, J = 5.6 Hz), 8.41-8.48 (1H, m), 7.77 (1H, brs), 7.53-7.68 (4H, m), 7.39-7.48 (3H, m), 7.30-7.34 (1H, m), 7.23 (1H, brs), 6.52 (1H, dd, J = 12.6, 1.2 Hz), 6.31 (1H, dd, J = 16.8, 10.0 Hz), 5.85 (1H, dd, J = 10.0, 1.2 Hz), 4.79- 4.91 (1H, m), 4.14-4.22 (1H, m), 3.68-3.75 (1H, m), 3.38 (3H, s), 3.14-3.22 (1H, m), 2.92-3.02 (1H, m), 1.81-2.13 (5H, m, overlap with water signal), 1.15-1.38 (9H, m).; LCMS: 100%, MS (ESI): 591.2 m/z [M + H]+. |
| 96 | 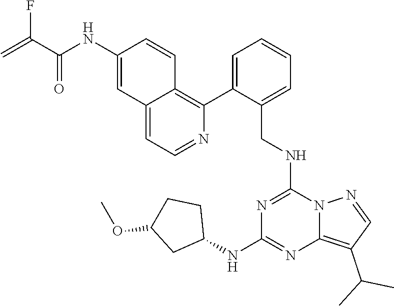 | Cis or trans form; yellow powder; ¹H NMR (CD₃OD, 400 MHz): δ 8.64-8.67 (1H, m), 8.43-8.51 (1H, m), 8.23-8.29 (1H, m), 7.78-7.93 (3H, m), 7.56-7.74 (4H, m), 5.88 (1H, dd, J = 46.0, 2.0 Hz), 5.48 (1H, dd, J = 15.2, 4.0 Hz), 4.97-5.09 (1H, m), 4.66-4.76 (1H, m), 4.16-4.25 (1H, m), 3.93-4.02 (1H, m), 3.39 (3H, s), 2.77- 2.84 (1H, m), 1.63-2.16 (5H, m), 1.53-1.62 (1H, m), 1.18- 1.26 (6H, m); LCMS: 97.9%, MS (ESI): 595.2 m/z [M + H]+ |
| 97 | 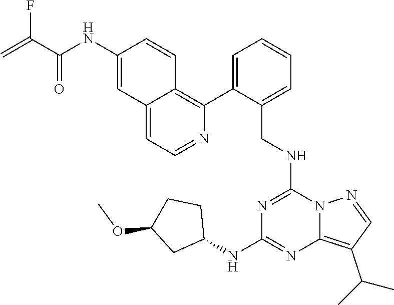 [SMILES: C=C(F)C(=O)Nc1ccc2c(-c3ccccc3CNc3nc(N[C@H]4CC[C@H](OC)C4)nc4c(C(C)C)cnn34)nccc2c1] | Cis or trans form; yellow powder; ¹H NMR (CD₃OD, 400 MHz): δ 8.64-8.67 (1H, m), 8.43-8.51 (1H, m), 8.23-8.29 (1H, m), 7.78-7.93 (3H, m), 7.56-7.74 (4H, m), 5.88 (1H, dd, J = 46.0, 2.0 Hz), 5.48 (1H, dd, J = 15.2, 4.0 Hz), 4.97-5.09 (1H, m), 4.66-4.76 (1H, m), 4.16-4.25 (1H, m), 3.93-4.02 (1H, m), 3.39 (3H, s), 2.77- 2.84 (1H, m), 1.63-2.16 (5H, m), 1.53-1.62 (1H, m), 1.18- 1.26 (6H, m); LCMS: 99.8%, MS (ESI): 595.2 m/z [M + H]+ |
| 98 | 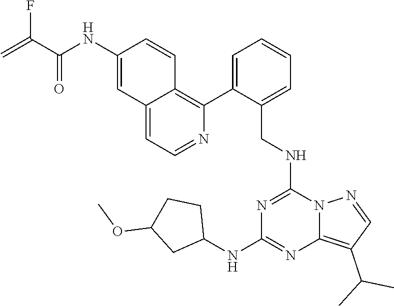 [SMILES: C=C(F)C(=O)Nc1ccc2c(-c3ccccc3CNc3nc(NC4CCC(OC)C4)nc4c(C(C)C)cnn34)nccc2c1] | Racemic mixture; yellow powder; yellow powder; ¹H NMR (CD₃OD, 400 MHz): δ 8.64-8.67 (1H, m), 8.43- 8.51 (1H, m), 8.23-8.29 (1H, m), 7.78-7.93 (3H, m), 7.56-7.74 (4H, m), 5.88 (1H, dd, J = 46.0, 2.0 Hz), 5.48 (1H, dd, J = 15.2, 4.0 Hz), 4.97-5.09 (1H, m), 4.66-4.76 (1H, m), 4.16-4.25 (1H, m), 3.93-4.02 (1H, m), 3.39 (3H, s), 2.77-2.84 (1H, m), 1.63-2.16 (5H, m), 1.53-1.62 (1H, m), 1.18-1.26 (6H, m); LCMS: 99.8%, MS (ESI): 595.2 m/z [M + H]+ |
| 99 | 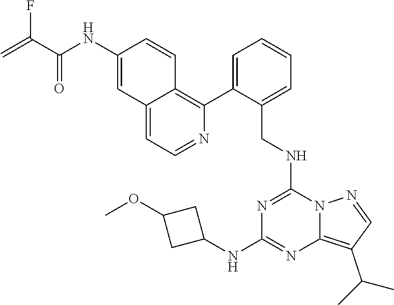 [SMILES: C=C(F)C(=O)Nc1ccc2c(-c3ccccc3CNc3nc(NC4CC(OC)C4)nc4c(C(C)C)cnn34)nccc2c1] | White solid; ¹H NMR (CD₃OD, 400 MHz,) δ 8.58-8.64 (1H, m), 8.39-8.42 (1H, m), 8.10-8.18 (1H, m), 7.78- 7.85 (3H, m), 7.61-7.69 (2H, m), 7.56-7.60 (2H, m), 5.85 (1H, dd, J = 46.0, 3.2 Hz), 5.45 (1H, dd, J = 14.8, 3.2 Hz), 4.98-5.05 (1H, m), 4.63-4.75 (1H, m), 3.67- 3.78 (2H, m), 3.28 (3H, s), 2.76-2.84 (2H, m), 2.53-2.61 (1H, m), 1.83-2.02 (2H, m), 1.17-1.22 (6H, m); LCMS: 100.0%, MS (ESI): m/z 581.2 [M + H]+. |
| 100 | 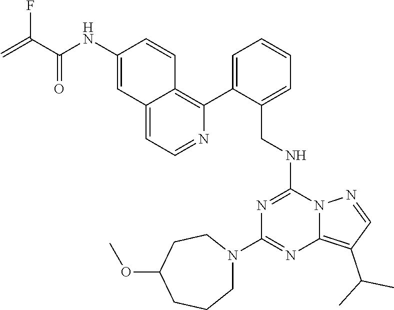 [SMILES: C=C(F)C(=O)Nc1ccc2c(-c3ccccc3CNc3nc(N4CCCC(OC)CC4)nc4c(C(C)C)cnn34)nccc2c1] | Yellow solid; ¹H NMR (CDCl₃, 400 MHz) δ 8.56-8.61 (1H, m), 8.44-8.53 (1H, m), 8.22-8.27 (1H, m), 7.92- 7.97 (1H, m), 7.81-7.85 (2H, m), 7.54-7.71 (4H, m), 5.87 (1H, dd, J = 46.0, 3.6 Hz), 5.46 (1H, dd, J = 15.2, 3.6 Hz), 3.42-3.96 (7H, m), 3.34 (3H, s), 3.03-3.15 (1H, m), 1.47-2.13 (5H, m), 1.20-1.24 (1H, m), 1.14-1.22 (6H, m); LCMS: 99.2%, MS (ESI): m/z 609.2 [M + H]+ |
| 101 | 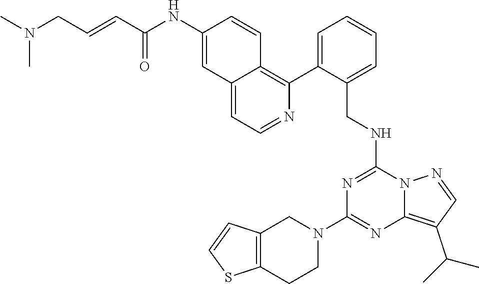 [SMILES: CC(C)c1cnn2c(NCc3ccccc3-c3nccc4cc(NC(=O)/C=C/CN(C)C)ccc34)nc(N3CCc4sccc4C3)nc12] | Yellow powder; ¹H NMR (CD₃OD, 400 MHz): δ 8.55 (1H, d, J = 2.0 Hz), 8.03-8.12 (1H, m), 7.97 (1H, d, J = 6.4 Hz), 7.82-7.85 (2H, m), 7.63-7.67 (2H, m), 7.50- 7.54 (3H, m), 7.29 (1H, d, J = 5.2 Hz), 6.93-7.02 (1H, m), 6.88 (1H, d, J = 4.8 Hz), 6.62 (1H, d, J = 15.2 Hz), 4.68-4.75 (1H, m), 4.01-4.09 (3H, m), 3.89-3.97 (1H, m), 3.56-3.61 (1H, m), 3.43-3.48 (1H, m), 2.91-3.02 (9H, m), 1.17-1.22 (6H, m).; LCMS: 100%, MS (ESI): m/z 658.2 [M + H]+ |
| 102 | 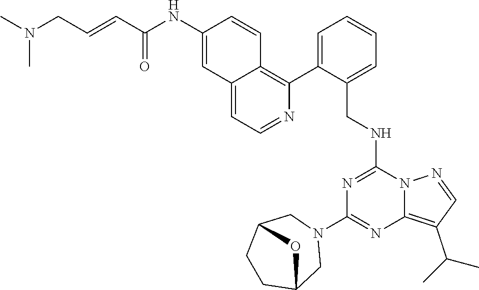 [SMILES: CC(C)c1cnn2c(NCc3ccccc3-c3nccc4cc(NC(=O)/C=C/CN(C)C)ccc34)nc(N3C[C@H]4CC[C@@H](C3)O4)nc12] | Yellow powder; ¹H NMR (400 MHz, CD₃OD) δ 8.67 (1H, d, J = 1.6 Hz), 8.33-8.42 (1H, m), 8.18-8.25 (1H, m), 7.80-7.91 (3H, m), 7.67-7.72 (1H, m), 7.57-7.65 (3H, m), 6.99-7.10 (1H, m), 6.76 (1H, d, J = 15.6 Hz), 4.73-4.84 (2H, m), 4.40-4.55 (2H, m), 4.09 (2H, d, J = 6.8 Hz), 3.97-4.06 (2H, m), 3.35-3.38 (2H, m, overlap with CD₃OD signal), 3.05-3.19 (1H, m), 2.98 (6H, s), 1.47-2.14 (4H, m), 1.14-1.26 (6H, m); HPLC (254 nm): 100% MS (ESI): m/z 632.2[M + H]+ |
| 103 | 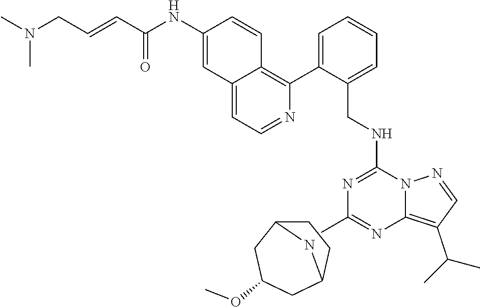 | off-white powder; ¹H NMR (CD₃OD, 400 MHz): δ 8.62 (1H, s), 8.27-8.46 (1H, m), 8.11-8.18 (1H, m), 7.70-7.88 (3H, m), 7.63-7.69 (1H, m), 7.50-7.58 (3H, m), 6.96- 7.07 (1H, m), 6.72 (1H, d, J = 15.2 Hz), 4.87-4.92 (2H, m, overlap with water signal), 4.63-4.72 (1H, m), 4.07 (2H, d, J = 6.8 Hz), 3.46-3.49 (1H, m), 3.30-3.33 (4H, m), 3.01-3.12 (1H, m), 2.96 (6H, s), 1.51-2.43 (8H, m), 1.17 (6H, d, J = 6.4 Hz); LCMS: 100%, MS (ESI): m/z 660.3 [M + H]+ |
| 104 | 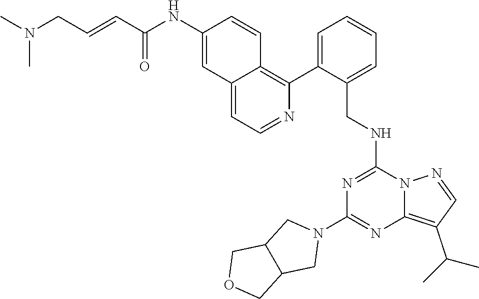 [SMILES: CC(C)c1cnn2c(NCc3ccccc3-c3nccc4cc(NC(=O)/C=C/CN(C)C)ccc34)nc(N3CC4COCC4C3)nc12] | Yellow solid; ¹H NMR (CD₃OD, 400 MHz): δ 8.62-8.66 (1H, m), 8.40 (1H, d, J = 6.4 Hz), 8.21 (1H, d, J = 6.4 Hz), 7.81-7.88 (3H, m), 7.68-7.72 (2H, m), 7.58-7.61 (2H, m), 6.99-7.07 (1H, m), 6.75 (1H, d, J = 14.8 Hz), 4.80-4.84 (1H, m, overlap with water signal), 4.72-4.75 (1H, m), 4.07 (2H, d, J = 6.8 Hz), 3.66-3.88 (9H, m), 3.08-3.15 (2H, m), 2.96 (6H, s), 1.16-1.21 (6H, m); LCMS: 100.0%, MS (ESI): 632.2m/z [M + H]+ |
| 105 | 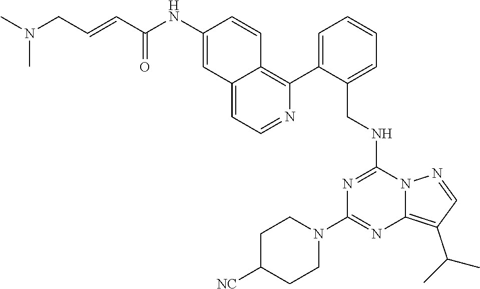 [SMILES: CC(C)c1cnn2c(NCc3ccccc3-c3nccc4cc(NC(=O)/C=C/CN(C)C)ccc34)nc(N3CCC(C#N)CC3)nc12] | White powder; ¹H NMR (CD₃OD, 400 MHz): δ 8.29- 8.33 (2H, m), 7.64-7.68 (1H, m), 7.57-7.62 (2H, m), 7.51-7.55 (1H, m), 7.32-7.43 (4H, m), 6.96-7.02 (1H, m), 6.32 (1H, d, J = 15.6 Hz), 4.92-4.95 (1H, m, overlap with water signal), 4.36-4.43 (1H, m), 3.86- 3.94 (2H, m), 3.38-3.47 (2H, m), 3.23 (2H, d, J = 6.8 Hz), 2.96-3.03 (1H, m), 2.83-2.90 (1H, m), 2.34 (6H, s), 1.80-1.92 (2H, m), 1.64-1.78 (2H, m), 1.17-1.23 (6H, m); LCMS: 99.2%, MS (ESI): 629.2 m/z [M + H]+ |
| 106 | 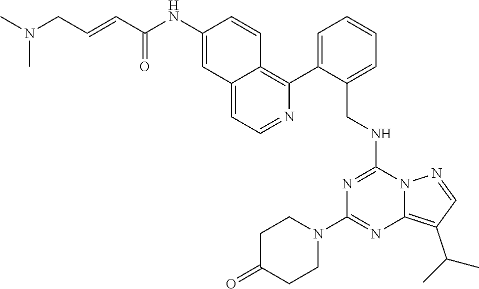 [SMILES: CC(C)c1cnn2c(NCc3ccccc3-c3nccc4cc(NC(=O)/C=C/CN(C)C)ccc34)nc(N3CCC(=O)CC3)nc12] | off-white solid; ¹H NMR (CD₃OD, 400 MHz) δ 8.58 (1H, s), 8.21-8.28 (1H, m), 7.99-8.04 (1H, m), 7.77- 7.81 (2H, m), 7.62-7.73 (2H, m), 7.52-7.59 (2H, m), 7.41-7.45 (1H, m), 6.97-7.06 (1H, m), 6.63-6.72 (1H, m), 4.67-4.73 (2H, m), 4.07 (2H, d, J = 6.8 Hz), 3.54- 3.70 (4H, m), 2.96 (6H, s), 1.93-2.16 (2H, m), 1.63- 1.78 (2H, m), 1.21-1.28 (6H, m); LCMS: 100.0%, MS (ESI): m/z 618.2 [M + H]+ |
| 107 | 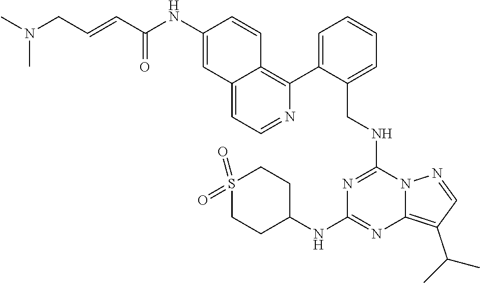 [SMILES: CC(C)c1cnn2c(NCc3ccccc3-c3nccc4cc(NC(=O)/C=C/CN(C)C)ccc34)nc(NC3CCS(=O)(=O)CC3)nc12] | White powder; ¹H NMR (CD₃OD, 400 MHz): δ 8.30- 8.35 (2H, m), 7.63-7.66 (1H, m), 7.55-7.62 (2H, m), 7.48-7.53 (1H, m), 7.40-7.45 (2H, m), 7.32-7.38 (2H, m), 6.91-7.01 (1H, m), 6.39 (1H, d, J = 15.6 Hz), 4.95- 4.98 (1H, m, overlap with water signal), 4.30-4.37 (1H, m), 3.88-3.95 (1H, m), 3.44 (2H, d, J = 6.4 Hz), 3.18- 3.24 (2H, m), 3.08-3.16 (2H, m), 2.1-2.90 (1H, m), 2.50 (6H, s), 2.26-2.35 (2H, m), 2.01-2.20 (2H, m), 1.16-1.22 (6H, m); LCMS: 100%, MS (ESI): m/z 668.3 [M + H]+ |
| 108 | 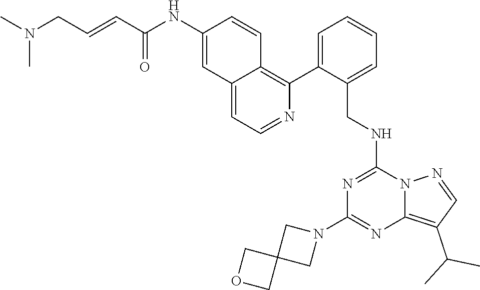 [SMILES: CC(C)c1cnn2c(NCc3ccccc3-c3nccc4cc(NC(=O)/C=C/CN(C)C)ccc34)nc(N3CC4(COC4)C3)nc12] | white solid; ¹H NMR (CD₃OD, 400 MHz) δ 8.30 (1H, d, J = 1.6 Hz), 8.26 (1H, d, J = 6.0 Hz), 7.63-7.66 (1H, m), 7.55-7.60 (2H, m), 7.48-7.53 (1H, m), 7.31-7.47 (4H, m), 6.91-7.02 (1H, m), 6.33 (1H, d, J = 15.6 Hz), 4.86-4.90 (1H, m, overlap with water signal), 4.79- 4.84 (4H, m), 4.35-4.41 (1H, m), 3.95-4.07 (4H, m), 3.28 (2H, d, J = 6.4 Hz), 2.88-2.97 (1H, m), 2.37 (6H, s), 1.16-1.21 (6H, m); LCMS: 100.0%, MS (ESI): m/z 618.1 [M + H]+ |
| 109 | 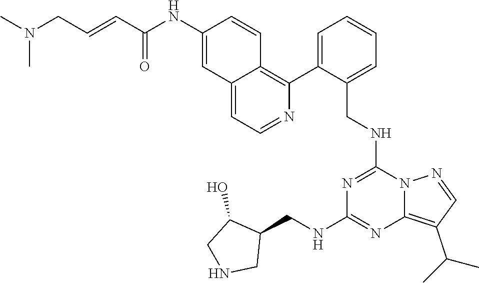 [SMILES: CC(C)c1cnn2c(NCc3ccccc3-c3nccc4cc(NC(=O)/C=C/CN(C)C)ccc34)nc(NC[C@H]3CNC[C@@H]3O)nc12] | Yellow solid; ¹H-NMR (CD₃OD, 400 MHz): δ 8.65-8.68 (1H, m), 8.42-8.47 (1H, m), 8.18-8.21 (1H, m), 7.80- 7.87 (3H, m), 7.67-7.73 (2H, m), 7.56-7.62 (2H, m), 6.98-7.05 (1H, m), 6.68-6.75 (1H, m), 5.03-5.18 (1H, m), 4.72-4.78 (1H, m), 4.28-4.36 (1H, m), 4.06 (2H, d, J = 7.2 Hz), 3.31-3.62 (4H, m), 3.12-3.23 (2H, m), 2.95 (6H, s), 2.83-2.92 (1H, m), 2.38-2.61 (1H, m), 1.18-1.22 (6H, m); LCMS: 100%, MS (ESI): 635.2 m/z [M + H]+ |
| 110 | 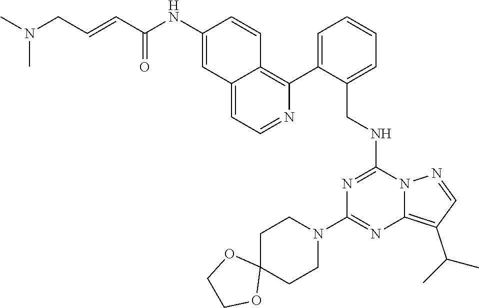 [SMILES: CC(C)c1cnn2c(NCc3ccccc3-c3nccc4cc(NC(=O)/C=C/CN(C)C)ccc34)nc(N3CCC4(CC3)OCCO4)nc12] | White solid; ¹H NMR (CD₃OD, 400 MHz) δ 8.28-8.32 (2H, m), 7.64-7.67 (1H, m), 7.54-7.61 (2H, m) 7.46-7.52 (1H, m), 7.31-7.39 (4H, m), 6.91-6.99 (1H, m), 6.37 (1H, d, J = 15.2 Hz), 4.79-4.84 (1H, m, overlap with water signal), 4.36-4.41 (1H, m), 3.98 (4H, s), 3.60-3.74 (4H, m), 3.37-3.43 (2H, m), 2.81-2.91 (1H, m), 2.44-2.52 (6H, m), 1.52-1.64 (4H, m), 1.19 (6H, d, J = 5.6 Hz); LCMS: 100.0%, MS (ESI): m/z 662.3 [M + H]+ |
| 111 | 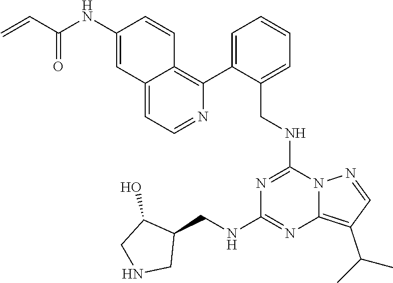 [SMILES: C=CC(=O)Nc1ccc2c(-c3ccccc3CNc3nc(NC[C@H]4CNC[C@@H]4O)nc4c(C(C)C)cnn34)nccc2c1] | Yellow solid; ¹H NMR (CD₃OD, 400 MHz): δ 8.58-8.64 (1H, m), 8.40-8.46 (1H, m), 8.19 (1H, d, J = 6.8 Hz), 7.63-7.89 (5H, m), 7.55-7.62 (2H, m), 6.48-6.52 (2H, m), 5.89-5.94 (1H, m), 5.06-5.23 (1H, m), 4.66-4.76 (1H, m), 4.26-4.36 (1H, m), 3.34-3.61 (4H, m), 3.12- 3.21 (2H, m), 2.78-2.85 (1H, m), 2.38-2.61 (1H, m), 1.15-1.21 (6H, m); LCMS: 100%, MS (ESI): 578.2 m/z [M + H]+ |
| 112 | 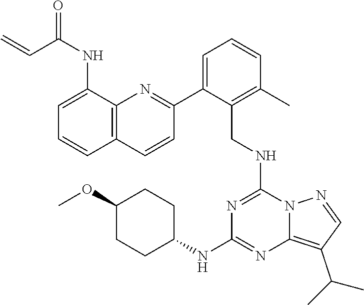 [SMILES: C=CC(=O)Nc1cccc2ccc(-c3cccc(C)c3CNc3nc(N[C@H]4CC[C@H](OC)CC4)nc4c(C(C)C)cnn34)nc12] | White solid; ¹H NMR (CDCl₃, 400 MHz): δ 9.77 (1H, s), 8.88 (1H, dd, J = 6.0, 2.8 Hz), 8.23 (1H, d, J = 8.4 Hz), 7.55-7.61 (4H, m), 7.34-7.43 (3H, m), 6.62 (1H, brs), 6.30-6.44 (2H, m), 5.58 (1H, dd, J = 9.2, 2.0 Hz), 4.68 (2H, d, J = 5.2 Hz), 4.58-4.63 (1H, m), 3.73-3.76 (1H, m), 3.35 (3H, s), 3.12-3.18 (1H, m), 2.96-3.03 (1H, m), 2.53 (3H, s), 2.03-2.13 (4H, m), 1.13-1.35 (10H, m); LCMS: 98.7%, MS (ESI): 606.3 m/z [(M + H)]+ |
| 113 | 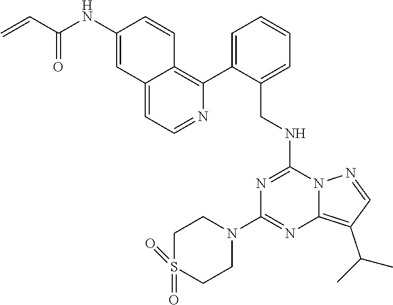 [SMILES: C=CC(=O)Nc1ccc2c(-c3ccccc3CNc3nc(N4CCS(=O)(=O)CC4)nc4c(C(C)C)cnn34)nccc2c1] | White solid; ¹H NMR (CDCl₃, 400 MHz) δ 8.56 (1H, d, J = 5.6 Hz), 8.39 (1H, d, J = 1.6 Hz), 7.68 (1H, d, J = 8.8 Hz), 7.60-7.63 (2H, m), 7.55-7.59 (1H, m), 7.45-7.49 (3H, m), 7.41-7.45 (1H, m), 7.37 (1H, dd, J = 8.8, 2.0 Hz), 6.54 (1H, dd, J = 16.8, 1.2 Hz), 6.30 (1H, dd, J = 16.8, 10.4 Hz), 5.88 (1H, dd, J = 10.4, 1.2 Hz), 4.74-4.85 (1H, m), 4.24-4.36 (5H, m), 2.93-3.05 (5H, m), 1.27 (6H, d, J = 6.8 Hz); LCMS: 100.0%, MS (ESI): m/z 597.1 [M + H]+ |
| 114 | 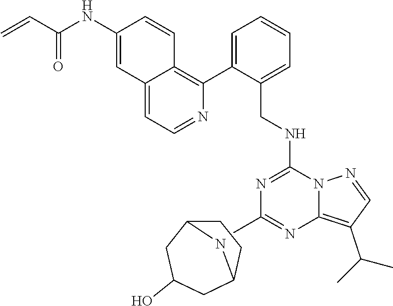 | Light yellow powder; ¹H NMR (CDCl₃, 400 MHz): δ 8.61 (1H, d, J = 5.6 Hz), 8.40 (1H, brs), 7.62-7.70 (3H, m), 7.53-7.57 (2H, m), 7.38-7.49 (3H, m), 7.28-7.35 (1H, m), 6.53 (1H, d, J = 16.8 Hz), 6.27-6.35 (1H, m), 5.87 (1H, d, J = 10.4 Hz), 4.80-4.86 (1H, m), 4.55-4.64 (2H, m), 4.21-4.27 (1H, m), 4.02-4.11 (1H, m), 2.95- 3.04 (1H, m), 2.15-2.26 (3H, m), 1.98-2.06 (2H, m), 1.71-1.75 (2H, m), 1.50-1.55 (1H, m), 1.26 (6H, d, J = 7.2 Hz); LCMS: 100.0%, MS (ESI): 589.1 m/z [(M + H)]+ |
| 115 | 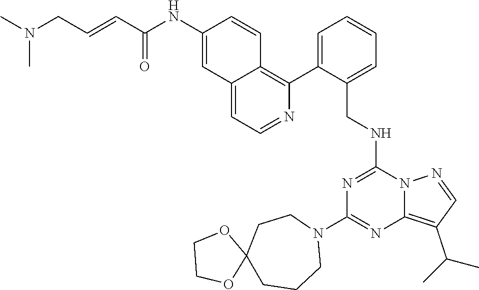 [SMILES: CC(C)c1cnn2c(NCc3ccccc3-c3nccc4cc(NC(=O)/C=C/CN(C)C)ccc34)nc(N3CCCC4(CC3)OCCO4)nc12] | Light yellow solid; ¹H NMR (CD₃OD, 400 MHz): δ 8.28-8.31 (2H, m), 7.43-7.65 (4H, m), 7.28-7.39 (4H, m), 6.92-7.00 (1H, m), 6.31 (1H, d, J = 15.2 Hz), 4.73-4.80 (1H, m, overlap with water signal), 4.36- 4.42 (1H, m), 3.89 (4H, s), 3.49-3.82 (4H, m), 3.23 (2H, d, J = 6.8 Hz), 2.78-2.87 (1H, m), 2.33 (6H, s), 1.56-1.91 (6H, m), 1.15-1.20 (6H, m); LCMS: 99.0%, MS (ESI): 676.2 m/z [M + H]+ |
| 116 | 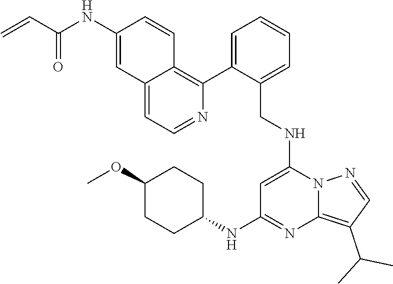 [SMILES: C=CC(=O)Nc1ccc2c(-c3ccccc3CNc3cc(N[C@H]4CC[C@H](OC)CC4)nc4c(C(C)C)cnn34)nccc2c1] | White powder; ¹H NMR (CDCl₃, 400 MHz): δ 8.49 (1H, d, J = 5.6 Hz), 8.38 (1H, brs), 7.55-7.65 (2H, m), 7.43- 7.52 (3H, m), 7.29-7.42 (3H, m), 7.14-7.18 (1H, m, overlap with CDCl₃ signal), 6.45 (1H, d, J = 16.8 Hz), 6.17-6.28 (2H, m), 5.78 (1H, d, J = 7.2 Hz), 4.71 (1H, s), 4.12-4.35 (2H, m), 3.21-3.50 (4H, m), 3.03-3.16 (1H, m), 2.89-2.97 (1H, m), 1.79-2.12 (4H, m), 1.02-1.33 (10H, m); LCMS: 99.9%, MS (ESI): 590.2 m/z [M + H]+ |
| 117 | 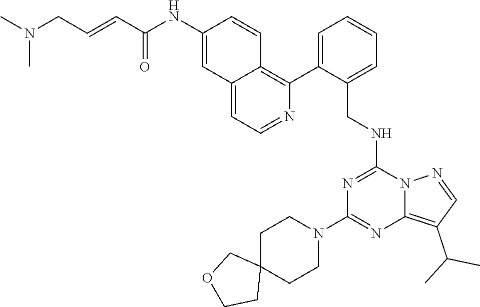 [SMILES: CC(C)c1cnn2c(NCc3ccccc3-c3nccc4cc(NC(=O)/C=C/CN(C)C)ccc34)nc(N3CCC4(CCOC4)CC3)nc12] | Yellow powder; ¹H NMR (400 MHz, CD₃OD) δ 8.65 (1H, d, J = 1.60 Hz), 8.35 (1H, d, J = 6.80 Hz), 8.15 (1H, d, J = 6.40 Hz), 7.79-7.88 (3H, m), 7.56-7.73 (4H, m), 6.97-7.12 (1H, m), 6.75 (1H, d, J = 15.20 Hz), 4.75 (2H, br d, J = 15.60 Hz), 4.09 (2H, d, J = 7.00 Hz), 3.93 (2H, t, J = 7.20 Hz) 3.60-3.78 (6H, m), 3.03-3.10 (1H, m), 2.98 (6H, s), 1.90 (2H, t, J = 7.20 Hz), 1.65 (4H, br s), 1.21 (6H, dd, J = 6.80, 5.60 Hz); HPLC: 100% MS (ESI): m/z 660.2[M + H]+ |
| 118 | 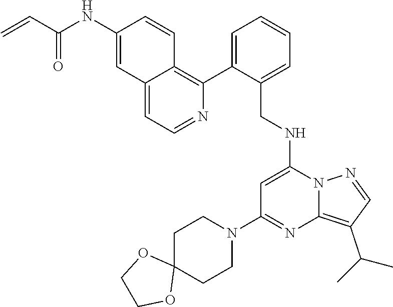 [SMILES: C=CC(=O)Nc1ccc2c(-c3ccccc3CNc3cc(N4CCC5(CC4)OCCO5)nc4c(C(C)C)cnn34)nccc2c1] | White powder; ¹H NMR (400 MHz, CD₃OD) δ 8.56 (1H, d, J = 5.60 Hz), 8.42 (1H, s), 7.36-7.68 (7H, m), 6.53 (1H, d, J = 17.20 Hz), 6.24-6.38 (2H, m), 5.87 (1H, d, J = 10.40 Hz), 5.16 (1H, s), 4.35 (2H, d, J = 5.60 Hz), 4.01 (4H, s), 3.62-3.74 (4H, m), 2.99-3.12 (1H, m), 1.68- 1.79 (4H, m), 1.30 (6H, d, J = 7.00 Hz); HPLC: 97.2% MS (ESI): m/z 604.1[M + H]+ |
| 119 | 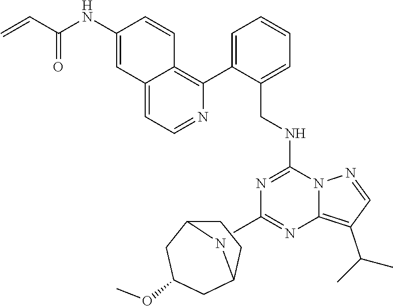 | White powder; ¹H NMR (CDCl₃, 400 MHz): δ 8.56 (1H, d, J = 5.6 Hz), 8.43 (1H, s), 7.36-7.65 (8H, m), 7.20 (1H, dd, J = 2.0 and 9.1 Hz), 6.52 (1H, d, J = 16.8 Hz), 6.24- 6.34 (2H, m), 5.86 (1H, d, J = 10.5 Hz), 5.00 (1H, s), 4.22-4.42 (4H, m), 3.72 (1H, br d, J = 3.6 Hz), 3.34 (1H, br s), 3.28 (3H, s), 3.02 (1H, quin, J = 6.8 Hz), 2.10 (2H, br d, J = 7.1 Hz), 1.91-2.05 (3H, m), 1.66-1.91 (3H, m), 1.27 (6H, d, J = 6.9 Hz); LCMS: 100%, MS (ESI): m/z 602.5 [M + H]+ |
| 120 | 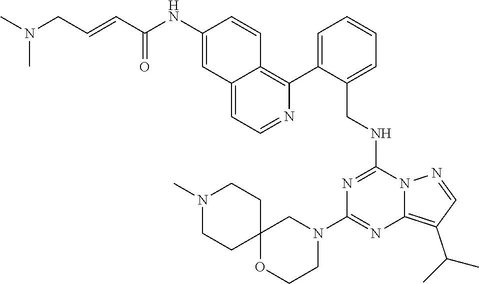 [SMILES: CC(C)c1cnn2c(NCc3ccccc3-c3nccc4cc(NC(=O)/C=C/CN(C)C)ccc34)nc(N3CCOC4(CCN(C)CC4)C3)nc12] | Yellow solid; ¹H NMR (CD₃OD, 400 MHz): δ 8.61 (1H, d, J = 2.0 Hz), 8.34 (1H, d, J = 6.8 Hz), 8.12 (1H, d, J = 6.8 Hz), 7.64-7.86 (4H, m), 7.53-7.60 (2H, m), 7.47 (1H, s), 6.97-7.06 (1H, m), 6.71 (1H, d, J = 15.2 Hz), 4.95-5.01 (1H, m), 4.68-4.74 (1H, m), 4.06 (2H, d, J = 7.2 Hz), 3.46-3.81 (6H, m), 3.34-3.40 (2H, m), 3.14-3.25 (2H, m), 2.93-3.02 (7H, m), 2.87 (3H, s), 2.09- 2.21 (2H, m), 1.79-1.92 (2H, m), 1.14-1.19 (6H, m); LCMS: 98.6%, MS (ESI): 689.4 m/z [M + H]+ |
| 121 | 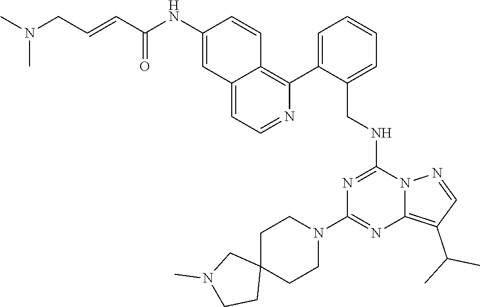 [SMILES: CC(C)c1cnn2c(NCc3ccccc3-c3nccc4cc(NC(=O)/C=C/CN(C)C)ccc34)nc(N3CCC4(CCN(C)C4)CC3)nc12] | Pale yellow solid; ¹H NMR (400 MHz, CD₃OD) δ 8.61 (1H, s), 8.39 (1H, d, J = 6.4 Hz), 8.17 (1H, d, J = 6.4 Hz), 7.79-7.81 (3H, m), 7.64-7.67 (1H, m), 7.54-7.63 (3H, m), 6.99-7.05 (1H, m), 6.76 (1H, d, J = 14.8 Hz), 4.95-4.99 (1H, m), 4.68-4.71 (1H, m), 4.08 (2H, d, J = 7.2 Hz), 3.76-3.81 (3H, m), 3.67-3.73 (3H, m), 3.02-3.06 (2H, m), 2.99 (3H, s), 2.96 (6H, s), 2.17-2.20 (1H, m), 2.06-2.11 (1H, m), 1.72-1.77 (4H, m), 1.19 (6H, d, J = 6.8 Hz); LCMS: 99.8%, MS (ESI): 673.4 m/z [M + H]+ |
| 122 | 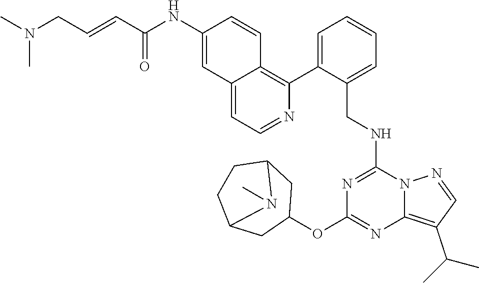 [SMILES: CC(C)c1cnn2c(NCc3ccccc3-c3nccc4cc(NC(=O)/C=C/CN(C)C)ccc34)nc(OC3CC4CCC(C3)N4C)nc12] | Yellow powder; ¹H NMR (CD₃OD, 400 MHz) δ 8.64 (1H, d, J = 2 Hz), 8.36 (1H, d, J = 6.8 Hz), 8.14 (1H, d, J = 6.4 Hz), 7.77-7.86 (2H, m), 7.69- 7.75 (1H, m), 7.61-7.69 (2H, m), 7.58 (1H, s), 7.55 (1H, d, J = 7.6 Hz), 6.97-7.08 (1H, m), 6.70 (1H, d, J = 14.8 Hz), 5.09 (1H, m), 5.01 (1H, d, J = 15.6 Hz), 4.64 (1H, d, J = 15.6 Hz), 4.07 (2H, d, J = 7.6 Hz), 3.93 (2H, m), 2.96 (6H, s), 2.89 (1H, m), 2.83 (3H, s), 2.03-2.58 (8H, m), 1.21 (6H, d, J = 6.8 Hz); LCMS: 100%, MS (ESI): m/z 660.5 [M + H]+ |
| 123 | 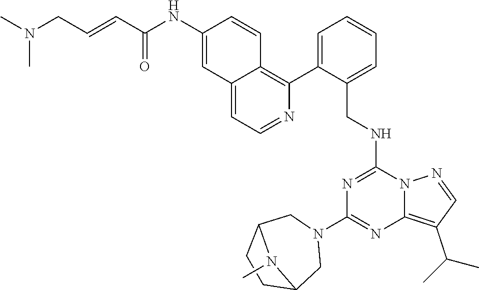 [SMILES: CC(C)c1cnn2c(NCc3ccccc3-c3nccc4cc(NC(=O)/C=C/CN(C)C)ccc34)nc(N3CC4CCC(C3)N4C)nc12] | White solid; ¹H NMR (CD₃OD, 400 MHz) δ 8.54 (1H, s), 8.32 (1H, d, J = 6.8 Hz), 8.09 (1H, d, J = 6.8 Hz), 7.77-7.82 (2H, m), 7.61-7.64 (2H, m), 7.49- 7.53 (2H, m), 7.33 (1H, s), 6.97-7.03 (1H, m), 6.71 (1H, d, J = 15.2 Hz), 4.95-5.00 (2H, m), 4.39-4.68 (2H, m), 4.00-4.08 (4H, m), 3.31-3.44 (1H, m), 2.96 (6H, s), 2.86 (3H, s), 2.81-2.86 (1H, m), 2.21- 2.26 (2H, m), 1.88-1.93 (1H, m), 1.67-1.72 (2H, m), 1.27-1.32 (2H, m), 1.18 (6H, d, J = 6.8 Hz); LCMS: 99.5%, MS (ESI): m/z 645.4 [M + H]+ |
| 124 | 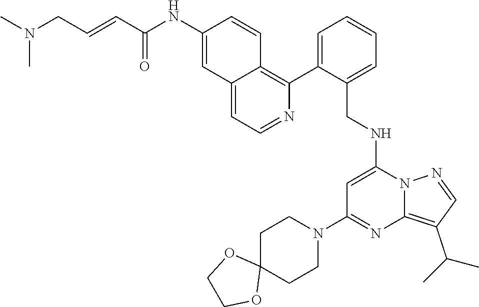 [SMILES: CC(C)c1cnn2c(NCc3ccccc3-c3nccc4cc(NC(=O)/C=C/CN(C)C)ccc34)cc(N3CCC4(CC3)OCCO4)nc12] | White powder; ¹H NMR (400 MHz, CD₃OD) δ 8.32- 8.47 (2H, m), 7.68-7.74 (2H, m), 7.56-7.62 (1H, m), 7.52 (1H, t, J = 7.60 Hz), 7.34-7.41 (4H, m), 6.99 (1H, dt, J = 15.37, 6.49 Hz), 6.34 (1H, d, J = 15.60 Hz), 5.03 (1H, s), 4.55 (1H, br d, J = 15.56 Hz), 4.37- 4.45 (1H, m), 4.00 (4H, s), 3.50-3.71 (4H, m), 3.24 (2H, d, J = 5.60 Hz), 2.91-2.99 (1H, m), 2.34 (6H, s), 1.56-1.71 (4H, m), 1.23 (6H, d, J = 8.00 Hz); HPLC: 99.4% MS (ESI): m/z 661.3[M + H]+ |
| 125 | 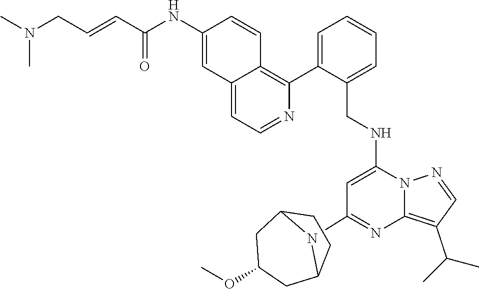 | Yellow powder; ¹H NMR (CD₃OD, 400 MHz): δ 8.57 (1H, d, J = 1.8 Hz), 8.37 (1H, d, J = 6.5 Hz), 8.07 (1H, d, J = 6.3 Hz), 7.84-7.90 (1H, m), 7.80 (1H, t, J = 7.2 Hz), 7.64-7.74 (2H, m), 7.49-7.58 (3H, m), 6.94-7.07 (1H, m), 6.73 (1H, d, J = 15.3 Hz), 4.99 (1H, s), 4.94 (1H, br s), 4.65-4.75 (1H, m), 4.49 (2H, br s), 4.06 (2H, d, J = 7.3 Hz), 3.46-3.52 (1H, m), 3.33 (3H, s), 3.11- 3.21 (1H, m), 2.96 (6H, s), 2.30 (2H, br d, J = 7.5 Hz), 1.92-2.11 (5H, m), 1.68 (1H, br s), 1.18 (6H, dd, J = 6.9 and 12.2 Hz).. LCMS: 98.2%, MS (ESI): m/z 659.5 [M + H]+ |
| 126 | 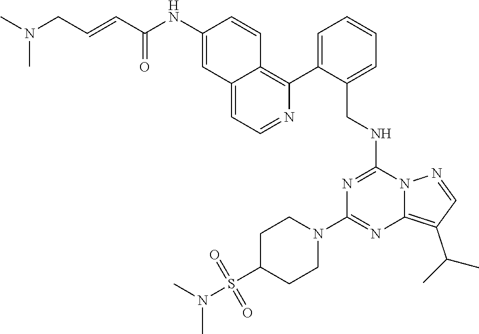 [SMILES: CC(C)c1cnn2c(NCc3ccccc3-c3nccc4cc(NC(=O)/C=C/CN(C)C)ccc34)nc(N3CCC(S(=O)(=O)N(C)C)CC3)nc12] | White powder; ¹H NMR (CD₃OD, 400 MHz): δ 8.65 (1H, s), 8.35-8.47 (1H, m), 8.22 (1H, d, J = 6.40 Hz), 7.78-7.92 (3H, m), 7.53-7.73 (4H, m), 6.94-7.11 (1H, m), 6.74 (1H, d, J = 14.40 Hz), 4.95-5.02 (1H, m), 4.75-4.82 (1H, m), 4.34-4.55 (2H, m), 4.07 (2H, d, J = 7.20 Hz), 3.51-3.63 (1H, m), 3.02-3.25 (3H, m), 2.96 (12H, s). 1.52-2.27 (4H, m), 1.18 (6H, t, J = 6.78 Hz); HPLC: 100%, MS (ESI): 711.5 m/z [M + H]+ |
| 127 | 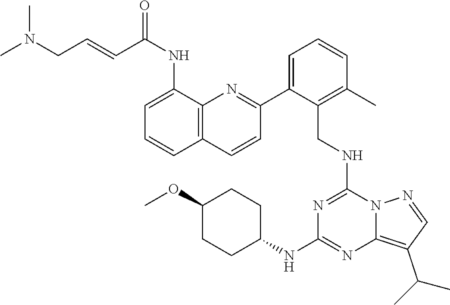 [SMILES: CO[C@H]1CC[C@H](Nc2nc(NCc3c(C)cccc3-c3ccc4cccc(NC(=O)/C=C/CN(C)C)c4n3)n3ncc(C(C)C)c3n2)CC1] | White powder; ¹H NMR (CD₃OD, 400 MHz) δ8.67 (1H, d, J = 8.0 Hz) 8.29 (1H, d, J = 8.4 Hz) 7.65 (2H, d, J = 9.2 Hz) 7.53-7.59 (2H, m) 7.40-7.44 (2H, m) 7.34-7.39 (1H, m) 6.78-6.91 (1H, m) 6.27 (1H, s) 4.76 (2H, s) 3.33 (3H, s) 3.14 (3H, s) 2.92 (1H, t, J = 8.4 Hz) 2.56 (3H, s) 2.33 (6H, s) 2.02 (4H, d, J = 12.0 Hz) 1.29 (2H, s) 1.24 (6H, d, J = 6.8 Hz) 1.17 (2H, s); LCMS: 92.5%, MS (ESI): m/z 662.5 [M + H]+ |
| 128 | 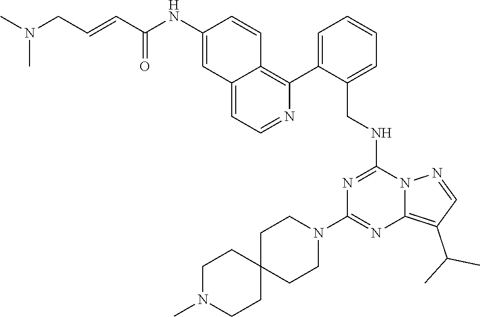 [SMILES: CC(C)c1cnn2c(NCc3ccccc3-c3nccc4cc(NC(=O)/C=C/CN(C)C)ccc34)nc(N3CCC4(CCN(C)CC4)CC3)nc12] | Yellow solid; ¹H NMR (CD₃OD, 400 MHz) δ 8.67 (1H, d, J = 1.2 Hz) 8.44 (1H, d, J = 6.4 Hz) 8.23 (1H, d, J = 6.4 Hz) 7.88 (1H, dd, J = 9.2, 1.6 Hz) 7.80-7.85 (2H, m) 7.65-7.72 (2H, m) 7.62 (1H, s) 7.58-7.61 (1H, m) 6.99- 7.09 (1H, m) 6.76 (1H, d, J = 15.56 Hz) 5.01 (2H, br d, J = 15.6 Hz) 4.73 (1H, d, J = 15.6 Hz) 4.08 (2H, d, J = 6.8 Hz) 3.77 (4H, s) 3.43 (2H, d, J = 12.0 Hz) 3.19-3.24 (2H, m) 3.08-3.16 (1H, m) 2.96 (6H, s) 2.92 (3H, s) 1.93-2.13 (2H, m) 1.84 (2H, s) 1.71-1.78 (2H, m) 1.54- 1.65 (2H, m) 1.18 (6H, dd, J = 6.8, 3.2 Hz); LCMS: 100.0%, MS (ESI): m/z 687.6 [M + H]+ |
| 129 | 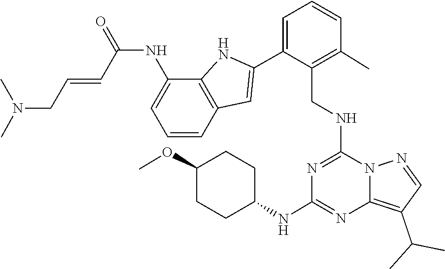 [SMILES: CO[C@H]1CC[C@H](Nc2nc(NCc3c(C)cccc3-c3cc4cccc(NC(=O)/C=C/CN(C)C)c4[nH]3)n3ncc(C(C)C)c3n2)CC1] | White powder; ¹H NMR (CD₃OD, 400 MHz): δ 7.72 (1H, s), 7.37 (3H, s), 7.25-7.34 (2H, m), 6.98 (1H, t, J = 7.80 Hz), 6.81-6.92 (1H, m), 6.70-6.78 (1H, m), 6.35 (1H, s), 5.00 (2H, s), 4.01 (2H, d, J = 7.03 Hz), 3.59-3.76 (1H, m), 3.32-3.38 (3H, m), 3.12-3.20 (1H, m) 2.94 (7H, s), 2.55 (3H, s), 1.85-2.05 (4H, m), 1.22- 1.31 (10H, m); HPLC: 98.0%, MS (ESI): 650.5 m/z [M + H]+ |
| 130 | 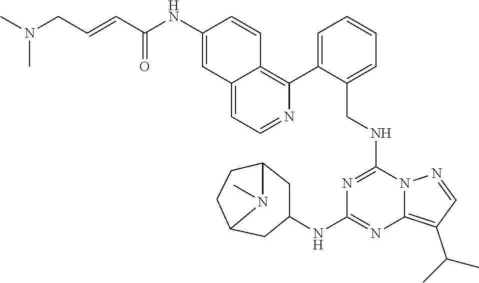 [SMILES: CC(C)c1cnn2c(NCc3ccccc3-c3nccc4cc(NC(=O)/C=C/CN(C)C)ccc34)nc(NC3CC4CCC(C3)N4C)nc12] | White powder; ¹H NMR (CD₃OD, 400 MHz): δ 8.60- 8.72 (1H, m), 8.41 (1H, d, J = 6.8 Hz), 8.20 (1H, d, J = 5.6 Hz), 7.78-7.89 (3H, m), 7.53-7.73 (4H, m), 6.96- 7.10 (1H, m), 6.71 (1H, d, J = 15.2 Hz), 5.01 (2H, m), 4.72 (1H, d, J = 15.6 Hz), 4.07 (2H, d, J = 6.8 Hz), 3.90-3.99 (2H, m), 2.96 (6H, s), 2.81-2.90 (4H, m), 2.06-2.66 (8H, m), 1.23 (6H, m); LCMS: 100%, MS (ESI): m/z 659.4 [M + H]+ |
| 131 | 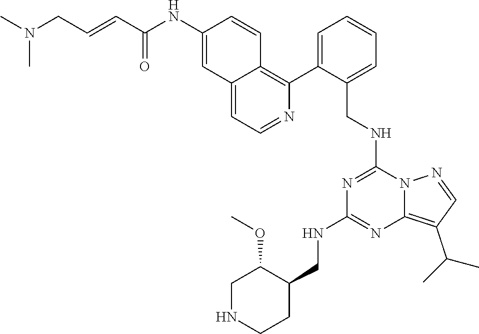 [SMILES: CO[C@H]1CNCC[C@@H]1CNc1nc(NCc2ccccc2-c2nccc3cc(NC(=O)/C=C/CN(C)C)ccc23)n2ncc(C(C)C)c2n1] | Yellow powder; ¹H NMR (400 MHz, CD₃OD) δ 8.57 (1H, s), 8.41 (1H, s), 8.06 (1H, s), 7.50-7.88 (7H, m), 6.92-7.08 (1H, m) 6.66 (1H, d, J = 15.20 Hz), 4.96-5.19 (2H, m), 4.62-4.80 (1H, m), 4.06 (2H, d, J = 7.20 Hz), 3.38-3.69 (7H, m), 2.81-3.03 (9H, m), 1.88-2.12 (2H, m), 1.58 (1H, s), 1.18 (6H, d, J = 6.80 Hz); HPLC: 99.8% MS (ESI): m/z 663.6[M + H]+ |
| 132 | 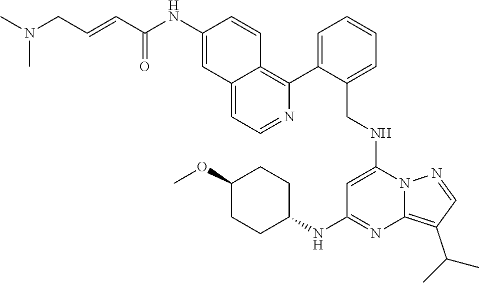 [SMILES: CO[C@H]1CC[C@H](Nc2cc(NCc3ccccc3-c3nccc4cc(NC(=O)/C=C/CN(C)C)ccc34)n3ncc(C(C)C)c3n2)CC1] | Yellow powder; ¹H NMR (400 MHz, CD₃OD) δ 8.55 (1H, s), 8.40 (1H, d, J = 6.40 Hz), 8.03 (1H, s), 7.76-7.89 (2H, m), 7.59-7.70 (2H, m), 7.43-7.57 (3H, m), 6.96- 7.03 (1H, m), 6.70 (1H, d, J = 15.20 Hz), 4.95-5.02 (2H, m) 4.65-4.75 (2H, m), 4.06 (2H, d, J = 7.20 Hz), 3.38 (3H, s) 3.21-3.28(1H, m), 2.91-3.01 (7H, m), 2.00-2.19 (3H, m), 1.72 (1H, s), 1.29-1.48 (4H, m) 1.18 (6H, dd, J = 14.00, 6.80 Hz); HPLC: 100% MS (ESI): m/z 647.4[M + H]+ |
| 133 | 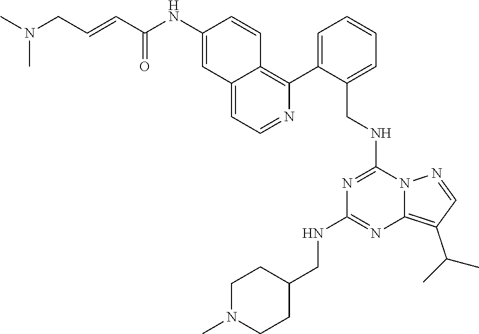 [SMILES: CC(C)c1cnn2c(NCc3ccccc3-c3nccc4cc(NC(=O)/C=C/CN(C)C)ccc34)nc(NCC3CCN(C)CC3)nc12] | Yellow powder; ¹H NMR (CD₃OD, 400 MHz): δ 8.65 (1H, s), 8.43 (1H, d, J = 6.4 Hz), 8.18 (1H, d, J = 6.8 Hz), 7.76-7.93 (3H, m), 7.54-7.74 (4H, m), 6.97-7.09 (1H, m), 6.72 (1H, d, J = 15.6 Hz), 5.05 (1H, d, J = 15.6 Hz), 4.70-4.79 (1H, m), 4.07 (2H, d, J = 7.2 Hz), 3.53 (2H, d, J = 10.4 Hz), 3.10-3.27 (2H, m), 3.01 (2H, m), 2.96 (6H, s), 2.86 (4H, m), 1.80-2.10 (3H, m), 1.61 (2H, m), 1.16-1.25 (6H, m); LCMS: 100%, MS (ESI): m/z 647.4 [M + H]+ |
| 134 | 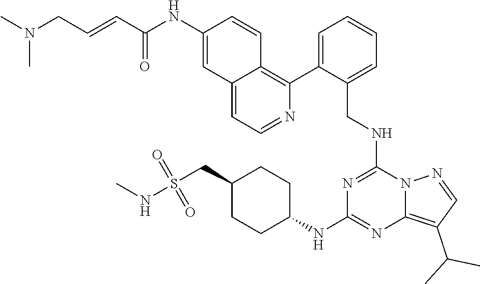 [SMILES: CNS(=O)(=O)C[C@H]1CC[C@H](Nc2nc(NCc3ccccc3-c3nccc4cc(NC(=O)/C=C/CN(C)C)ccc34)n3ncc(C(C)C)c3n2)CC1] | White powder; ¹H NMR (CD₃OD, 400 MHz) δ 8.68 (1H, s), 8.39 (1H, d, J = 6.8 Hz), 8.24 (1H, d, J = 6.8 Hz), 7.78-7.92 (3H, m), 7.68 (1H, m), 7.52-7.65 (3H, m), 7.02 (1H, m), 6.71 (1H, d, J = 14.8 Hz), 4.94-4.95 (1H, m), 4.81-4.82 (1H, m), 4.07 (2H, d, J = 7.2 Hz), 3.05 (2H, d, J = 5.2 Hz), 2.96 (6H, s), 2.77-2.91 (2H, m), 2.74 (3H, s), 1.70-2.16 (5H, m), 1.28-1.52 (4H, m), 1.13-1.22 (6H, m); LCMS: 100%, MS (ESI): m/z 725.4 [M + H]+ |
| 135 | 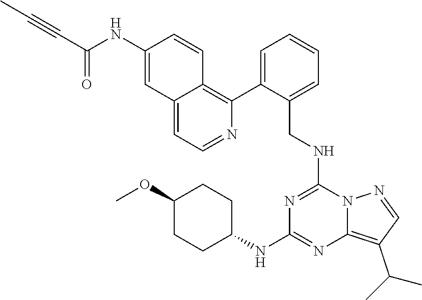 [SMILES: CC#CC(=O)Nc1ccc2c(-c3ccccc3CNc3nc(N[C@H]4CC[C@H](OC)CC4)nc4c(C(C)C)cnn34)nccc2c1] | White powder; ¹H NMR (CD₃OD, 400 MHz): δ 8.47 (1H, s), 8.40 (1H, d, J = 6.4 Hz), 8.22 (1H, d, J = 6.4 Hz), 7.78-7.88 (2H, m), 7.63-7.72 (2H, m), 7.52-7.61 (3H, m), 4.97 (1H, s), 4.75 (1H, d, J = 16 Hz), 3.45- 3.60 (1H, m), 3.40 (3H, s), 3.25 (1H, s), 2.77-2.91 (1H, m), 2.16 (1H, m), 2.09 (3H, s), 2.05 (2H, m), 1.65-1.81 (1H, m), 1.27-1.52 (4H, m), 1.17-1.27 (6H, m); LCMS: 98%, MS (ESI): m/z 603.3 [M + H]+ |
| 136 | 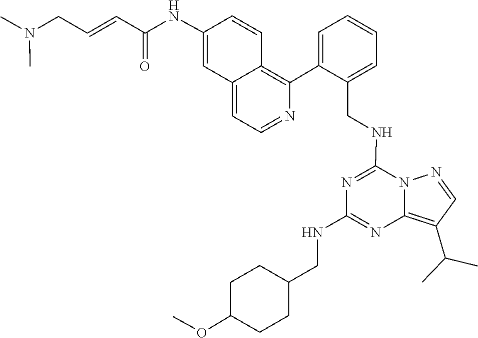 [SMILES: COC1CCC(CNc2nc(NCc3ccccc3-c3nccc4cc(NC(=O)/C=C/CN(C)C)ccc34)n3ncc(C(C)C)c3n2)CC1] | Yellow powder; ¹H NMR (400 MHz, CD₃OD) δ ppm 8.66 (1H, d, J = 1.60 Hz), 8.43 (1H, d, J = 6.40 Hz), 8.18-8.32 (1H, m), 7.80-7.87 (3H, m) 7.65-7.75 (1H, m), 7.53-7.63 (3H, m), 6.95-7.09 (1H, m), 6.72 (1H, d, J = 15.20 Hz), 4.92-5.01 (1H, m), 4.76-4.82 (1H, m), 4.07 (2H, d, J = 7.20 Hz), 3.36 (3H, s), 3.22-3.26 (1H, m), 3.11-3.20 (2H, m), 3.01-3.09 (2H, m), 2.96 (6H, s), 2.78-2.88 (1H, m), 1.96-2.16 (3H, m), 1.70- 1.85 (2H, m), 1.32-1.51 (2H, m), 1.18 (6H, t, J = 6.40 Hz), 1.00-1.12 (2H, m); HPLC: 96.4% MS (ESI): m/z 662.4[M + H]+ |
| 137 | 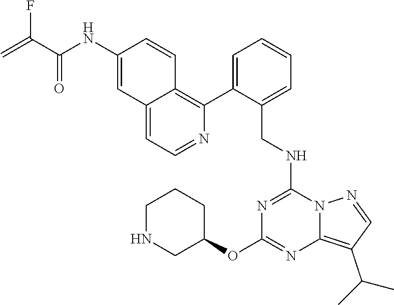 [SMILES: C=C(F)C(=O)Nc1ccc2c(-c3ccccc3CNc3nc(O[C@@H]4CCCNC4)nc4c(C(C)C)cnn34)nccc2c1] | White powder; ¹H NMR (CDCl₃, 400 MHz): δ 8.56 (1H, m), 8.40 (2H, m), 7.41-7.88 (9H, m), 5.81-6.01 (1H, m), 5.33 (1H, dd, J = 15.2, 3.6 Hz), 4.74-5.21 (2H, m), 4.19 (1H, m), 2.84-3.36 (5H, m), 1.91 (4H, m), 1.26 (6H, d, J = 6.8 Hz); LCMS: 100%, MS (ESI): m/z 581.3 [M + H]+ |
| 138 | 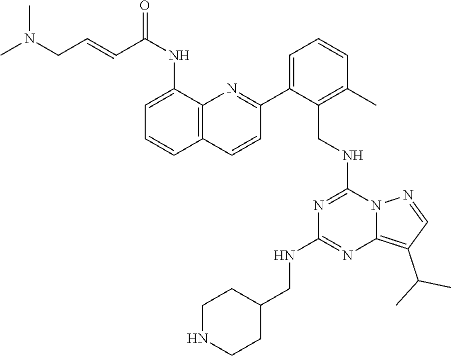 [SMILES: Cc1cccc(-c2ccc3cccc(NC(=O)/C=C/CN(C)C)c3n2)c1CNc1nc(NCC2CCNCC2)nc2c(C(C)C)cnn12] | Yellow solid; ¹H NMR (400 MHz, CD₃OD) δ = 8.74 (d, J = 7.2 Hz, 1H), 8.25 (d, J = 8.4 Hz, 1H), 7.71-7.64 (m, 2H), 7.64-7.58 (m, 2H), 7.49-7.41 (m, 2H), 7.31 (d, J = 6.8 Hz, 1H), 6.94-6.84 (m, 1H), 6.78-6.71 (m, 1H), 4.91 (s, 1H), 4.83 (br s, 1H), 4.02 (d, J = 7.0 Hz, 2H), 3.37 (m, 2H), 3.11 (br d, J = 5.8 Hz, 2H), 2.97-2.84 (m, 9H), 2.62 (s, 3H), 1.84 (br d, J = 13.8 Hz, 2H), 1.48-1.37 (m, 2H), 1.26 (d, J = 7.2 Hz, 6H); LCMS: 100%, MS (ESI): m/z 647.4[M + H]+ |
| 139 | 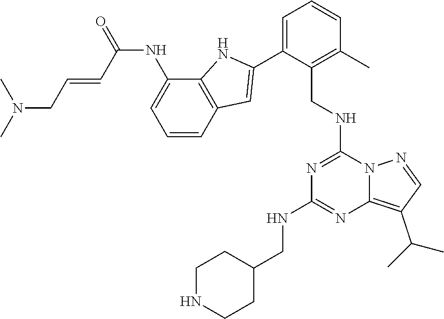 [SMILES: Cc1cccc(-c2cc3cccc(NC(=O)/C=C/CN(C)C)c3[nH]2)c1CNc1nc(NCC2CCNCC2)nc2c(C(C)C)cnn12] | White solid; ¹H NMR (400 MHz, CD₃OD) δ = 7.83 (s, 1H), 7.43 (m, 1H), 7.42-7.38 (m, 2H), 7.37-7.31 (m, 2H), 7.04 (t, J = 7.8 Hz, 1H), 6.94-6.82 (m, 1H), 6.80- 6.73 (m, 1H), 6.43 (s, 1H), 5.03 (s, 2H), 4.04 (d, J = 7.0 Hz, 2H), 3.39-3.34 (m, 2H), 3.24 (br d, J = 12.5 Hz, 2H), 3.00 (br d, J = 6.8 Hz, 1H), 2.96 (s, 6H), 2.73 (m, 2H), 2.57 (s, 3H), 1.82 (m, 3H), 1.38 (m, 2H), 1.31 (d, J = 7.0 Hz, 6H); LCMS: 100%, MS (ESI): m/z 635.4[M + H]+ |
| 140 | 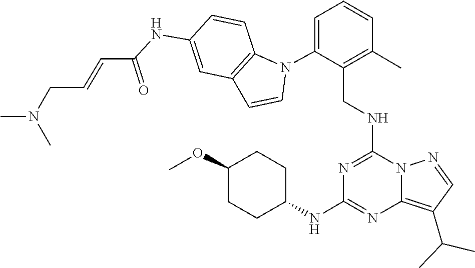 [SMILES: CO[C@H]1CC[C@H](Nc2nc(NCc3c(C)cccc3-n3ccc4cc(NC(=O)/C=C/CN(C)C)ccc43)n3ncc(C(C)C)c3n2)CC1] | White powder; ¹H NMR (400 MHz, CD₃OD) δ = 7.96 (d, J = 1.6 Hz, 1H), 7.76 (s, 1H), 7.54-7.51 (m, 1H), 7.48-7.45 (m, 1H), 7.25-7.17 (m, 2H), 6.95 (m, 1H), 6.88-6.83 (m, 1H), 6.77 (d, J = 8.4 Hz, 1H), 6.62 (m, 1H), 6.50 (d, J = 3.2 Hz, 1H), 4.84-4.79 (m, 1H), 4.66 (m, 1H), 4.08-3.97 (m, 3H), 3.38 (s, 3H), 3.22 (br s, 1H), 2.97 (s, 6H), 2.90-2.84 (m, 1H), 2.67-2.61 (m, 3H), 2.18-2.06 (m, 2H), 1.99 (m, 2H), 1.31-1.22 (m, 10H); LCMS: 100%, MS (ESI): m/z 650.4 [M + H]+ |
| 141 | 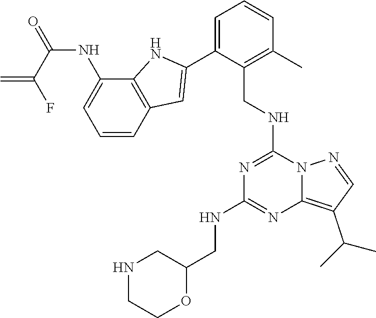 [SMILES: C=C(F)C(=O)Nc1cccc2cc(-c3cccc(C)c3CNc3nc(NCC4CNCCO4)nc4c(C(C)C)cnn34)[nH]c12] | Gray solid; ¹H NMR (400 MHz, CD₃OD) δ = 7.85 (s, 1H), 7.47-7.35 (m, 4H), 7.19 (d, J = 7.9 Hz, 1H), 7.11-7.02 (m, 1H), 6.49 (br s, 1H), 5.57-5.39 (m, 1H), 5.24 (dd, J = 3.4, 15.0 Hz, 1H), 4.95 (m, 2H), 4.12- 4.00 (m, 1H), 3.86 m, 1H), 3.75 (m, 1H), 3.54-3.41 (m, 2H), 3.21 (m, 2H), 3.04 (m, 2H), 2.83 (s, 1H), 2.54 (s, 3H), 1.31 (d, J = 6.9 Hz, 6H); LCMS: 100%, MS (ESI): m/z 598.3 [M + H]+ |
| 142 | 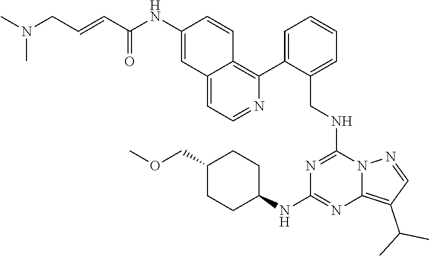 [SMILES: COC[C@H]1CC[C@H](Nc2nc(NCc3ccccc3-c3nccc4cc(NC(=O)/C=C/CN(C)C)ccc34)n3ncc(C(C)C)c3n2)CC1] | Yellow powder; ¹H NMR (400 MHz, CD₃OD) δ ppm 8.68 (1H, s), 8.41 (1H, d, J = 6.40 Hz), 8.25 (1H, d, J = 6.40 Hz), 7.78-7.90 (3H, m), 7.68 (1H, t, J = 6.40 Hz), 7.51-7.61 (3H, m), 6.95-7.11 (1H, m), 6.72 (1H, d, J = 15.20 Hz), 4.95-5.02 (1H, m) 4.73-4.83 (1H, m), 4.07 (2H, d, J = 7.20 Hz), 3.43-3.54 (1H, m), 3.37 (3H, s) 3.25-3.31 (2H, m), 2.96 (6H, s), 2.77-2.87 (1H, m) 1.55- 2.11 (5H, m), 1.2-1.44 (H, m), 1.06-1.22 (8H, m); HPLC: 98.5% MS (ESI): m/z 662.4[M + H]+ |
| 143 | 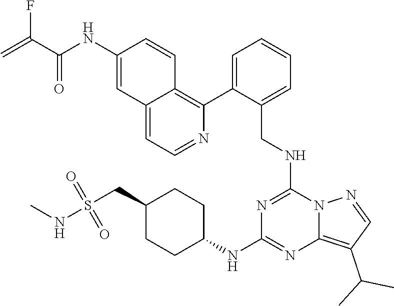 [SMILES: C=C(F)C(=O)Nc1ccc2c(-c3ccccc3CNc3nc(N[C@H]4CC[C@H](CS(=O)(=O)NC)CC4)nc4c(C(C)C)cnn34)nccc2c1] | Yellow powder; ¹H NMR (400 MHz, CD₃OD) δ 8.62 (1H, s) 8.45 (1H, d, J = 6.40 Hz), 8.28 (1H, d, J = 6.80 Hz), 7.84-7.93 (3H, m), 7.66-7.72 (1H, m), 7.59 (2H, d, J = 8.00 Hz), 7.52 (1H, s), 5.78-5.95 (1H, m), 5.46 (1H, dd, J = 14.80, 3.60 Hz), 4.95-5.02 (1H, m), 4.71-4.82 (1H, m), 3.50-3.59 (1H,m), 3.06 (2H, d, J = 6.40 Hz), 2.78-2.87 (1H, m), 2.74 (3H, s), 1.68-1.78 (1H, m), 1.25- 1.53 (4H, m), 1.12-1.23 (6H, m), 1.87-2.18 (4H, m); HPLC: 98.6% MS (ESI): m/z 686.3[M + H]+ |
| 144 | 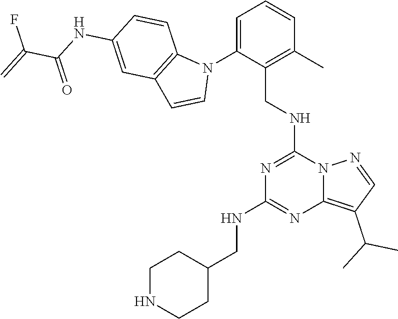 | White solid; ¹H NMR (CDCl₃, 400 MHz,) δ9.43 (1H, s) 8.53 (1H, d, J = 5.2 Hz) 8.47 (1H, d, J = 9.2 Hz) 8.30 (1H, d, J = 2.8 Hz) 7.60 (1H, dd, J = 9.2, 2.8 Hz) 7.56 (1H, s) 7.46 (1H, s) 6.47 (1H, d, J = 5.2 Hz) 4.07 (6H, s) 3.10-3.13 (1H, m) 3.08 (1H, d, J = 3.2 Hz) 3.05 (2H, d, J = 8.8 Hz) 2.62-2.75 (2H, m) 1.73-1.80 (2H, m) 1.02 (3H, t, J = 7.6 Hz); LCMS: 98.0%, MS (ESI): m/z 531.3 [M + H]+ |
| 145 | 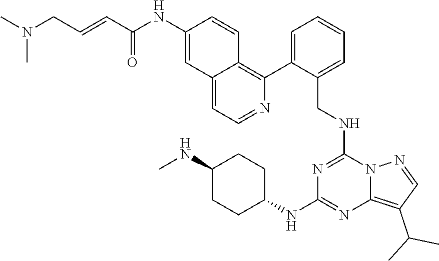 [SMILES: CN[C@H]1CC[C@H](Nc2nc(NCc3ccccc3-c3nccc4cc(NC(=O)/C=C/CN(C)C)ccc34)n3ncc(C(C)C)c3n2)CC1] | Yellow solid; ¹H NMR (400 MHz, CD₃OD) δ = 8.70 (s, 1H), 8.45 (d, J = 6.5 Hz, 1H), 8.26 (d, J = 6.8 Hz, 1H), 7.91 (br s, 2H), 7.81 (dd, J = 2.0, 9.0 Hz, 1H), 7.74-7.68 (m, 1H), 7.64-7.58 (m, 2H), 7.56 (s, 1H), 7.04 (td, J = 7.2, 15.0 Hz, 1H), 6.73 (d, J = 15.3 Hz, 1H), 5.08 (m, 1H), 4.85- 4.78 (m, 1H), 4.09 (d, J = 7.3 Hz, 2H), 3.69 (m, 1H), 3.17-3.06 (m, 1H), 2.98 (s, 6H), 2.91-2.81 (m, 1H), 2.78 (s, 3H), 2.28 m, 1H), 2.19 (m, 2H), 1.85 (m, 1H), 1.77-1.66 (m, 1H), 1.58 (m, 2H), 1.52-1.38 (m, 1H), 1.23-1.16 (m, 6H); LCMS: 100%, MS (ESI): m/z 647.4 [M + H]+ |
| 146 | 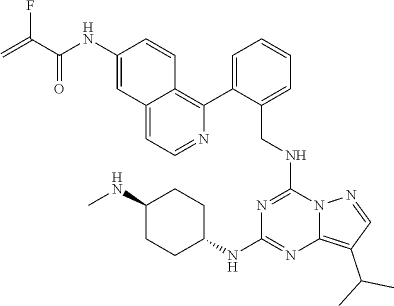 [SMILES: C=C(F)C(=O)Nc1ccc2c(-c3ccccc3CNc3nc(N[C@H]4CC[C@H](NC)CC4)nc4c(C(C)C)cnn34)nccc2c1] | Yellow powder; ¹H NMR (400 MHz, CD₃OD) δ 8.59 (1H, s), 8.45 (1H, d, J = 6.00 Hz), 8.20 (1H, d, J = 5.20 Hz), 7.77-7.96 (3H, m), 7.64-7.71 (1H, m), 7.48- 7.61 (3H, m), 5.73-5.94 (1H, m), 5.45 (1H, dd, J = 14.80, 3.60 Hz), 5.05-5.15 (1H, d, J = 15.56 Hz), 4.70-4.78 (1H, m), 3.60-3.75 (1H, m), 3.04-3.16 (1H, m), 2.82-2.88 (1H, m), 2.76 (3H, s), 2.07-2.35 (3H, m), 1.37-1.92 (5H, m), 1.18 (6H, d, J = 6.80 Hz); HPLC: 100% MS (ESI): m/z 608.3[M + H]+ |
| 147 | 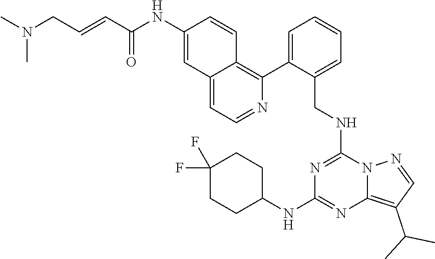 [SMILES: CC(C)c1cnn2c(NCc3ccccc3-c3nccc4cc(NC(=O)/C=C/CN(C)C)ccc34)nc(NC3CCC(F)(F)CC3)nc12] | Light yellow solid; ¹H NMR (400 MHz, CD₃OD) δ = 8.67 (d, J = 2.0 Hz, 1H), 8.42 (d, J = 6.8 Hz, 1H), 8.24 (br d, J = 6.6 Hz, 1H), 7.90-7.78 (m, 3H), 7.69 (t, J = 7.4 Hz, 1H), 7.64-7.55 (m, 3H), 7.07-6.98 (m, 1H), 6.71 (d, J = 15.4 Hz, 1H), 5.06-4.95 (m, 1H), 4.79 (br s, 1H), 4.07 (d, J = 7.3 Hz, 2H), 2.96 (s, 6H), 2.84 (br d, J = 6.3 Hz, 1H), 2.18-1.90 (m, 5H), 1.82-1.55 (m, 3H), 1.22-1.15 (m, 6H); LCMS: 94.6%, MS (ESI): 654.6 m/z [M + H]+ |
| 148 | 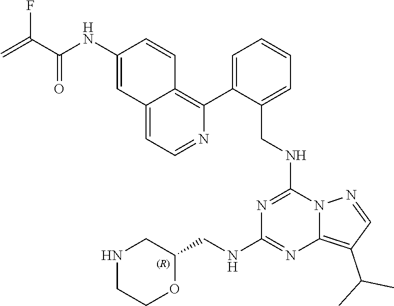 | R form; white powder; ¹H-NMR (CD³OD, 400 MHz): δ 8.62-8.65 (1H, m), 8.44-8.49 (1H, m), 8.20-8.24 (1H, m), 7.79-7.90 (3H, m), 7.65-7.71 (2H, m), 7.54-7.61 (2H, m), 5.85 (1H, dd, J = 46.4, 3.6 Hz), 5.44 (1H, dd, J = 14.8, 3.6 Hz), 5.03-5.13 (1H, m), 4.65-4.76 (1H, m), 3.81-4.12 (3H, m), 3.32-3.61 (3H, m), 2.78-3.21 (4H, m), 1.16-1.21 (6H, m); HPLC: 97.5%, MS (ESI): 596.5 m/z [M + H]+ |
| 149 | 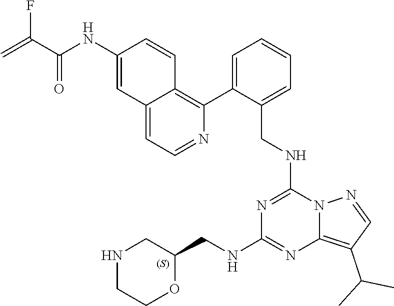 | S form; white powder; ¹H-NMR (CD₃OD, 400 MHz): δ 8.62-8.65 (1H, m), 8.44-8.49 (1H, m), 8.20-8.24 (1H, m), 7.79-7.90 (3H, m), 7.65-7.71 (2H, m), 7.54-7.61 (2H, m), 5.85 (1H, dd, J = 46.4, 3.6 Hz), 5.44 (1H, dd, J = 14.8, 3.6 Hz), 5.03-5.13 (1H, m), 4.65-4.76 (1H, m), 3.81-4.12 (3H, m), 3.32-3.61 (3H, m), 2.78-3.21 (4H, m), 1.16-1.21 (6H, m); HPLC: 95.4%, MS (ESI): 596.5 m/z [M + H]+ |
| 150 | 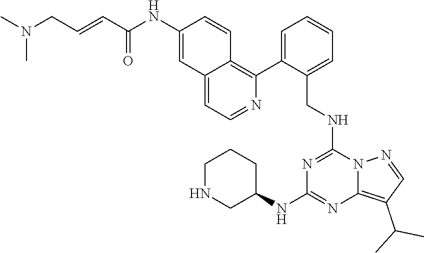 [SMILES: CC(C)c1cnn2c(NCc3ccccc3-c3nccc4cc(NC(=O)/C=C/CN(C)C)ccc34)nc(N[C@@H]3CCCNC3)nc12] | White solid; ¹H NMR (400 MHz, CD₃OD) δ ppm 1.19- 1.28 (m, 6H) 1.69-2.05 (m, 2H) 2.07-2.32 (m, 2H) 2.84-2.94 (m, 1H) 2.98 (s, 6H) 3.04-3.12 (m, 1H) 3.36-3.60 (m, 2H) 4.09 (d, J = 7.03 Hz, 2H) 4.32 (br s, 1H) 4.70 (br d, J = 16.06 Hz, 0.5H) 4.94-5.10 (m, 2H) 5.35 (br d, J = 14.81 Hz, 0.5H) 6.68-6.77 (m, 1H) 7.00-7.10 (m, 1H) 7.58-7.67 (m, 2H) 7.67-7.77 (m, 2H) 7.79-7.97 (m, 3H) 8.20-8.33 (m, 1H) 8.47 (dd, J = 16.31, 6.78 Hz, 1H) 8.71 (s, 1H); LCMS: 100.0%, MS (ESI): m/z 619.3[M + H]+ |
| 151 | 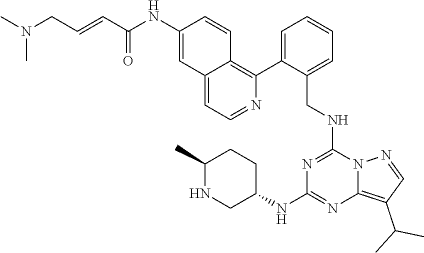 [SMILES: CC(C)c1cnn2c(NCc3ccccc3-c3nccc4cc(NC(=O)/C=C/CN(C)C)ccc34)nc(N[C@H]3CC[C@H](C)NC3)nc12] | White powder; ¹H NMR (400 MHz, CD₃OD) δ 8.67 (s, 1H), 8.40-8.50 (m, 1H), 8.13-8.29 (m, 1H) 7.82-7.88 (m, 2H), 7.70-7.76 (m, 1H), 7.60-7.67 (m, 1H), 7.51-7.58 (m, 1H), 7.41-7.48 (m, 1H), 7.30-7.39 (m, 1H), 6.95-7.11 (m, 1H), 6.64-6.80 (m, 1H), 5.30-5.42 (m, 1H), 4.99- 5.09 (m, 1H), 4.96-5.09 (m, 1H), 4.20-4.42 (m, 1H), 4.07 (d, J = 7.20 Hz, 2H), 2.80-3.05 (m, 9H), 2.15-2.25 (m, 1H), 1.99-2.08 (m, 1H), 1.41-1.47 (m, 3H), 1.31-1.36 (m, 2H), 1.21-1.26 (m, 6H); HPLC: 97.5% MS (ESI): m/z 633.4[M + H]+ |
| 152 | 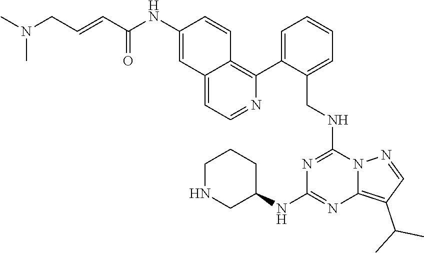 [SMILES: CC(C)c1cnn2c(NCc3ccccc3-c3nccc4cc(NC(=O)/C=C/CN(C)C)ccc34)nc(N[C@@H]3CCCNC3)nc12] | Yellow powder; ¹H NMR (CD₃OD, 400 MHz): δ 8.61 (1H, dd, J = 14.8, 2 Hz), 8.39 (1H, m), 8.16 (1H, t, J = 6.4 Hz), 7.77-7.85 (2H, m), 7.62-7.77 (3H, m), 7.50- 7.61 (2H, m), 6.97-7.08 (1H, m), 6.72 (1H, d, J = 15.6 Hz), 5.23 (1H, m), 5.00 (1H, t, J = 15.6 Hz), 4.62 (1H, dd, J = 15.6, 2.8 Hz), 4.07 (2H, d, J = 7.2 Hz), 3.37- 3.56 (2H, m), 3.11-3.28 (2H, m), 2.96 (6H, s), 2.82-2.91 (1H, m), 1.78-2.21 (4H, m), 1.12-1.27 (6H, m); LCMS: 99%, MS (ESI): m/z 620.3 [M + H]+ |
| 153 | 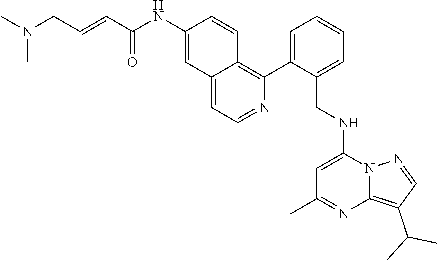 [SMILES: Cc1cc(NCc2ccccc2-c2nccc3cc(NC(=O)/C=C/CN(C)C)ccc23)n2ncc(C(C)C)c2n1] | Brown powder; ¹H NMR (400 MHz, CD₃OD) δ 8.40- 8.49 (m, 2H), 7.86 (s, 1H), 7.80 (d, J = 6.00 Hz, 1H), 7.71-7.76 (m, 1H), 7.67 (t, J = 7.60 Hz, 1H), 7.60 (t, J = 7.20 Hz, 1H), 7.50 (s, 2H), 7.43 (d, J = 6.80 Hz, 1H), 6.90-7.05 (m, 1H), 6.66 (d, J = 15.20 Hz, 1H), 5.80 (s, 1H), 4.90-5.05 (m, 1H) 4.55-4.65 (m, 1H), 4.05 (d, J = 7.20 Hz, 2H), 3.03-3.14 (m, 1H), 2.96 (s, 6H), 2.46 (s, 3H), 1.26 (d, J = 6.80 Hz, 6H); HPLC: 100% MS (ESI): m/z 534.3[M + H]+ |
| 154 | 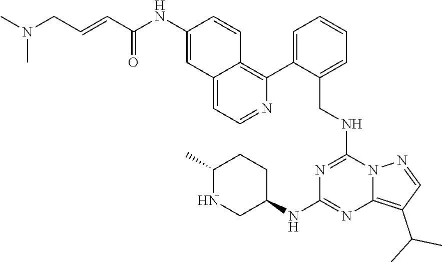 [SMILES: CC(C)c1cnn2c(NCc3ccccc3-c3nccc4cc(NC(=O)/C=C/CN(C)C)ccc34)nc(N[C@@H]3CC[C@@H](C)NC3)nc12] | Yellow solid; ¹H NMR (400 MHz, CD₃OD) δ ppm 1.20- 1.25 (m, 6H) 1.43 (br s, 3H) 1.63-1.90 (m, 2H) 2.06 (s, 3H) 2.11-2.26 (m, 1H) 2.79-2.93 (m, 2H) 2.98 (s, 6H) 3.20-3.28 (m, 1H) 4.09 (d, J = 7.28 Hz, 2H) 4.93- 5.39 (m, 2H) 6.71 (dd, J = 15.31, 6.53 Hz, 1H) 6.94- 7.12 (m, 1H) 7.57 (br s, 1H) 7.68 (br s, 3H) 7.82 (br s, 2H) 8.12 (br s, 1H) 8.38 (br s, 1H) 8.64 (s, 1H); LCMS: 100.0%, MS (ESI): m/z 633.4[M + H]+ |
| 155 | 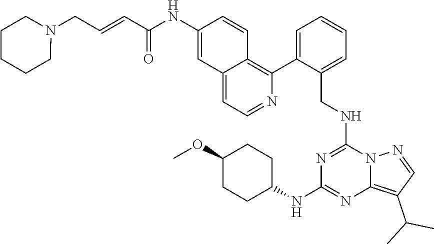 | Yellow powder; ¹H NMR (400 MHz, CD₃OD) δ = 8.49 (s, 1H), 8.32 (br d, J = 6.3 Hz, 1H), 7.86 (br d, J = 6.0 Hz, 1H), 7.76-7.68 (m, 2H), 7.62-7.54 (m, 2H), 7.51 (br s, 2H), 7.44 (br d, J = 7.8 Hz, 1H), 7.05-6.94 (m, 1H), 6.64 (d, J = 15.1 Hz, 1H), 5.00 (br d, J = 15.1 Hz, 1H), 4.57 (br s, 1H), 4.02 (d, J = 7.3 Hz, 2H), 3.55 (br s, 3H), 3.38 (s, 3H), 3.28-3.21 (m, 1H), 3.23 (br s, 1H), 3.12-2.96 (m, 1H), 3.03 (br s, 1H), 2.84 (br s, 1H), 2.20-1.93 (m, 5H), 1.85 (br s, 3H), 1.56 (br s, 1H), 1.45-1.25 (m, 5H), 1.19 (d, J = 7.0 Hz, 6H); LCMS: 100%, MS (ESI): 688.4 m/z [M + H]+ |
| 156 | 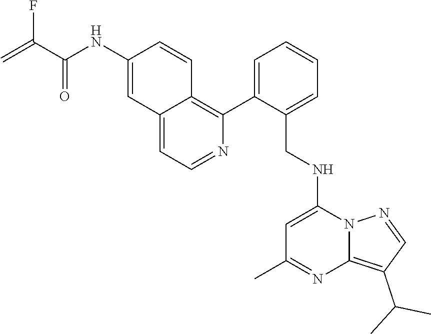 [SMILES: C=C(F)C(=O)Nc1ccc2c(-c3ccccc3CNc3cc(C)nc4c(C(C)C)cnn34)nccc2c1] | White solid; ¹H NMR (400 MHz, CD₃OD) δ = 8.57 (s, 1H), 8.47 (d, J = 6.5 Hz, 1H), 8.23 (br s, 1H), 7.93-7.79 (m, 3H), 7.78-7.65 (m, 2H), 7.59 (br d, J = 9.0 Hz, 2H), 5.95-5.86 (m, 2H), 5.81 (d, J = 3.6 Hz, 1H), 5.46 (dd, J = 3.6, 14.9 Hz, 1H), 4.95-4.94 (m, 1H), 4.96-4.93 (m, 1H), 4.97-4.93 (m, 1H), 4.93 (br s, 1H), 4.95-4.92 (m, 1H), 4.95-4.91 (m, 1H), 4.87-4.86 (m, 1H), 4.86-4.86 (m, 1H), 4.83 (br s, 1H), 4.84-4.82 (m, 1H), 4.85-4.81 (m, 1H), 4.84-4.81 (m, 1H), 3.04 (td, J = 7.0, 13.7 Hz, 1H), 2.49 (s, 3H), 1.25 (dd, J = 4.3, 6.8 Hz, 6H); LCMS: 100%, MS (ESI): 495.1 m/z [M + H]+ |
| 157 | 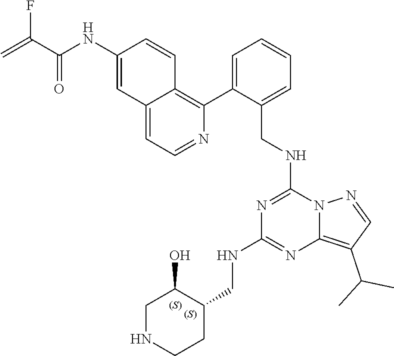 | 3S, 4S form, white solid; ¹H NMR (400 MHz, CD₃OD) δ ppm 1.11-1.25 (m, 6H) 1.28-1.64 (m, 1H) 1.53 (br s, 1H) 1.87 (br d, J = 11.04 Hz, 1H) 1.96-2.14 (m, 1H) 2.88 (br d, J = 6.02 Hz, 3H) 3.35-3.78 (m, 5H) 4.57-4.91 (m, 2H) 5.11 (br t, J = 15.56 Hz, 2H) 5.46 (dd, J = 14.93, 3.39 Hz, 1H) 5.81 (br s, 1H) 5.92 (br s, 1H) 7.52-7.93 (m, 7H) 8.19 (br s, 1H) 8.47 (br s, 1H) 8.62 (br s, 1H); LCMS: 100%, MS (ESI): 610.1 m/z [M + H]+ |
| 158 | 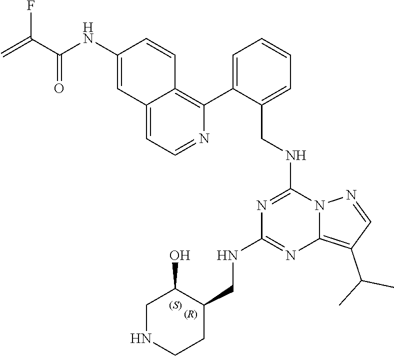 | 3S, 4R form, yellow powder; ¹H NMR (400 MHz, CD₃OD) δ ppm 8.57 (s, 1H), 8.44 (s, 1H), 8.11 (d, J = 6.40 Hz, 1H), 7.74-7.88 (m, 3H), 7.58-7.70 (m, 2H), 7.51-7.56 (m, 2H), 5.75-5.95 (m, 1H), 5.44 (dd, J = 14.80, 3.60 Hz, 1H), 4.99-5.17 (m, 1H), 4.65 (d, J = 14.40 Hz, 1H), 4.05 (d, J = 12.80 Hz, 1H), 3.34-3.48 (m, 2H), 3.20-3.30 (m, 2H), 2.82-3.15 (m, 3H), 1.78- 2.01 (m, 2H), 1.63-1.73 (m, 1H), 1.16-1.22 (m, 6H); HPLC: 96.2% MS (ESI): m/z 610.3[M + H]+ |
| 159 | 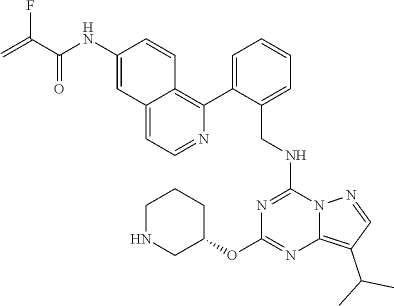 [SMILES: C=C(F)C(=O)Nc1ccc2c(-c3ccccc3CNc3nc(O[C@H]4CCCNC4)nc4c(C(C)C)cnn34)nccc2c1] | Yellow powder; ¹H NMR (400 MHz, CD₃OD) δ ppm 8.58 (dd, J = 19.60, 2.00 Hz, 1H), 8.36-8.43 (m, 1H), 8.13 (d, J = 6.40 Hz, 1H), 7.72-7.82 (m, 3H), 7.59-7.69 (m, 2H), 7.51-7.57 (m, 2H), 5.74-5.95 (m, 1H), 5.44 (dd, J = 14.80, 3.60 Hz, 1H), 5.20 (s, 1H), 5.03 (d, J = 15.20 Hz, 1H), 4.56 (t, J = 16.00 Hz, 1H), 3.37-3.54 (m, 2H), 3.10-3.27 (m, 2H), 2.80-2.92 (m, 1H), 1.84-2.15 (m, 4H) 1.16-1.26 (m, 6H); HPLC: 99.3% MS (ESI): m/z 581.1[M + H]+ |
| 160 | 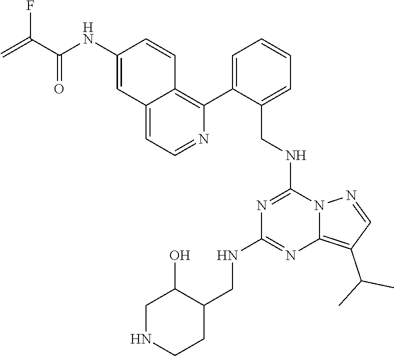 [SMILES: C=C(F)C(=O)Nc1ccc2c(-c3ccccc3CNc3nc(NCC4CCNCC4O)nc4c(C(C)C)cnn34)nccc2c1] | 3R,4R and 3S, 4S form mixture; yellow solid; ¹H NMR (400 MHz, CD₃OD) δ ppm 8.59 (s, 1H), 8.46 (s, 1H), 8.05-8.25 (m, 1H), 7.75-7.87 (m, 3H), 7.62-7.70 (m, 2H), 7.49-7.58 (m, 2H), 5.71-5.98 (m, 1H), 5.44 (dd, J = 14.80, 3.60 Hz, 1H), 5.12 (d, J = 14.80 Hz, 1H), 4.50-4.65 (m, 1H), 3.51-3.79 (m, 2H). 3.35-3.46 (m, 3H), 2.95-3.05 (m, 1H), 2.75-2.84 (m, 2H), 2.02 (d, J = 11.20 Hz, 1H), 1.75-1.84 (m, 1H), 1.50-1.62 (m, 1H), 1.15-1.30 (m, 6H); HPLC: 99.2% MS (ESI): m/z 610.3[M + H]+ |
| 161 | 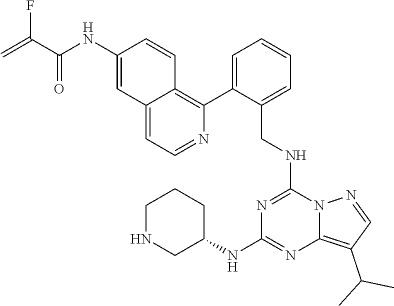 [SMILES: C=C(F)C(=O)Nc1ccc2c(-c3ccccc3CNc3nc(N[C@H]4CCCNC4)nc4c(C(C)C)cnn34)nccc2c1] | Yellow solid; ¹H NMR (400 MHz, DMSO-d6) δ ppm 10.54 (s, 1H) 8.85 (br s, 2H) 8.52 (d, J = 5.77 Hz, 1H) 8.44-8.49 (m, 1H) 8.47 (d, J = 1.76 Hz, 1H) 8.13 (br s, 1H) 7.86 (dd, J = 9.03, 2.01 Hz, 1H) 7.81 (d, J = 6.02 Hz, 1H) 7.58-7.64 (m, 3H) 7.51-7.57 (m, 1H) 7.45-7.51 (m, 1H) 7.39 (d, J = 7.53 Hz, 1H) 6.58 (br s, 1H) 5.73- 5.91 (m, 1H) 5.47 (dd, J = 15.69, 3.89 Hz, 1H) 4.49 (br s, 2H) 4.04 (br s, 1H) 3.22-3.28 (m, 2H) 2.75-2.99 (m, 4H) 1.93 (m, 2H) 1.76 (m, 1H) 1.60 (m, 1H) 1.24 (d, J = 7.03 Hz, 6H); HPLC: 95.8%, MS (ESI): 580.1 m/z [M + H]+ |
| 162 | 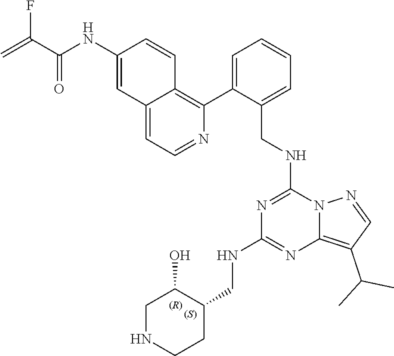 | 3R, 4S form white solid; ¹H NMR (400 MHz, CD₃OD) δ ppm 8.67 (s, 1H) 8.52 (d, J = 6.78 Hz, 1H) 8.31 (d, J = 6.53 Hz, 1H) 7.80-8.00 (m, 3H) 7.54-7.76 (m, 4H) 5.80-5.98 (m, 1H) 5.48 (dd, J = 14.81, 3.76 Hz, 1H) 5.12 (br dd, J = 15.56, 7.28 Hz, 1H) 4.74 (br dd, J = 15.69, 2.89 Hz, 1H) 4.13 (br s, 1H) 3.35-3.54 (m, 3H) 2.96-3.23 (m, 2H) 2.87 (dt, J = 13.61, 6.87 Hz, 1H) 1.59-2.12 (m, 3H) 1.14-1.28 (m, 6H) 1.14-1.28 (m, 1H); HPLC: 97.8% (254 nm), MS (ESI): m/z 610.2 [M + H]+ |
| 163 | 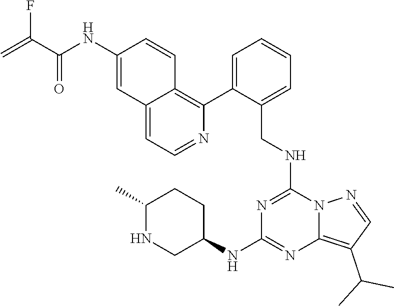 [SMILES: C=C(F)C(=O)Nc1ccc2c(-c3ccccc3CNc3nc(N[C@@H]4CC[C@@H](C)NC4)nc4c(C(C)C)cnn34)nccc2c1] | Yellow solid; ¹H NMR (400 MHz, CD₃OD) δ = 8.67 (br s, 1H), 8.52-8.45 (m, 1H), 8.33-8.20 (m, 1H), 7.95 (br s, 1H), 7.90-7.66 (m, 4H), 7.65-7.57 (m, 2H), 5.96- 5.79 (m, 1H), 5.46 (dd, J = 4.0, 13.6 Hz, 1H), 5.02 (br s, 1H), 5.15-4.93 (m, 1H), 4.62 (br d, J = 15.3 Hz, 1H), 4.34 (br s, 1H), 3.62-3.35 (m, 1H), 3.29-3.21 (m, 1H), 3.09-2.79 (m, 2H), 2.35-2.10 (m, 1H), 2.08-1.65 (m, 3H), 1.44 (br d, J = 6.3 Hz, 3H), 1.25-1.17 (m, 6H); LCMS: 100%, MS (ESI): m/z 594.3 [M + H]+ |
| 164 | 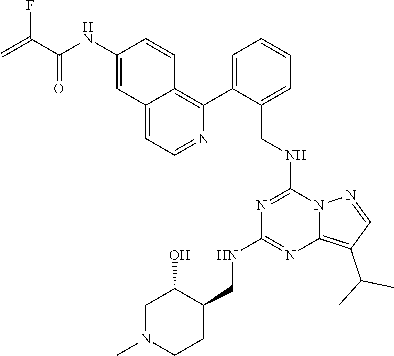 [SMILES: C=C(F)C(=O)Nc1ccc2c(-c3ccccc3CNc3nc(NC[C@H]4CCN(C)C[C@@H]4O)nc4c(C(C)C)cnn34)nccc2c1] | White powder; ¹H NMR (DMSO-d6, 400 MHz) δ10.43 (1H, s) 8.51 (1H, d, J = 6.0) 8.41 (1H, d, J = 2.0 Hz) 7.91 (1H, s) 7.73-7.81 (2H, m) 7.56-7.62 (3H, m) 7.41-7.52 (2H, m) 7.31-7.37 (1H, m) 6.22 (1H, s) 5.68-5.86 (1H, m) 5.44 (1H, dd, J = 15.6, 3.6 Hz) 4.89 (1H, s) 4.45 (2H, s) 3.31-3.35 (2H, m) 2.88 (2H, dq, J = 14.0, 7.2 Hz) 2.70-2.80 (1H, m) 2.26 (3H, s) 1.80- 2.05 (2H, m) 1.62 (1H, d, J = 13.2 Hz) 1.23-1.43 (2H, m) 1.21 (6H, d, J = 6.8 Hz); LCMS: 100%, MS (ESI): m/z 624.3 [M + H]+ |
| 165 | 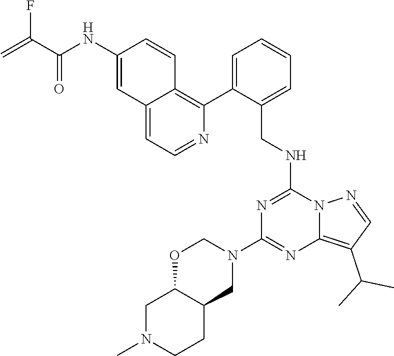 [SMILES: C=C(F)C(=O)Nc1ccc2c(-c3ccccc3CNc3nc(N4CO[C@H]5CN(C)CC[C@@H]5C4)nc4c(C(C)C)cnn34)nccc2c1] | White powder; ¹H NMR (DMSO-d6, 400 MHz) δ10.72 (1H, s) 8.79 (1H, d, J = 6.0 Hz) 8.71 (1H, d, J = 2.4 Hz) 8.40 (1H, s) 8.06 (1H, dd, J = 9.2, 2.0 Hz) 8.01 (1H, d, J = 6.0 Hz) 7.93 (1H, s) 7.82-7.90 (2H, m) 7.70-7.82 (2H, m) 7.64 (1H, dd, J = 7.2, 1.6 Hz) 6.18 (1H, d, J = 9.6 Hz) 5.99-6.15 (1H, m) 5.76 (1H, , J = 15.6, 3.6 Hz) 4.69-4.84 (3H, m) 4.58-4.66 (1H, m) 3.54-3.68 (2H, m) 3.12-3.26 (2H, m) 3.04 (1H, d, J = 11.2 Hz) 2.91-2.99 (1H, m) 2.50 (3H, s) 2.13-2.24 (1H, m) 2.05 (1H, t, J = 10.0 Hz) 1.70 (1H, d, J = 10.4 Hz) 1.56 (1H, s) 1.54- 1.59 (1H, m) 1.53 (6H, d, J = 7.2 Hz); LCMS: 100%, MS (ESI): m/z 636.3 [M + H]+ |
| 166 | 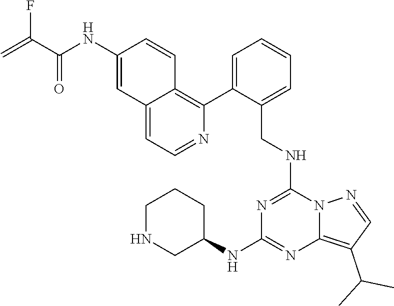 [SMILES: C=C(F)C(=O)Nc1ccc2c(-c3ccccc3CNc3nc(N[C@@H]4CCCNC4)nc4c(C(C)C)cnn34)nccc2c1] | White powder; ¹H NMR (400 MHz, DMSO-d6) δ ppm 1.18 (6H, d, J = 6.78 Hz) 1.61 (1H, br d, J = 9.03 Hz) 1.81-1.99 (3H, m) 2.86 (4H, dt, J = 13.68, 6.96 Hz) 4.10 (1H, br s) 4.67 (2H, br s) 5.51 (1H, dd, J = 15.56, 3.76 Hz) 5.79-5.98 (1H, m) 7.47 (1H, d, J = 7.53 Hz) 7.53- 7.59 (3H, m) 7.68 (1H, t, J = 7.40 Hz) 7.73-7.79 (1H, m) 7.93 (1H, dd, J = 9.29, 2.01 Hz) 8.12 (1H, d, J = 6.27 Hz) 8.47 (1H, d, J = 6.27 Hz) 8.56 (1H, d, J = 1.76 Hz) 9.11 (2H, br s) 10.80 (1H, br s); LCMS: 99.72%, MS (ESI): m/z 580.3 [M + H]+ |
| 167 | 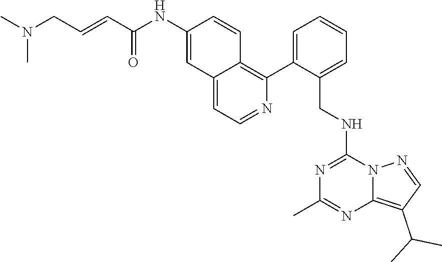 [SMILES: Cc1nc(NCc2ccccc2-c2nccc3cc(NC(=O)/C=C/CN(C)C)ccc23)n2ncc(C(C)C)c2n1] | White solid; ¹H NMR (400 MHz, CD₃OD) δ ppm 8.27 (d, J = 6.02 Hz, 1H) 8.24 (d, J = 1.76 Hz, 1H) 7.59-7.64 (m, 1H) 7.57 (t, J = 2.89 Hz, 2H) 7.54 (td, J = 7.59, 1.38 Hz, 1H) 7.46 (td, J = 7.53, 1.25 Hz, 1H) 7.40-7.44 (m, 1H) 7.34-7.39 (m, 1H) 7.29 (dd, J = 7.53, 1.00 Hz, 1H) 6.81-6.93 (m, 1H) 6.53 (d, J = 15.06 Hz, 1H) 5.03 (br d, J = 14.81 Hz, 1H) 4.32 (d, J = 14.81 Hz, 1H) 3.96 (dd, J = 7.28, 1.00 Hz, 2H) 2.96 (quin, J = 6.90 Hz, 1H) 2.88 (s, 6H) 2.12 (s, 3H) 1.15 (t, J = 7.03 Hz, 6H); HPLC: 98.1%, MS (ESI): 535.1 m/z [M + H]+ |
| 168 | 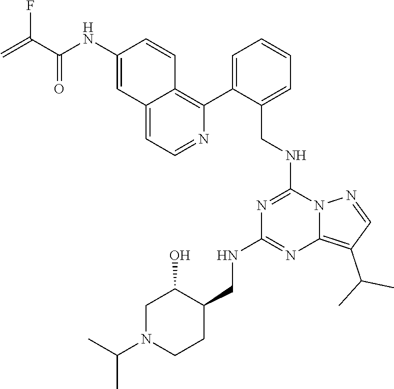 [SMILES: C=C(F)C(=O)Nc1ccc2c(-c3ccccc3CNc3nc(NC[C@H]4CCN(C(C)C)C[C@@H]4O)nc4c(C(C)C)cnn34)nccc2c1] | White powder; ¹H NMR (400 MHz, DMSO-d6) δ 11.11 (1H, s), 10.67-11.00 (1H, d, J = 44.0 Hz), 9.63 (1H, s), 8.32-8.78 (3H, m), 7.32-8.00 (7H, m), 5.95 (1H, dd, J = 15.60, 4.00 Hz), 5.60 (1H, dd, J = 15.60, 4.00 Hz), 4.79-5.01 (1H, m), 4.44-4.65 (1H, m), 3.80-3.95 (1H, m), 3.04-3.41 (5H, m), 2.75-2.90 (2H, m), 2.60- 2.70 (1H, m), 1.57-1.93 (3H, m), 1.28 (6H, br d, J = 6.40 Hz) 1.13 (6H, t, J = 6.40 Hz); HPLC: 97.9%, MS (ESI): m/z 652.4 [M + H]+ |
| 169 | 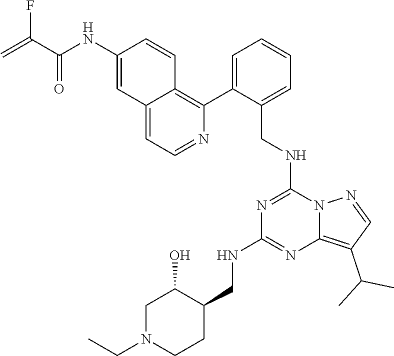 [SMILES: C=C(F)C(=O)Nc1ccc2c(-c3ccccc3CNc3nc(NC[C@H]4CCN(CC)C[C@@H]4O)nc4c(C(C)C)cnn34)nccc2c1] | White powder; ¹H NMR (400 MHz, DMSO-d6) δ 10.90- 11.31 (2H, m), 10.11 (1H, br s), 9.40 (1H, br s), 8.59- 8.69 (1H, m), 8.44-8.55 (1H, m), 8.17-8.29 (1H, m), 7.95-8.09 (1H, m), 7.83 (1H, d, J = 7.6 Hz), 7.73 (1H, t, J = 7.6 Hz), 7.46-7.66 (4H, m), 5.83-6.12 (1H, m), 5.53 (1H, dd, J = 15.6, 4.0 Hz), 3.75-3.94 (1H, m), 3.64 (1H, d, J = 12.0 Hz), 3.32-3.45 (2H, m), 3.02-3.24 (3H, m), 2.88-2.96 (1H, m), 2.58-2.84 (2H, m), 1.92 (1H, d, J = 10.0 Hz), 1.60-1.75 (2H, m), 1.33 (3H, t, J = 4.0 Hz), 1.19 (6H, d, J = 7.2 Hz); HPLC: 100%, MS (ESI): m/z 638.4 [M + H]+ |
| 170 | 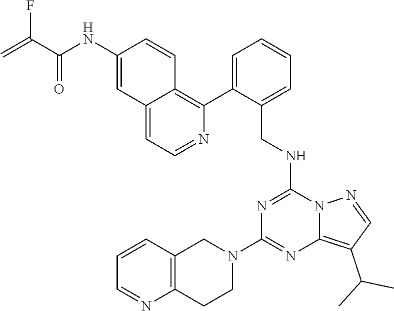 [SMILES: C=C(F)C(=O)Nc1ccc2c(-c3ccccc3CNc3nc(N4CCc5ncccc5C4)nc4c(C(C)C)cnn34)nccc2c1] | White powder; ¹H NMR (400 MHz, DMSO-d6) δ ppm 1.25 (6H, d, J = 6.78 Hz) 2.86 (2H, t, J = 6.02 Hz) 2.93 (1H, dt, J = 13.80, 6.90 Hz) 3.89 (2H, t, J = 6.02 Hz) 4.52 (2H, br s) 4.70 (2H, s) 5.44 (1H, dd, J = 15.56, 3.76 Hz) 5.68-5.88 (1H, m) 7.18 (1H, dd, J = 7.78, 4.77 Hz) 7.35 (1H, dd, J = 7.40, 1.38 Hz) 7.41-7.53 (3H, m) 7.56 (1H, d, J = 9.29 Hz) 7.59-7.63 (2H, m) 7.68 (1H, d, J = 5.77 Hz) 7.77 (1H, dd, J = 9.16, 2.13 Hz) 8.08 (1H, br s) 8.33-8.37 (1H, m) 8.42 (1H, d, J = 2.01 Hz) 8.47 (1H, d, J = 5.77 Hz) 10.41 (1H, br s); LCMS: 100% MS (ESI): m/z 614.3[M + H]+ |
| 171 | 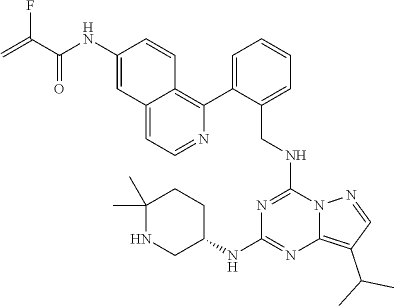 [SMILES: C=C(F)C(=O)Nc1ccc2c(-c3ccccc3CNc3nc(N[C@H]4CCC(C)(C)NC4)nc4c(C(C)C)cnn34)nccc2c1] | Yellow powder; ¹H NMR (400 MHz, DMSO-d6) δ 10.75 (1H, s), 9.05-9.30 (2H, m), 8.56 (1H, d, J = 1.6 Hz), 8.48 (1H, d, J = 6.4 Hz), 8.07 (1H, d, J = 6.4 Hz) 7.94 (1H, dd, J = 9.2, 2.0 Hz), 7.73 (1H, d, J = 7.6 Hz), 7.51-7.68 (4H, m), 7.50-7.51 (1H, m), 7.44-7.47 (1H, m), 5.85 (1H, dd, J = 48.0, 4.0 Hz), 5.50 (1H, dd, J = 16.0, 4.0 Hz), 4.50-4.75 (2H, m), 4.01-4.23 (1H, m), 2.80-3.00 (3H, m), 1.71-1.89 (4H, m), 1.38 (6H, d, J = 16.0 Hz), 1.19 (6H, d, J = 8.0 Hz); HPLC: 98.5%, MS (ESI): m/z 608.3 [M + H]+ |
| 172 | 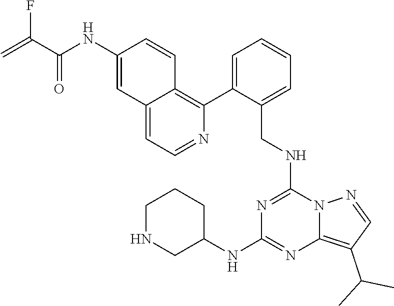 [SMILES: C=C(F)C(=O)Nc1ccc2c(-c3ccccc3CNc3nc(NC4CCCNC4)nc4c(C(C)C)cnn34)nccc2c1] | Yellow powder; ¹H NMR (400 MHz, DMSO-d6) δ ppm 10.71 (1H, s) 9.04 (2H, br s) 8.55 (1H, d, J = 1.76 Hz) 8.49 (1H, d, J = 6.27 Hz) 8.02 (1H, d, J = 6.27 Hz) 7.92 (1H, dd, J = 9.16, 1.88 Hz) 7.68-7.74 (1H, m) 7.50-7.67 (4H, m) 7.42-7.47 (1H, m) 5.75-5.95 (1H, m) 5.50 (1H, dd, J = 15.56, 4.02 Hz) 4.60 (2H, br s) 4.07 (1H, br s) 2.88 (4H, dt, J = 13.80, 6.90 Hz) 1.93 (2H, br d, J = 10.29 Hz) 1.80 (1H, br d, J = 13.30 Hz) 1.61 (1H, br d, J = 9.54 Hz) 1.21 (7H, d, J = 7.03 Hz); LCMS: 100%, MS (ESI): m/z 580.1 [M + H]+ |
| 173 | 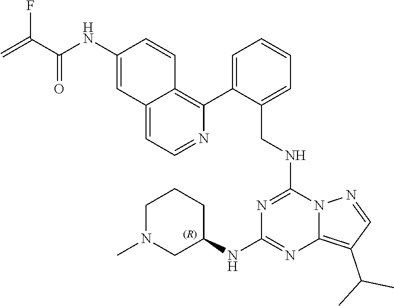 | Pink powder; ¹H NMR (400 MHz, DMSO-d6) δ ppm 1.20 (6H, br d, J = 6.78 Hz) 1.38-1.82 (4H, m) 1.83- 2.44 (6H, m) 2.76-3.00 (2H, m) 3.49-3.93 (1H, m) 4.42 (2H, br s) 5.52 (1H, dd, J = 15.81, 3.76 Hz) 5.73- 5.89 (1H, m) 5.94-6.77 (1H, m) 7.34 (1H, br d, J = 7.28 Hz) 7.41-7.55 (3H, m) 7.63 (2H, s) 7.76-7.86 (2H, m) 8.26-8.61 (3H, m) 10.70 (1H, br s); LCMS: 100%, MS (ESI): m/z 594.3 [M + H]+ |
| 174 | 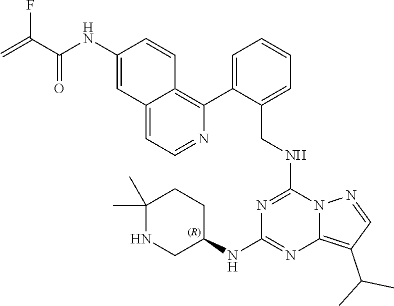 | Yellow solid; ¹H NMR (DMSO-d6, 400 MH) δ 8.52 (1H, d, J = 5.6 Hz) 8.44 (1H, d, J = 2.0 Hz) 7.92 (1H, s) 7.83 (1H, dd, J = 9.2, 2.4 Hz) 7.74 (1H, d, J = 5.6 Hz) 7.55- 7.66 (3H, m) 7.40-7.53 (2H, m) 7.35 (1H, dd, J = 7.6, 1.2 Hz) 6.04 (1H, s) 5.68-5.87 (1H, m) 5.45 (1H, dd, J = 15.6, 3.6 Hz) 4.46 (2H, s) 3.59 (1H, s) 2.89 (2H, dt, J = 14.0, 6.8 Hz) 2.55-2.61 (1H, m) 1.69 (1H, s) 1.57 (1H, q, J = 9.2 Hz) 1.42-1.51 (1H, m) 1.25-1.36 (1H, m) 1.22 (6H, d, J = 6.8 Hz) 1.06 (6H, d, J = 4.8 Hz); LCMS: 100%, MS (ESI): m/z 608.3 [M + H]+ |
| 175 | 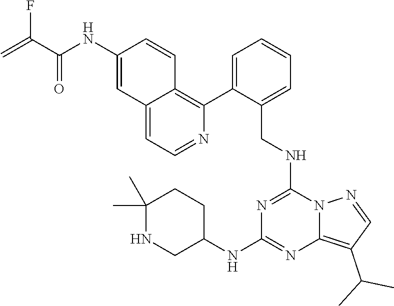 [SMILES: C=C(F)C(=O)Nc1ccc2c(-c3ccccc3CNc3nc(NC4CCC(C)(C)NC4)nc4c(C(C)C)cnn34)nccc2c1] | White powder; ¹H NMR (DMSO-d6, 400 MHz) δ8.52 (1H, d, J = 6.0 Hz) 8.44 (1H, d, J = 1.2 Hz) 7.93 (1H, s) 7.83 (1H, dd, J = 8.8, 2.0 Hz) 7.74 (1H, d, J = 5.6 Hz) 7.56-7.65 (3H, m) 7.41-7.52 (2H, m) 7.35 (1H, d, J = 6.4 Hz) 6.05 (1H, s) 5.69-5.87 (1H, m) 5.45 (1H, dd, J = 15.6, 3.6 Hz) 4.46 (2H, s) 3.59 (1H, s) 2.86-2.93 (2H, m) 2.57 (1H, dd, J = 12.4, 7.2 Hz) 1.69 (1H, s) 1.51-1.63 (1H, m) 1.43- 1.51 (1H, m) 1.25-1.33 (1H, m) 1.22 (6H, d, J = 7.2 Hz) 1.06 (6H, d, J = 4.0 Hz); LCMS: 98.4%, MS (ESI): m/z 608.3 [M + H]+ |
| 176 | 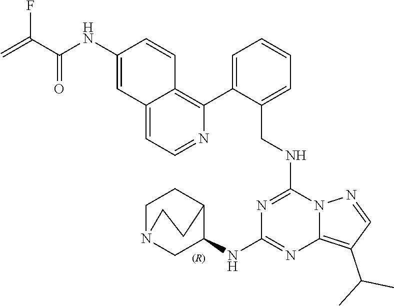 | Yellow powder; ¹H NMR (400 MHz, DMSO-d6) δ ppm 1.19-1.28 (8H, m) 1.73 (1H, br s) 1.90 (2H, br s) 1.99- 2.23 (3H, m) 2.87 (1H, dt, J = 14.05, 7.03 Hz) 3.11- 3.14 (3H, m) 3.54-3.69 (1H, m) 4.11 (1H, br s) 4.56 (2H, br s) 5.50 (1H, dd, J = 15.56, 4.02 Hz) 5.72-5.95 (1H, m) 7.28-7.34 (1H, m) 7.40-7.44 (1H, m) 7.51 (1H, t, J = 7.28 Hz) 7.57-7.64 (4H, m) 7.85-7.94 (2H, m) 8.33 (1H, s) 8.44-8.55 (2H, m) 9.99-10.30 (1H, m) 10.66 (1H, br s); LCMS: 99.0%, MS (ESI): m/z 606.3 [M + H]+ |
| 177 | 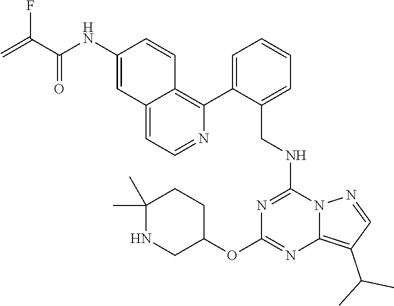 [SMILES: C=C(F)C(=O)Nc1ccc2c(-c3ccccc3CNc3nc(OC4CCC(C)(C)NC4)nc4c(C(C)C)cnn34)nccc2c1] | Yellow powder; ¹H NMR (400 MHz, DMSO-d6) δ ppm 1.22 (6H, d, J = 6.78 Hz) 1.26 (1H, s) 1.38-1.44 (6H, m) 1.70-1.89 (3H, m) 1.99-2.07 (1H, m) 2.90 (1H, dt, J = 13.80, 6.90 Hz) 3.14 (1H, br s) 3.37 (1H, br d, J = 11.54 Hz) 5.06-5.20 (1H, m) 5.53 (1H, dd, J = 15.69, 3.89 Hz) 5.84-5.99 (1H, m) 7.46-7.51 (1H, m) 7.53- 7.64 (2H, m) 7.64-7.75 (3H, m) 7.97 (1H, br d, J = 9.29 Hz) 8.14 (1H, d, J = 6.53 Hz) 8.50 (1H, d, J = 6.27 Hz) 8.60 (1H, d, J = 2.01 Hz) 8.63 (1H, br s) 9.04 (1H, br s) 9.52 (1H, br s) 10.87 (1H, s); LCMS: 98.77%, MS (ESI): m/z 609.2 [M + H]+ |
| 178 | 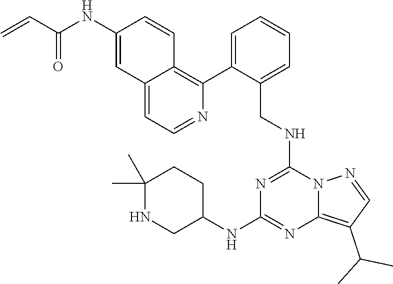 [SMILES: C=CC(=O)Nc1ccc2c(-c3ccccc3CNc3nc(NC4CCC(C)(C)NC4)nc4c(C(C)C)cnn34)nccc2c1] | Yellow powder; ¹H NMR (DMSO-d6, 400 MHz) δ 10.39 (1H, br s), 8.49 (1H, d, J = 4.0 Hz), 8.45 (1H, d, J = 4.0 Hz), 8.04 (1H, br s), 7.73 (1H, d, J = 4.0 Hz), 7.67-7.71 (1H, m), 7.55-7.63 (3H, m), 7.43-7.53 (2H, m), 7.35 (1H, dd, J = 8.0, 4.0 Hz), 6.47-6.66 (1H, m) 6.23-6.38 (2H, m), 5.81 (1H, dd, J = 8.0, 2.0 Hz), 4.47 (2H, br s), 3.81 (1H, br s), 2.78-2.90 (3H, m), 1.63-1.79 (2H, m), 1.43-1.53 (2H, m), 1.13-1.29 (12H, m); HPLC: 97.1%, MS (ESI): m/z 590.3 [M + H]+ |
| 179 | 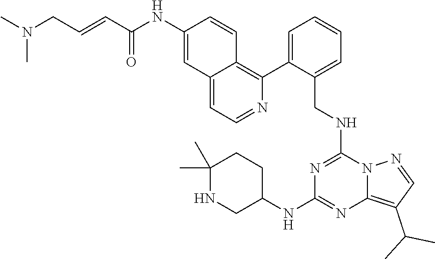 [SMILES: CC(C)c1cnn2c(NCc3ccccc3-c3nccc4cc(NC(=O)/C=C/CN(C)C)ccc34)nc(NC3CCC(C)(C)NC3)nc12] | White powder; ¹H NMR (DMSO-d6, 400 MHz) δ ppm 10.30 (1H, s) 8.48 (1H, d, J = 5.6 Hz) 8.43 (1H, s) 8.01 (1H, s) 7.65-7.74 (2H, m) 7.54-7.62 (3H, m) 7.42- 7.52 (2H, m) 7.35 (1H, d, J = 6.8 Hz) 6.78-6.90 (1H, m) 6.35 (1H, d, J = 15.6 Hz) 6.18 (1H, s) 4.46 (2H, s) 3.69 (1H, s) 2.85-2.95 (1H, m) 2.67 (2H, s) 2.50 (17H, s) 2.22 (6H, s) 1.90 (1H, s) 1.71 (1H, s) 1.49-1.66 (3H, m) 1.40 (1H, d, J = 9.6 Hz) 1.22 (6H, d, J = 6.8 Hz) 1.13 (6H, s); LCMS: 100%, MS (ESI): m/z 647.3 [M + H]+ |
| 180 | 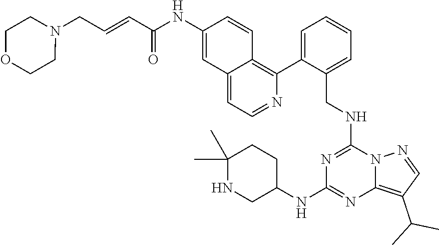 [SMILES: CC(C)c1cnn2c(NCc3ccccc3-c3nccc4cc(NC(=O)/C=C/CN5CCOCC5)ccc34)nc(NC3CCC(C)(C)NC3)nc12] | White powder; ¹H NMR (DMSO-d6, 400 MHz) δ ppm 10.33 (1H, s) 8.48 (1H, d, J = 5.6 Hz) 8.43 (1H, d, J = 2.0 Hz) 8.06 (1H, s) 7.66-7.74 (2H, m) 7.54-7.62 (3H, m) 7.42-7.52 (2H, m) 7.35 (1H, dd, J = 7.6, 1.2 Hz) 6.84 (1H, dt, J = 15.6, 6.0 Hz) 6.30-6.42 (2H, m) 4.46 (2H, s) 3.85 (1H, s) 3.59-3.66 (4H, m) 2.90 (1H, dt, J = 13.6, 6.8 Hz) 2.43-2.46 (4H, m) 1.91 (1H, s) 1.78 (1H, s) 1.65 (2H, d, J = 13.2 Hz) 1.54 (1H, d, J = 10.4 Hz) 1.22- 1.27 (12H, m); LCMS: 100%, MS (ESI): m/z 689.4 [M + H]+ |
| 181 | 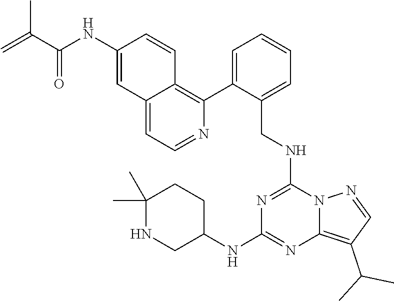 [SMILES: C=C(C)C(=O)Nc1ccc2c(-c3ccccc3CNc3nc(NC4CCC(C)(C)NC4)nc4c(C(C)C)cnn34)nccc2c1] | White powder; ¹H NMR (400 MHz, DMSO-d6) δ 9.99 (1H, s), 8.49 (1H, d, J = 4.0 Hz), 8.43 (1H, s), 7.99 (1H, s), 7.80 (1H, d, J = 8.0 Hz), 7.72 (1H, d, J = 4.0 Hz), 7.55-7.63 (3H, m), 7.41-7.52 (2H, m), 7.35 (1H, d, J = 8.0 Hz,), 6.13 (1H, s), 5.89 (1H, s), 5.57 (s, 1H), 4.46 (2H, s), 3.63 (1H, s), 2.83-3.00 (2H, m), 2.60-2.70 (1H, m), 2.01 (3H, s), 1.62-1.69 (1H, m), 1.56-1.59 (1H, m), 1.46-1.55 (1H, m), 1.30-1.35 (1H, m), 1.23 (6H, d, J = 4.0 Hz), 1.09 (6H, s); LCMS: 100%, MS (ESI): m/z 604.3 [M + H]+ |
| 182 | 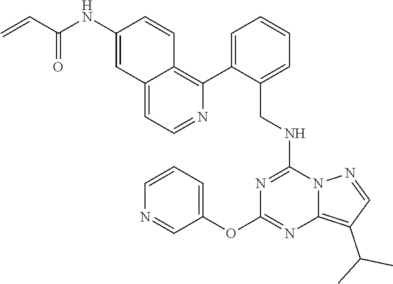 [SMILES: C=CC(=O)Nc1ccc2c(-c3ccccc3CNc3nc(Oc4cccnc4)nc4c(C(C)C)cnn34)nccc2c1] | Yellow powder; ¹H NMR (400 MHz, DMSO-d6) δ ppm 10.29 (s, 1H) 8.72 (br s, 1H) 8.28-8.52 (m, 4H) 7.76- 7.90 (m, 1H) 7.68 (d, J = 5.60 Hz, 1H) 7.55-7.65 (m, 2H) 7.49-7.53 (m, 3H) 7.43-7.48 (m, 1H) 7.40 (dd, J = 8.40, 4.64 Hz, 1H) 7.33 (d, J = 6.40 Hz, 1H) 6.40- 6.59 (m, 1H) 6.26-6.39 (m, 1H) 5.69-5.88 (m, 1H) 4.49 (br s, 2H) 1.17 (d, J = 7.20 Hz, 6H); LCMS: 100%, MS (ESI): m/z 557.5 [M + H]+ |
| 183 | 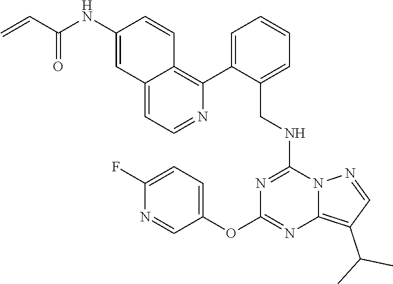 | Yellow powder; ¹H NMR (400 MHz, DMSO-d6) δ ppm 10.40 (br s, 1H) 8.37-8.45 (m, 2H) 8.19-8.26 (m, 1H) 8.10 (d, J = 1.20 Hz, 1H) 7.77-7.87 (m, 2H) 7.67 (d, J = 5.60 Hz, 1H) 7.57-7.62 (m, 1H) 7.41-7.54 (m, 4H) 7.37 (m, 1H) 7.26-7.37 (m, 1H) 7.13 (dd, J = 8.80, 3.20 Hz, 1H) 6.42-6.60 (m, 1H) 6.25-6.40 (m, 1H) 5.72- 5.84 (m, 1H) 4.47 (br s, 2H) 2.77-2.93 (m, 1H) 1.16 (d, J = 7.20 Hz, 6H); LCMS: 100%, MS (ESI): m/z 1011.5 [M + H]+ |
| 184 | 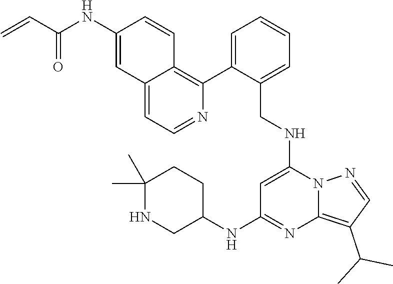 [SMILES: C=CC(=O)Nc1ccc2c(-c3ccccc3CNc3cc(NC4CCC(C)(C)NC4)nc4c(C(C)C)cnn34)nccc2c1] | White powder; ¹H NMR (400 MHz, DMSO-d6) δ10.39 (1H, s), 8.53 (1H, d, J = 4.0 Hz), 8.47 (1H, s), 7.76 (1H, d, J = 4.0 Hz), 7.68-7.73 (1H, m), 7.60-7.64 (1H, m), 7.43-7.57 (4H, m), 7.37 (1H, d, J = 8.0 Hz), 7.12-7.24 (1H, m), 6.47-6.59 (1H, m), 6.27-6.39 (1H, m), 6.17 (1H, br d, J = 8.0 Hz), 5.82 (1H, d, J = 12.0 Hz), 5.03 (1H, s), 4.25 (2H, br d, J = 4.0 Hz), 3.86 (1H, br s), 2.90- 2.97 (2H, m), 2.73-2.88 (1H, m), 1.45-1.90 (4H, m), 1.25 (6H, d, J = 8.0 Hz), 1.21 (6H, s); HPLC: 98.8% (254 nm), MS (ESI): m/z 589.3 [M + H]+ |
| 185 | 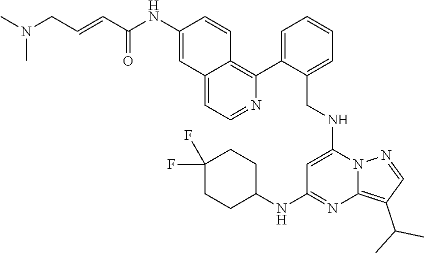 [SMILES: CC(C)c1cnn2c(NCc3ccccc3-c3nccc4cc(NC(=O)/C=C/CN(C)C)ccc34)cc(NC3CCC(F)(F)CC3)nc12] | White powder; ¹H NMR (400 MHz, DMSO-d6) δ ppm 1.25 (6H, d, J = 6.78 Hz) 1.52-1.64 (3H, m) 1.92 (3H, br s) 1.99-2.09 (3H, m) 2.22 (5H, s) 2.96 (1H, dt, J = 13.55, 6.78 Hz) 3.87 (2H, br s) 4.24 (2H, br d, J = 6.27 Hz) 5.05 (1H, s) 6.21 (1H, br d, J = 6.78 Hz) 6.32-6.38 (1H, m) 6.84 (1H, dt, J = 15.43, 5.96 Hz) 7.11 (1H, br s) 7.35-7.38 (1H, m) 7.43-7.48 (1H, m) 7.48-7.51 (1H, m) 7.52 (1H, s) 7.53-7.57 (1H, m) 7.58-7.61 (1H, m) 7.65-7.69 (1H, m) 7.74 (1H, d, J = 5.52 Hz) 8.45 (1H, d, J = 2.01 Hz) 8.51 (1H, d, J = 5.77 Hz) 10.29 (1H, s); LCMS: 97.46%, MS (ESI): m/z 653.3 [M + H]+ |
| 186 | 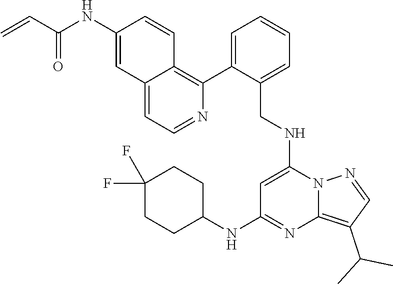 [SMILES: C=CC(=O)Nc1ccc2c(-c3ccccc3CNc3cc(NC4CCC(F)(F)CC4)nc4c(C(C)C)cnn34)nccc2c1] | Pink powder; ¹H NMR (400 MHz, DMSO-d6) δ 10.37 (1H, s), 8.53 (1H, d, J = 4.0 Hz), 8.47 (1H, d, J = 2.0 Hz), 7.76 (1H, d, J = 8.0 Hz), 7.67-7.72 (1H, m), 7.59- 7.65 (1H, m), 7.43-7.58 (4H, m), 7.37 (1H, d, J = 8.0 Hz) 7.08-7.15 (1H, m), 6.47-6.60 (1H, m), 6.30-6.42 (1H, m), 6.22 (1H, d, J = 8.0 Hz), 5.82 (1H, dd, J = 10.00, 2.0 Hz), 5.06 (1H, s), 4.25 (2H, d, J = 8.0 Hz), 3.82-3.94 (1H, m), 2.91-3.00 (1H, m), 1.85-2.10 (6H, m), 1.51-1.65 (2H, m), 1.26 (6H, d, J = 4.0 Hz); HPLC: 98.0% (254 nm), MS (ESI): m/z 596.3 [M + H]+ |
| 187 | 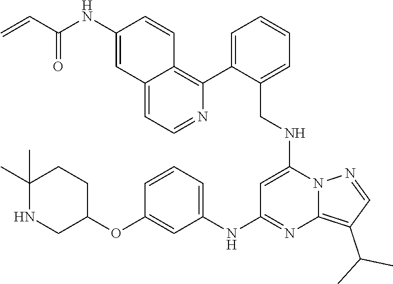 [SMILES: C=CC(=O)Nc1ccc2c(-c3ccccc3CNc3cc(Nc4cccc(OC5CCC(C)(C)NC5)c4)nc4c(C(C)C)cnn34)nccc2c1] | White powder; ¹H NMR (DMSO-d6, 400 MHz) δ10.36 (1H, s) 8.72 (1H, s) 8.52 (1H, d, J = 5.6 Hz) 8.46 (1H, d, J = 2.0 Hz) 7.72-7.77 (2H, m) 7.62-7.70 (3H, m) 7.45-7.57 (3H, m) 7.38 (1H, d, J = 7.2 Hz) 7.32 (1H, s) 7.08-7.12 (2H, m) 6.46-6.56 (2H, m) 6.30-6.40 (1H, m) 5.78-5.85 (1H, m) 5.35 (1H, s) 4.32 (2H, d, J = 6.0 Hz) 4.18-4.25 (1H, m) 2.81 (1H, dd, J = 13.2, 7.2 Hz) 1.90-1.96 (1H, m) 1.67-1.79 (1H, m) 1.56-1.65 (1H, m) 1.35-1.42 (1H, m) 1.32 (6H, dd, J = 6.8, 0.8 Hz) 1.26 (1H, d, J = 3.2 Hz) 1.11 (6H, d, J = 12.0 Hz); LCMS: 99.6%, MS (ESI): m/z 681.4 [M + H]+ |
| 188 | 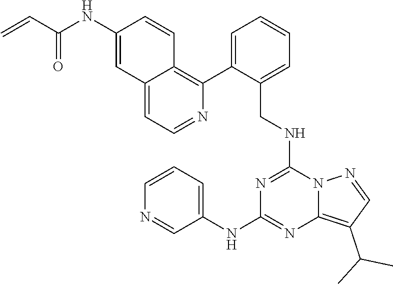 [SMILES: C=CC(=O)Nc1ccc2c(-c3ccccc3CNc3nc(Nc4cccnc4)nc4c(C(C)C)cnn34)nccc2c1] | White powder; ¹H NMR (400 MHz, DMSO-d6) δ ppm 10.39 (s, 1H) 9.10 (br s, 1H) 8.99 (d, J = 2.51 Hz, 1H) 8.54 (d, J = 5.77 Hz, 1H) 8.48 (s, 1H) 8.35 (br s, 1H) 8.22-8.26 (m, 1H) 8.20-8.22 (m, 1H) 7.80 (s, 1H) 7.75 (d, J = 5.77 Hz, 1H) 7.68-7.73 (m, 2H) 7.67 (s, 1H) 7.59 (td, J = 7.47, 1.38 Hz, 1H) 7.52-7.57 (m, 1H) 7.44 (dd, J = 7.40, 1.38 Hz, 1H) 7.30 (dd, J = 8.41, 4.64 Hz, 1H) 6.52-6.64 (m, 1H) 6.36-6.47 (m, 1H) 5.85-5.93 (m, 1H) 4.65 (br d, J = 5.02 Hz, 2H) 3.02-3.10 (m, 1H) 1.37 (d, J = 6.78 Hz, 6H); HPLC: 98.7%, MS (ESI): 556.1 m/z [M + H]+ |
| 189 | 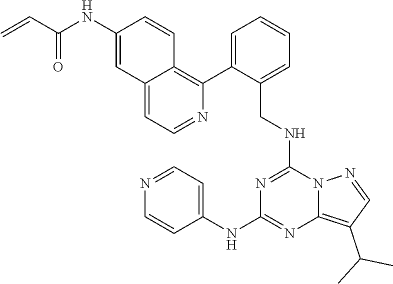 [SMILES: C=CC(=O)Nc1ccc2c(-c3ccccc3CNc3nc(Nc4ccncc4)nc4c(C(C)C)cnn34)nccc2c1] | White powder; ¹H NMR (DMSO-d6, 400 MHz) δ 8.85 (2H, d, J = 7.6 Hz) 8.23-8.30 (2H, m) 7.98 (1H, s) 7.67 (1H, d, J = 7.2 Hz) 7.39-7.56 (5H, m) 7.32 (1H, d, J = 7.2 Hz) 7.01 (2H, d, J = 7.2 Hz) 6.49-6.59 (1H, m) 6.33 (1H, dd, J = 16.8, 2.0 Hz) 5.80 (1H, dd, J = 10.0, 2.0 Hz) 4.71 (2H, s) 1.31 (6H, d, J = 6.8 Hz); LCMS: 97.2%, MS (ESI): m/z 556.3 [M + H]+ |
| 190 | 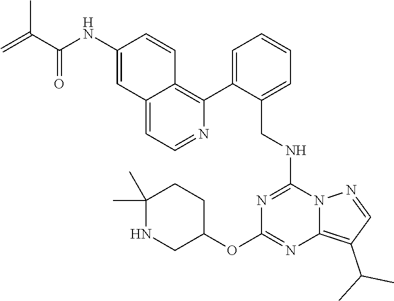 [SMILES: C=C(C)C(=O)Nc1ccc2c(-c3ccccc3CNc3nc(OC4CCC(C)(C)NC4)nc4c(C(C)C)cnn34)nccc2c1] | White powder; ¹H NMR (400 MHz, DMSO-d6) δ ppm 1.09 (6H, d, J = 8.78 Hz) 1.25 (6H, d, J = 7.03 Hz) 1.32- 1.40 (1H, m) 1.51-1.58 (1H, m) 1.64-1.74 (1H, m) 1.84- 1.95 (1H, m) 2.01 (3H, s) 2.76 (1H, dd, J = 12.92, 7.40 Hz) 2.94-3.00 (2H, m) 4.51 (2H, br s) 4.69 (1H, tt, J = 7.78, 4.02 Hz) 5.57 (1H, s) 5.89 (1H, s) 7.34 (1H, d, J = 7.03 Hz) 7.43-7.52 (2H, m) 7.53-7.60 (2H, m) 7.68 (1H, d, J = 5.77 Hz) 7.73-7.79 (2H, m) 8.40 (1H, s) 8.45 (1H, d, J = 5.52 Hz) 9.97 (1H, br s); LCMS: 100%, MS (ESI): m/z 605.4 [M + H]+ |
| 191 | 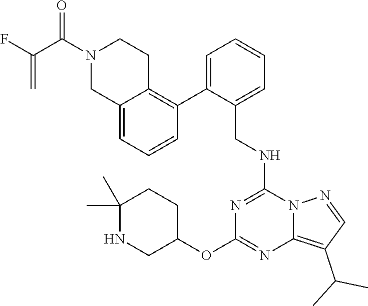 [SMILES: C=C(F)C(=O)N1CCc2c(cccc2-c2ccccc2CNc2nc(OC3CCC(C)(C)NC3)nc3c(C(C)C)cnn23)C1] | White powder; ¹H NMR (400 MHz, DMSO-d6) δ ppm 8.65 (br s, 1H) 7.87 (s, 1H) 7.41-7.48 (m, 1H) 7.31- 7.41 (m, 2H) 7.19-7.25 (m, 1H) 7.15-7.19 (m, 1H) 7.06-7.15 (m, 2H) 5.13-5.32 (m, 2H) 5.08 (br s, 1H) 4.76 (br d, J = 17.07 Hz, 1H) 4.53 (m 2H) 4.32-4.45 (m, 1H) 3.74 (m, 1H) 3.50 (m, 1H) 3.36 (m, 1H) 3.15-3.18 (m, 1H) 2.99-3.03 (m, 1H) 2.54 (m, 2H) 2.04 (m, 1H) 1.72-1.90 (m, 2H) 1.56-1.67 (m, 1H) 1.34 (m, 6H) 1.29 (d, J = 7.03 Hz, 6H); HPLC: 100%, MS (ESI): 598.4 m/z [M + H]+ |
| 192 | 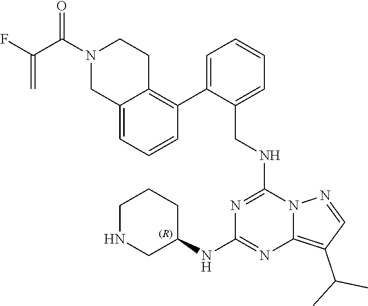 | White powder; ¹H NMR (400 MHz, DMSO-d6) δ ppm 9.23 (br s, 1H) 9.09 (br s, 1H) 8.15-8.83 (m, 1H) 7.75 (s, 1H) 7.47 (m, 1H) 7.31-7.42 (m, 2H) 7.19-7.26 (m, 1H) 7.06-7.19 (m, 3H) 5.10-5.32 (m, 2H) 4.74 (m, 1H) 4.32-4.70 (m, 3H) 4.16 (br s, 1H) 3.65-3.75 (m, 2H) 3.34 (m, 1H) 3.15 (m, 1H) 2.98 (dt, J = 13.80, 6.90 Hz, 1H) 2.87 (m, 2H) 2.53-2.60 (m, 1H) 2.42-2.48 (m, 1H) 1.85-2.06 (m, 2H) 1.71-1.85 (m, 1H) 1.55- 1.69 (m, 1H) 1.27 (d, J = 6.78 Hz, 6H); HPLC: 100%, MS (ESI): 569.1 m/z [M + H]+ |
| 193 | 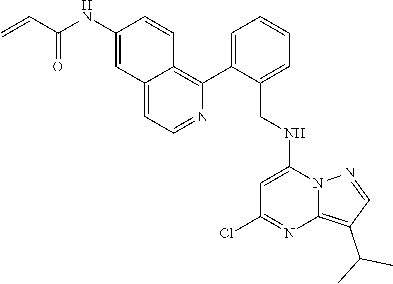 [SMILES: C=CC(=O)Nc1ccc2c(-c3ccccc3CNc3cc(Cl)nc4c(C(C)C)cnn34)nccc2c1] | White solid; ¹H NMR (400 MHz, CDCl₃) δ ppm 8.59 (1H, d, J = 5.52 Hz) 8.36 (1H, d, J = 1.76 Hz) 7.72 (1H, s) 7.39-7.67 (7H, m) 7.31 (1H, dd, J = 9.03, 2.01 Hz) 7.03 (1H, br t, J = 6.15 Hz) 6.52 (1H, dd, J = 16.81, 1.00 Hz) 6.24-6.34 (1H, m) 5.87 (1H, dd, J = 10.29, 1.00 Hz) 5.61 (1H, s) 4.28-4.59 (2H, m) 3.13-3.23 (1H, m) 1.28 (7H, d, J = 6.78 Hz); LCMS: 100.0%, MS (ESI): m/z 497.2 [M + H]+ |
| 194 | 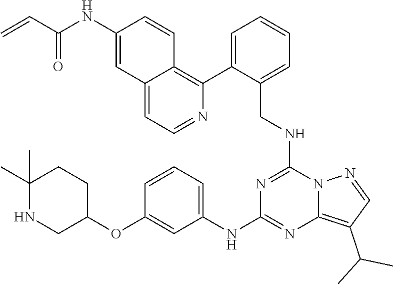 [SMILES: C=CC(=O)Nc1ccc2c(-c3ccccc3CNc3nc(Nc4cccc(OC5CCC(C)(C)NC5)c4)nc4c(C(C)C)cnn34)nccc2c1] | White powder; ¹H NMR (400 MHz, DMSO-d6) δ ppm 10.35 (br s, 1H) 8.77 (s, 1H) 8.48 (d, J = 5.77 Hz, 1H) 8.42 (s, 1H) 8.22 (br s, 1H) 7.67-7.72 (m, 2H) 7.59- 7.66 (m, 2H) 7.49-7.55 (m, 1H) 7.44-7.49 (m, 1H) 7.37 (br d, J = 7.28 Hz, 1H) 7.23 (br d, J = 9.29 Hz, 1H) 7.10 (t, J = 8.16 Hz, 1H) 5.82 (br dd, J = 10.16, 1.88 Hz, 1H) 4.57 (br s, 2H) 4.21 (br d, J = 3.76 Hz, 1H) 3.00- 3.06 (m, 2H) 2.79 (br dd, J = 12.80, 7.03 Hz, 1H) 1.89- 2.07 (m, 2H) 1.67-1.77 (m, 1H) 1.54-1.64 (m, 1H) 1.18-1.35 (m, 8H) 1.10 (d, J = 13.80 Hz, 4H); HPLC: 96.8%, MS (ESI): 682.3 m/z [M + H]+ |
| 195 | 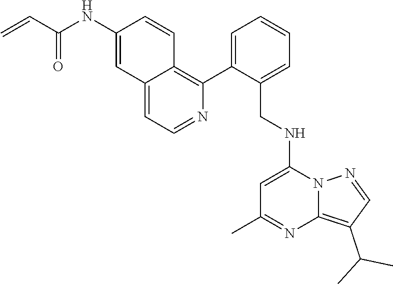 [SMILES: C=CC(=O)Nc1ccc2c(-c3ccccc3CNc3cc(C)nc4c(C(C)C)cnn34)nccc2c1] | Yellow solid; ¹H NMR (DMSO-d6, 400 MHz) δ10.37 (1H, s) 8.54 (1H, d, J = 5.77 Hz) 8.48 (1H, d, J = 1.76 Hz) 7.70-7.79 (3H, m) 7.58-7.69 (3H, m) 7.48 (2H, dtd, J = 18.54, 7.42, 7.42, 1.51 Hz) 7.38 (1H, dd, J = 7.53, 1.51 Hz) 6.47-6.56 (1H, m) 6.31-6.40 (1H, m) 5.82 (1H, dd, J = 10.16, 1.88 Hz) 5.52 (1H, s) 4.33 (2H, d, J = 6.53 Hz) 2.21 (3H, s) 1.27 (7H, d, J = 6.78 Hz); LCMS: 100%, MS (ESI): m/z 477.1 [M + H]+ |
| 196 | 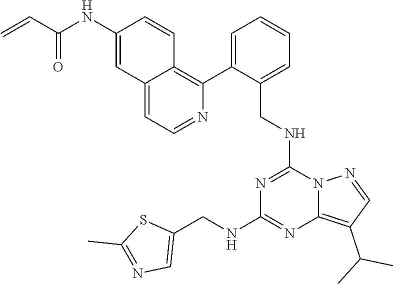 [SMILES: C=CC(=O)Nc1ccc2c(-c3ccccc3CNc3nc(NCc4cnc(C)s4)nc4c(C(C)C)cnn34)nccc2c1] | White solid; ¹H NMR (400 MHz, DMSO-d6) δ ppm 10.32 (1H, br s) 8.36-8.56 (3H, m) 8.14 (2H, br s) 7.68 (1H, d, J = 5.77 Hz) 7.61-7.65 (2H, m) 7.52-7.60 (2H, m) 7.40-7.51 (2H, m) 7.30-7.39 (2H, m) 6.94 (2H, br s) 6.26-6.59 (4H, m) 5.81 (1H, dd, J = 10.16, 1.88 Hz) 4.38-4.53 (4H, m) 2.63-2.73 (1H, m) 2.52 (3H, br s) 1.18-1.30 (6H, m); LCMS: 100.0%, MS (ESI): m/z 590.1 [M + H]+ |
| 197 | 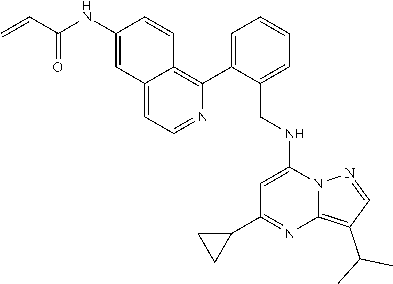 [SMILES: C=CC(=O)Nc1ccc2c(-c3ccccc3CNc3cc(C4CC4)nc4c(C(C)C)cnn34)nccc2c1] | White powder; ¹H NMR (400 MHz, DMSO-d6) δ ppm 10.37 (1H, s) 8.54 (1H, d, J = 5.77 Hz) 8.46 (1H, d, J = 1.51 Hz) 7.77 (1H, d, J = 5.77 Hz) 7.54-7.75 (5H, m) 7.43-7.53 (2H, m) 7.33-7.40 (1H, m) 6.45-6.56 (1H, m) 6.29-6.39 (1H, m) 5.81 (1H, dd, J = 10.04, 1.76 Hz) 5.55 (1H, s) 4.33 (2H, br d, J = 6.02 Hz) 2.95-3.06 (1H, m) 1.73 (1H, quin, J = 6.40 Hz) 1.25 (6H, d, J = 7.03 Hz) 0.86 (4H, d, J = 6.53 Hz); HPLC: 98.32%, MS (ESI): m/z 503.1 [M + H]+ |
| 198 | 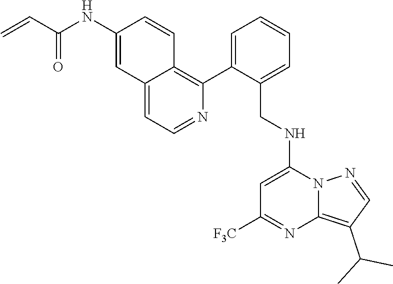 [SMILES: C=CC(=O)Nc1ccc2c(-c3ccccc3CNc3cc(C(F)(F)F)nc4c(C(C)C)cnn34)nccc2c1] | White powder; ¹H NMR (400 MHz, DMSO-d6) δ ppm 1.30 (6H, d, J = 6.78 Hz) 3.15 (1H, dt, J = 13.74, 7.06 Hz) 4.50 (2H, br s) 5.79-5.83 (1H, m) 6.12 (1H, s) 6.31- 6.37 (1H, m) 6.46-6.55 (1H, m) 7.37 (1H, dd, J = 7.40, 1.38 Hz) 7.45-7.54 (3H, m) 7.57-7.62 (1H, m) 7.64 (1H, d, J = 7.28 Hz) 7.71 (1H, d, J = 5.77 Hz) 7.99 (1H, s) 8.42 (1H, d, J = 1.76 Hz) 8.49 (1H, d, J = 5.77 Hz) 10.32 (1H, s); HPLC: 99.78% MS (ESI): m/z 531.2 [M + H]+ |
| 199 | 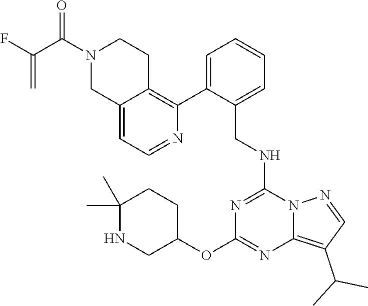 [SMILES: C=C(F)C(=O)N1CCc2c(ccnc2-c2ccccc2CNc2nc(OC3CCC(C)(C)NC3)nc3c(C(C)C)cnn23)C1] | White powder; ¹H NMR (400 MHz, DMSO-d6) δ ppm 8.37 (2H, d, J = 5.2 Hz), 7.80 (1H, s), 7.43-7.54 (1H, m), 7.32-7.43 (2H, m), 7.22 (1H, dd, J = 8.0, 2.0 Hz), 7.17 (1H, d, J = 4.0 Hz), 5.11-5.34 (2H, m), 4.58-4.81 (3H, m), 4.49 (2H, br s), 3.61 (2H, br s), 2.91-3.06 (2H, m), 2.70-2.81 (1H, m), 2.57-2.63 (2H, m), 1.91- 2.09 (2H, m), 1.63-1.76 (1H, m), 1.50-1.57 (1H, m), 1.32-1.41 (1H, m), 1.26 (6H, d, J = 8.0 Hz), 1.07 (6H, d, J = 12.0 Hz); HPLC (254 nm): 98.4%, MS (ESI): m/z 599.3 [M + H]+ |
| 200 | 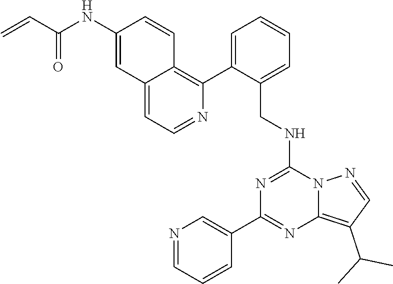 [SMILES: C=CC(=O)Nc1ccc2c(-c3ccccc3CNc3nc(-c4cccnc4)nc4c(C(C)C)cnn34)nccc2c1] | White powder; ¹H NMR (400 MHz, DMSO-d6) δ 9.27 (1H, br s), 8.62 (1H, br d, J = 8.0 Hz), 8.38 (1H, br d, J = 8.0 Hz), 8.25-8.33 (2H, m), 7.88 (1H, br s), 7.67 (1H, br d, J = 8.0 Hz), 7.44-7.55 (5H, m), 7.32 (1H, br d, J = 8.0 Hz), 6.45-6.52 (2H, m), 6.27-6.35 (1H, m), 5.75-5.85 (1H, m), 2.85-2.95 (1H, m), 4.65-4.76 (2H, m) 1.33 (6H, d, J = 8.0 Hz); HPLC: 98.8%(254 nm), MS (ESI): m/z 541.3 [M + H]+ |
| 201 | 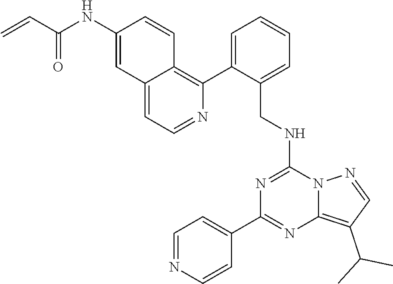 [SMILES: C=CC(=O)Nc1ccc2c(-c3ccccc3CNc3nc(-c4ccncc4)nc4c(C(C)C)cnn34)nccc2c1] | Yellow powder; ¹H NMR (400 MHz, DMSO-d6) δ 8.60- 8.72 (2H, m), 8.39 (1H, d, J = 4.0 Hz), 8.32 (1H, d, J = 1.6 Hz), 7.88-7.98 (2H, m), 7.67 (1H, d, J = 8.0 Hz), 7.58-7.64 (1H, m), 7.29-7.57 (6H, m), 6.42-6.58 (1H, m), 6.28-6.39 (1H, m), 5.81 (1H, dd, J = 12.0, 2.0 Hz), 5.14 (1H, s), 5.11-5.19 (1H, m), 4.65-4.76 (2H, m), 3.25-3.33 (1H, m), 2.91-2.99 (1H, m), 1.34 (6H, d, J = 8.0 Hz); HPLC: 97.6%(254 nm), MS (ESI): m/z 541.3 [M + H]+ |
| 202 | 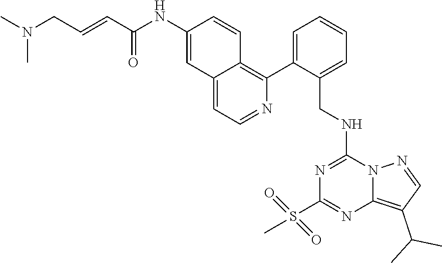 [SMILES: CC(C)c1cnn2c(NCc3ccccc3-c3nccc4cc(NC(=O)/C=C/CN(C)C)ccc34)nc(S(C)(=O)=O)nc12] | White solid; ¹H NMR (400 MHz, DMSO-d6) δ ppm 10.35 (1H, br s) 9.22 (1H, br s) 8.45 (1H, d, J = 5.77 Hz) 8.36 (1H, s) 8.05 (1H, s) 7.66 (1H, d, J = 6.02 Hz) 7.55- 7.62 (2H, m) 7.44-7.54 (2H, m) 7.30-7.40 (2H, m) 6.84 (1H, dt, J = 15.43, 6.46 Hz) 6.45 (1H, s) 6.41 (1H, s) 6.38-6.47 (1H, m) 4.65 (2H, br d, J = 14.56 Hz) 3.41- 3.56 (2H, m) 3.13-3.14 (1H, m) 3.14 (2H, s) 3.01- 3.03 (2H, m) 2.95-3.03 (7H, m) 1.29 (6H, d, J = 6.78 Hz); LCMS: 97%, MS (ESI): m/z 599.1 [M + H]+ |
| 203 | 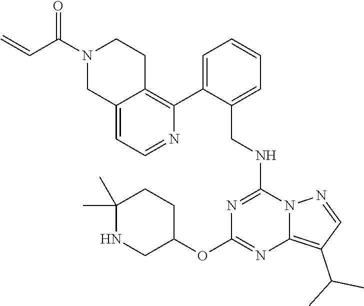 [SMILES: C=CC(=O)N1CCc2c(ccnc2-c2ccccc2CNc2nc(OC3CCC(C)(C)NC3)nc3c(C(C)C)cnn23)C1] | White powder; ¹H NMR (400 MHz, DMSO-d6) δ8.51 (1H, br s), 8.37 (1H, d, J = 4.0 Hz), 7.82 (1H, s), 7.43- 7.49 (1H, m), 7.33-7.48 (1H, m), 7.22 (1H, br d, J = 12.0 Hz), 7.16 (1H, d, J = 4.0 Hz), 6.70-6.85 (1H, m), 6.12 (1H, d, J = 16.0 Hz), 5.69 (1H, br d, J = 8.0 Hz), 4.90 (1H, br s), 4.67 (2H, br s), 4.45-4.55 (2H, m), 3.61 (2H, br s), 2.99 (3H, br d, J = 7.28 Hz), 1.95-2.05 (2H, m), 1.77-1.82 (1H, m), 1.58-1.68 (1H, m), 1.42-1.54 (2H, m), 1.12-1.30 (12H, m); HPLC (254 nm): 98.8%, MS (ESI): m/z 581.4 [M + H]+ |
| 204 | 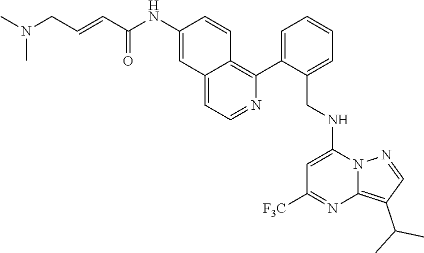 [SMILES: CC(C)c1cnn2c(NCc3ccccc3-c3nccc4cc(NC(=O)/C=C/CN(C)C)ccc34)cc(C(F)(F)F)nc12] | White powder; ¹H NMR (400 MHz, DMSO-d6) δ ppm 1.29 (5H, d, J = 6.78 Hz) 2.22 (6H, s) 2.93 (1H, br s) 3.30 (2H, br s) 4.49 (2H, br s) 6.09 (1H, s) 6.33 (1H, br d, J = 15.31 Hz) 6.84 (1H, dt, J = 15.37, 5.87 Hz) 7.35 (1H, d, J = 7.28 Hz) 7.44-7.54 (3H, m) 7.54-7.59 (1H, m) 7.64 (1H, d, J = 7.53 Hz) 7.69 (1H, d, J = 5.52 Hz) 7.98 (1H, s) 8.39 (1H, s) 8.47 (1H, d, J = 5.52 Hz) 10.24 (1H, br s); LCMS: 95.92%, MS (ESI): m/z 588.3 [M + H]+ |
| 205 | 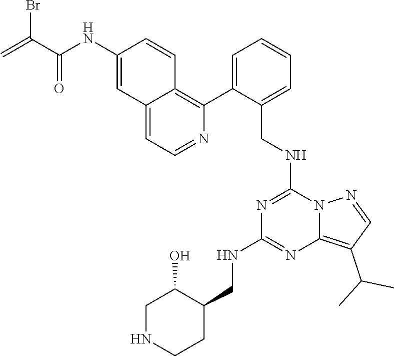 [SMILES: C=C(Br)C(=O)Nc1ccc2c(-c3ccccc3CNc3nc(NC[C@H]4CCNC[C@@H]4O)nc4c(C(C)C)cnn34)nccc2c1] | Yellow solid; ¹H NMR (DMSO-d6, 400 MHz) δ10.50 (1H, s) 8.48-8.68 (2H, m) 8.40 (2H, s) 8.21 (1H, s) 7.82 (1H, d, J = 6.0 Hz) 7.76 (1H, dd, J = 8.8, 2.4 Hz) 7.61 (3H, t, J = 5.2 Hz) 7.54 (1H, t, J = 7.2 Hz) 7.48 (1H, t, J = 7.2 Hz) 7.38 (1H, d, J = 7.6 Hz) 6.82 (1H, d, J = 2.8 Hz) 6.36 (1H, d, J = 3.2 Hz) 5.34 (1H, t, J = 4.4 Hz) 4.49 (2H, s) 3.54 (1H, td, J = 9.2, 4.0 Hz) 3.43 (2H, d, J = 14.4 Hz) 2.87-2.91 (1H, m) 2.81 (1H, s) 2.57 (1H, s) 1.95-2.07 (2H, m) 1.84 (1H, d, J = 12.8 Hz) 1.71 (1H, s) 1.40-1.52 (2H, m) 1.26 (6H, s); LCMS: 96.6%, MS (ESI): m/z 672.2 [M + H]+ |
| 206 | 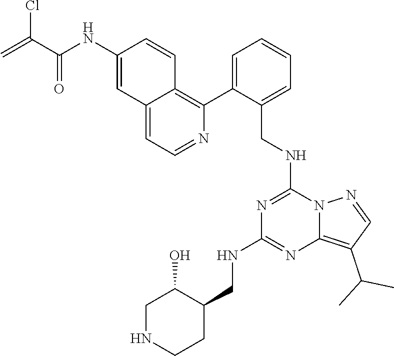 [SMILES: C=C(Cl)C(=O)Nc1ccc2c(-c3ccccc3CNc3nc(NC[C@H]4CCNC[C@@H]4O)nc4c(C(C)C)cnn34)nccc2c1] | Yellow solid; ¹H NMR (DMSO-d6, 400 MHz) δ 10.46 (1H, s) 8.61 (1H, s) 8.51 (1H, d, J = 6.0 Hz) 8.43 (2H, d, J = 1.6 Hz) 7.75-7.87 (2H, m) 7.62 (2H, t, J = 4.2 Hz) 7.43-7.59 (2H, m) 7.39 (1H, d, J = 6.4 Hz) 6.51 (1H, d, J = 2.4 Hz) 6.14 (1H, d, J = 2.8 Hz) 4.50 (2H, s) 3.51-3.57 (1H, m) 3.44 (1H, d, J = 10.8 Hz) 3.26-3.35 (1H, m) 3.24 (1H, d, J = 10.8 Hz) 3.17 (1H, d, J = 12.8 Hz) 2.85- 2.92 (1H, m) 2.81 (1H, s) 1.94-2.12 (1H, m) 1.84 (1H, d, J = 13.2 Hz) 1.69 (1H, d, J = 10.8 Hz) 1.33-1.51 (1H, m) 1.26 (1H, s) 1.21 (6H, d, J = 7.2 Hz); LCMS: 99.7%, MS (ESI): m/z 626.3 [M + H]+ |
| 207 | 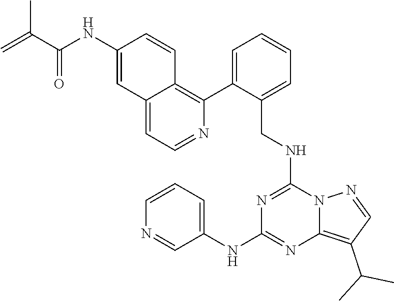 [SMILES: C=C(C)C(=O)Nc1ccc2c(-c3ccccc3CNc3nc(Nc4cccnc4)nc4c(C(C)C)cnn34)nccc2c1] | White solid; ¹H NMR (400 MHz, DMSO-d6) δ ppm 9.95 (1H, br s) 9.02 (1H, br d, J = 5.52 Hz) 8.90 (1H, d, J = 2.26 Hz) 8.46 (1H, d, J = 5.77 Hz) 8.39 (1H, d, J = 1.76 Hz) 8.27 (1H, br s) 8.11- 8.19 (2H, m) 7.71-7.79 (2H, m) 7.66 (1H, d, J = 5.77 Hz) 7.60 (2H, d, J = 8.78 Hz) 7.43-7.54 (2H, m) 7.36 (1H, dd, J = 7.40, 1.38 Hz) 7.22 (1H, dd, J = 8.41, 4.64 Hz) 5.88 (1H, s) 5.57 (1H, s) 4.57 (2H, br d, J = 5.02 Hz) 2.99 (1H, dt, J = 13.87, 7.00 Hz) 2.01 (3H, s) 1.29 (6H, d, J = 6.78 Hz); LCMS: 99%, MS (ESI): m/z 570.3 [M + H]+ |
| 208 | 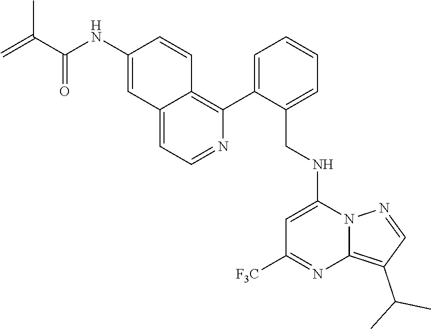 [SMILES: C=C(C)C(=O)Nc1ccc2c(-c3ccccc3CNc3cc(C(F)(F)F)nc4c(C(C)C)cnn34)nccc2c1] | Yellow powder; ¹H NMR (DMSO-d6, 400 MHz) δ9.97 (1H, s) 8.48 (2H, d, J = 5.6 Hz) 8.41 (1H, s) 8.00 (1H, s) 7.69-7.76 (2H, m) 7.64 (1H, d, J = 7.2 Hz) 7.44-7.54 (3H, m) 7.34-7.38 (1H, m) 6.14 (1H, s) 5.89 (1H, s) 5.57 (1H, s) 4.49 (2H, s) 3.04-3.08 (1H, m) 2.01 (3H, s) 1.30 (6H, d, J = 7.2 Hz); LCMS: 97.9%, MS (ESI): m/z 545.2 [M + H]+ |
| 209 | 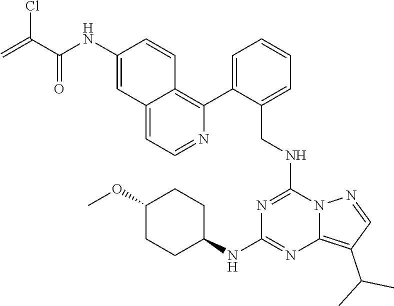 [SMILES: C=C(Cl)C(=O)Nc1ccc2c(-c3ccccc3CNc3nc(N[C@H]4CC[C@H](OC)CC4)nc4c(C(C)C)cnn34)nccc2c1] | White solid; ¹H NMR (400 MHz, DMSO-d6) δ ppm 10.38 (1H, br s) 8.50 (1H, d, J = 5.52 Hz) 8.40 (1H, s) 7.93 (1H, br s) 7.72-7.80 (2H, m) 7.54-7.65 (3H, m) 7.41-7.52 (2H, m) 7.34 (1H, d, J = 7.28 Hz) 6.49 (1H, d, J = 2.51 Hz) 6.11 (1H, d, J = 2.51 Hz) 5.91 (1H, br s) 4.45 (2H, br s) 3.55 (1H, br s) 3.24 (3H, s) 3.08 (1H, br s) 2.88 (1H, quin, J = 6.96 Hz) 1.83-2.01 (4H, m) 1.25 (2H, br d, J = 7.03 Hz) 1.21 (6H, d, J = 6.78 Hz) 1.13-1.18 (2H, m); LCMS: 100%, MS (ESI): m/z 625.1 [M + H]+ |
| 210 | 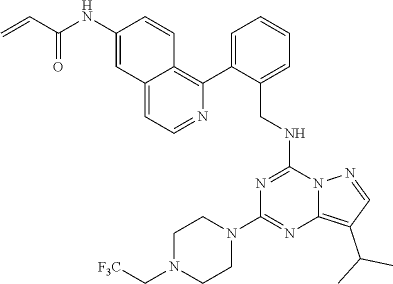 [SMILES: C=CC(=O)Nc1ccc2c(-c3ccccc3CNc3nc(N4CCN(CC(F)(F)F)CC4)nc4c(C(C)C)cnn34)nccc2c1] | White powder; ¹H NMR (400 MHz, DMSO-d6) δ 10.33 (1H, br s), 8.47 (1H, d, J = 5.6 Hz), 8.42 (1H, s), 8.15 (1H, br s), 7.71 (1H, d, J = 5.6 Hz), 7.52-7.67 (3H, m), 7.40-7.51 (2H, m), 7.32-7.37 (1H, m), 6.45-6.59 (1H, m), 6.26-6.42 (1H, m), 5.76-5.88 (1H, m), 4.46 (2H, br s), 3.44-3.61 (4H, m), 3.13-3.20 (2H, m), 2.85- 2.95 (1H, m), 2.53-2.59 (4H, m) 1.16-1.30 (6H, m); HPLC (254 nm): 97.9%, MS (ESI): m/z 630.1 [M + H]+ |
| 211 | 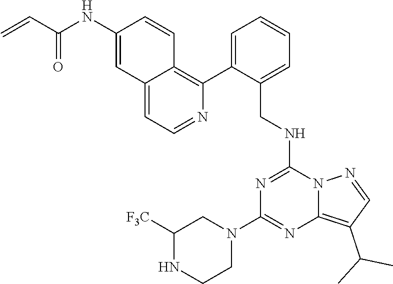 [SMILES: C=CC(=O)Nc1ccc2c(-c3ccccc3CNc3nc(N4CCNC(C(F)(F)F)C4)nc4c(C(C)C)cnn34)nccc2c1] | Yellow powder; ¹H NMR (400 MHz, DMSO-d6) δ 10.34 (1H, br s), 8.40-8.55 (2H, m), 8.11 (1H, br s), 7.70 (1H, d, J = 4.8 Hz), 7.41-7.64 (5H, m), 7.31-7.37 (1H, m), 6.46-6.61 (1H, m), 6.26-6.39 (1H, m), 5.82 (1H, dd, J = 10.0, 2.0 Hz), 4.48 (2H, br s), 4.22-4.35 (1H, m), 3.96 (1H, d, J = 12.0 Hz), 3.22-3.35 (1H, m), 2.83- 2.96 (2H, m), 2.53-2.73 (3H, m), 1.23 (6H, d, J = 8.0 Hz); HPLC (254 nm): 98.2%, MS (ESI): m/z 616.3 [M + H]+ |
| 212 | 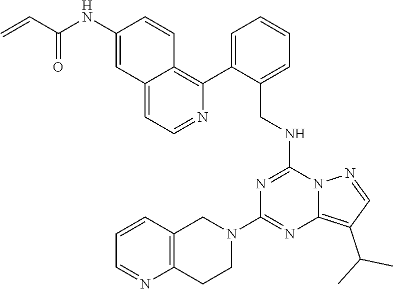 [SMILES: C=CC(=O)Nc1ccc2c(-c3ccccc3CNc3nc(N4CCc5ncccc5C4)nc4c(C(C)C)cnn34)nccc2c1] | Yellow solid; ¹H NMR (DMSO-d6, 400 MHz) δ 10.34 (1H, s) 8.40-8.49 (2H, m) 8.36 (1H, d, J = 4.52 Hz) 8.14 (1H, br s) 7.67 (1H, d, J = 5.77 Hz) 7.59-7.63 (1H, m) 7.53-7.57 (1H, m) 7.42-7.53 (3H, m) 7.35 (1H, d, J = 6.27 Hz) 7.19 (1H, dd, J = 7.53, 4.77 Hz) 6.46-6.55 (1H, m) 6.30-6.37 (1H, m) 5.81 (1H, dd, J = 10.29, 1.51 Hz) 4.71 (2H, s) 4.52 (2H, s) 3.89 (2H, t, J = 5.90 Hz) 2.94 (1H, dt, J = 13.68, 6.71 Hz) 2.86 (2H, t, J = 5.77 Hz) 1.25 (6H, d, J = 6.78 Hz); LCMS: 97.7%, MS (ESI): m/z 596.4 [M + H]+ |
| 213 | 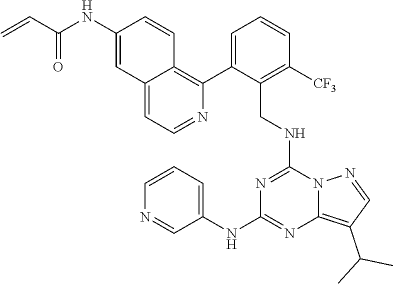 [SMILES: C=CC(=O)Nc1ccc2c(-c3cccc(C(F)(F)F)c3CNc3nc(Nc4cccnc4)nc4c(C(C)C)cnn34)nccc2c1] | White powder; ¹H NMR (400 MHz, DMSO-d6) δ 10.23 (1H, s) 8.95 (1H, s) 8.88 (1H, d, J = 2.0 Hz) 8.29 (1H, d, J = 5.6 Hz) 8.25 (1H, s) 8.06-8.18 (2H, m) 7.97 (1H, d, J = 7.6 Hz) 7.83 (1H, s) 7.70-7.76 (1H, m) 7.63-7.68 (1H, m) 7.58 (1H, s) 7.45-7.55 (3H, m) 7.24 (1H, dd, J = 8.0, 4.4 Hz) 7.21-7.28 (1H, m) 6.42-6.53 (1H, m) 6.25-6.36 (1H, m) 5.79 (1H, dd, J = 10.4, 1.6 Hz) 5.24 (1H, d, J = 14.0 Hz) 4.26 (1H, d, J = 14.4 Hz) 2.90 (1H, m) 1.24 (6H, d, J = 6.8 Hz); HPLC (254 nm): 98.2%, MS (ESI): m/z 624.2 [M + H]⁺ |
| 214 | 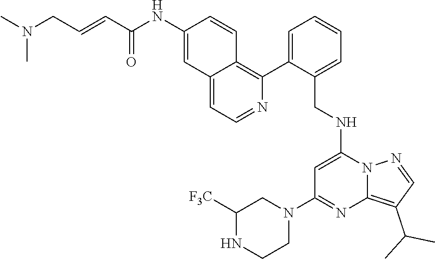 [SMILES: CC(C)c1cnn2c(NCc3ccccc3-c3nccc4cc(NC(=O)/C=C/CN(C)C)ccc34)cc(N3CCNC(C(F)(F)F)C3)nc12] | Yellow powder; ¹H NMR (400 MHz, DMSO-d6) δ ppm 10.46 (1H, br s) 8.39-8.51 (2H, m) 7.66-7.78 (2H, m) 7.58-7.64 (2H, m) 7.40-7.56 (4H, m) 7.32-7.37 (1H, m) 6.84 (1H, dt, J = 15.43, 5.96 Hz) 6.40 (1H, br d, J = 15.56 Hz) 5.34 (1H, s) 4.26-4.34 (3H, m) 3.76 (1H, br d, J = 12.30 Hz) 3.27-3.39 (4H, m) 2.92-3.05 (2H, m) 2.66-2.73 (2H, m) 2.23 (6H, s) 1.91-2.09 (1H, m) 1.25 (6H, d, J = 6.78 Hz); LCMS: 98.05%, MS (ESI): m/z 672.3 [M + H]⁺ |
| 215 | 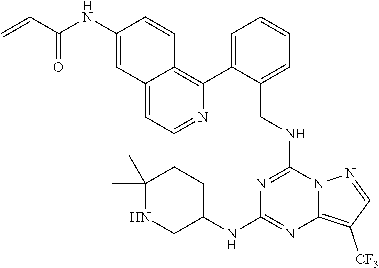 [SMILES: C=CC(=O)Nc1ccc2c(-c3ccccc3CNc3nc(NC4CCC(C)(C)NC4)nc4c(C(F)(F)F)cnn34)nccc2c1] | Off-white powder; ¹H NMR (400 MHz, DMSO-d6) δ ppm 10.32 (1H, br s) 8.47 (1H, br d, J = 5.77 Hz) 8.41 (1H, s) 7.92-8.05 (1H, m) 7.62-7.77 (2H, m) 7.58 (1H, br d, J = 7.78 Hz) 7.41-7.54 (2H, m) 7.35 (1H, br d, J = 7.03 Hz) 6.50 (1H, br dd, J = 16.94, 10.16 Hz) 6.33 (1H, br dd, J = 17.07, 1.76 Hz) 5.75-5.86 (1H, m) 4.50 (2H, br s) 3.19 (3H, br s) 3.04 (3H, br s) 2.45 (2H, br s) 1.94-2.10 (2H, m) 1.49 (2H, br s) 1.29-1.36 (1H, m) 1.26 (3H, s) 1.08 (3H, s); LCMS: 100%, MS (ESI): m/z 616.4 [M + H]⁺ |
| 216 | 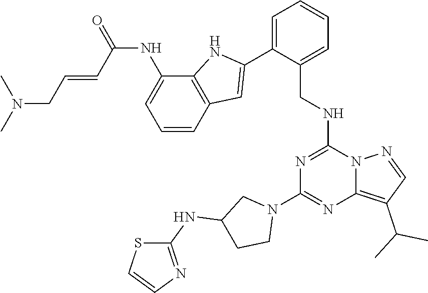 [SMILES: CC(C)c1cnn2c(NCc3ccccc3-c3cc4cccc(NC(=O)/C=C/CN(C)C)c4[nH]3)nc(N3CCC(Nc4nccs4)C3)nc12] | Off-white powder; ¹H NMR (400 MHz, DMSO-d6) δ ppm 11.50 (br s, 1H) 10.43 (br s, 1H) 8.53 (br s, 1H) 7.75 (br d, J = 8.28 Hz, 1H) 7.71 (s, 1H) 7.55-7.64 (m, 2H) 7.37 (m, 3H) 7.13 (br d, J = 3.51 Hz, 1H) 7.01 (br t, J = 7.78 Hz, 1H) 6.79-6.87 (m, 2H) 6.71 (br d, J = 3.26 Hz, 1H) 6.64 (d, J = 2.01 Hz, 1H) 4.96 (br d, J = 6.53 Hz, 2H) 4.25 (br s, 1H) 3.95 (br d, J = 5.77 Hz, 2H) 3.66 (br s, 1H) 3.45-3.51 (m, 1H) 3.40 (m, 2H) 2.93- 3.00 (m, 1H) 2.82 (s, 6H) 2.13 (m, 1H) 1.89 (m, 1H) 1.25 (d, J = 7.03 Hz, 6H); HPLC: 96.6%, MS (ESI): 698.4 m/z [M + Na]⁺ |

### EXAMPLES

The invention is now further described by reference to the following examples which are intended to illustrate, not to limit the scope of the invention.

### Example 1: Enzymatic Assay for CDK₁, CDK₂, CDK₅ and CDK₇

Enzymatic Binding Assay Protocol for CDK₁, CDK₂, CDK₅ and CDK₇

Compounds are synthesised as described in WO2019/197546. Inhibition activity of the respective compound on CDK kinases under Km value of ATP was tested in a FRET-based The LANCE® Ultra kinase assay (Perkin Elmer) using a ULight™-labeled peptide substrate and an appropriate Europium-labeled anti-phospho-antibody. Test compounds were resuspended in DMSO solution, and then 4-fold serial dilutions for 8 doses were prepared using automated liquid handler (POD™810, Labcyte) and 8onL/well of diluted compound solutions were added into the 384-well plates (Greiner, Cat #784075). And then 68 nM of ULight-MBP peptide (Perkin Elmer, Cat #TRF0109-M) and 5 ul/well of ATP (Sigma, Cat #A7699) were added to the plate. After 1 min centrifugation at 1000 rpm, purified CDKs/Cyclin complex were added at the following concentrations respectively. 24 uM for CDK₁/Cyclin B (Invitrogen, Cat #PR4768C), 22 uM for CDK₂/Cyclin A (Invitrogen, Cat #PV6290), 10 uM for CDK₅/p25 (Invitrogen, Cat #PR8543B) and 400 uM for CDK₇/Cyclin H/MNAT1 (Invitrogen, Cat #PR6749B) were added to each corresponding plate for CDK₁, CDK₂, CDK₅ and CDK₇. Incubate at 23° C. for 60 min and then Eu-labeled anti-phospho-Myelin Basic Protein (PE, Cat #TRF0201-M) and EDTA (Invitrogen, Cat #15575038) mixture in Lance Detection Buffer (Perkin Elmer, Cat #CR97100) was added in each well. After additional incubation at 23° C. for 60 min, the fluorescence of the test articles was measured using Envision leader (Perkin Elmer, USA) [Laser as excitation light; APC 615 nm and Europium 665 as the first and the second emission filter]. Data were analyzed using XL Fit software.

The results are shown in FIG. 1 for selected compounds. It becomes clear therefrom that the compounds according to the present invention show a highly selective inhibition against cyclin-dependent kinase 7 over other cyclin-dependent kinases.

### Example 2: HCMV GFP-Based Antiviral Assay

### Cell Culture and Virus

Primary human foreskin fibroblasts (HFF) were cultured in minimal essential medium (MEM) containing 5% (vol/vol) fetal calf serum. Infection analysis was restricted to cell passage numbers below 20. HCMV strain AD169 was grown in HFF and quantitated for infectivity by a plaque reduction assays (PRA). Aliquots were stored at −80° C.

### Construction of Recombinant AD169-GFP Human Cytomegalovirus

For construction of a recombination vector, two linker sequences were inserted into the pBlueScribe vector pBS1 (Stratagene): the first contained restriction sites for NheI, SpeI, PacI, and BglII followed by a loxP sequence (ATAACTTCGTATAGCATACATTATACGAAGTTAT) (SEQ ID NO:1) and was introduced into PstI/XbaI sites of the vector; the second contained another loxP sequence followed by restriction sites HpaI, ClaI, and PmeI and was introduced into BamHI/Asp718 sites. A gene cassette consisting of a “humanized” version of the open reading frame (ORF) coding for GFP (gfp-h) under the control of the HCMV enhancer/promoter and the Ptk/PY441 enhancer-driven neoR selection marker was excised from plasmid pTR-UF5 and inserted into the recombination vector via BglII sites. At the 59 and 39 positions of this loxP-flanked gene cassette, two HCMV sequences with homology to the gene region containing open reading frames US9 and US10 were inserted. For this, viral sequences were amplified from template pCM49 via PCR in a 35-cycle program (denaturation for 45 s at 95° C., annealing for 45 s at 55° C., and elongation for 2 min at 72° C.) by use of Vent DNA polymerase (New England Biolabs). A US10-specific sequence of 1,983 bp was generated using primers US10-39SpeI (GCTCACTAGTGGCCTAGCCTGGCTCATGGCC) (SEQ ID NO:2) and US10-59PacI (GTCCTTAATTAAGACGTGGTTGTGGTCACCGAA) (SEQ ID NO:3) and inserted at the vector 59 cloning position via SpeI/PacI restriction sites (boldfaced). A US9-specific sequence of 2,010 bp was generated using primers US9-39PmeI (CTCGGTITAAACGACGTGAGGCGCTCCGTCACC) (SEQ ID NO:4) and US-59ClaI (TTGCATCGATACGGTGTGAGATACCACGATG) (SEQ ID NO:5) and inserted at the vector 39 cloning position via PmeI/ClaI restriction sites (boldfaced). The resulting construct, pHM673, was linearized by use of restriction enzyme NheI and transfected into HFF via the electroporation method using a Gene Pulser (Bio-Rad; 280 V, 960 mF, 400 V). After 24 h of cultivation, cells were used for infection with 1 PFU of HCMV strain AD169/ml. Selection with 200 mg of Geneticin (ICN)/ml was started 24 h postinfection. Following 3 weeks of passage in the presence of Geneticin, GFP fluorescence could be detected in most of the infected cells. Plaque assays were performed with infectious culture supernatant on HFF, and single virus plaques were grown by transfer to fresh HFF cultured in 48-well plates. DNA was isolated from infected HFF (fluorescence-positive wells) and confirmed for the presence of recombinant virus by PCR. For this, primers US9[198789](TGACGCGAGTATTACGTGTC) (SEQ ID NO:6) and US10[199100](CTCCTCCTGATATGCGGTT) (SEQ ID NO:7) were used, resulting in an amplification product of 312 bp for wild-type AD169 virus and approximately 3.5 kb for recombinant virus.

### Isolation of Ganciclovir (GCV)-Resistant Virus, HCMV ADP169-CFP314

A series of laboratory variants of AD169-GFP virus that are resistant to GCV was generated. HFF were infected in 12-well plates at a multiplicity of infection (MOI) of 0.002 and incubated with 1 mM Ganciclovir. GFP expression in infected cells was monitored microscopically, and the supernatants from the positive wells were transferred to fresh cells weekly. Thereby Ganciclovir concentrations were increased stepwise (a 1 mM increase at each step) up to the point where total virus replication became critical and resistant virus was grown in individual wells. Using supernatants from these wells, two rounds of plaque purifications were performed in HFF. Finally, Ganciclovir-resistant viral clones (AD169-GFP314) which were able to replicate in the presence of 10 mM GCV were isolated.

### Neutral Red Uptake Assay (NRA) (Cytotoxicity Assay)

Cytotoxicity of the analyzed compounds was determined by the approved dye uptake assay using Neutral Red (NRA). Human foreskin fibroblast (HFF) cells were seeded in 96-well plates one day prior to testing, cultured overnight until cells were ˜80% confluent and then incubated 37° C. under a 5% CO2 atmosphere for 7 days with test compounds. The NRA was performed using 40 μg/mL of neutral red. The neutral red treated plate was incubated at 37° C. for 3 hr and then washed with 150 μl of PBS. Neutral red distaining solution (1% acetic acid in 50% of EtOH) was added and then plate was incubated at room temperature for 10 min to stop reaction. The amount of incorporated Neutral Red was quantitated in Victor 1420 Multilabel Counter (Wallac) by fluorescence measurement using 560/630 nm for excitation/emission, respectively. The cytotoxicity of compounds to viral host cells, HFF, was determined by CC₅₀ (50% cytotoxic concentration).

### HCMV-GFP-Based Antiviral Assay

HFF were cultured to 90% confluency in 12-well plates and used for infection with AD169-GFP HCMV-virus at a tissue culture infective dose of 0.5 (GFP-TCID50 0.5, referring to an MOI of 0.002 as determined by plaque assay titration). Virus inoculation was performed for 90 min at 37° C. with occasional shaking before virus was removed and the cell layers were rinsed with phosphate-buffered saline (PBS). Then infected cell layers were incubated with 2.5 ml of MEM containing 5% (vol/vol) fetal calf serum with or without a dilution of one of the respective test compounds. Infected cells were incubated at 37° C. under a 5% CO2 atmosphere for 7 days. For lysis, 200 ml of lysis buffer (25 mM Tris [pH 7.8], 2 mM dithiothreitol [DTT], 2 mM trans-1,2-diaminocyclohexane-N,N,N9,N9-tetraacetic acid, 1% Triton X-100, 10% glycerol) was added to each well and incubated for 10 min at 37° C., followed by a 30-min incubation at room temperature on a shaker. Lysates were centrifuged for 5 min at 15,000 rpm in an Eppendorf centrifuge to remove cell debris. One hundred microliters of the supernatants were transferred to an opaque 96-well plate for automated measuring of GFP signals in a Victor 1420 Multilabel Counter (Wallac). The cytotoxicity of compounds against virus replication was determined by EC₅₀ (50% effective concentration).

FIG. 2 shows the results of the HCMV-GFP assay for compounds 174 and 177 with hCMV-AD169 strain and FIG. 3 summarizes, inter alia, the safety index (SI), defined as the ratio of CC₅₀ to EC₅₀ with EC₅₀ (μM) for antiviral activity against hCMV-AD169 strain and, CC₅₀ for toxicity to the host cell, HFF. Compounds inhibited HCMV replication in a concentration-dependent manner with 50% effective concentration (EC₅₀) values in the subnanomolar range. At the same time, the cytotoxicity against host cells occurred only in the micromolar range. Safety index of the compounds was extremely high, which indicates that antiviral activities of compounds are observed at extremely low concentrations but at the same time the compounds are safe towards the host cells.

FIG. 4 shows the results of the HCMV-GFP assay for compounds 174 and 177 with ganciclovir (GCV)-resistant HCMV strain ADP169-GFP314 strain and FIG. 5 summarizes the antiviral activity against GCV-resistant HCMV strain, cytotoxicity to host cells, and safety index. As can be seen, the compounds show high inhibitory activity against HCMV replication in the nano-to pico-molar range of EC₅₀ values. Moreover, the compounds show a high safety index (SI=CC₅₀/EC₅₀) indicating a wide therapeutic window. Especially, Compounds 174, 175 and 177 were highly effective against viruses resistant to nucleobase analogue drugs as well as non-resistant viruses.

### Example 3: Antiviral Efficacy in HSV-1

### Construction of HSV-1 GFP Recombinant Virus

The two 400-bp flanking sequences of the HSV-1 UL49 gene were amplified together by PCR from purified genomic DNA to construct a single 800-bp fragment incorporating an EcoRI site at one end, an XbaI site at the other, and a BamHI site engineered in place of the UL49 gene. This was inserted into plasmid pSP72 as an EcoRI/XbaI fragment to produce plasmid pGE120. A GFP-UL49 cassette contained on a BamHI fragment was then inserted into the BamHI site of pGE120 to produce plasmid pGE166, which consisted of GFP-UL49 surrounded by the UL49 flanking sequences and hence driven by the UL49 promoter. Equal amounts (2 mg) of plasmid pGE166 and infectious HSV-1 strain 17 DNA were transfected into COS-1 cells (10⁶) grown in a 60-mm-diameter dish by using the calcium phosphate precipitation technique modified with BES [N,N-bis(2 hydroxyl)-2 aminoethanesulfonic acid]-buffered saline in place of HEPES-buffered saline. Four days later, the infected cells were harvested into the cell medium and subjected three times to freeze-thawing, and the resulting virus was titrated on HFF cells. Around 6,000 plaques were then plated onto HFF cells and screened for possible recombinants by GFP fluorescence.

### HSV-1 GFP-Based Antiviral Assay

HFF were cultured to 90% confluency in 12-well plates and HSV-1 GFP inoculation was performed for 90 min at 37° C. with occasional shaking before virus was removed and the cell layers were rinsed with phosphate-buffered saline (PBS). Then infected cell layers were incubated with 2.5 ml of MEM containing 5% (vol/vol) fetal calf serum with or without a dilution of one of the respective test compounds. Infected cells were incubated at 37° C. under a 5% CO2 atmosphere for 7 days. For lysis, 200 ml of lysis buffer (25 mM Tris [pH 7.8], 2 mM dithiothreitol [DTI], 2 mM trans-1,2-diaminocyclohexane-N,N,N9,N9-tetraacetic acid, 1% Triton X-100, 10% glycerol) was added to each well and incubated for 10 min at 37° C., followed by a 30-min incubation at room temperature on a shaker. Lysates were centrifuged for 5 min at 15,000 rpm in an Eppendorf centrifuge to remove cell debris. One hundred microliters of the supernatants were transferred to an opaque 96-well plate for automated measuring of GFP signals in a Victor 1420 Multilabel Counter (Wallac).

Results of this assay are shown in FIG. 6. As can be seen here, the compounds show high inhibitory activity against HSV-1 replication with a nano- to pico-molar ranges of EC₅₀ values. Moreover, the compounds show a high safety index (SI=CC₅₀/EC₅₀) indicating a wide therapeutic window.

### Example 4: Antiviral Efficacy in EBV

### Akata-BX1-g Cell Culture

Akata-BX1-g is a lymphoma cell line engineered to express GFP in the EBV virus genome, replacing BXLF1 (thymidine kinase). Cells were cultured in suspension in a cell growth medium (RPMI, supplemented with 10% heat-inactivated FBS, Penicillin/Streptomycin, L-Glutamine, and 0.4 mg/mL G418) in a T225 flask in a 370C humidified 5% CO2 incubator. Cells were passaged every 3 to 4 days at a density of 0.5×10⁶/mL using cell growth medium to keep the cells under 2×10⁶ cells/mL.

### EBV-GFP Based Antiviral Assay

Akata-BX1-g cells were seeded into each well from 0.18×10⁶ to 4×10⁶ using 2 mL Medium in 12-well plates and then cultured to 90% confluency. Then cell layers were incubated with 2.5 ml of RPMI containing 10% (vol/vol) heat-inactivated FBS, Penicillin/Streptomycin, L-Glutamine, and 0.4 mg/mL G418 with or without a dilution of one of each test compound. Cells were incubated at 37° C. under a 5% CO2 atmosphere for 4 days. For lysis, 200 ml of lysis buffer (25 mM Tris [pH 7.8], 2 mM dithiothreitol [DTT], 2 mM trans-1,2-diaminocyclohexane-N,N,N9,N9-tetraacetic acid, 1% Triton X-100, 10% glycerol) was added to each well and incubated for 10 min at 37° C., followed by a 30-min incubation at room temperature on a shaker. Lysates were centrifuged for 5 min at 15,000 rpm in an Eppendorf centrifuge to remove cell debris. One hundred microliters of the supernatants were transferred to an opaque 96-well plate for automated measuring of GFP signals in a Victor 1420 Multilabel Counter (Wallac).

### Neutral Red Uptake Assay (NRA) (Cytotoxicity Assay)

Cytotoxicity of the analyzed compounds was determined by the approved dye uptake assay using Neutral Red (NRA). Akata-BX1-g cells were seeded into 96-well plates one day prior to testing, incubated overnight until cells were ˜80% confluent and then incubated with test compounds at 37° C. under a 5% CO2 atmosphere for 3 days. The NRA was performed using 40 μg/mL of neutral red. The neutral red treated plate was incubated at 37° C. for 3 hr and then washed with 150 μl of PBS. Neutral red distaining solution (1% acetic acid in 50% of EtOH) was added and then plate was incubated at room temperature for 10 min to stop reaction. The amount of incorporated Neutral Red was quantitated in Victor 1420 Multilabel Counter (Wallac) by fluorescence measurement using 560/630 nm for excitation/emission, respectively. The cytotoxicity of compounds to viral host cells, Akata-BX1-g, was determined by CC₅₀ (50% cytotoxic concentration).

Results of this assay are shown in FIG. 7. As can be seen here, the compounds show high inhibitory activity against EBV replication with the nano molar range of EC₅₀ values. Moreover, the compounds show a high safety index (SI=CC₅₀/EC₅₀) indicating a wide therapeutic window.

### Example 5: Antiviral Efficacy in pUL97 Mutated HCMVs Resistant Against to the GCV and MBV

### Cell Culture and Virus

Primary human foreskin fibroblasts (HFF) were cultured in minimal essential medium (MEM) containing 5% (vol/vol) fetal calf serum. Infection analysis was restricted to cell passage numbers below 20. HCMV strain AD169 was grown in HFF and quantitated for infectivity by a plaque reduction assays (PRA). Aliquots were stored at −80° C.

Generation of Recombinant pUL97 Mutated HCMVs

BACmid TB40E IE2-YFP was used for the generation of resistance-conferring ORF-UL97 point mutations. To this end, primers complementary to up- and downstream areas of the region to be deleted or exchanged within pUL97 were used to amplify a resistance cassette conferring kanamycin resistance. Subsequent homologous recombination of the cassette with the target sequence led to deletion or exchange of the desired sequence. Positive clones were identified by the kanamycin-resistance marker and, after sequencing, were used for the second recombination step. Then, arabinose-dependent induction of the restriction enzyme I-SceI and cleavage of the DNA resulted in a second round of recombination, thereby again deleting the resistance cassette. The successful deletion of the desired sequence was again verified via sequencing. Recombinant viruses were reconstituted by transfection of HFF, using the Fugene transfection reagent according to the manufacturer's protocol (Promega, Madison, WI, USA). The correctness of reconstituted viral DNA was again verified by sequencing. Briefly, the pUL97-C592G, H520Q, C603W, H469V, M460I and A594V mutants were applied as GCV resistant and the pUL97-L397R, T409 M, H411Y were as MBV resistant strains. pUL-F342S was subjected to a common resistant strain to GCV and MBV.

### HCMV-GFP-Based Antiviral Assay

HFF were cultured to 90% confluency in 96-well plates and used for infection with parental and pUL97 point mutation harbouring AD169-GFP HCMVs at a tissue culture infective dose of 0.25. Virus inoculation was performed for 90 min at 37° C. with occasional shaking before virus was removed and the cell layers were rinsed with phosphate-buffered saline (PBS). Then infected cell layers were incubated with 2.5 ml of MEM containing 5% (vol/vol) fetal calf serum with or without a dilution of one of the respective test compounds. Infected cells were incubated at 37° C. under a 5% CO2 atmosphere for 7 days. For lysis, 200 ml of lysis buffer (25 mM Tris [pH 7.8], 2 mM dithiothreitol [DTT], 2 mM trans-1,2-diaminocyclohexane-N,N,N9,N9-tetraacetic acid, 1% Triton X-100, 10% glycerol) was added to each well and incubated for 10 min at 37° C., followed by a 30-min incubation at room temperature on a shaker. Lysates were centrifuged for 5 min at 15,000 rpm in an Eppendorf centrifuge to remove cell debris. One hundred microliters of the supernatants were transferred to an opaque 96-well plate for automated measuring of GFP signals in a Victor 1420 Multilabel Counter (Wallac). The cytotoxicity of compounds against virus replication was determined by EC₅₀ (50% effective concentration).

Results of this assay are shown in FIGS. 8 and 9. As shown here, the compounds show high inhibitory activity against replication of pUL97 mutated HCMVs, which are able to induce resistance to Ganciclovir (GCV) (FIG. 8) or Maribavir (MBV), (FIG. 9) respectively, with the nano molar range of EC₅₀ values. Thus, effectively, the compounds according to the present invention effectively reverse such resistance.

### Example 6: Antiviral Efficacy in Papilloma Virus

### C-33 a Cell Culture

C-33 A, mouse embryo fibroblast cells were obtained from ATCC and maintained in standard growth medium of MEM with Earl's salts supplemented with 10% FBS (Hyclone, Inc. Logan UT), L-glutamine, penicillin, and gentamycin.

### Antiviral Assay for Human Papilloma Virus (HPV)

HPV genome replicon assay was developed and expresses the essential E1 and E2 proteins from the native promoter. The E2 origin binding protein interacts with the virus origin of replication and recruits the E1 replicative helicase which unwinds the DNA and helps to recruit the cellular DNA replication machinery (including DNA polymerases, type I and type II topoisomerases, DNA ligase, single-stranded DNA binding proteins, proliferating cell nuclear antigen). The replication complex then drives the amplification of the replicon which can be assessed by the expression of a destabilized NanoLuc reporter gene carried on the replicon. In the HPV11 assay, the replicon (pMP619) is transfected into C-33 A cells grown as monolayers in 384-well plates. At 48 h post transfection, the enzymatic activity of the destabilized NanoLuc reporter is assessed with NanoGlo reagent. The reference compound for this assay is PMEG and its EC50 value is within the prescribed range of 2-9.2 μM. Analysis of HPV genome replication in specific types of HPV, such as HPV6, HPV11, or HPV31 is performed with plasmid systems that utilize each HPV type.

Results of this assay are shown in FIG. 10. As can be seen here, the compounds show high inhibitory activities against HPV6, HPV11 and HPV31 replication with EC₅₀ values in the sub-micromolar range. Moreover, the compounds show a high safety index (SI=CC₅₀/EC₅₀), again, indicating a wide therapeutic window.

The features of the present invention disclosed in the specification, the claims, and/or in the accompanying figures may, both separately and in any combination thereof, be material for realizing the invention in various forms thereof.

1. A method for treating a DNA-virus infection in a subject, wherein the method comprises administering to the subject a compound having the general formula I 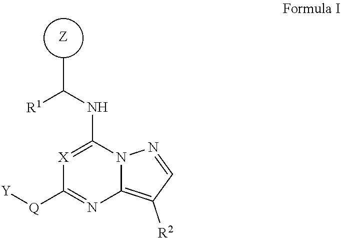
or an enantiomer, stereoisomeric form, mixture of enantiomers, diastereomer, mixture of diastereomers, racemate or a pharmaceutically acceptable salt thereof;
wherein in said compound
X is, independently at each occurrence, selected from CH and N;
Q is either absent or independently, at each occurrence, selected from the group consisting of —NH—, —NH(CH₂)—, —NH(CH₂)₂—, —NH(C═O)—, —NHSO₂—, —O—, —O(CH₂)—, —(C═O)—, —(C═O)NH— and —(C═O)(CH₂)—;
Y is, independently at each occurrence, selected from the group consisting of halogen, C1-C3 haloalkyl, C3-C8 cycloalkyl, aryl, heteroaryl, heterocyclyl, —S(═O)₂R³, C1-C6 alkyl and C1-C6 alkyl substituted with one or two of —OR⁵, —N(R⁵)R⁵, aryl, heteroaryl and heterocyclyl;
wherein C3-C8 cycloalkyl is optionally substituted with one or two of R³, R⁴ and —(C═O)R⁵, wherein heterocyclyl is optionally substituted with one or two of R³, R⁴ and —(C═O)R⁵, and wherein aryl or heteroaryl is optionally substituted with one or two of R³, C1-C6 alkyl, —OR⁵, —N(R⁵)R⁵, —(C═O)R⁵, halogen, heteroaryl and heterocyclyl;
R¹ is, at each occurrence, independently selected from the group consisting of hydrogen and methyl;
R² is, at each occurrence, independently selected from the group consisting of halogen, C1-C6 alkyl, C3-C10 cycloalkyl, —CN, —(C═O)CH₃ and C1-C3 haloalkyl, any of which is optionally substituted;
R³ is either absent or independently, at each occurrence, selected from the group consisting of hydrogen, —OR⁵, halogen, C1-C3 haloalkyl, —CN, —N(R⁵)R⁵, (═O), —NH(C═O)R⁵, —(C═O)NH₂, —S(═O)₂N(R⁵)R⁵, aryl, heteroaryl, heterocyclyl, C1-C6 alkyl and C1-C6 alkyl substituted with —OR₅, —NH₂ or —S(═O)₂N(R⁵)R⁵;
R⁴ is, independently, at each occurrence, selected from the group consisting of hydrogen, halogen, C1-C3 haloalkyl, —CN, —OR⁵, —N(R⁵)R⁵, (═O), S(═O)₂N(R⁵)R⁵, aryl, heteroaryl, heterocyclyl, C1-C6 alkyl and C1-C6 alkyl substituted with —OH, —NH₂ or —S(═O)₂N(R⁵)R⁵;
wherein both R³ and R⁴ are (═O) if attached to a single sulfur atom that forms part of Y being a heterocycle;
or wherein R³ and R⁴, together with the structure to which they are attached, form an aromatic ring, a heteroaromatic ring, a saturated or unsaturated heterocyclic ring, or a fused or bridged ring structure of any of an aromatic ring, a heteroaromatic ring, and a saturated or unsaturated heterocyclic ring;
R⁵ is, at each occurrence, independently selected from the group consisting of hydrogen, C1-C6 alkyl, C3-C6 cycloalkyl, C1-C3 haloalkyl, heteroaryl, heterocyclyl, heteroaryl substituted with one or two of halogen, —OR¹¹, —N(R¹¹)R¹¹, C1-C6 alkyl and C1-C6 alkyl substituted with —OH, —NH₂; heterocyclyl substituted with one or two of halogen, —OR¹¹, —N(R¹¹)R¹¹, C1-C6 alkyl and C1-C6 alkyl substituted with —OH or —NH₂;
Z is any structure of the following group A; 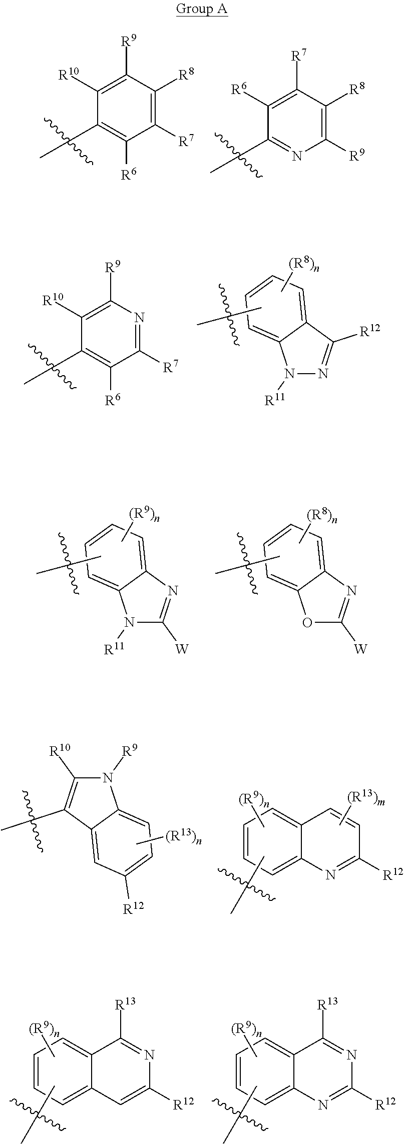
wherein n=1, 2, or 3; m=1, or 2;
R⁶ and R⁷ are, at each occurrence, independently selected from the group consisting of hydrogen, —NH(C═O)R¹⁴, —NHR¹⁴, —OR¹⁴ and any structure of the following group B, with the
proviso that, when Z is 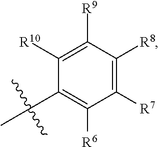
one of R⁶ and R⁷ is not H; 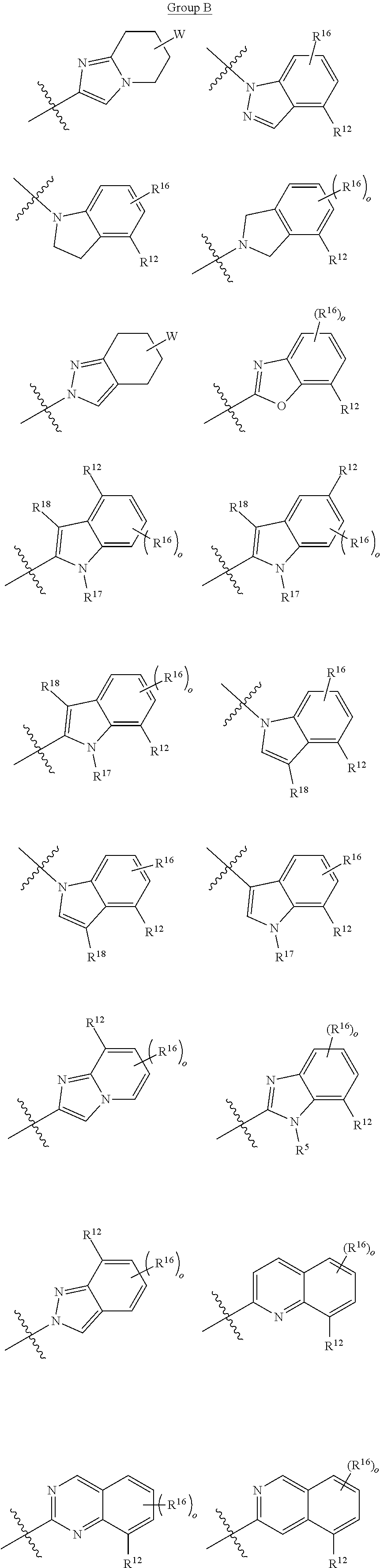 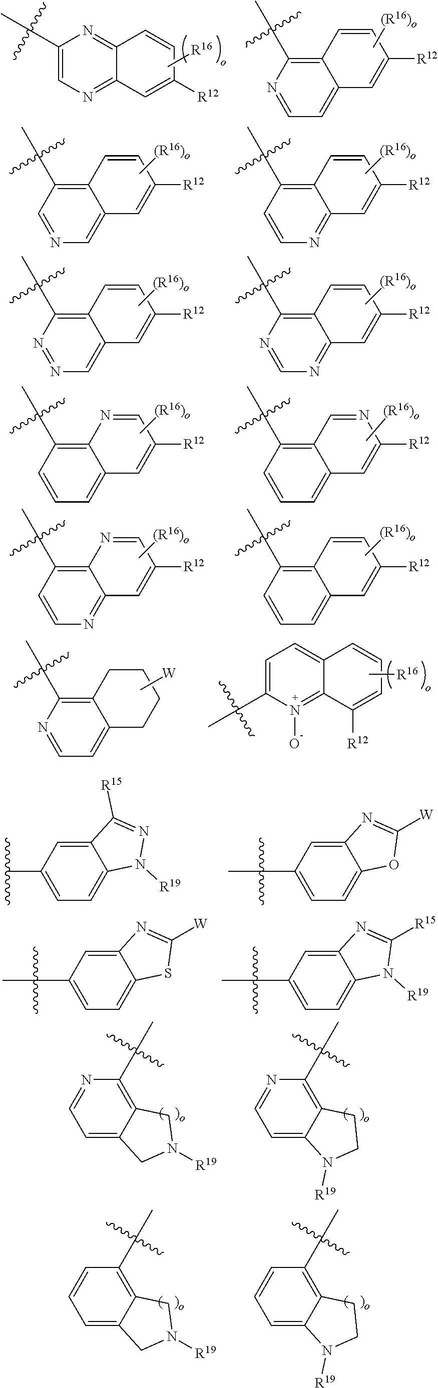
wherein o is, independently at each occurrence, selected from 1, 2 and 3;
W is any structure of the following group C; 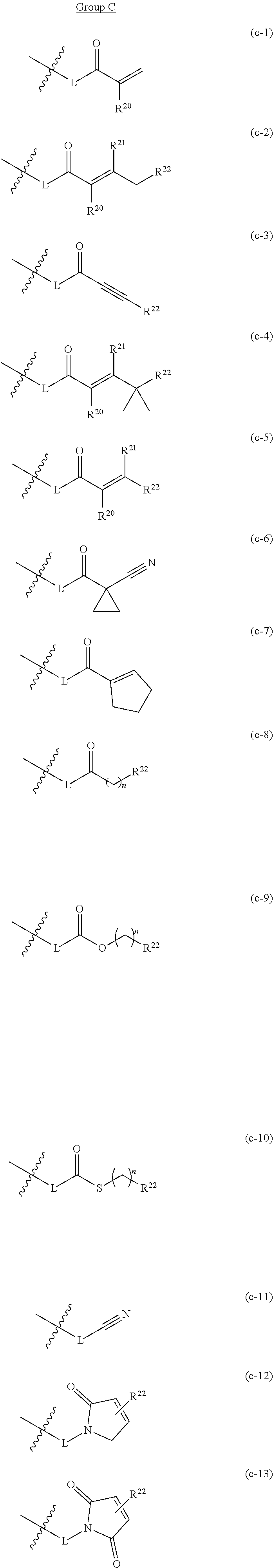 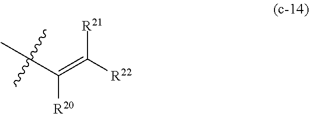
L is absent or, at each occurrence, independently selected from the group consisting of —O— and —NH—;
wherein n is, independently at each occurrence, selected from 1, 2 and 3;
R⁸, R⁹ and R¹⁰ are, at each occurrence, independently selected from the group consisting of hydrogen, halogen, C1-C6 alkyl, C1-C3 haloalkyl, —OR⁵, —CN and C1-C6 alkyl substituted with —OH, —OR⁵ or —NHR⁵;
R¹¹ is, at each occurrence, independently selected from the group consisting of hydrogen, C1-C6 alkyl, C3-C10 cycloalkyl and W, as defined above;
R¹² is, at each occurrence, independently selected from hydrogen and W, as defined above;
wherein if R¹¹ is W, R¹² is hydrogen;
R¹³ is, at each occurrence, independently selected from the group consisting of hydrogen, halogen, C1-C6 alkyl, C1-C3 haloalkyl, —NH₂, —OR⁵, —CN and W, as defined above;
wherein if R¹³ is W, R¹² is hydrogen;
R¹⁴ is any structure of group D; 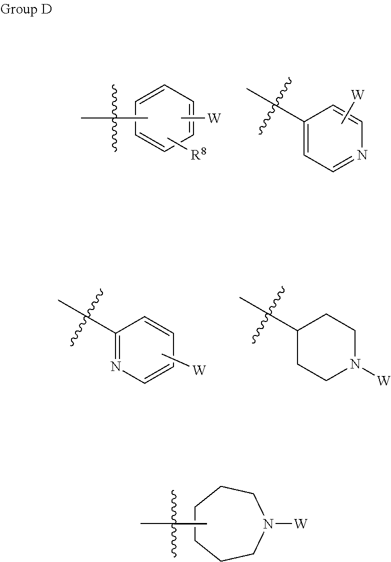
R¹⁵ is, at each occurrence, independently selected from hydrogen and W, as defined above;
R¹⁶ is, at each occurrence, independently selected from the group consisting of hydrogen, halogen, C1-C6 alkyl, C1-C3 haloalkyl, —NH₂, —OR⁵, —CN and W, as defined above;
wherein if R¹⁶ is W, R¹² is hydrogen;
R¹⁷ is, at each occurrence, independently selected from the group consisting of hydrogen, C1-C6 alkyl and C1-C3 haloalkyl;
R¹⁸ is, at each occurrence, independently selected from the group consisting of hydrogen, halogen, C1-C6 alkyl, C1-C3 haloalkyl, —NH₂, —OR⁵ and —CN;
R¹⁹ is, at each occurrence, independently selected from the group consisting of hydrogen, C1-C6 alkyl, C3-C10 cycloalkyl and W, as defined above;
wherein if R¹⁹ is W, R¹⁵ is hydrogen;
R²⁰ and R²¹ are, at each occurrence, independently selected from the group consisting of hydrogen, halogen, C1-C6 alkyl, C1-C3 haloalkyl, —OR⁵, heterocyclyl and —CN; and
R²² is, at each occurrence, independently selected from the group consisting of hydrogen, halogen, C1-C6 alkyl, C3-C10 cycloalkyl, —N(R⁵)₂, —NR¹⁹R²⁰, —NR¹⁹CH₂(CO)NH₂, heterocyclyl, —OR⁵ and —CN.

2. The method according to claim 1, wherein said DNA-virus infection is a Herpesviridae infection, and said Herpesviridae infection is an infection by a member from a Herpesviridae subfamily, such subfamily being selected from Alphaherpesvirinae, Betaherpesvirinae, and Gammaherpesvirinae.

3. The method according to claim 1, wherein said DNA-virus infection is a Herpesviridae infection, and said Herpesviridae infection is an infection by human cytomegalovirus (HCMV).

4. The method according to claim 1, wherein said DNA-virus infection is a Herpesviridae infection, and said Herpesviridae infection is an infection by human Herpes-simplex-virus-1 (HSV-1).

5. The method according to claim 1, wherein said DNA-virus infection is a Herpesviridae infection, and said Herpesviridae infection is an infection by Epstein-Barr-Virus (EBV).

6. The method according to claim 1, wherein said DNA-virus infection is a Herpesviridae infection by a virus that is resistant against nucleobase analogues or nuceloside analogues or inhibitors of viral DNA synthesis.

7. The method according to claim 6, wherein said DNA-virus infection is a human cytomegalovirus (HCMV) infection by an HCM virus (HCMV), or is a human Herpes-simplex-virus-1 (HSV-1) infection or a human Herpes-simplex-virus-2 (HSV-2) infection, or is a Epstein-Barr-Virus (EBV), wherein said HCMV, said HSV-1, said HSV-2 and said EBV is resistant against a guanine analogue, or against maribavir; or wherein said HCMV, said HSV-1 and said HSV-2 is resistant against didanosine, vidarabine, galidesivir, remdesivir, cytarabine, gemcitabine, emtricitabine, lamivudine, zalcitabine, abacavir, entecavir, stavudine, telbivudine, zidovudine, idoxuridine, or trifluridine.

8. The method according to claim 1, wherein said DNA-virus infection is a Papillomaviridae infection, and said Papillomaviridae infection is an infection by a human papillomavirus (HPV), selected from alphapapillomavirus, betapapillomavirus and gammapapillomavirus.

9. The method according to claim 8, wherein said method is used for the treatment and/or prevention of a cancer caused by or associated with HPV, said cancer being selected from cervical cancer, oropharyngeal cancer, anal cancer, penile cancer, vaginal cancer and vulvar cancer.

10. The method according to claim 9, wherein said method is performed on a subject who is a non-responder, or fails to respond adequately, to HPV-vaccination, or said method is performed on a subject who cannot be vaccinated against HPV.

11. The method according to claim 1, wherein said method comprises administering a compound having the general formula I 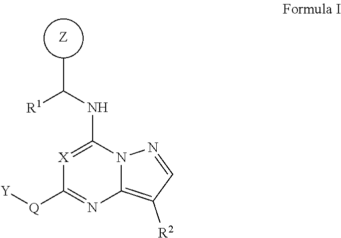
as defined in claim 1, to a subject having, or suspected of having, a Herpesviridae infection or a Papillomaviridae infection.

12. The method according to claim 1, wherein said compound is administered at an early stage of infection in said subject and/or prior to onset of any symptoms in said subject.

13. The method according to claim 1, wherein said subject is a non-responder to a previous course of treatment with a nucleobase analogue or a nucleoside analogue or an inhibitor of viral DNA synthesis.

14. The method according to claim 1, wherein said compound is administered systemically or topically.

15. The method according to claim 1, wherein said compound is a compound having the general formula Ia 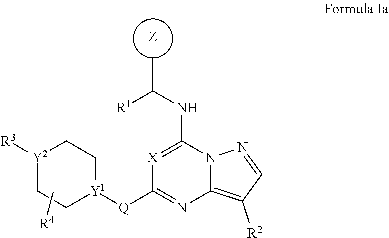
wherein
X is, independently at each occurrence, selected from CH and N;
Y¹ is, independently at each occurrence, selected from CH, C(OH) and N;
Y² is, independently at each occurrence, selected from CH, C(OH) and N;
Q is absent or, at each occurrence, independently selected from the group consisting of —NH—, —NH(CH₂)—, —NH(C═O)—, —NHSO₂—, —O—, —O(CH₂)—, —(C═O)— and —(C═O)(CH₂)—;
R¹ is, at each occurrence, independently selected from the group consisting of hydrogen and methyl;
R² is, at each occurrence, independently selected from the group consisting of halogen, C1-C6 alkyl, C3-C10 cycloalkyl, —CN, —(C═O)CH₃ and C1-C3 haloalkyl, any of which is optionally substituted;
R³ is either absent or independently, at each occurrence, selected from the group consisting of hydrogen, —OR⁵, halogen, C1-C3 haloalkyl, —CN, —N(R⁵)R⁵, (═O), —NH(C═O)R⁵, —(C═O)NH₂, —S(═O)₂N(R⁵)R⁵, aryl, heteroaryl, heterocyclyl, C1-C6 alkyl and C1-C6 alkyl substituted with —OR₅, —NH₂ or —S(═O)₂N(R⁵)R⁵;
R⁴ is, independently, at each occurrence, selected from the group consisting of hydrogen, halogen, C1-C3 haloalkyl, —CN, —OR⁵, —N(R⁵)R⁵, (═O), S(═O)₂N(R⁵)R⁵, aryl, heteroaryl, heterocyclyl, C1-C6 alkyl and C1-C6 alkyl substituted with —OH, —NH₂ or —S(═O)₂N(R⁵)R⁵;
wherein both R³ and R⁴ are (═O) if attached to a single sulfur atom that forms part of Y being a heterocycle;
or wherein R³ and R⁴, together with the structure to which they are attached, form an aromatic ring, a heteroaromatic ring, a saturated or unsaturated heterocyclic ring, or a fused or bridged ring structure of any of an aromatic ring, a heteroaromatic ring, and a saturated or unsaturated heterocyclic ring;
R⁵ is, at each occurrence, independently selected from the group consisting of hydrogen, C1-C6 alkyl, C3-C6 cycloalkyl, C1-C3 haloalkyl, heteroaryl, heterocyclyl, heteroaryl substituted with one or two of halogen, —OR¹¹, —N(R¹¹)R¹¹, C1-C6 alkyl and C1-C6 alkyl substituted with —OH, —NH₂; heterocyclyl substituted with one or two of halogen, —OR¹¹, —N(R¹¹)R¹¹, C1-C6 alkyl and C1-C6 alkyl substituted with —OH or —NH₂;
Z is any structure of the following group A; 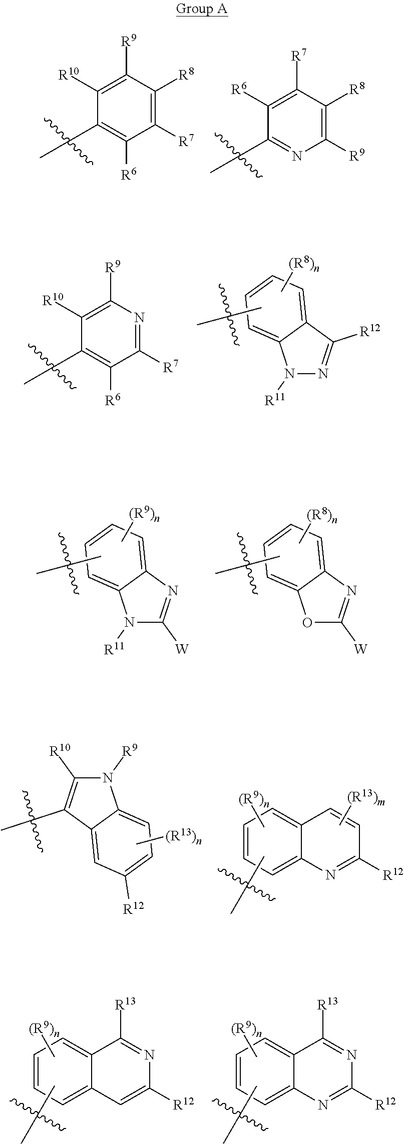
wherein n=1, 2, or 3; m=1, or 2;
R⁶ and R⁷ are, at each occurrence, independently selected from the group consisting of hydrogen, —NH(C═O)R¹⁴, —NHR¹⁴, —OR¹⁴ and any structure of the following group B, with the
proviso that, when Z is 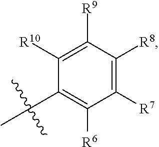
one of R6 and R7 is not H; 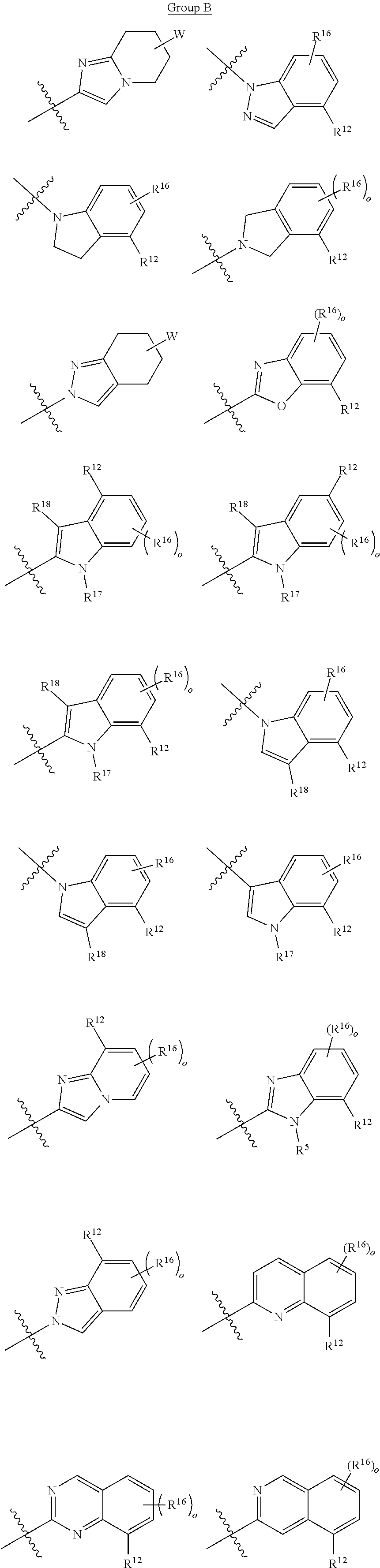 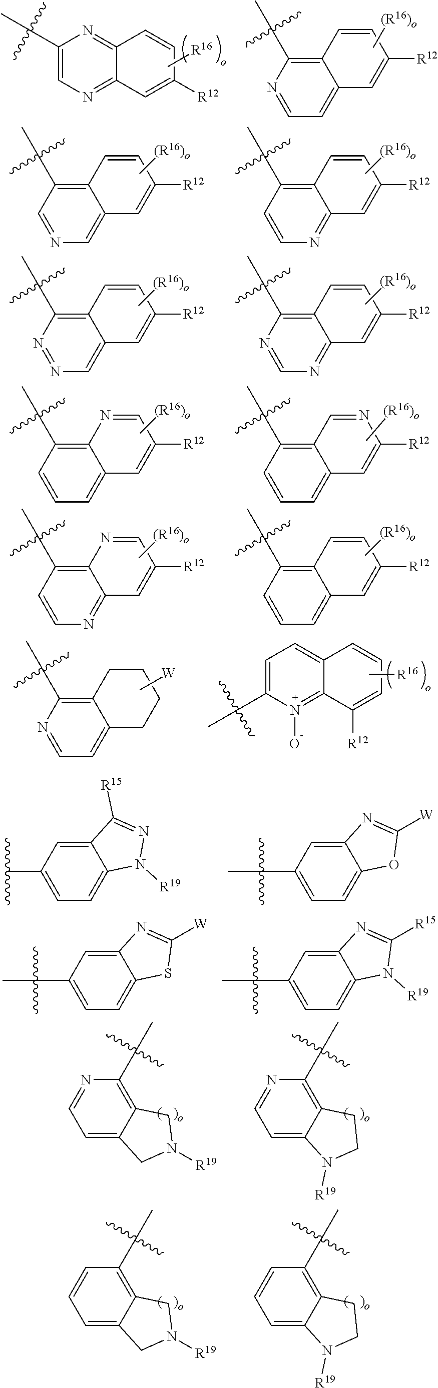
wherein o=1, 2 or 3;
W is any structure of the following group C; 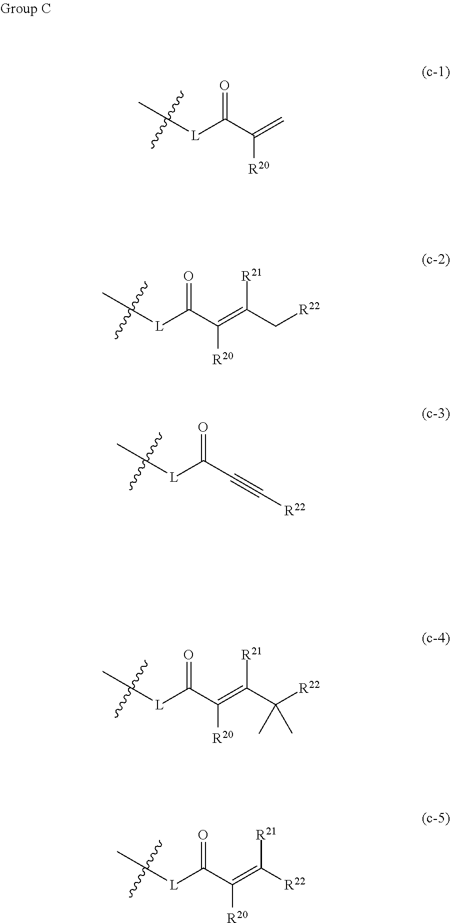
L is absent or, at each occurrence, independently selected from the group consisting of —O— and —NH—;
R⁸, R⁹ and R¹⁰ are, at each occurrence, independently selected from the group consisting of hydrogen, halogen, C1-C6 alkyl, C1-C3 haloalkyl, —OR⁵, —CN and C1-C6 alkyl substituted with —OH, —R or —NHPR⁵;
R¹¹ is, at each occurrence, independently selected from the group consisting of hydrogen, C1-C6 alkyl, C3-C10 cycloalkyl and W, as defined above;
R¹² is, at each occurrence, independently selected from hydrogen and W, as defined above;
wherein if R¹¹ is W, R¹² is hydrogen;
R¹³ is, at each occurrence, independently selected from the group consisting of hydrogen, halogen, C1-C6 alkyl, C1-C3 haloalkyl, —NH₂, —OR⁵, —CN and W, as defined above;
wherein if R¹³ is W, R¹² is hydrogen;
R¹⁴ is any structure of group D; 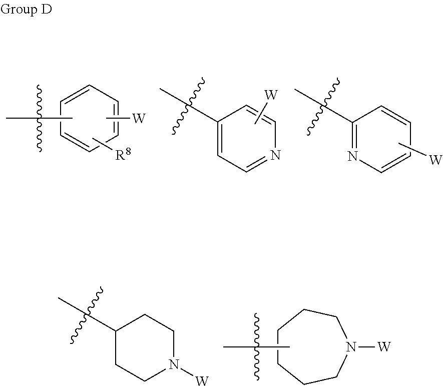
R¹⁵ is, at each occurrence, independently selected from hydrogen and W, as defined above;
R¹⁶ is, at each occurrence, independently selected from the group consisting of hydrogen, halogen, C1-C6 alkyl, C1-C3 haloalkyl, —NH₂, —OR⁵, —CN and W, as defined above;
wherein if R¹⁶ is W, R¹² is hydrogen;
R¹⁷ is, at each occurrence, independently selected from the group consisting of hydrogen, C1-C6 alkyl and C1-C3 haloalkyl;
R¹⁸ is, at each occurrence, independently selected from the group consisting of hydrogen, halogen, C1-C6 alkyl, C1-C3 haloalkyl, —NH₂, —OR⁵ and —CN;
R¹⁹ is, at each occurrence, independently selected from the group consisting of hydrogen, C1-C6 alkyl, C3-C10 cycloalkyl and W, as defined above;
wherein if R¹⁹ is W, R¹⁵ is hydrogen;
R²⁰ and R²¹ are, at each occurrence, independently selected from the group consisting of hydrogen, halogen, C1-C6 alkyl, C1-C3 haloalkyl, —OR⁵, heterocyclyl and —CN; and
R²² is, at each occurrence, independently selected from the group consisting of hydrogen, halogen, C1-C6 alkyl, C3-C10 cycloalkyl, —N(R⁵)₂, —NR¹⁹R²⁰, heterocyclyl, —OR⁵ and —CN.

16. The method according to claim 1,
wherein at least one of Z, R⁶, R⁷, R¹¹, R¹², R¹³, R¹⁵, R¹⁶ and R¹⁹ is W, as defined in claim 1, or is a structure containing W, as defined in claim 1.

17. The method according to claim 1,
wherein exactly one of Z, R⁶, R⁷, R¹¹, R¹², R¹³, R¹⁵, R¹⁶ and R¹⁹ is W, as defined in claim 1, or is a structure containing W, as defined in claim 1.

18. The method according to claim 1,
wherein R¹ is hydrogen and the compound has the general formula II 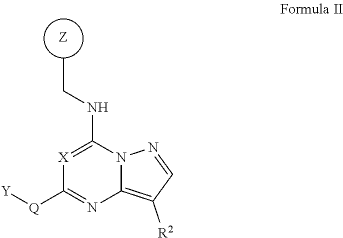
wherein X, Y, Z, R² and Q are as defined in claim 1.

19. The method according to claim 1,
wherein said compound has the general formula III 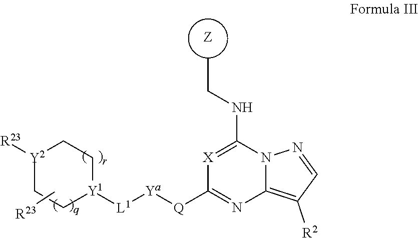
wherein X, Z, R² and Q are as defined in claim 1, and
Y^{a} is either absent or independently, at each occurrence, selected from the group consisting of aryl, heteroaryl, heterocyclyl, aryl substituted with one or two of C1-C6 alkyl, —OR⁵, —N(R⁵)R⁵, and halogen, heteroaryl substituted with one or two of C1-C6 alkyl, —OR⁵, N(R⁵)R⁵ and halogen, heterocyclyl substituted with one or two of R²³ and R²⁴;
R²³ is either absent or independently, at each occurrence, selected from the group consisting of hydrogen, —OR⁵, halogen, —N(R⁵)R⁵, —NH(C═O)R⁵, —(C═O)NH₂, aryl, heteroaryl, heterocyclyl, C1-C6 alkyl and C1-C6 alkyl substituted with —OH or —NH₂;
R²⁴ is, independently, at each occurrence, selected from the group consisting of hydrogen, halogen, —OR⁵, —N(R⁵)R⁵, (═O), aryl, heteroaryl, heterocyclyl, C1-C6 alkyl and C1-C6 alkyl substituted with —OH or —NH₂;
wherein R⁵ is as defined in claim 1;
L¹ is either absent or independently, at each occurrence, selected from the group consisting of —NH—, —NH(CH₂)—, —NH(C═O)—, —NHSO₂—, —O—, —O(CH₂)—, —(C═O)—, —(C═O)NH— and —(C═O)(CH₂)—;
Y¹ is, independently at each occurrence, selected from CH, C(OH) and N;
Y² is, independently at each occurrence, selected from CH, C(OH), O and N;
q is, independently at each occurrence, selected from 0, 1 and 2; and
r is, independently at each occurrence, selected from 0, 1, 2 and 3.

20. The method according to claim 1,
wherein Z is Z¹, and Z¹ is any structure of the following group E; 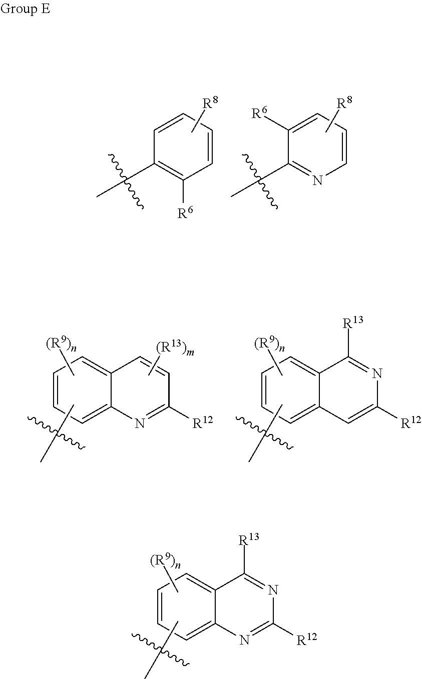
wherein m is, independently at each occurrence, selected from 1 and 2; and
n is as defined in claim 1;
R⁸, R⁹, R¹² and R¹³ are as defined in claim 1; and
R⁶ is any structure of group B as defined in claim 1.

21. The method according to claim 1,
wherein
Z is 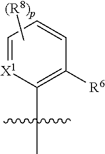
p is, independently at each occurrence, selected from 0, 1, 2 and 3;
X¹ is, independently at each occurrence, selected from CR⁸ and N;
R⁶ is any structure of group B, as defined in claim 1; and
R⁸ is as defined in claim 1.

22. The method according to claim 1,
wherein
Z is 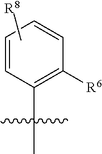
or Z is 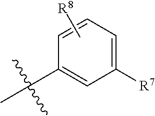
wherein R⁶-R⁸ are as defined in claim 1.

23. The method according to claim 1,
wherein
Z is 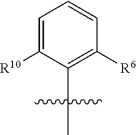
and wherein R⁶ and R¹⁰ are as defined in claim 1.

24. The method according to claim 1,
wherein R¹ is hydrogen, Z is 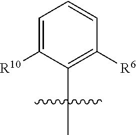
and wherein R⁶ and R¹⁰ are as defined in claim 1.

25. The method according to claim 1,
wherein said compound has the general formula IV 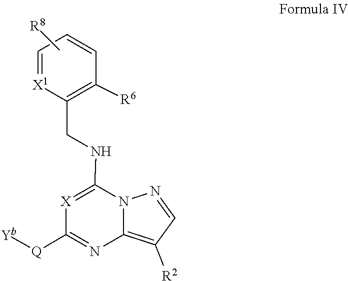
wherein X, X¹, R⁶, R⁸ and Q are as defined in claim 1, and
X¹ is independently at each occurrence, selected from CR⁸ and N;
wherein Y^{b} is any structure of the following group F; 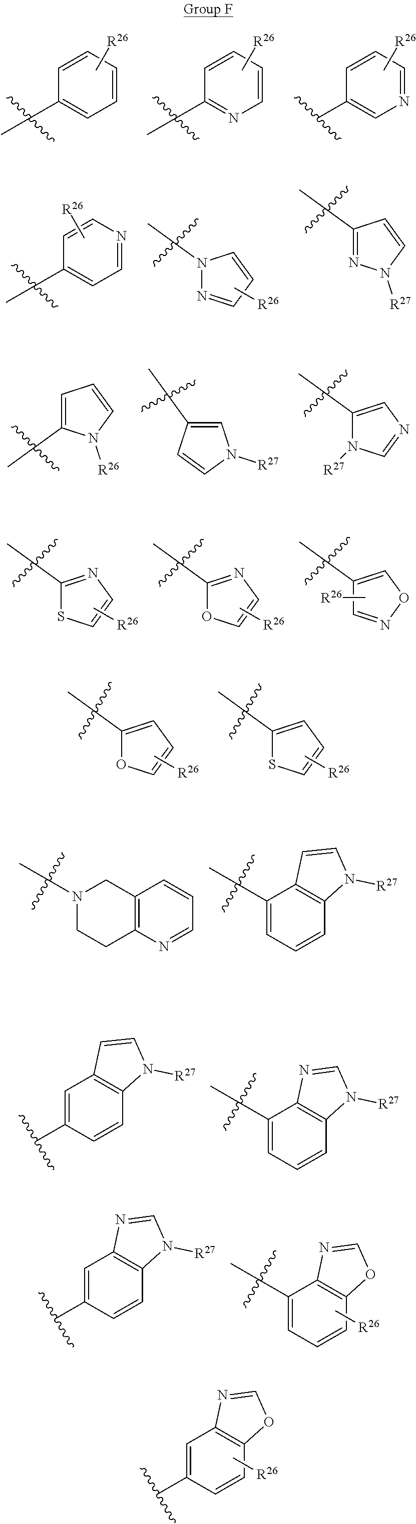
R²⁶ and R²⁷ is either absent or independently, at each occurrence, selected from the group consisting of hydrogen, —OR⁵, halogen, —N(R⁵)R⁵, —NH(C═O)R⁵, —(C═O)NH₂, aryl, heteroaryl, heterocyclyl, C1-C6 alkyl and C1-C6 alkyl substituted with —OH or —NH₂;
wherein R⁵ is as defined in claim 1.

26. The method according to claim 1, wherein R² is C1-C6 alkyl or C1-C3 haloalkyl.

27. The method according to claim 1, wherein R⁶ is

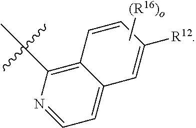

28. The method according to claim 27, wherein R¹⁶ is hydrogen; o is 1; R¹² is W; W is (c−1) or (c−2) or (c−3); L is —NH—; R²⁰ and R²¹ are, independently, at each occurrence, hydrogen, halogen, or C1-C6 alkyl; and wherein R²² is hydrogen, halogen, C1-C6 alkyl, —N(R⁵)₂, or —NR¹⁹R²⁰.

29. The method according to claim 1, wherein said compound is a compound having a structure selected from structures 1-216, as defined in the following table:

| #cpds | Structure |
|---|---|
| 1 | 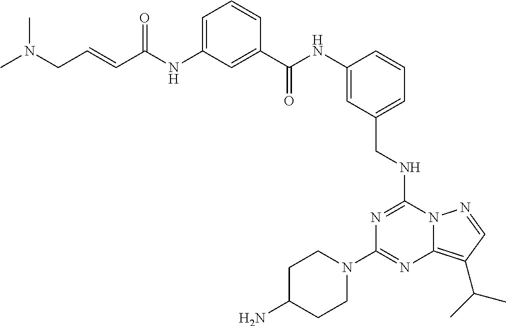 [SMILES: CC(C)c1cnn2c(NCc3cccc(NC(=O)c4cccc(NC(=O)/C=C/CN(C)C)c4)c3)nc(N3CCC(N)CC3)nc12] |
| 2 | 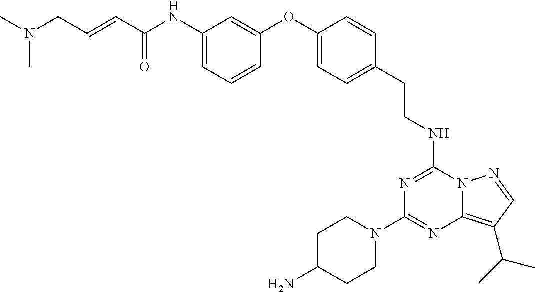 [SMILES: CC(C)c1cnn2c(NCCc3ccc(Oc4cccc(NC(=O)/C=C/CN(C)C)c4)cc3)nc(N3CCC(N)CC3)nc12] |
| 3 | 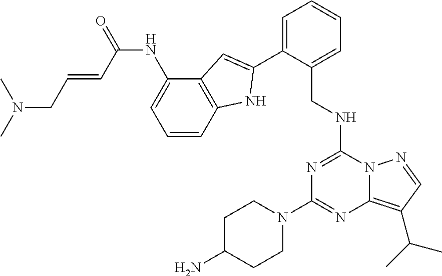 [SMILES: CC(C)c1cnn2c(NCc3ccccc3-c3cc4c(NC(=O)/C=C/CN(C)C)cccc4[nH]3)nc(N3CCC(N)CC3)nc12] |
| 4 | 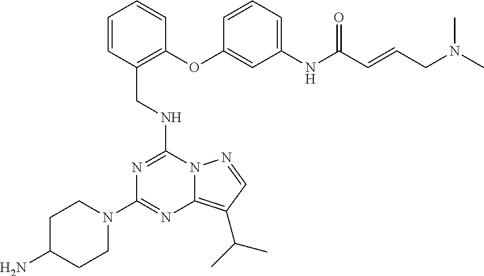 [SMILES: CC(C)c1cnn2c(NCc3ccccc3Oc3cccc(NC(=O)/C=C/CN(C)C)c3)nc(N3CCC(N)CC3)nc12] |
| 5 | 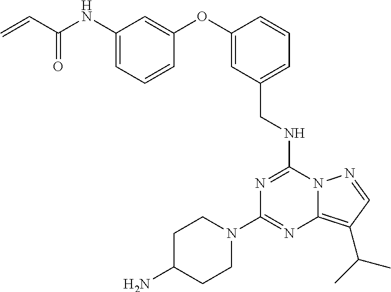 [SMILES: C=CC(=O)Nc1cccc(Oc2cccc(CNc3nc(N4CCC(N)CC4)nc4c(C(C)C)cnn34)c2)c1] |
| 7 | 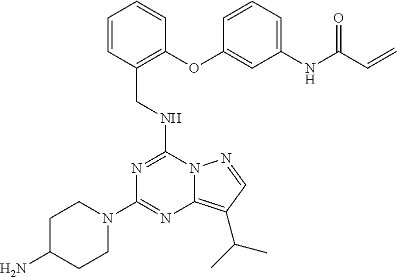 [SMILES: C=CC(=O)Nc1cccc(Oc2ccccc2CNc2nc(N3CCC(N)CC3)nc3c(C(C)C)cnn23)c1] |
| 8 | 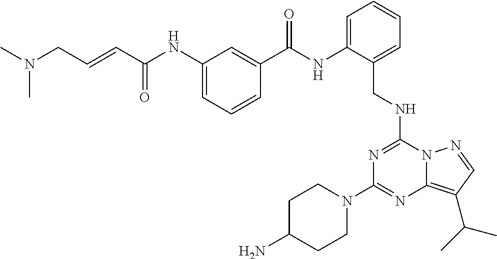 [SMILES: CC(C)c1cnn2c(NCc3ccccc3NC(=O)c3cccc(NC(=O)/C=C/CN(C)C)c3)nc(N3CCC(N)CC3)nc12] |
| 9 | 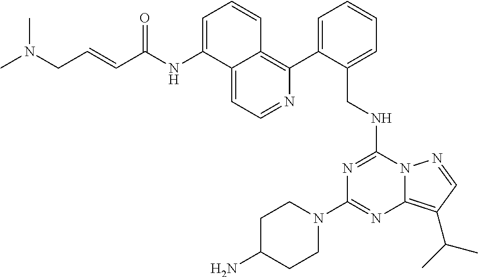 [SMILES: CC(C)c1cnn2c(NCc3ccccc3-c3nccc4c(NC(=O)/C=C/CN(C)C)cccc34)nc(N3CCC(N)CC3)nc12] |
| 10 | 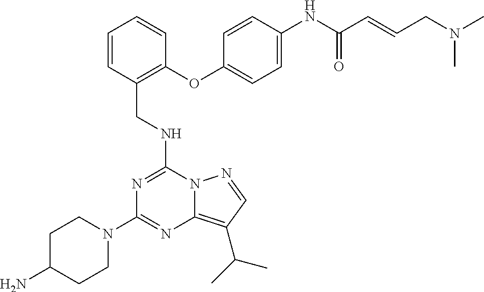 [SMILES: CC(C)c1cnn2c(NCc3ccccc3Oc3ccc(NC(=O)/C=C/CN(C)C)cc3)nc(N3CCC(N)CC3)nc12] |
| 11 | 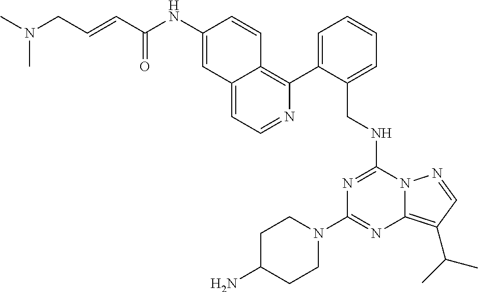 [SMILES: CC(C)c1cnn2c(NCc3ccccc3-c3nccc4cc(NC(=O)/C=C/CN(C)C)ccc34)nc(N3CCC(N)CC3)nc12] |
| 6 | 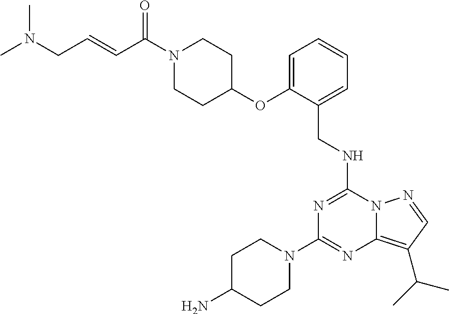 [SMILES: CC(C)c1cnn2c(NCc3ccccc3OC3CCN(C(=O)/C=C/CN(C)C)CC3)nc(N3CCC(N)CC3)nc12] |
| 13 | 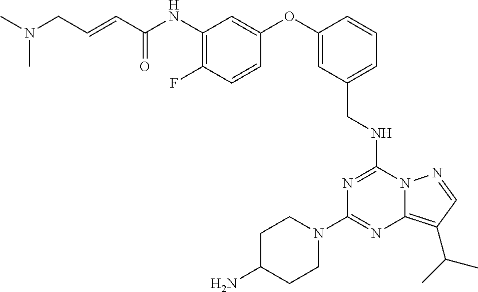 [SMILES: CC(C)c1cnn2c(NCc3cccc(Oc4ccc(F)c(NC(=O)/C=C/CN(C)C)c4)c3)nc(N3CCC(N)CC3)nc12] |
| 14 | 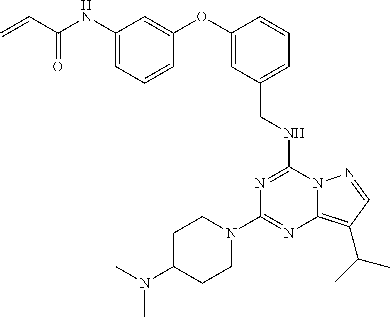 [SMILES: C=CC(=O)Nc1cccc(Oc2cccc(CNc3nc(N4CCC(N(C)C)CC4)nc4c(C(C)C)cnn34)c2)c1] |
| 15 | 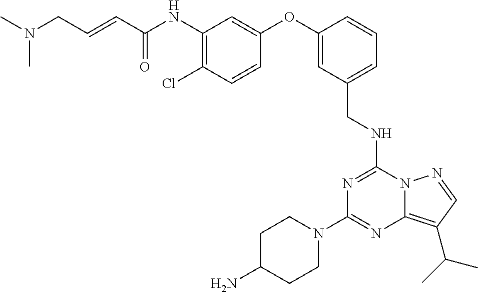 [SMILES: CC(C)c1cnn2c(NCc3cccc(Oc4ccc(Cl)c(NC(=O)/C=C/CN(C)C)c4)c3)nc(N3CCC(N)CC3)nc12] |
| 16 | 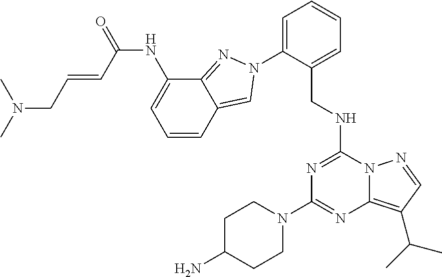 [SMILES: CC(C)c1cnn2c(NCc3ccccc3-n3cc4cccc(NC(=O)/C=C/CN(C)C)c4n3)nc(N3CCC(N)CC3)nc12] |
| 12 | 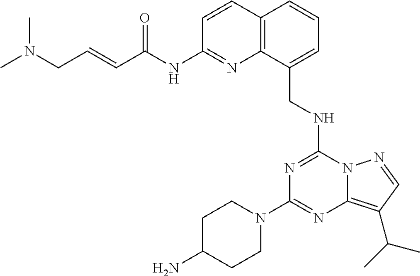 [SMILES: CC(C)c1cnn2c(NCc3cccc4ccc(NC(=O)/C=C/CN(C)C)nc34)nc(N3CCC(N)CC3)nc12] |
| 19 | 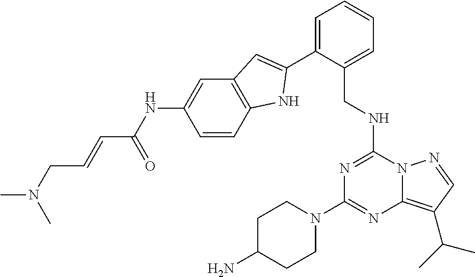 [SMILES: CC(C)c1cnn2c(NCc3ccccc3-c3cc4cc(NC(=O)/C=C/CN(C)C)ccc4[nH]3)nc(N3CCC(N)CC3)nc12] |
| 20 | 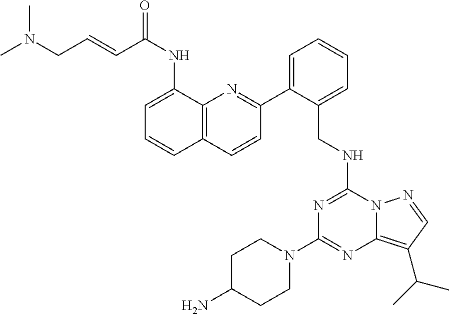 [SMILES: CC(C)c1cnn2c(NCc3ccccc3-c3ccc4cccc(NC(=O)/C=C/CN(C)C)c4n3)nc(N3CCC(N)CC3)nc12] |
| 21 | 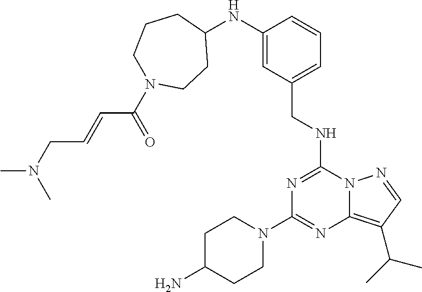 [SMILES: CC(C)c1cnn2c(NCc3cccc(NC4CCCN(C(=O)/C=C/CN(C)C)CC4)c3)nc(N3CCC(N)CC3)nc12] |
| 22 | 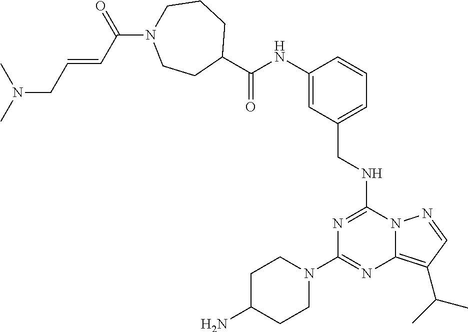 [SMILES: CC(C)c1cnn2c(NCc3cccc(NC(=O)C4CCCN(C(=O)/C=C/CN(C)C)CC4)c3)nc(N3CCC(N)CC3)nc12] |
| 17 | 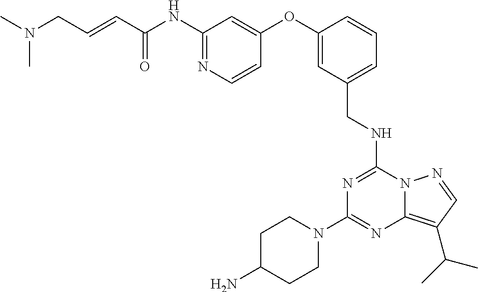 [SMILES: CC(C)c1cnn2c(NCc3cccc(Oc4ccnc(NC(=O)/C=C/CN(C)C)c4)c3)nc(N3CCC(N)CC3)nc12] |
| 18 | 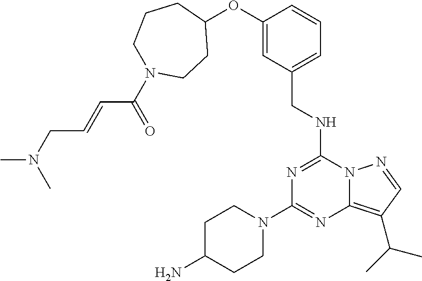 [SMILES: CC(C)c1cnn2c(NCc3cccc(OC4CCCN(C(=O)/C=C/CN(C)C)CC4)c3)nc(N3CCC(N)CC3)nc12] |
| 25 | 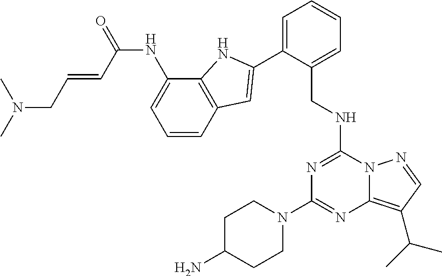 [SMILES: CC(C)c1cnn2c(NCc3ccccc3-c3cc4cccc(NC(=O)/C=C/CN(C)C)c4[nH]3)nc(N3CCC(N)CC3)nc12] |
| 26 | 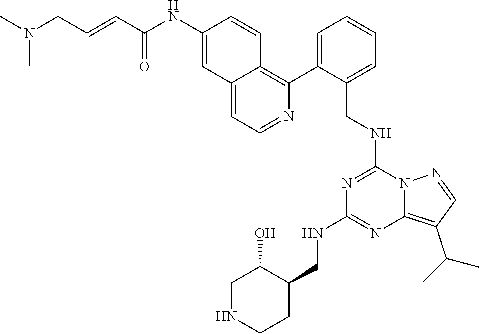 [SMILES: CC(C)c1cnn2c(NCc3ccccc3-c3nccc4cc(NC(=O)/C=C/CN(C)C)ccc34)nc(NC[C@H]3CCNC[C@@H]3O)nc12] |
| 27 | 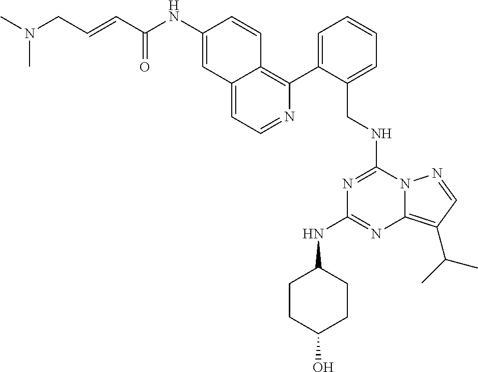 |
| 23 | 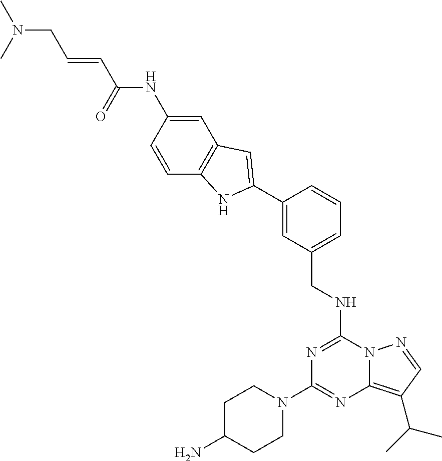 [SMILES: CC(C)c1cnn2c(NCc3cccc(-c4cc5cc(NC(=O)/C=C/CN(C)C)ccc5[nH]4)c3)nc(N3CCC(N)CC3)nc12] |
| 24 | 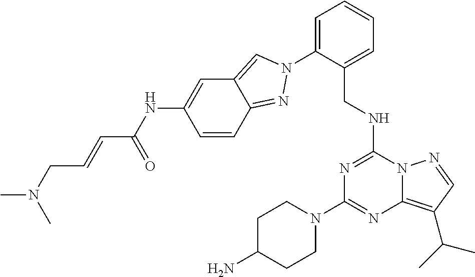 [SMILES: CC(C)c1cnn2c(NCc3ccccc3-n3cc4cc(NC(=O)/C=C/CN(C)C)ccc4n3)nc(N3CCC(N)CC3)nc12] |
| 31 | 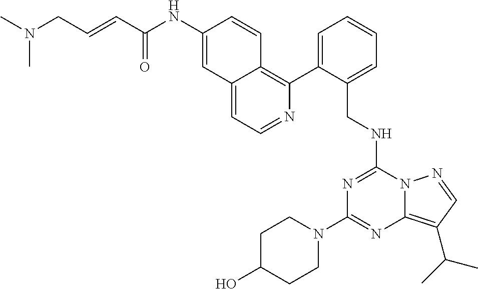 [SMILES: CC(C)c1cnn2c(NCc3ccccc3-c3nccc4cc(NC(=O)/C=C/CN(C)C)ccc34)nc(N3CCC(O)CC3)nc12] |
| 32 | 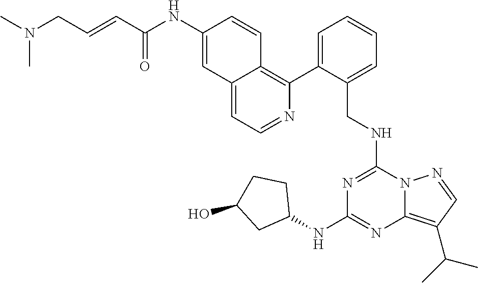 |
| 33 | 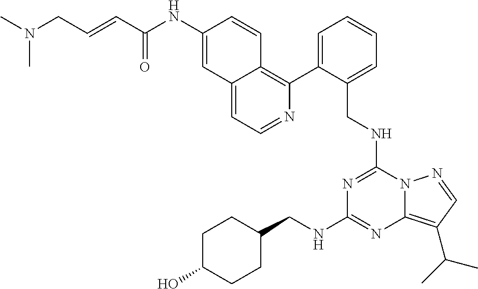 |
| 28 | 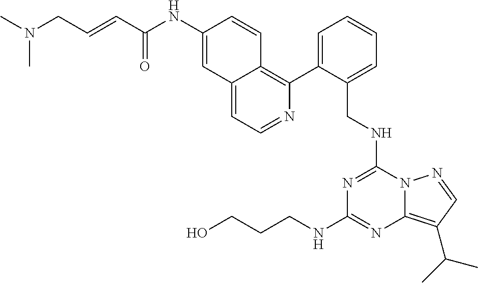 [SMILES: CC(C)c1cnn2c(NCc3ccccc3-c3nccc4cc(NC(=O)/C=C/CN(C)C)ccc34)nc(NCCCO)nc12] |
| 29 | 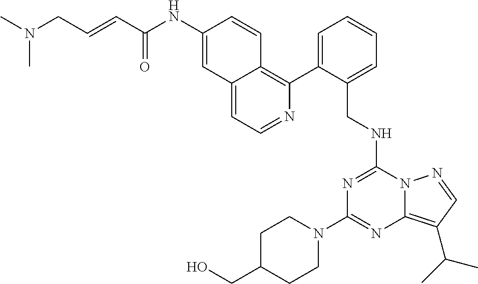 [SMILES: CC(C)c1cnn2c(NCc3ccccc3-c3nccc4cc(NC(=O)/C=C/CN(C)C)ccc34)nc(N3CCC(CO)CC3)nc12] |
| 30 | 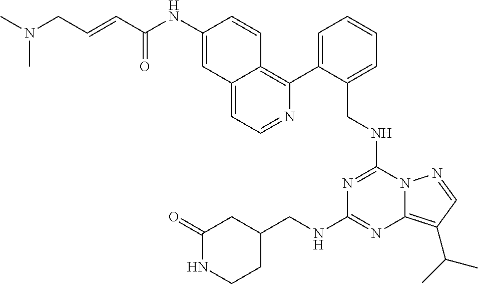 [SMILES: CC(C)c1cnn2c(NCc3ccccc3-c3nccc4cc(NC(=O)/C=C/CN(C)C)ccc34)nc(NCC3CCNC(=O)C3)nc12] |
| 37 | 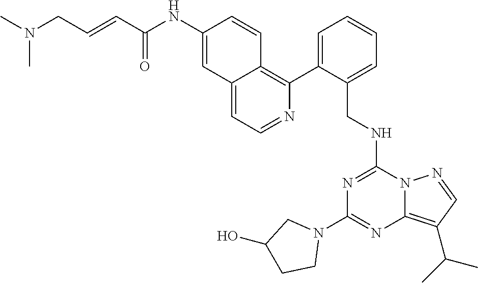 [SMILES: CC(C)c1cnn2c(NCc3ccccc3-c3nccc4cc(NC(=O)/C=C/CN(C)C)ccc34)nc(N3CCC(O)C3)nc12] |
| 38 | 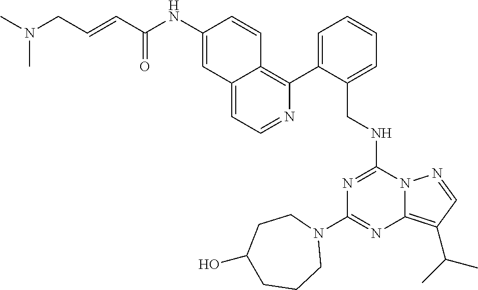 [SMILES: CC(C)c1cnn2c(NCc3ccccc3-c3nccc4cc(NC(=O)/C=C/CN(C)C)ccc34)nc(N3CCCC(O)CC3)nc12] |
| 34 | 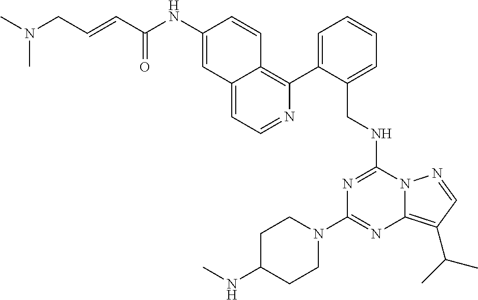 [SMILES: CNC1CCN(c2nc(NCc3ccccc3-c3nccc4cc(NC(=O)/C=C/CN(C)C)ccc34)n3ncc(C(C)C)c3n2)CC1] |
| 35 | 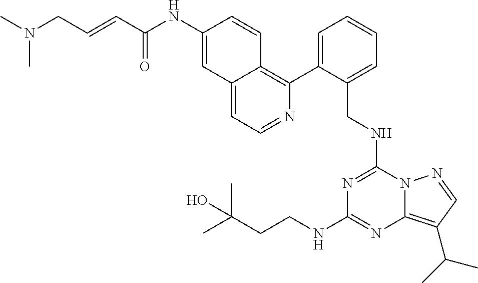 [SMILES: CC(C)c1cnn2c(NCc3ccccc3-c3nccc4cc(NC(=O)/C=C/CN(C)C)ccc34)nc(NCCC(C)(C)O)nc12] |
| 36 | 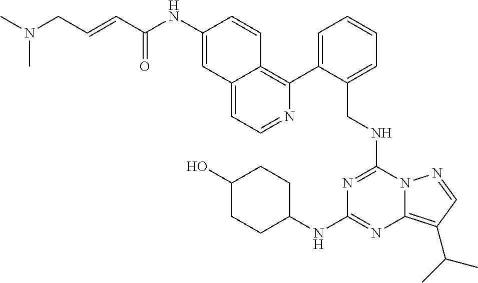 [SMILES: CC(C)c1cnn2c(NCc3ccccc3-c3nccc4cc(NC(=O)/C=C/CN(C)C)ccc34)nc(NC3CCC(O)CC3)nc12] |
| 43 | 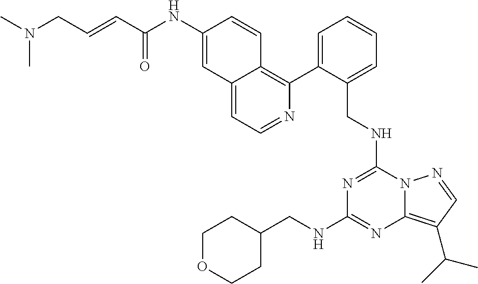 [SMILES: CC(C)c1cnn2c(NCc3ccccc3-c3nccc4cc(NC(=O)/C=C/CN(C)C)ccc34)nc(NCC3CCOCC3)nc12] |
| 44 | 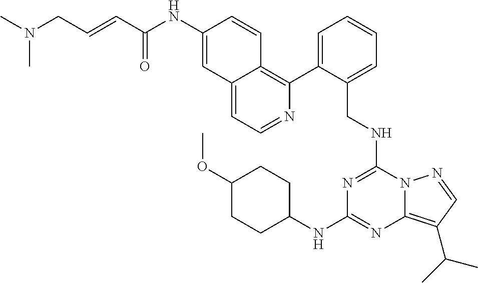 [SMILES: COC1CCC(Nc2nc(NCc3ccccc3-c3nccc4cc(NC(=O)/C=C/CN(C)C)ccc34)n3ncc(C(C)C)c3n2)CC1] |
| 39 | 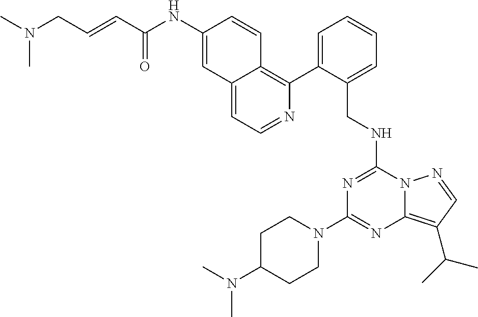 [SMILES: CC(C)c1cnn2c(NCc3ccccc3-c3nccc4cc(NC(=O)/C=C/CN(C)C)ccc34)nc(N3CCC(N(C)C)CC3)nc12] |
| 40 | 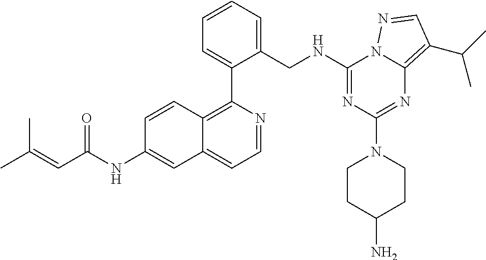 [SMILES: CC(C)=CC(=O)Nc1ccc2c(-c3ccccc3CNc3nc(N4CCC(N)CC4)nc4c(C(C)C)cnn34)nccc2c1] |
| 41 | 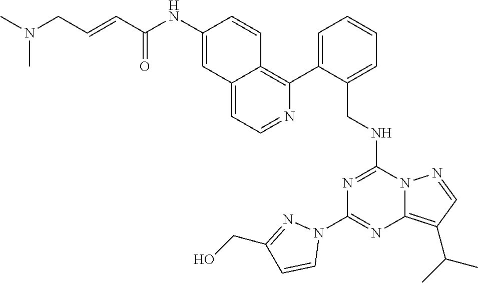 [SMILES: CC(C)c1cnn2c(NCc3ccccc3-c3nccc4cc(NC(=O)/C=C/CN(C)C)ccc34)nc(-n3ccc(CO)n3)nc12] |
| 42 | 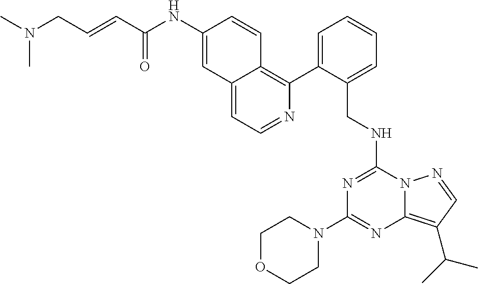 [SMILES: CC(C)c1cnn2c(NCc3ccccc3-c3nccc4cc(NC(=O)/C=C/CN(C)C)ccc34)nc(N3CCOCC3)nc12] |
| 49 | 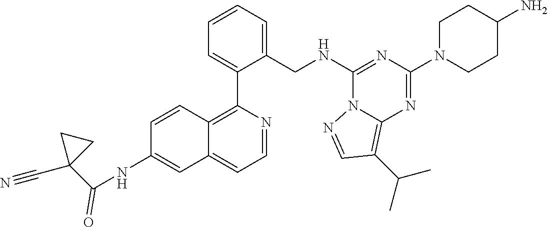 [SMILES: CC(C)c1cnn2c(NCc3ccccc3-c3nccc4cc(NC(=O)C5(C#N)CC5)ccc34)nc(N3CCC(N)CC3)nc12] |
| 45 | 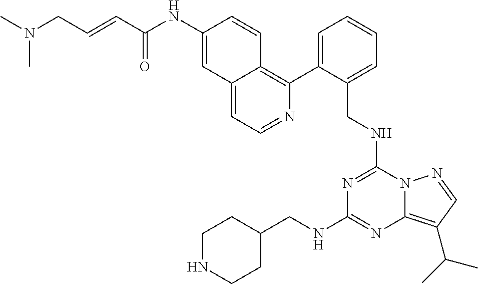 [SMILES: CC(C)c1cnn2c(NCc3ccccc3-c3nccc4cc(NC(=O)/C=C/CN(C)C)ccc34)nc(NCC3CCNCC3)nc12] |
| 46 | 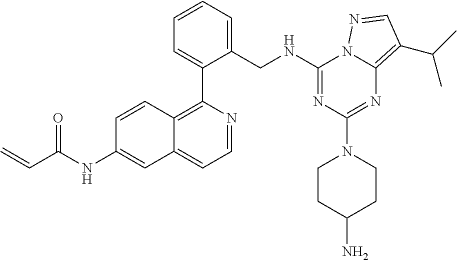 [SMILES: C=CC(=O)Nc1ccc2c(-c3ccccc3CNc3nc(N4CCC(N)CC4)nc4c(C(C)C)cnn34)nccc2c1] |
| 47 | 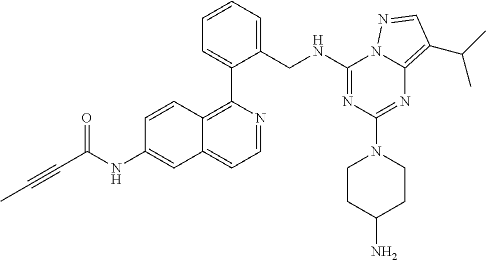 [SMILES: CC#CC(=O)Nc1ccc2c(-c3ccccc3CNc3nc(N4CCC(N)CC4)nc4c(C(C)C)cnn34)nccc2c1] |
| 48 | 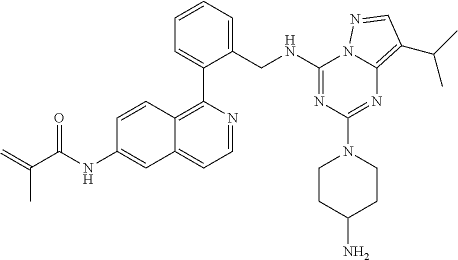 [SMILES: C=C(C)C(=O)Nc1ccc2c(-c3ccccc3CNc3nc(N4CCC(N)CC4)nc4c(C(C)C)cnn34)nccc2c1] |
| 55 | 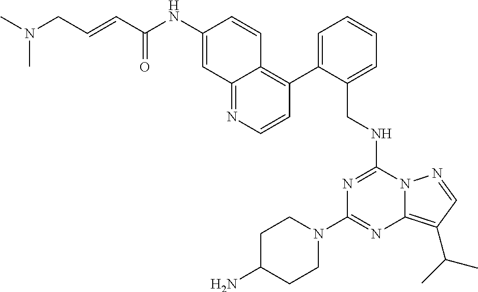 [SMILES: CC(C)c1cnn2c(NCc3ccccc3-c3ccnc4cc(NC(=O)/C=C/CN(C)C)ccc34)nc(N3CCC(N)CC3)nc12] |
| 50 | 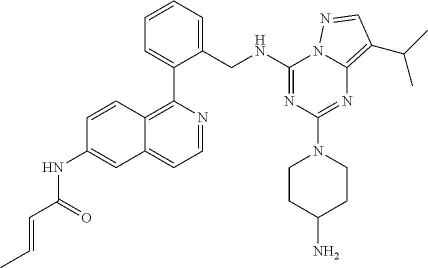 [SMILES: C/C=C/C(=O)Nc1ccc2c(-c3ccccc3CNc3nc(N4CCC(N)CC4)nc4c(C(C)C)cnn34)nccc2c1] |
| 51 | 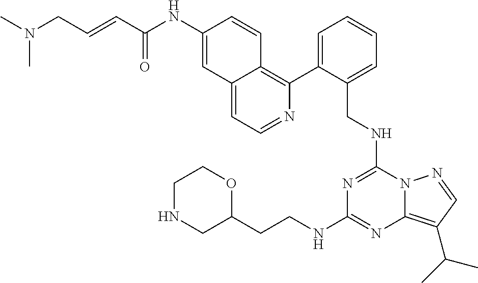 [SMILES: CC(C)c1cnn2c(NCc3ccccc3-c3nccc4cc(NC(=O)/C=C/CN(C)C)ccc34)nc(NCCC3CNCCO3)nc12] |
| 52 | 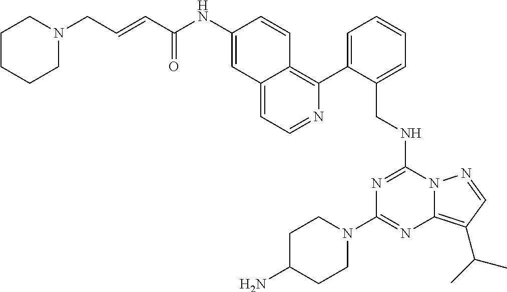 [SMILES: CC(C)c1cnn2c(NCc3ccccc3-c3nccc4cc(NC(=O)/C=C/CN5CCCCC5)ccc34)nc(N3CCC(N)CC3)nc12] |
| 53 | 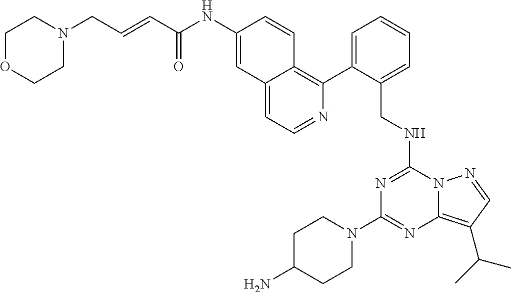 [SMILES: CC(C)c1cnn2c(NCc3ccccc3-c3nccc4cc(NC(=O)/C=C/CN5CCOCC5)ccc34)nc(N3CCC(N)CC3)nc12] |
| 54 | 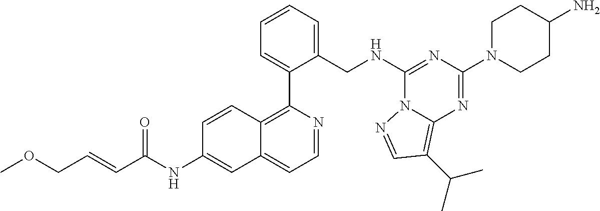 [SMILES: COC/C=C/C(=O)Nc1ccc2c(-c3ccccc3CNc3nc(N4CCC(N)CC4)nc4c(C(C)C)cnn34)nccc2c1] |
| 56 | 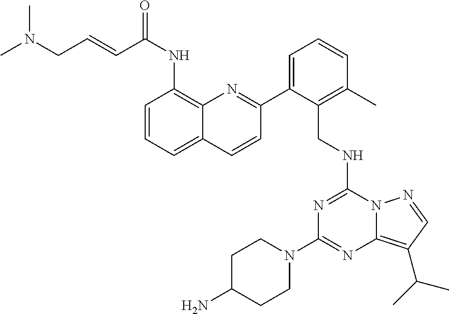 [SMILES: Cc1cccc(-c2ccc3cccc(NC(=O)/C=C/CN(C)C)c3n2)c1CNc1nc(N2CCC(N)CC2)nc2c(C(C)C)cnn12] |
| 57 | 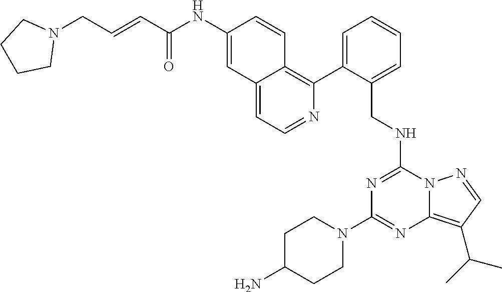 [SMILES: CC(C)c1cnn2c(NCc3ccccc3-c3nccc4cc(NC(=O)/C=C/CN5CCCC5)ccc34)nc(N3CCC(N)CC3)nc12] |
| 58 | 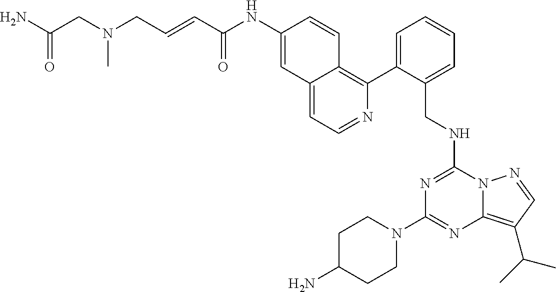 [SMILES: CC(C)c1cnn2c(NCc3ccccc3-c3nccc4cc(NC(=O)/C=C/CN(C)CC(N)=O)ccc34)nc(N3CCC(N)CC3)nc12] |
| 59 | 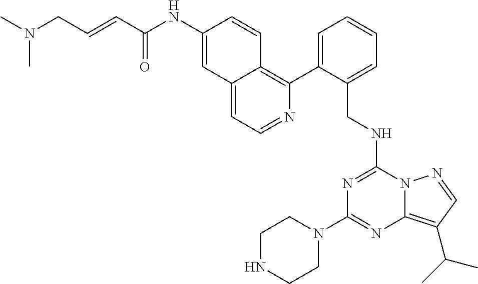 [SMILES: CC(C)c1cnn2c(NCc3ccccc3-c3nccc4cc(NC(=O)/C=C/CN(C)C)ccc34)nc(N3CCNCC3)nc12] |
| 60 | 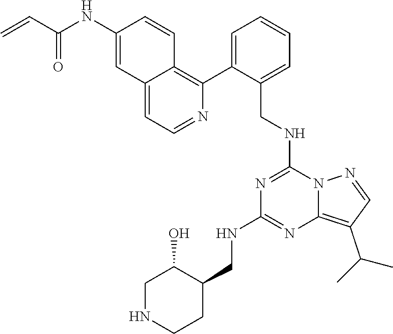 [SMILES: C=CC(=O)Nc1ccc2c(-c3ccccc3CNc3nc(NC[C@H]4CCNC[C@@H]4O)nc4c(C(C)C)cnn34)nccc2c1] |
| 61 | 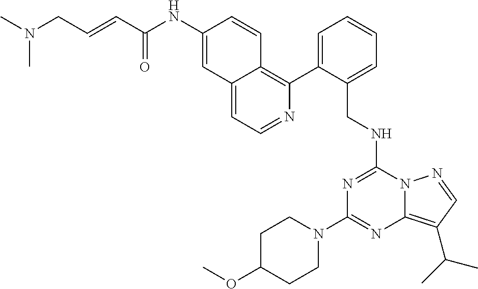 [SMILES: COC1CCN(c2nc(NCc3ccccc3-c3nccc4cc(NC(=O)/C=C/CN(C)C)ccc34)n3ncc(C(C)C)c3n2)CC1] |
| 62 | 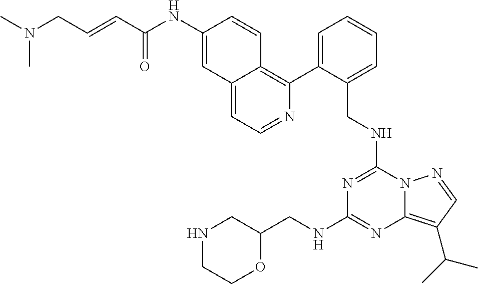 [SMILES: CC(C)c1cnn2c(NCc3ccccc3-c3nccc4cc(NC(=O)/C=C/CN(C)C)ccc34)nc(NCC3CNCCO3)nc12] |
| 63 | 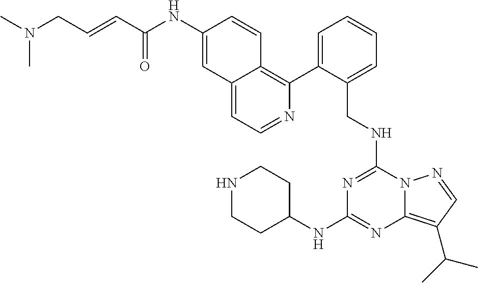 [SMILES: CC(C)c1cnn2c(NCc3ccccc3-c3nccc4cc(NC(=O)/C=C/CN(C)C)ccc34)nc(NC3CCNCC3)nc12] |
| 64 | 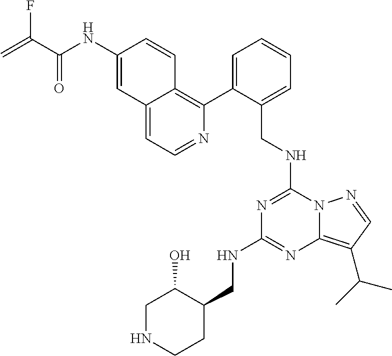 [SMILES: C=C(F)C(=O)Nc1ccc2c(-c3ccccc3CNc3nc(NC[C@H]4CCNC[C@@H]4O)nc4c(C(C)C)cnn34)nccc2c1] |
| 65 | 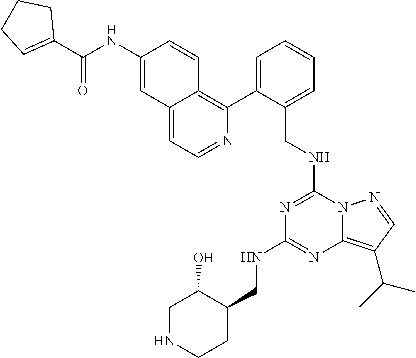 [SMILES: CC(C)c1cnn2c(NCc3ccccc3-c3nccc4cc(NC(=O)C5=CCCC5)ccc34)nc(NC[C@H]3CCNC[C@@H]3O)nc12] |
| 67 | 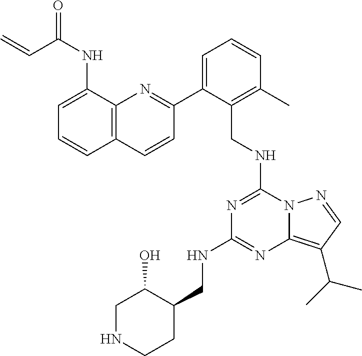 [SMILES: C=CC(=O)Nc1cccc2ccc(-c3cccc(C)c3CNc3nc(NC[C@H]4CCNC[C@@H]4O)nc4c(C(C)C)cnn34)nc12] |
| 68 | 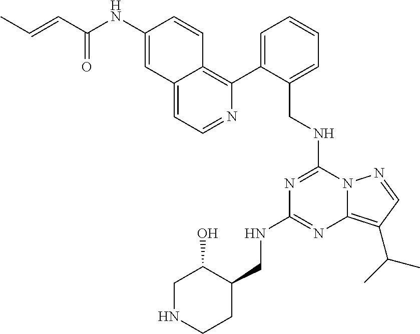 [SMILES: C/C=C/C(=O)Nc1ccc2c(-c3ccccc3CNc3nc(NC[C@H]4CCNC[C@@H]4O)nc4c(C(C)C)cnn34)nccc2c1] |
| 69 | 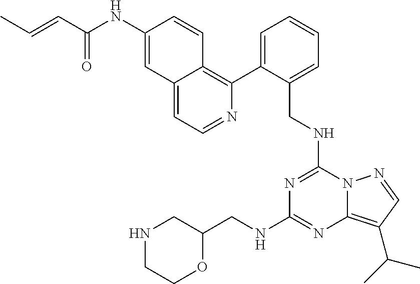 [SMILES: C/C=C/C(=O)Nc1ccc2c(-c3ccccc3CNc3nc(NCC4CNCCO4)nc4c(C(C)C)cnn34)nccc2c1] |
| 70 | 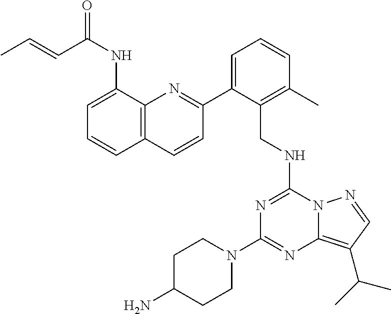 [SMILES: C/C=C/C(=O)Nc1cccc2ccc(-c3cccc(C)c3CNc3nc(N4CCC(N)CC4)nc4c(C(C)C)cnn34)nc12] |
| 71 | 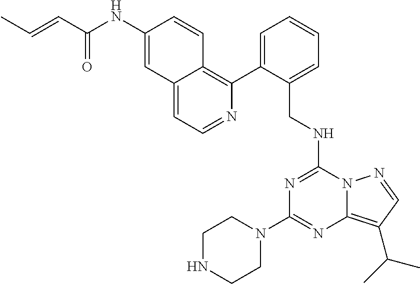 [SMILES: C/C=C/C(=O)Nc1ccc2c(-c3ccccc3CNc3nc(N4CCNCC4)nc4c(C(C)C)cnn34)nccc2c1] |
| 66 | 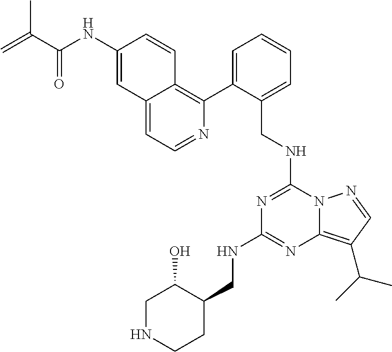 [SMILES: C=C(C)C(=O)Nc1ccc2c(-c3ccccc3CNc3nc(NC[C@H]4CCNC[C@@H]4O)nc4c(C(C)C)cnn34)nccc2c1] |
| 73 | 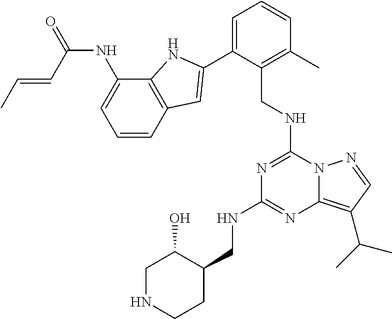 [SMILES: C/C=C/C(=O)Nc1cccc2cc(-c3cccc(C)c3CNc3nc(NC[C@H]4CCNC[C@@H]4O)nc4c(C(C)C)cnn34)[nH]c12] |
| 74 | 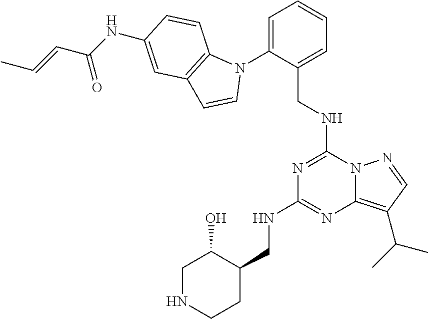 [SMILES: C/C=C/C(=O)Nc1ccc2c(ccn2-c2ccccc2CNc2nc(NC[C@H]3CCNC[C@@H]3O)nc3c(C(C)C)cnn23)c1] |
| 75 | 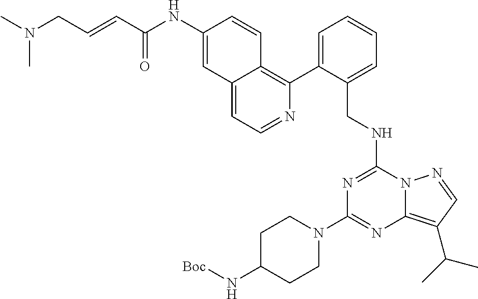 [SMILES: CC(C)c1cnn2c(NCc3ccccc3-c3nccc4cc(NC(=O)/C=C/CN(C)C)ccc34)nc(N3CCC(NC(=O)OC(C)(C)C)CC3)nc12] |
| 76 | 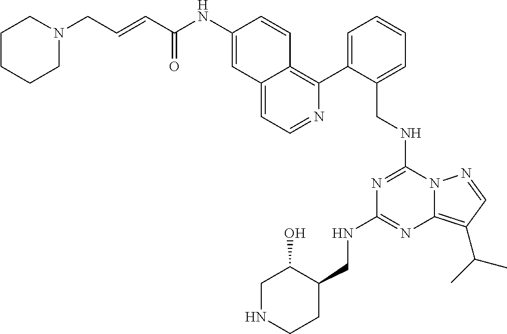 [SMILES: CC(C)c1cnn2c(NCc3ccccc3-c3nccc4cc(NC(=O)/C=C/CN5CCCCC5)ccc34)nc(NC[C@H]3CCNC[C@@H]3O)nc12] |
| 72 | 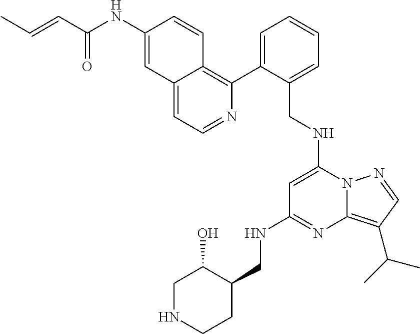 [SMILES: C/C=C/C(=O)Nc1ccc2c(-c3ccccc3CNc3cc(NC[C@H]4CCNC[C@@H]4O)nc4c(C(C)C)cnn34)nccc2c1] |
| 79 | 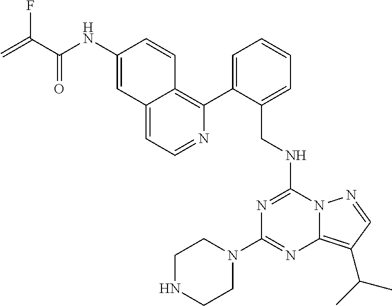 [SMILES: C=C(F)C(=O)Nc1ccc2c(-c3ccccc3CNc3nc(N4CCNCC4)nc4c(C(C)C)cnn34)nccc2c1] |
| 80 | 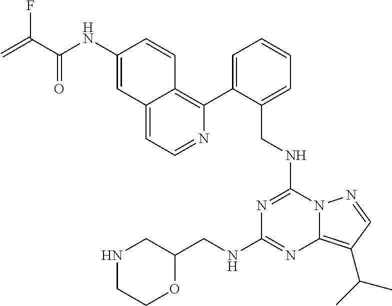 [SMILES: C=C(F)C(=O)Nc1ccc2c(-c3ccccc3CNc3nc(NCC4CNCCO4)nc4c(C(C)C)cnn34)nccc2c1] |
| 81 | 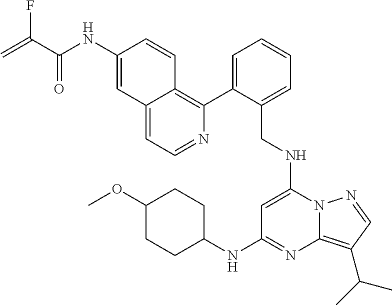 [SMILES: C=C(F)C(=O)Nc1ccc2c(-c3ccccc3CNc3cc(NC4CCC(OC)CC4)nc4c(C(C)C)cnn34)nccc2c1] |
| 82 | 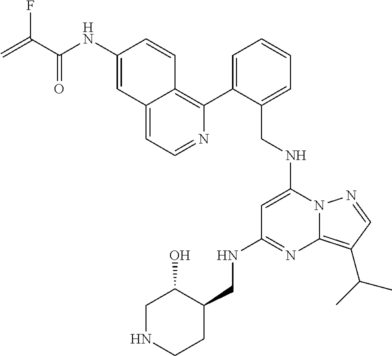 [SMILES: C=C(F)C(=O)Nc1ccc2c(-c3ccccc3CNc3cc(NC[C@H]4CCNC[C@@H]4O)nc4c(C(C)C)cnn34)nccc2c1] |
| 77 | 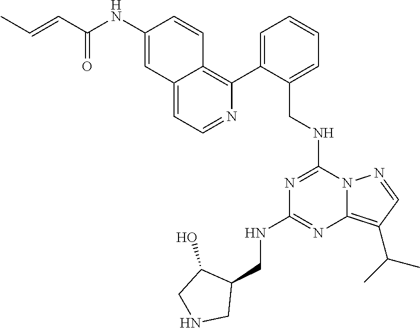 [SMILES: C/C=C/C(=O)Nc1ccc2c(-c3ccccc3CNc3nc(NC[C@H]4CNC[C@@H]4O)nc4c(C(C)C)cnn34)nccc2c1] |
| 78 | 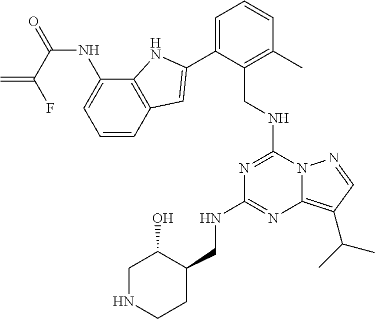 [SMILES: C=C(F)C(=O)Nc1cccc2cc(-c3cccc(C)c3CNc3nc(NC[C@H]4CCNC[C@@H]4O)nc4c(C(C)C)cnn34)[nH]c12] |
| 85 | 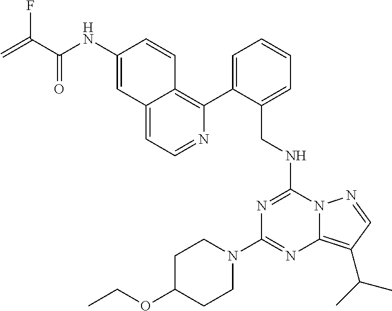 [SMILES: C=C(F)C(=O)Nc1ccc2c(-c3ccccc3CNc3nc(N4CCC(OCC)CC4)nc4c(C(C)C)cnn34)nccc2c1] |
| 86 | 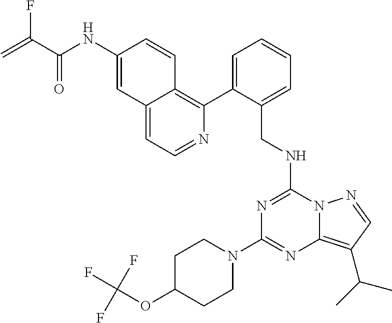 [SMILES: C=C(F)C(=O)Nc1ccc2c(-c3ccccc3CNc3nc(N4CCC(OC(F)(F)F)CC4)nc4c(C(C)C)cnn34)nccc2c1] |
| 87 | 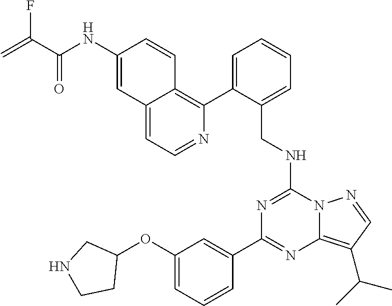 [SMILES: C=C(F)C(=O)Nc1ccc2c(-c3ccccc3CNc3nc(-c4cccc(OC5CCNC5)c4)nc4c(C(C)C)cnn34)nccc2c1] |
| 83 | 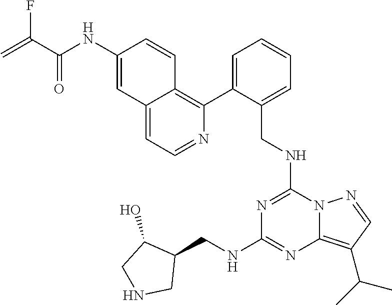 [SMILES: C=C(F)C(=O)Nc1ccc2c(-c3ccccc3CNc3nc(NC[C@H]4CNC[C@@H]4O)nc4c(C(C)C)cnn34)nccc2c1] |
| 84 | 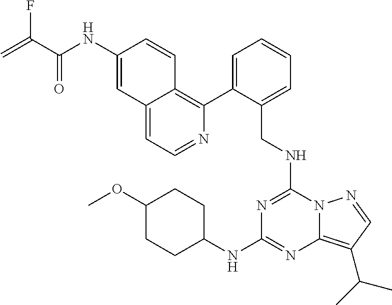 [SMILES: C=C(F)C(=O)Nc1ccc2c(-c3ccccc3CNc3nc(NC4CCC(OC)CC4)nc4c(C(C)C)cnn34)nccc2c1] |
| 92 | 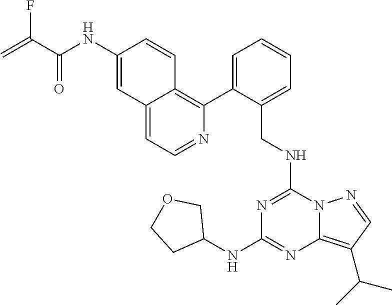 [SMILES: C=C(F)C(=O)Nc1ccc2c(-c3ccccc3CNc3nc(NC4CCOC4)nc4c(C(C)C)cnn34)nccc2c1] |
| 93 | 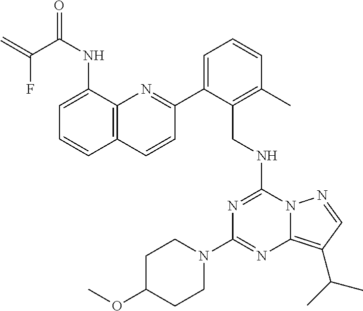 [SMILES: C=C(F)C(=O)Nc1cccc2ccc(-c3cccc(C)c3CNc3nc(N4CCC(OC)CC4)nc4c(C(C)C)cnn34)nc12] |
| 94 | 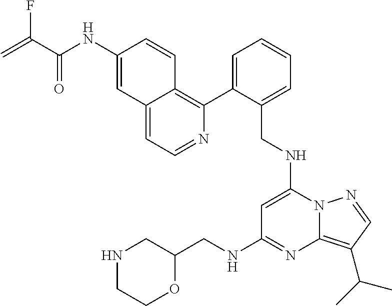 [SMILES: C=C(F)C(=O)Nc1ccc2c(-c3ccccc3CNc3cc(NCC4CNCCO4)nc4c(C(C)C)cnn34)nccc2c1] |
| 88 | 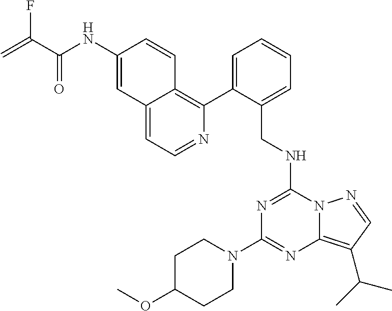 [SMILES: C=C(F)C(=O)Nc1ccc2c(-c3ccccc3CNc3nc(N4CCC(OC)CC4)nc4c(C(C)C)cnn34)nccc2c1] |
| 89 | 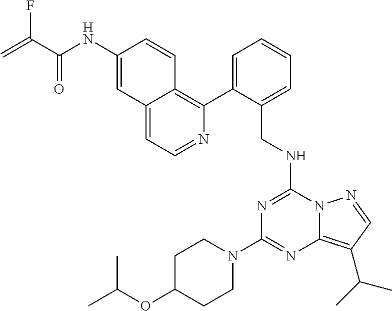 [SMILES: C=C(F)C(=O)Nc1ccc2c(-c3ccccc3CNc3nc(N4CCC(OC(C)C)CC4)nc4c(C(C)C)cnn34)nccc2c1] |
| 90 | 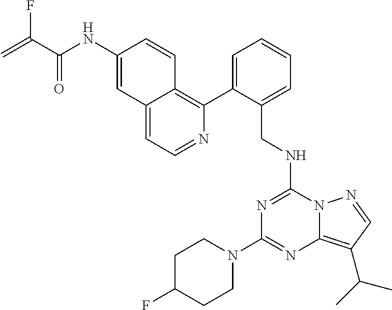 [SMILES: C=C(F)C(=O)Nc1ccc2c(-c3ccccc3CNc3nc(N4CCC(F)CC4)nc4c(C(C)C)cnn34)nccc2c1] |
| 91 | 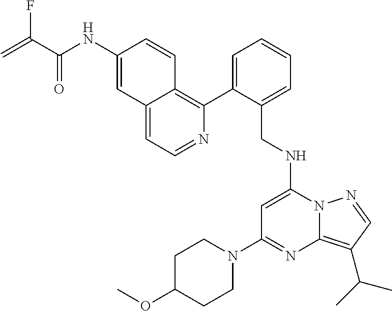 [SMILES: C=C(F)C(=O)Nc1ccc2c(-c3ccccc3CNc3cc(N4CCC(OC)CC4)nc4c(C(C)C)cnn34)nccc2c1] |
| 99 | 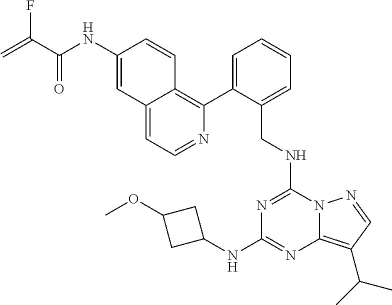 [SMILES: C=C(F)C(=O)Nc1ccc2c(-c3ccccc3CNc3nc(NC4CC(OC)C4)nc4c(C(C)C)cnn34)nccc2c1] |
| 100 | 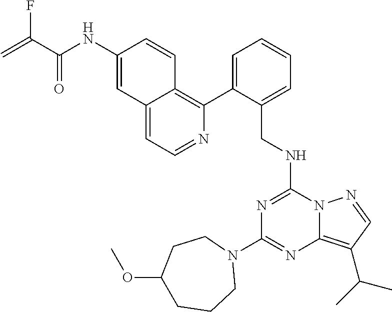 [SMILES: C=C(F)C(=O)Nc1ccc2c(-c3ccccc3CNc3nc(N4CCCC(OC)CC4)nc4c(C(C)C)cnn34)nccc2c1] |
| 95 | 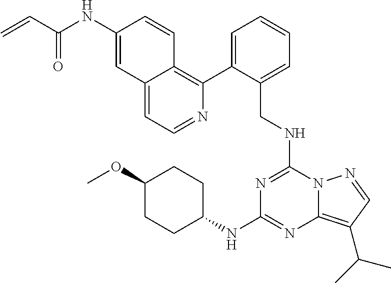 [SMILES: C=CC(=O)Nc1ccc2c(-c3ccccc3CNc3nc(N[C@H]4CC[C@H](OC)CC4)nc4c(C(C)C)cnn34)nccc2c1] |
| 96 | 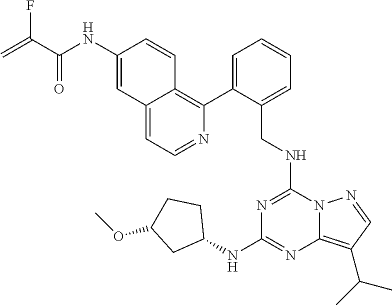 |
| 97 | 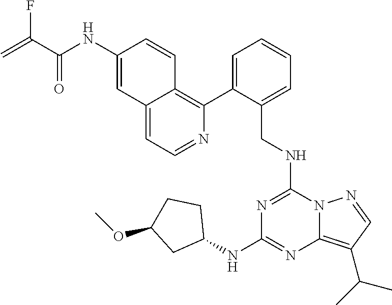 [SMILES: C=C(F)C(=O)Nc1ccc2c(-c3ccccc3CNc3nc(N[C@H]4CC[C@H](OC)C4)nc4c(C(C)C)cnn34)nccc2c1] |
| 98 | 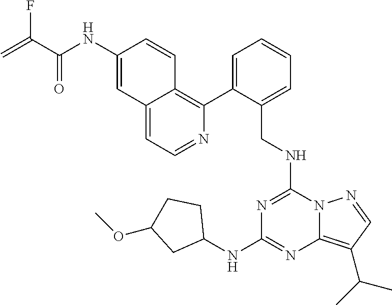 [SMILES: C=C(F)C(=O)Nc1ccc2c(-c3ccccc3CNc3nc(NC4CCC(OC)C4)nc4c(C(C)C)cnn34)nccc2c1] |
| 106 | 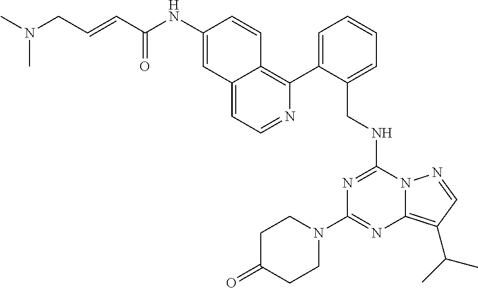 [SMILES: CC(C)c1cnn2c(NCc3ccccc3-c3nccc4cc(NC(=O)/C=C/CN(C)C)ccc34)nc(N3CCC(=O)CC3)nc12] |
| 107 | 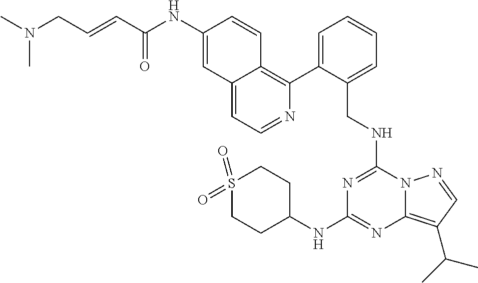 [SMILES: CC(C)c1cnn2c(NCc3ccccc3-c3nccc4cc(NC(=O)/C=C/CN(C)C)ccc34)nc(NC3CCS(=O)(=O)CC3)nc12] |
| 101 | 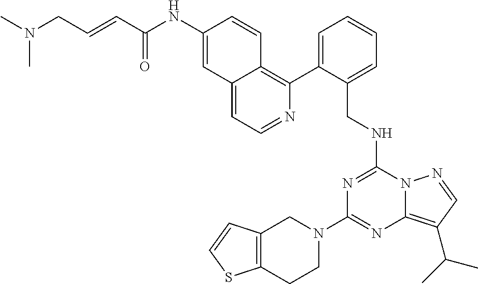 [SMILES: CC(C)c1cnn2c(NCc3ccccc3-c3nccc4cc(NC(=O)/C=C/CN(C)C)ccc34)nc(N3CCc4sccc4C3)nc12] |
| 102 | 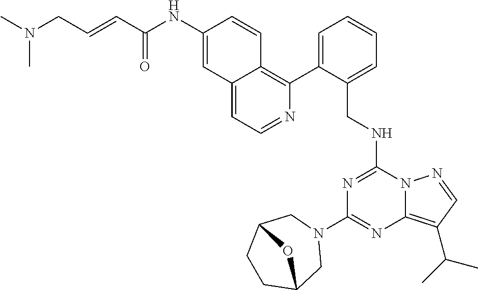 [SMILES: CC(C)c1cnn2c(NCc3ccccc3-c3nccc4cc(NC(=O)/C=C/CN(C)C)ccc34)nc(N3C[C@H]4CC[C@@H](C3)O4)nc12] |
| 103 | 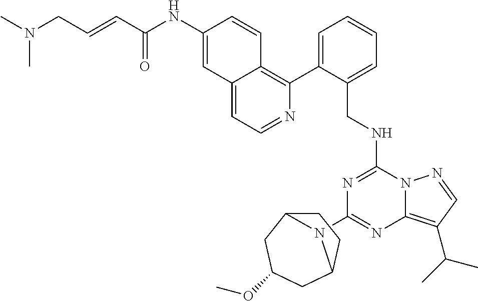 |
| 104 | 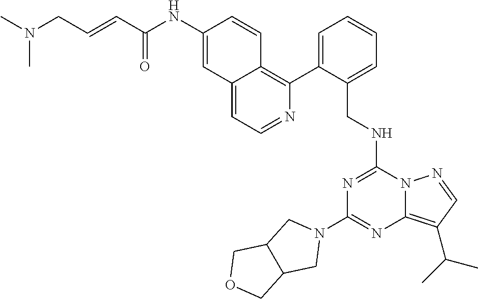 [SMILES: CC(C)c1cnn2c(NCc3ccccc3-c3nccc4cc(NC(=O)/C=C/CN(C)C)ccc34)nc(N3CC4COCC4C3)nc12] |
| 105 | 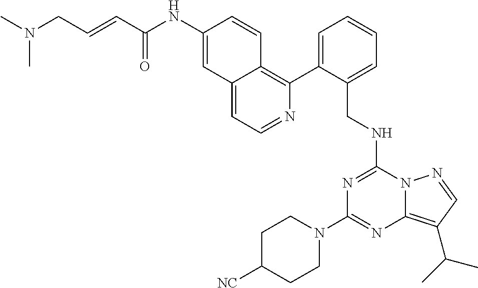 [SMILES: CC(C)c1cnn2c(NCc3ccccc3-c3nccc4cc(NC(=O)/C=C/CN(C)C)ccc34)nc(N3CCC(C#N)CC3)nc12] |
| 113 | 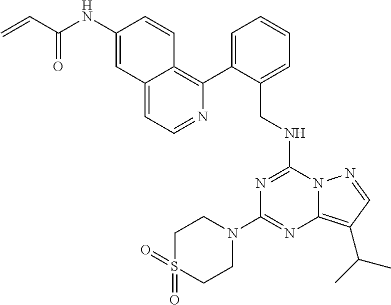 [SMILES: C=CC(=O)Nc1ccc2c(-c3ccccc3CNc3nc(N4CCS(=O)(=O)CC4)nc4c(C(C)C)cnn34)nccc2c1] |
| 108 | 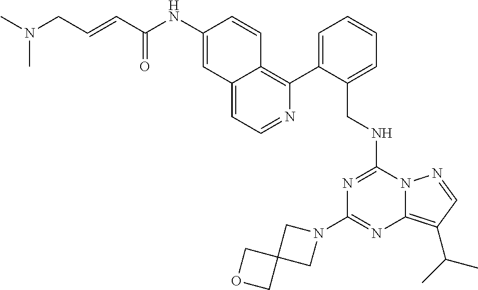 [SMILES: CC(C)c1cnn2c(NCc3ccccc3-c3nccc4cc(NC(=O)/C=C/CN(C)C)ccc34)nc(N3CC4(COC4)C3)nc12] |
| 109 | 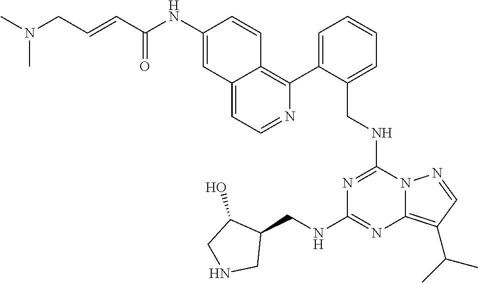 [SMILES: CC(C)c1cnn2c(NCc3ccccc3-c3nccc4cc(NC(=O)/C=C/CN(C)C)ccc34)nc(NC[C@H]3CNC[C@@H]3O)nc12] |
| 110 | 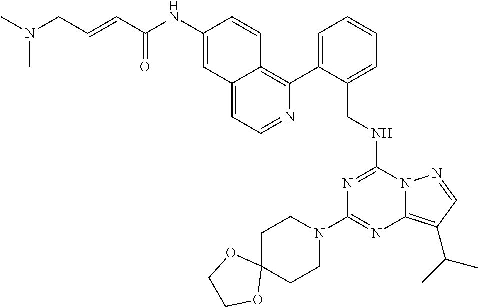 [SMILES: CC(C)c1cnn2c(NCc3ccccc3-c3nccc4cc(NC(=O)/C=C/CN(C)C)ccc34)nc(N3CCC4(CC3)OCCO4)nc12] |
| 111 | 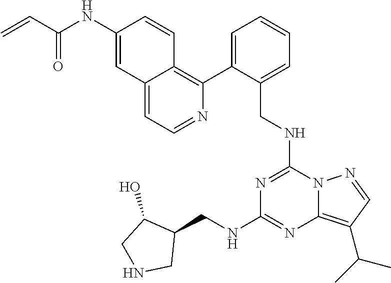 [SMILES: C=CC(=O)Nc1ccc2c(-c3ccccc3CNc3nc(NC[C@H]4CNC[C@@H]4O)nc4c(C(C)C)cnn34)nccc2c1] |
| 112 | 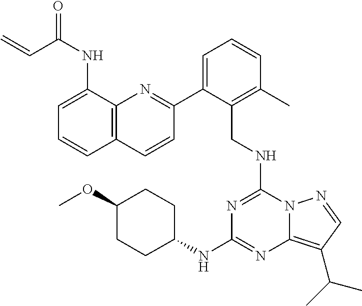 [SMILES: C=CC(=O)Nc1cccc2ccc(-c3cccc(C)c3CNc3nc(N[C@H]4CC[C@H](OC)CC4)nc4c(C(C)C)cnn34)nc12] |
| 120 | 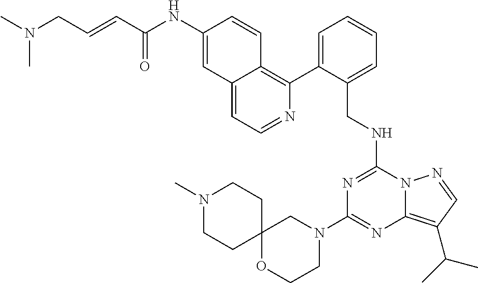 [SMILES: CC(C)c1cnn2c(NCc3ccccc3-c3nccc4cc(NC(=O)/C=C/CN(C)C)ccc34)nc(N3CCOC4(CCN(C)CC4)C3)nc12] |
| 114 | 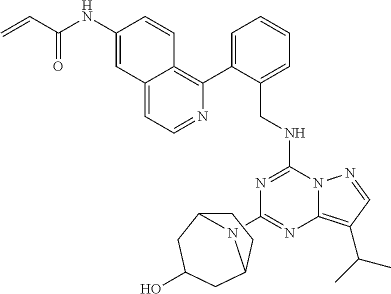 [SMILES: C=CC(=O)Nc1ccc2c(-c3ccccc3CNc3nc(N4C5CCC4CC(O)C5)nc4c(C(C)C)cnn34)nccc2c1] |
| 115 | 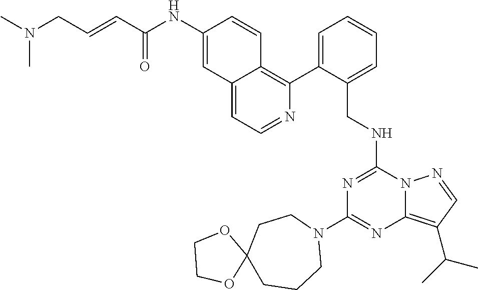 [SMILES: CC(C)c1cnn2c(NCc3ccccc3-c3nccc4cc(NC(=O)/C=C/CN(C)C)ccc34)nc(N3CCCC4(CC3)OCCO4)nc12] |
| 116 | 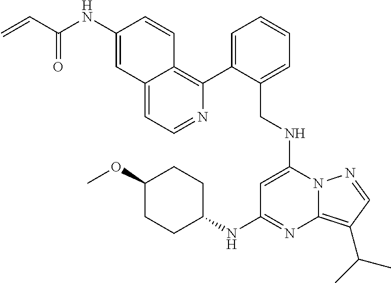 [SMILES: C=CC(=O)Nc1ccc2c(-c3ccccc3CNc3cc(N[C@H]4CC[C@H](OC)CC4)nc4c(C(C)C)cnn34)nccc2c1] |
| 117 | 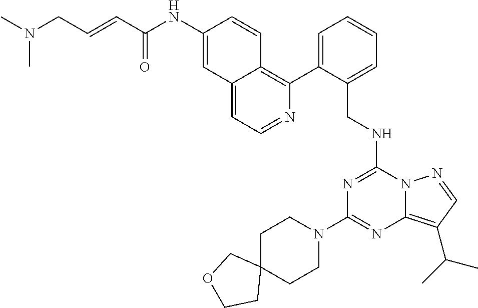 [SMILES: CC(C)c1cnn2c(NCc3ccccc3-c3nccc4cc(NC(=O)/C=C/CN(C)C)ccc34)nc(N3CCC4(CCOC4)CC3)nc12] |
| 118 | 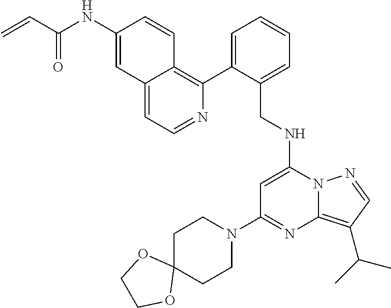 [SMILES: C=CC(=O)Nc1ccc2c(-c3ccccc3CNc3cc(N4CCC5(CC4)OCCO5)nc4c(C(C)C)cnn34)nccc2c1] |
| 119 | 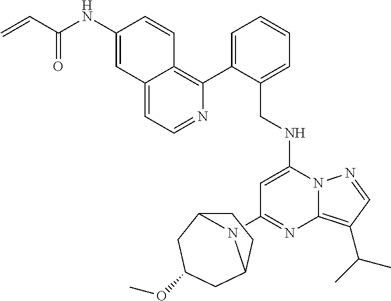 |
| 121 | 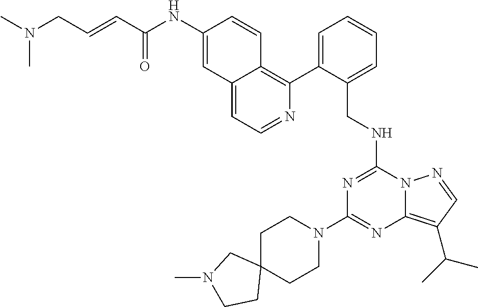 [SMILES: CC(C)c1cnn2c(NCc3ccccc3-c3nccc4cc(NC(=O)/C=C/CN(C)C)ccc34)nc(N3CCC4(CCN(C)C4)CC3)nc12] |
| 122 | 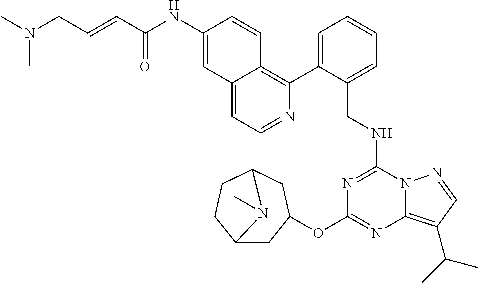 [SMILES: CC(C)c1cnn2c(NCc3ccccc3-c3nccc4cc(NC(=O)/C=C/CN(C)C)ccc34)nc(OC3CC4CCC(C3)N4C)nc12] |
| 123 | 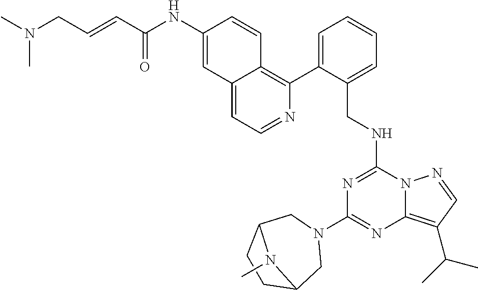 [SMILES: CC(C)c1cnn2c(NCc3ccccc3-c3nccc4cc(NC(=O)/C=C/CN(C)C)ccc34)nc(N3CC4CCC(C3)N4C)nc12] |
| 124 | 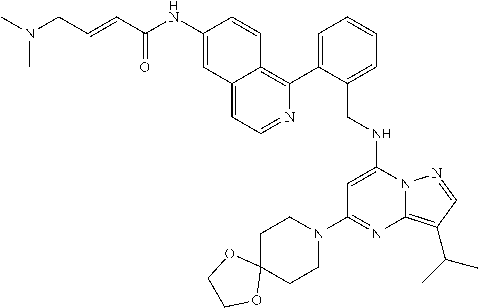 [SMILES: CC(C)c1cnn2c(NCc3ccccc3-c3nccc4cc(NC(=O)/C=C/CN(C)C)ccc34)cc(N3CCC4(CC3)OCCO4)nc12] |
| 125 | 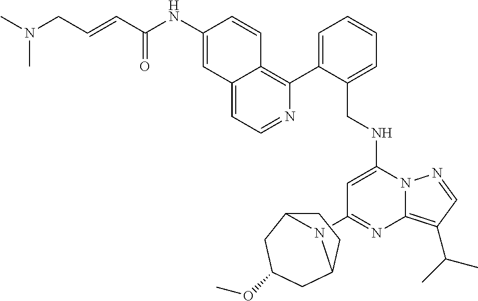 |
| 126 | 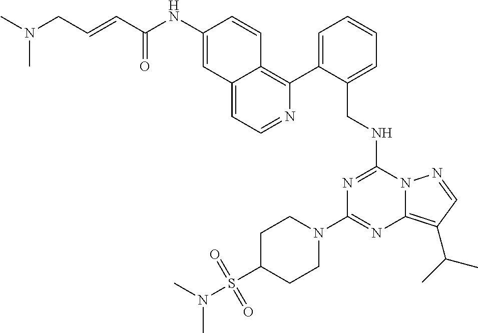 [SMILES: CC(C)c1cnn2c(NCc3ccccc3-c3nccc4cc(NC(=O)/C=C/CN(C)C)ccc34)nc(N3CCC(S(=O)(=O)N(C)C)CC3)nc12] |
| 127 | 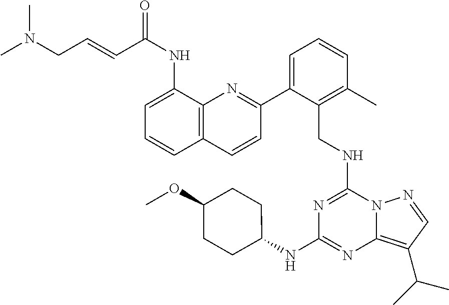 [SMILES: CO[C@H]1CC[C@H](Nc2nc(NCc3c(C)cccc3-c3ccc4cccc(NC(=O)/C=C/CN(C)C)c4n3)n3ncc(C(C)C)c3n2)CC1] |
| 128 | 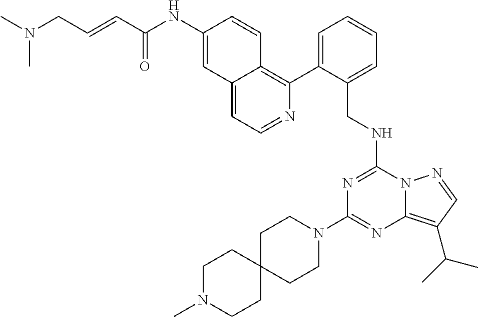 [SMILES: CC(C)c1cnn2c(NCc3ccccc3-c3nccc4cc(NC(=O)/C=C/CN(C)C)ccc34)nc(N3CCC4(CCN(C)CC4)CC3)nc12] |
| 129 | 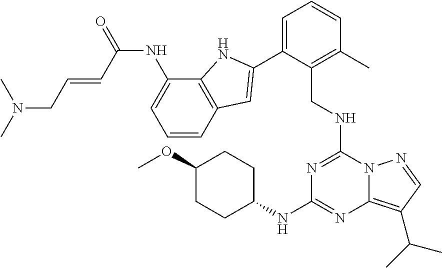 [SMILES: CO[C@H]1CC[C@H](Nc2nc(NCc3c(C)cccc3-c3cc4cccc(NC(=O)/C=C/CN(C)C)c4[nH]3)n3ncc(C(C)C)c3n2)CC1] |
| 130 | 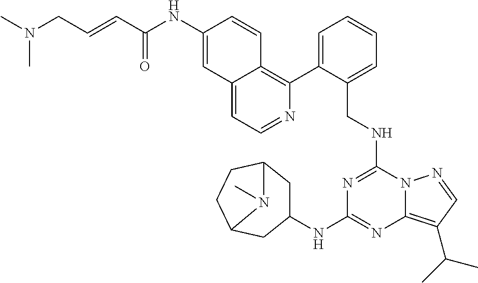 [SMILES: CC(C)c1cnn2c(NCc3ccccc3-c3nccc4cc(NC(=O)/C=C/CN(C)C)ccc34)nc(NC3CC4CCC(C3)N4C)nc12] |
| 131 | 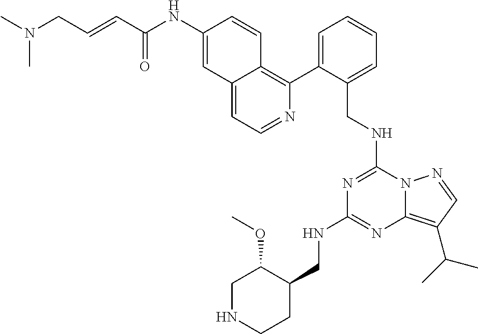 [SMILES: CO[C@H]1CNCC[C@@H]1CNc1nc(NCc2ccccc2-c2nccc3cc(NC(=O)/C=C/CN(C)C)ccc23)n2ncc(C(C)C)c2n1] |
| 132 | 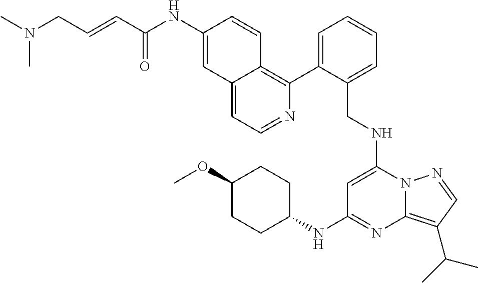 [SMILES: CO[C@H]1CC[C@H](Nc2cc(NCc3ccccc3-c3nccc4cc(NC(=O)/C=C/CN(C)C)ccc34)n3ncc(C(C)C)c3n2)CC1] |
| 134 | 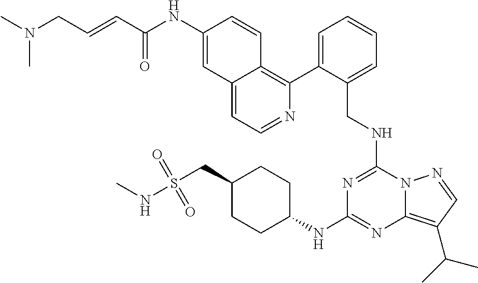 [SMILES: CNS(=O)(=O)C[C@H]1CC[C@H](Nc2nc(NCc3ccccc3-c3nccc4cc(NC(=O)/C=C/CN(C)C)ccc34)n3ncc(C(C)C)c3n2)CC1] |
| 135 | 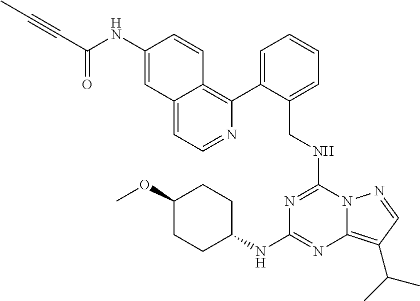 [SMILES: CC#CC(=O)Nc1ccc2c(-c3ccccc3CNc3nc(N[C@H]4CC[C@H](OC)CC4)nc4c(C(C)C)cnn34)nccc2c1] |
| 136 | 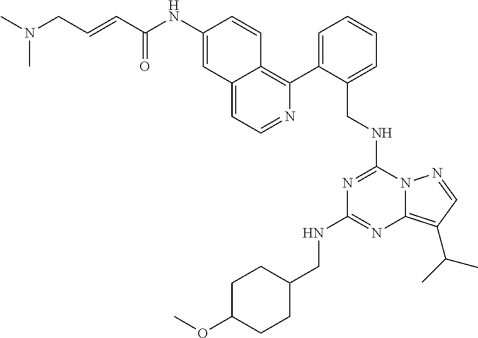 [SMILES: COC1CCC(CNc2nc(NCc3ccccc3-c3nccc4cc(NC(=O)/C=C/CN(C)C)ccc34)n3ncc(C(C)C)c3n2)CC1] |
| 137 | 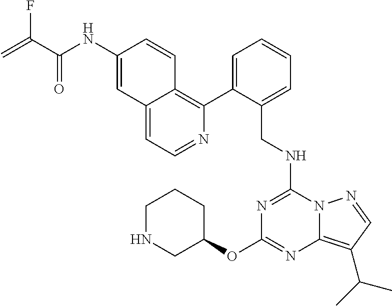 [SMILES: C=C(F)C(=O)Nc1ccc2c(-c3ccccc3CNc3nc(O[C@@H]4CCCNC4)nc4c(C(C)C)cnn34)nccc2c1] |
| 138 | 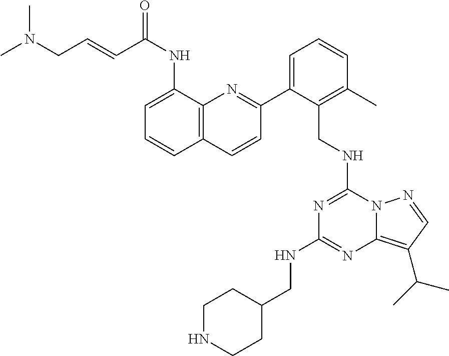 [SMILES: Cc1cccc(-c2ccc3cccc(NC(=O)/C=C/CN(C)C)c3n2)c1CNc1nc(NCC2CCNCC2)nc2c(C(C)C)cnn12] |
| 139 | 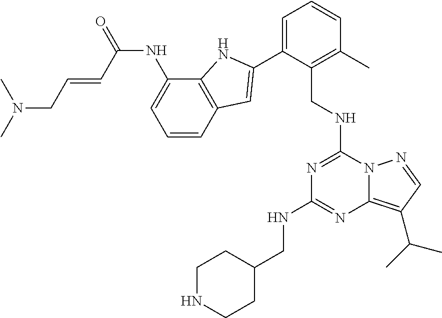 [SMILES: Cc1cccc(-c2cc3cccc(NC(=O)/C=C/CN(C)C)c3[nH]2)c1CNc1nc(NCC2CCNCC2)nc2c(C(C)C)cnn12] |
| 133 | 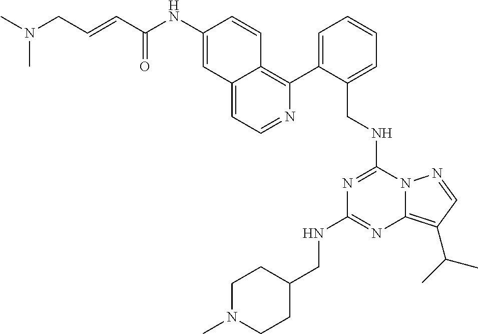 [SMILES: CC(C)c1cnn2c(NCc3ccccc3-c3nccc4cc(NC(=O)/C=C/CN(C)C)ccc34)nc(NCC3CCN(C)CC3)nc12] |
| 141 | 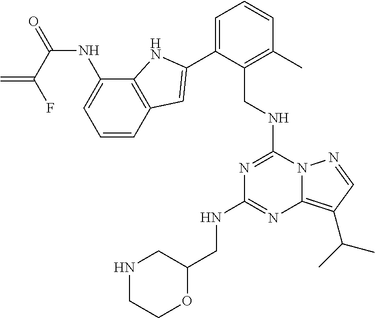 [SMILES: C=C(F)C(=O)Nc1cccc2cc(-c3cccc(C)c3CNc3nc(NCC4CNCCO4)nc4c(C(C)C)cnn34)[nH]c12] |
| 142 | 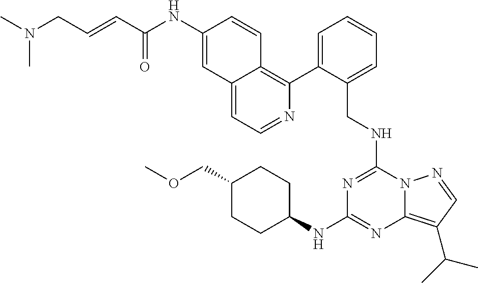 [SMILES: COC[C@H]1CC[C@H](Nc2nc(NCc3ccccc3-c3nccc4cc(NC(=O)/C=C/CN(C)C)ccc34)n3ncc(C(C)C)c3n2)CC1] |
| 143 | 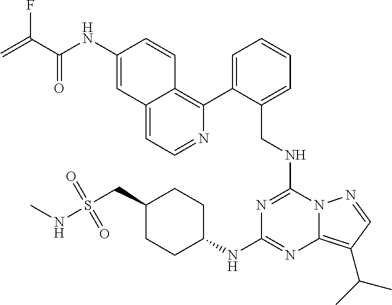 [SMILES: C=C(F)C(=O)Nc1ccc2c(-c3ccccc3CNc3nc(N[C@H]4CC[C@H](CS(=O)(=O)NC)CC4)nc4c(C(C)C)cnn34)nccc2c1] |
| 144 | 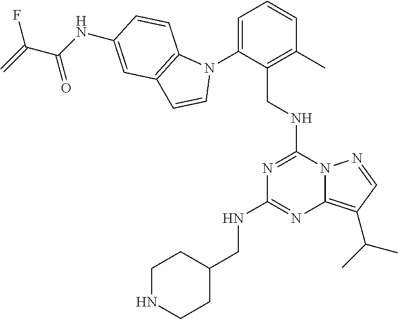 [SMILES: C=C(F)C(=O)Nc1ccc2c(ccn2-c2cccc(C)c2CNc2nc(NCC3CCNCC3)nc3c(C(C)C)cnn23)c1] |
| 145 | 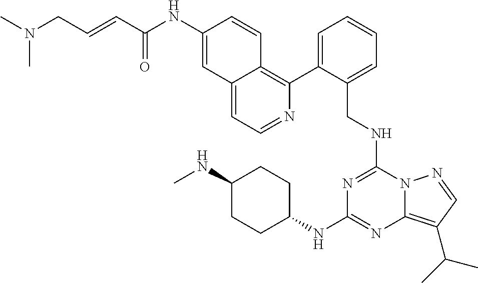 [SMILES: CN[C@H]1CC[C@H](Nc2nc(NCc3ccccc3-c3nccc4cc(NC(=O)/C=C/CN(C)C)ccc34)n3ncc(C(C)C)c3n2)CC1] |
| 140 | 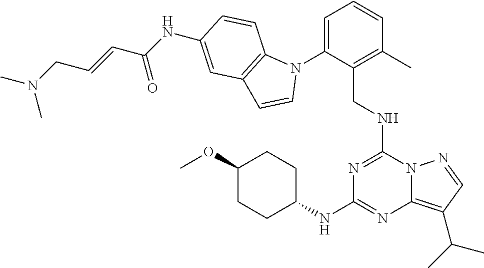 [SMILES: CO[C@H]1CC[C@H](Nc2nc(NCc3c(C)cccc3-n3ccc4cc(NC(=O)/C=C/CN(C)C)ccc43)n3ncc(C(C)C)c3n2)CC1] |
| 148 | 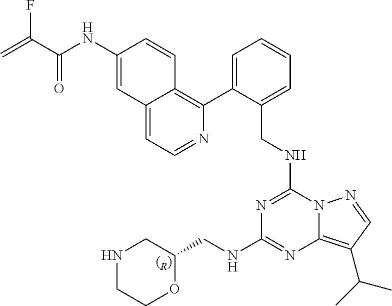 |
| 149 | 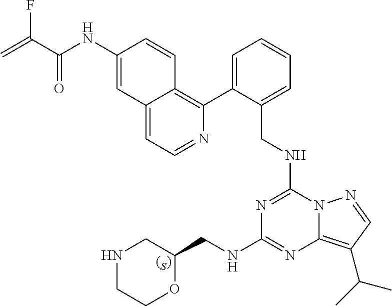 |
| 150 | 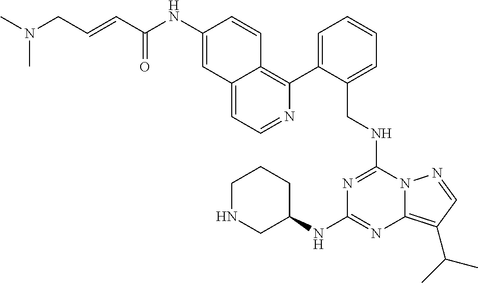 [SMILES: CC(C)c1cnn2c(NCc3ccccc3-c3nccc4cc(NC(=O)/C=C/CN(C)C)ccc34)nc(N[C@@H]3CCCNC3)nc12] |
| 151 | 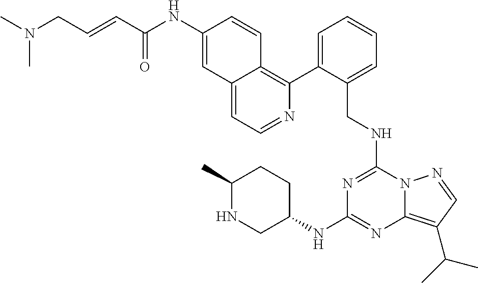 [SMILES: CC(C)c1cnn2c(NCc3ccccc3-c3nccc4cc(NC(=O)/C=C/CN(C)C)ccc34)nc(N[C@H]3CC[C@H](C)NC3)nc12] |
| 152 | 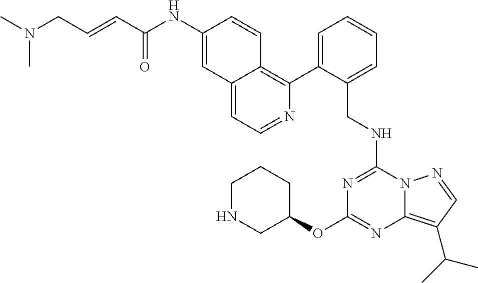 [SMILES: CC(C)c1cnn2c(NCc3ccccc3-c3nccc4cc(NC(=O)/C=C/CN(C)C)ccc34)nc(O[C@@H]3CCCNC3)nc12] |
| 146 | 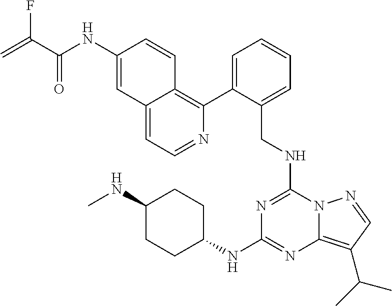 [SMILES: C=C(F)C(=O)Nc1ccc2c(-c3ccccc3CNc3nc(N[C@H]4CC[C@H](NC)CC4)nc4c(C(C)C)cnn34)nccc2c1] |
| 147 | 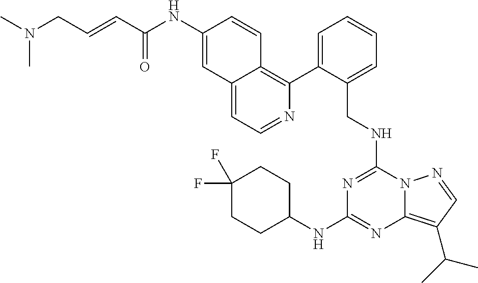 [SMILES: CC(C)c1cnn2c(NCc3ccccc3-c3nccc4cc(NC(=O)/C=C/CN(C)C)ccc34)nc(NC3CCC(F)(F)CC3)nc12] |
| 155 | 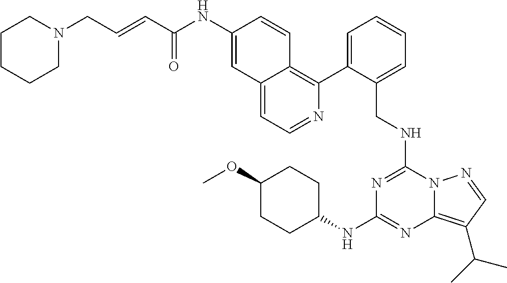 |
| 156 | 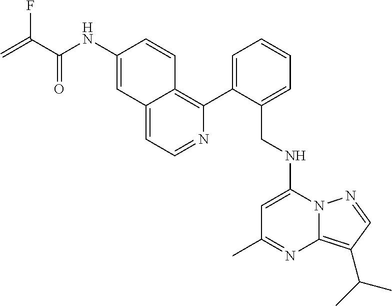 [SMILES: C=C(F)C(=O)Nc1ccc2c(-c3ccccc3CNc3cc(C)nc4c(C(C)C)cnn34)nccc2c1] |
| 157 | 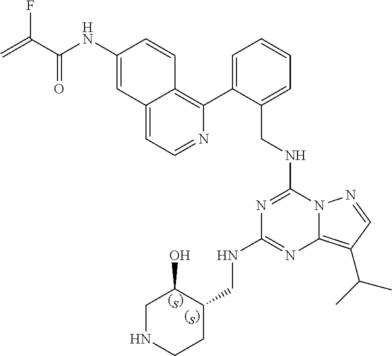 |
| 158 | 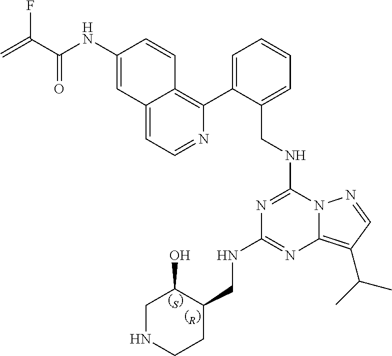 |
| 153 | 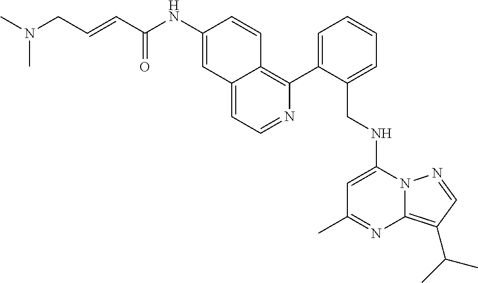 [SMILES: Cc1cc(NCc2ccccc2-c2nccc3cc(NC(=O)/C=C/CN(C)C)ccc23)n2ncc(C(C)C)c2n1] |
| 154 | 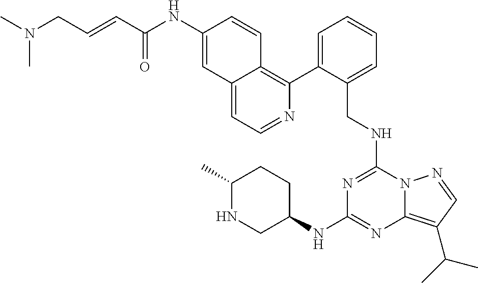 [SMILES: CC(C)c1cnn2c(NCc3ccccc3-c3nccc4cc(NC(=O)/C=C/CN(C)C)ccc34)nc(N[C@@H]3CC[C@@H](C)NC3)nc12] |
| 162 | 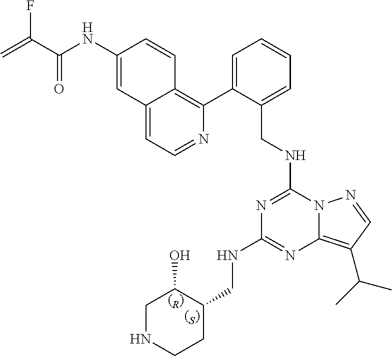 |
| 163 | 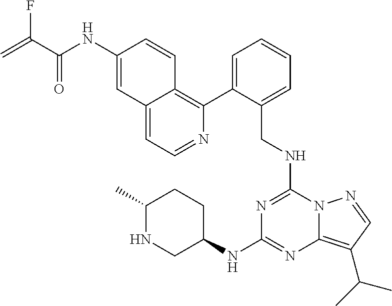 [SMILES: C=C(F)C(=O)Nc1ccc2c(-c3ccccc3CNc3nc(N[C@@H]4CC[C@@H](C)NC4)nc4c(C(C)C)cnn34)nccc2c1] |
| 164 | 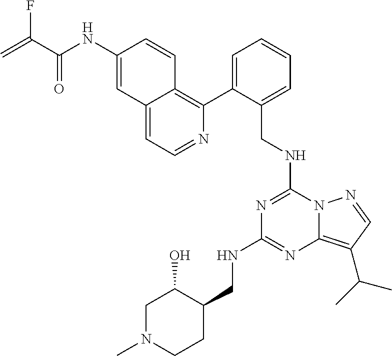 [SMILES: C=C(F)C(=O)Nc1ccc2c(-c3ccccc3CNc3nc(NC[C@H]4CCN(C)C[C@@H]4O)nc4c(C(C)C)cnn34)nccc2c1] |
| 165 | 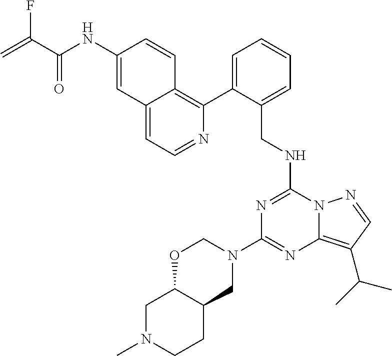 [SMILES: C=C(F)C(=O)Nc1ccc2c(-c3ccccc3CNc3nc(N4CO[C@H]5CN(C)CC[C@@H]5C4)nc4c(C(C)C)cnn34)nccc2c1] |
| 159 | 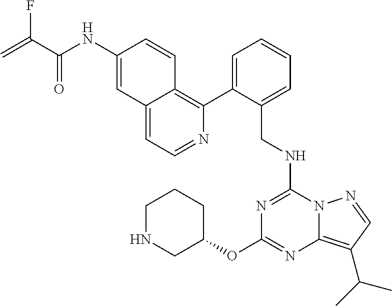 [SMILES: C=C(F)C(=O)Nc1ccc2c(-c3ccccc3CNc3nc(O[C@H]4CCCNC4)nc4c(C(C)C)cnn34)nccc2c1] |
| 160 | 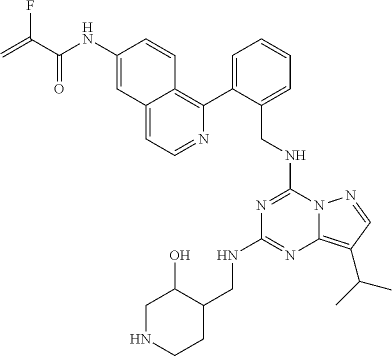 [SMILES: C=C(F)C(=O)Nc1ccc2c(-c3ccccc3CNc3nc(NCC4CCNCC4O)nc4c(C(C)C)cnn34)nccc2c1] |
| 161 | 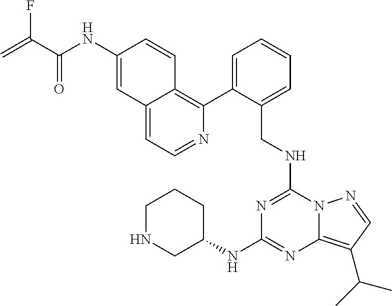 [SMILES: C=C(F)C(=O)Nc1ccc2c(-c3ccccc3CNc3nc(N[C@H]4CCCNC4)nc4c(C(C)C)cnn34)nccc2c1] |
| 169 | 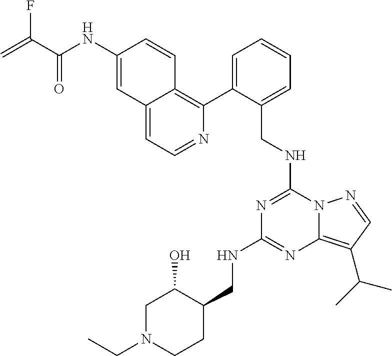 [SMILES: C=C(F)C(=O)Nc1ccc2c(-c3ccccc3CNc3nc(NC[C@H]4CCN(CC)C[C@@H]4O)nc4c(C(C)C)cnn34)nccc2c1] |
| 170 | 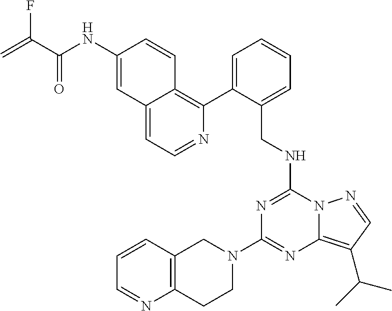 [SMILES: C=C(F)C(=O)Nc1ccc2c(-c3ccccc3CNc3nc(N4CCc5ncccc5C4)nc4c(C(C)C)cnn34)nccc2c1] |
| 171 | 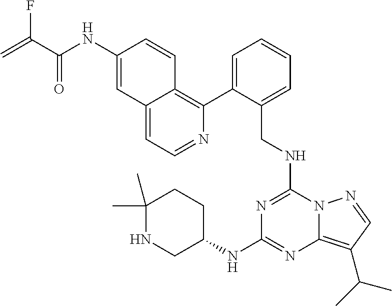 [SMILES: C=C(F)C(=O)Nc1ccc2c(-c3ccccc3CNc3nc(N[C@H]4CCC(C)(C)NC4)nc4c(C(C)C)cnn34)nccc2c1] |
| 166 | 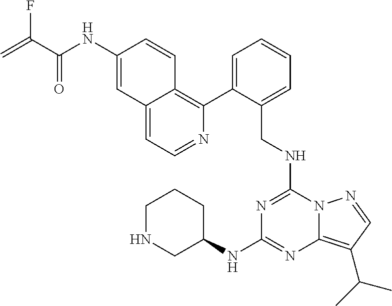 [SMILES: C=C(F)C(=O)Nc1ccc2c(-c3ccccc3CNc3nc(N[C@@H]4CCCNC4)nc4c(C(C)C)cnn34)nccc2c1] |
| 167 | 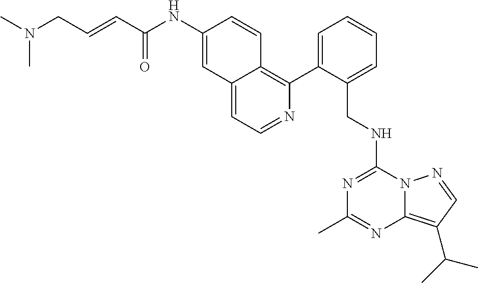 [SMILES: Cc1nc(NCc2ccccc2-c2nccc3cc(NC(=O)/C=C/CN(C)C)ccc23)n2ncc(C(C)C)c2n1] |
| 168 | 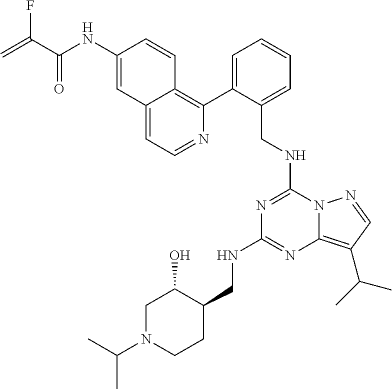 [SMILES: C=C(F)C(=O)Nc1ccc2c(-c3ccccc3CNc3nc(NC[C@H]4CCN(C(C)C)C[C@@H]4O)nc4c(C(C)C)cnn34)nccc2c1] |
| 176 | 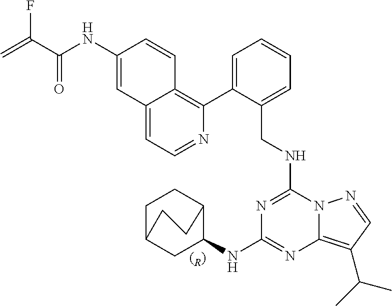 |
| 177 | 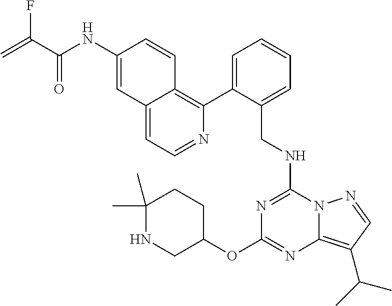 [SMILES: C=C(F)C(=O)Nc1ccc2c(-c3ccccc3CNc3nc(OC4CCC(C)(C)NC4)nc4c(C(C)C)cnn34)nccc2c1] |
| 178 | 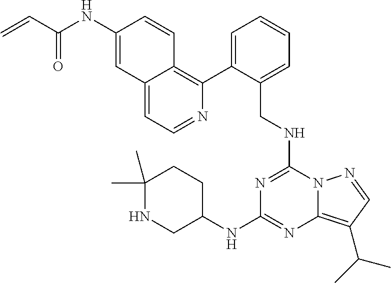 [SMILES: C=CC(=O)Nc1ccc2c(-c3ccccc3CNc3nc(NC4CCC(C)(C)NC4)nc4c(C(C)C)cnn34)nccc2c1] |
| 172 | 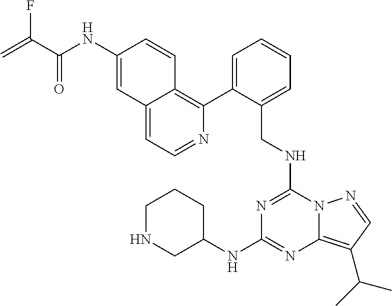 [SMILES: C=C(F)C(=O)Nc1ccc2c(-c3ccccc3CNc3nc(NC4CCCNC4)nc4c(C(C)C)cnn34)nccc2c1] |
| 173 | 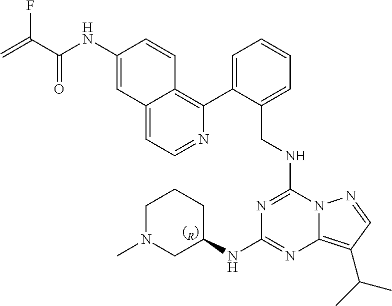 |
| 174 | 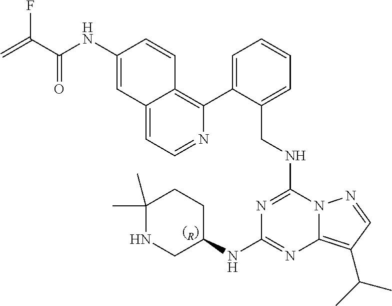 |
| 175 | 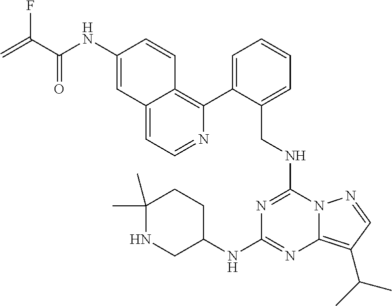 [SMILES: C=C(F)C(=O)Nc1ccc2c(-c3ccccc3CNc3nc(NC4CCC(C)(C)NC4)nc4c(C(C)C)cnn34)nccc2c1] |
| 183 | 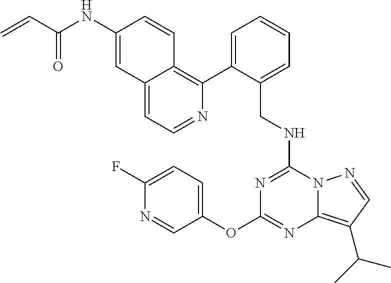 [SMILES: C=CC(=O)Nc1ccc2c(-c3ccccc3CNc3nc(Oc4ccc(F)nc4)nc4c(C(C)C)cnn34)nccc2c1] |
| 184 | 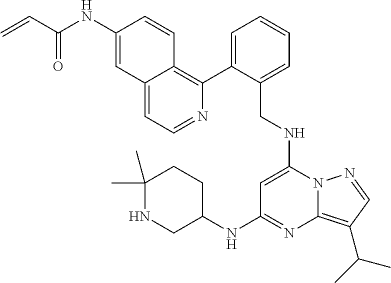 [SMILES: C=CC(=O)Nc1ccc2c(-c3ccccc3CNc3cc(NC4CCC(C)(C)NC4)nc4c(C(C)C)cnn34)nccc2c1] |
| 179 | 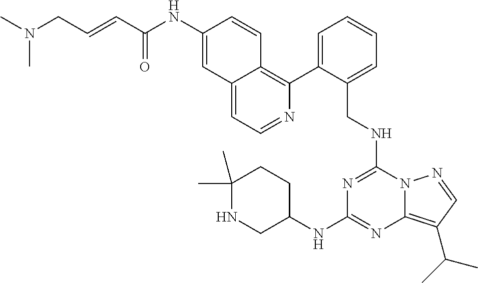 [SMILES: CC(C)c1cnn2c(NCc3ccccc3-c3nccc4cc(NC(=O)/C=C/CN(C)C)ccc34)nc(NC3CCC(C)(C)NC3)nc12] |
| 180 | 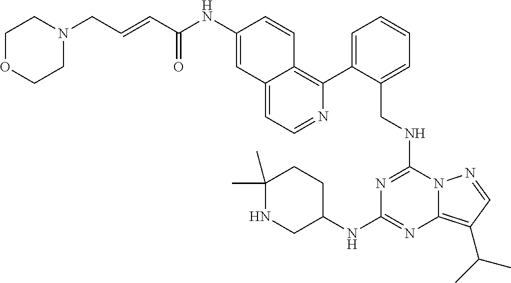 [SMILES: CC(C)c1cnn2c(NCc3ccccc3-c3nccc4cc(NC(=O)/C=C/CN5CCOCC5)ccc34)nc(NC3CCC(C)(C)NC3)nc12] |
| 181 | 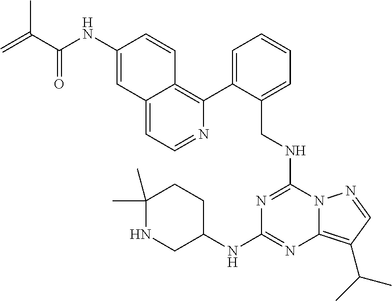 [SMILES: C=C(C)C(=O)Nc1ccc2c(-c3ccccc3CNc3nc(NC4CCC(C)(C)NC4)nc4c(C(C)C)cnn34)nccc2c1] |
| 182 | 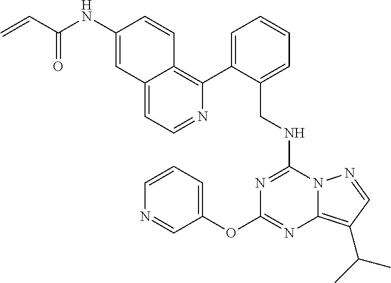 [SMILES: C=CC(=O)Nc1ccc2c(-c3ccccc3CNc3nc(Oc4cccnc4)nc4c(C(C)C)cnn34)nccc2c1] |
| 190 | 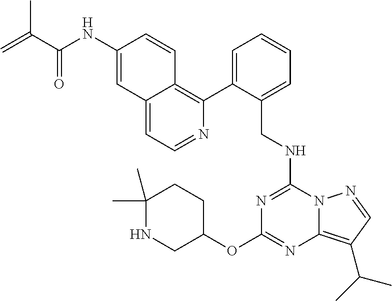 [SMILES: C=C(C)C(=O)Nc1ccc2c(-c3ccccc3CNc3nc(OC4CCC(C)(C)NC4)nc4c(C(C)C)cnn34)nccc2c1] |
| 191 | 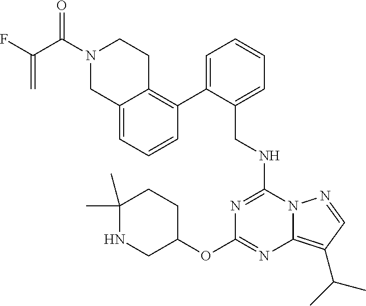 [SMILES: C=C(F)C(=O)N1CCc2c(cccc2-c2ccccc2CNc2nc(OC3CCC(C)(C)NC3)nc3c(C(C)C)cnn23)C1] |
| 185 | 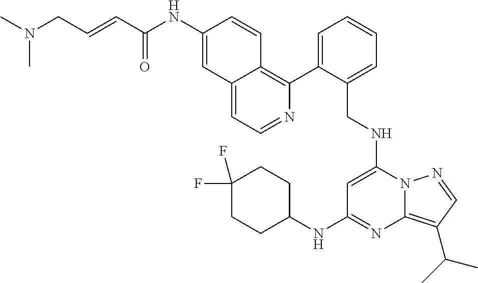 [SMILES: CC(C)c1cnn2c(NCc3ccccc3-c3nccc4cc(NC(=O)/C=C/CN(C)C)ccc34)cc(NC3CCC(F)(F)CC3)nc12] |
| 186 | 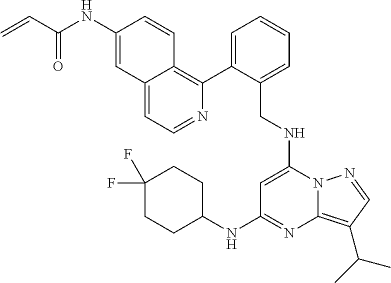 [SMILES: C=CC(=O)Nc1ccc2c(-c3ccccc3CNc3cc(NC4CCC(F)(F)CC4)nc4c(C(C)C)cnn34)nccc2c1] |
| 187 | 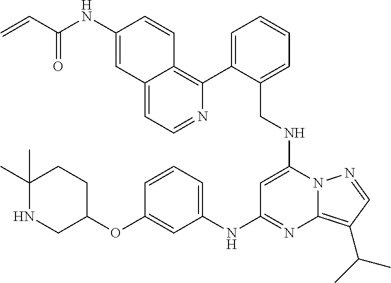 [SMILES: C=CC(=O)Nc1ccc2c(-c3ccccc3CNc3cc(Nc4cccc(OC5CCC(C)(C)NC5)c4)nc4c(C(C)C)cnn34)nccc2c1] |
| 188 | 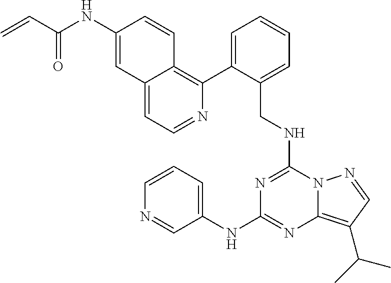 [SMILES: C=CC(=O)Nc1ccc2c(-c3ccccc3CNc3nc(Nc4cccnc4)nc4c(C(C)C)cnn34)nccc2c1] |
| 189 | 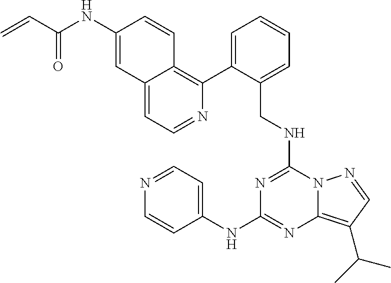 [SMILES: C=CC(=O)Nc1ccc2c(-c3ccccc3CNc3nc(Nc4ccncc4)nc4c(C(C)C)cnn34)nccc2c1] |
| 197 | 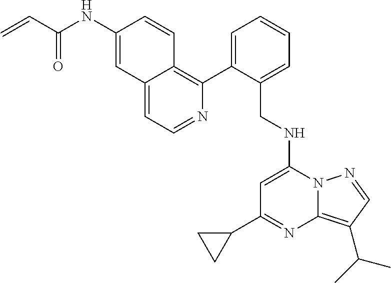 [SMILES: C=CC(=O)Nc1ccc2c(-c3ccccc3CNc3cc(C4CC4)nc4c(C(C)C)cnn34)nccc2c1] |
| 192 | 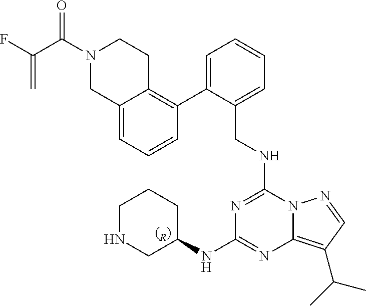 |
| 193 | 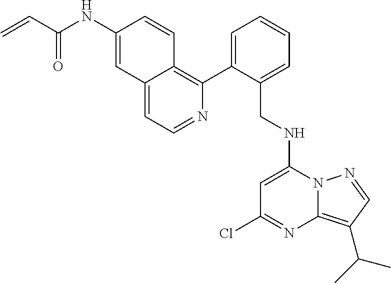 [SMILES: C=CC(=O)Nc1ccc2c(-c3ccccc3CNc3cc(Cl)nc4c(C(C)C)cnn34)nccc2c1] |
| 194 | 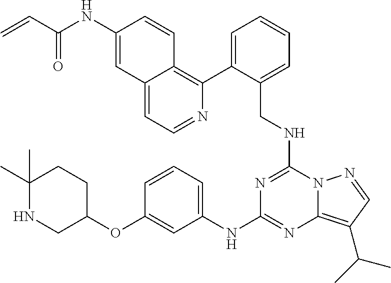 [SMILES: C=CC(=O)Nc1ccc2c(-c3ccccc3CNc3nc(Nc4cccc(OC5CCC(C)(C)NC5)c4)nc4c(C(C)C)cnn34)nccc2c1] |
| 195 | 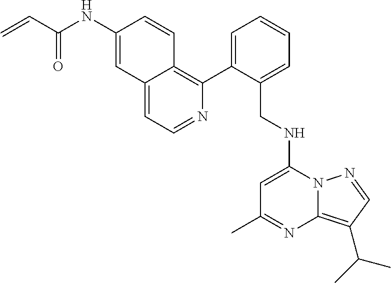 [SMILES: C=CC(=O)Nc1ccc2c(-c3ccccc3CNc3cc(C)nc4c(C(C)C)cnn34)nccc2c1] |
| 196 | 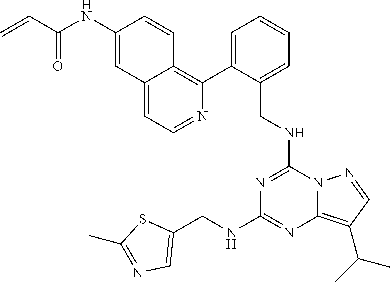 [SMILES: C=CC(=O)Nc1ccc2c(-c3ccccc3CNc3nc(NCc4cnc(C)s4)nc4c(C(C)C)cnn34)nccc2c1] |
| 204 | 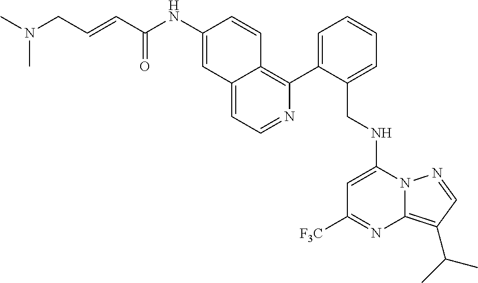 [SMILES: CC(C)c1cnn2c(NCc3ccccc3-c3nccc4cc(NC(=O)/C=C/CN(C)C)ccc34)cc(C(F)(F)F)nc12] |
| 198 | 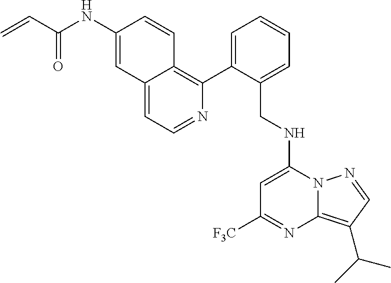 [SMILES: C=CC(=O)Nc1ccc2c(-c3ccccc3CNc3cc(C(F)(F)F)nc4c(C(C)C)cnn34)nccc2c1] |
| 199 | 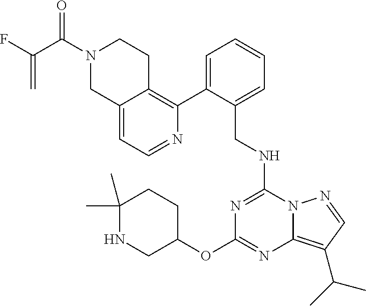 [SMILES: C=C(F)C(=O)N1CCc2c(ccnc2-c2ccccc2CNc2nc(OC3CCC(C)(C)NC3)nc3c(C(C)C)cnn23)C1] |
| 200 | 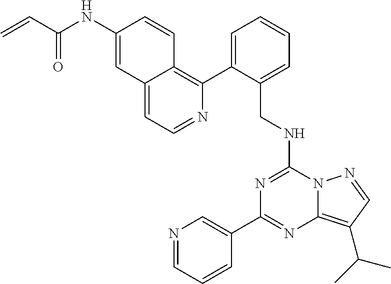 [SMILES: C=CC(=O)Nc1ccc2c(-c3ccccc3CNc3nc(-c4cccnc4)nc4c(C(C)C)cnn34)nccc2c1] |
| 201 | 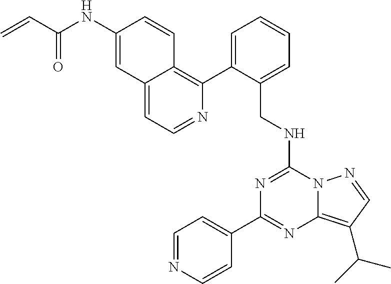 [SMILES: C=CC(=O)Nc1ccc2c(-c3ccccc3CNc3nc(-c4ccncc4)nc4c(C(C)C)cnn34)nccc2c1] |
| 202 | 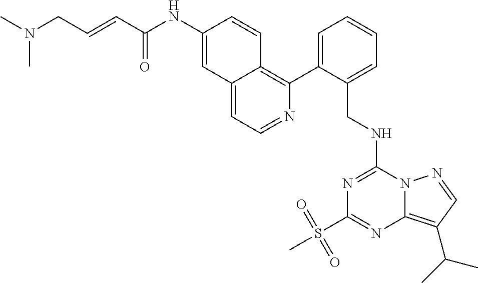 [SMILES: CC(C)c1cnn2c(NCc3ccccc3-c3nccc4cc(NC(=O)/C=C/CN(C)C)ccc34)nc(S(C)(=O)=O)nc12] |
| 203 | 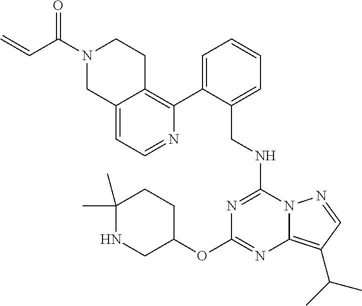 [SMILES: C=CC(=O)N1CCc2c(ccnc2-c2ccccc2CNc2nc(OC3CCC(C)(C)NC3)nc3c(C(C)C)cnn23)C1] |
| 205 | 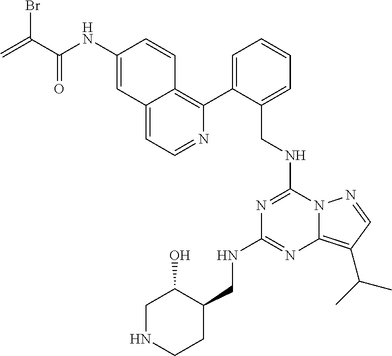 [SMILES: C=C(Br)C(=O)Nc1ccc2c(-c3ccccc3CNc3nc(NC[C@H]4CCNC[C@@H]4O)nc4c(C(C)C)cnn34)nccc2c1] |
| 206 | 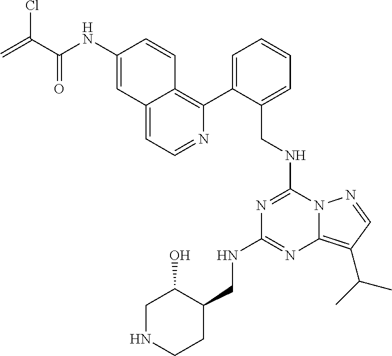 [SMILES: C=C(Cl)C(=O)Nc1ccc2c(-c3ccccc3CNc3nc(NC[C@H]4CCNC[C@@H]4O)nc4c(C(C)C)cnn34)nccc2c1] |
| 207 | 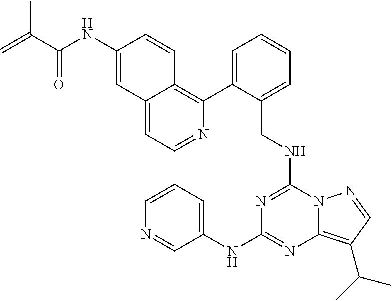 [SMILES: C=C(C)C(=O)Nc1ccc2c(-c3ccccc3CNc3nc(Nc4cccnc4)nc4c(C(C)C)cnn34)nccc2c1] |
| 208 | 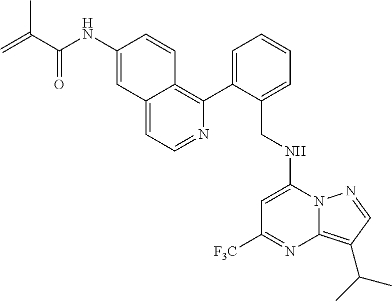 [SMILES: C=C(C)C(=O)Nc1ccc2c(-c3ccccc3CNc3cc(C(F)(F)F)nc4c(C(C)C)cnn34)nccc2c1] |
| 209 | 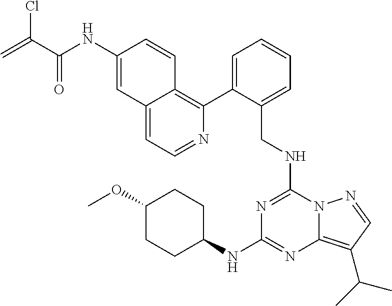 [SMILES: C=C(Cl)C(=O)Nc1ccc2c(-c3ccccc3CNc3nc(N[C@H]4CC[C@H](OC)CC4)nc4c(C(C)C)cnn34)nccc2c1] |
| 210 | 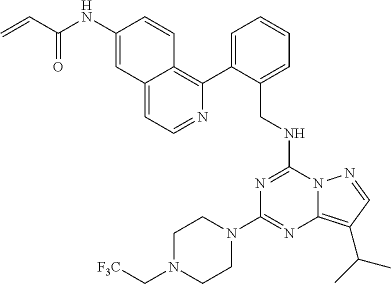 [SMILES: C=CC(=O)Nc1ccc2c(-c3ccccc3CNc3nc(N4CCN(CC(F)(F)F)CC4)nc4c(C(C)C)cnn34)nccc2c1] |
| 211 | 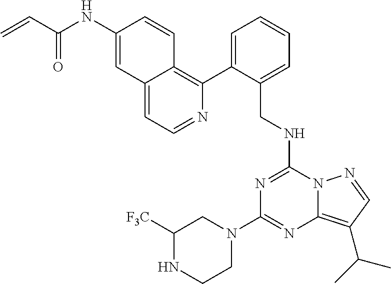 [SMILES: C=CC(=O)Nc1ccc2c(-c3ccccc3CNc3nc(N4CCNC(C(F)(F)F)C4)nc4c(C(C)C)cnn34)nccc2c1] |
| 212 | 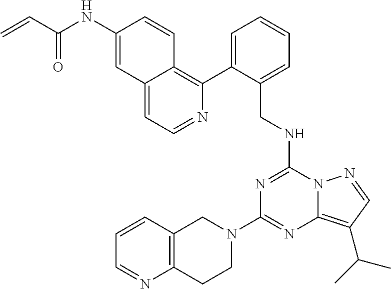 [SMILES: C=CC(=O)Nc1ccc2c(-c3ccccc3CNc3nc(N4CCc5ncccc5C4)nc4c(C(C)C)cnn34)nccc2c1] |
| 213 | 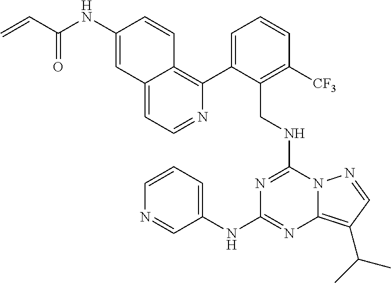 [SMILES: C=CC(=O)Nc1ccc2c(-c3cccc(C(F)(F)F)c3CNc3nc(Nc4cccnc4)nc4c(C(C)C)cnn34)nccc2c1] |
| 214 | 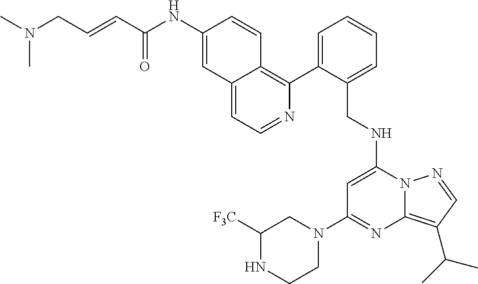 [SMILES: CC(C)c1cnn2c(NCc3ccccc3-c3nccc4cc(NC(=O)/C=C/CN(C)C)ccc34)cc(N3CCNC(C(F)(F)F)C3)nc12] |
| 215 | 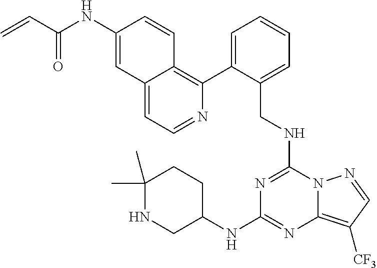 [SMILES: C=CC(=O)Nc1ccc2c(-c3ccccc3CNc3nc(NC4CCC(C)(C)NC4)nc4c(C(F)(F)F)cnn34)nccc2c1] |
| 216 | 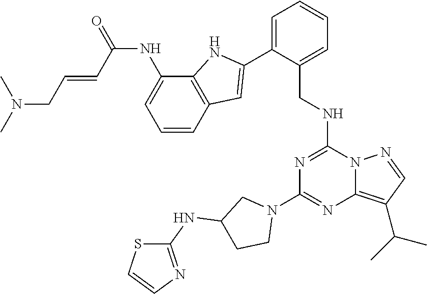 [SMILES: CC(C)c1cnn2c(NCc3ccccc3-c3cc4cccc(NC(=O)/C=C/CN(C)C)c4[nH]3)nc(N3CCC(Nc4nccs4)C3)nc12] |

30. The method according to claim 29, wherein said compound is a compound having a structure selected from structures 44, 64, 95, 134, 147, 164, 174, 175, 177, and 178.

31-34. (canceled)

35. The method according to claim 28, wherein W is c−1, R²⁰ is halogen, and R²² is N(R⁵)₂ or —NR¹⁹R²⁰.

36. The method according to claim 29, wherein said compound is a compound having a structure selected from structures 64, 134, 164, 174, 175, 177 and 178, as defined in claim 29.

37. The method according to claim 29, wherein said compound is a compound having a structure selected from 174, 175 and 177, as defined in claim 29.
